(19) United States
(12) Patent Application Publication
(10) Pub. No.: US 2025/0382585 A1
(43) Pub. Date: Dec. 18, 2025

(54) TUMOR AND TISSUE PRESERVATION REAGENT

(71) Applicants: NIPPON BECTON DICKINSON COMPANY LTD, Tokyo (JP); National Cancer Center Japan, Tokyo (JP)

(72) Inventors: Tamiyo Kobayashi, Tokyo (JP); Hiroyoshi Nishikawa, Tokyo (JP)

(21) Appl. No.: 19/235,338

(22) Filed: Jun. 11, 2025

Related U.S. Application Data
(60) Provisional application No. 63/659,635, filed on Jun. 13, 2024.

(51) Int. Cl. C12N 5/00; C12Q 1/6886

(52) U.S. Cl. CPC C12N 5/525; C12N 5/562; C12Q 1/6886; C12Q 2600/154; C12Q 2600/156; C12Q 2600/158

(57) ABSTRACT

Disclosed herein include methods, compositions, and kits suitable for use in preservation (e.g., cryopreservation) of samples. The composition (e.g., cryopreservation reagent) can comprise: one or more cryoprotective agent(s), one or more culture media component(s), one or more carbohydrate(s), and/or one or more amino acid(s). The composition can be capable of maintaining one or more cellular properties and/or cell viability of a plurality of cells in a sample for a period of time under a storage condition. Also provided are methods of sample preservation and sample analysis. The method can comprise contacting a sample with the cryopreservation reagent, thereby generating a shielded sample. The method can comprise exposing the shielded sample to a freezing storage condition, thereby generating a cryopreserved sample.

### RELATED APPLICATIONS

This application claims the benefit under 35 U.S.C. § 119 (e) of U.S. Provisional Patent Application Ser. No. 63/659,635, filed Jun. 13, 2024, the content of this related application is incorporated herein by reference in its entirety for all purposes.

### BACKGROUND

### Field

The present disclosure relates generally to the field of sample preservation.

### Description of the Related Art

The tumor microenvironment (TME) in tumor tissue is quite complex, with various types of cells residing there, including fibroblasts and immune cells (e.g., granulocytes, lymphocytes, macrophages) in addition to tumor cells. The elements of the TME have been shown to participate directly in all aspects of cancer biology, including initiation, progression and metastatic dissemination. The plasticity and heterogeneity of the cancer immune response has been implicated in disease prognosis. Several specific aspects of tumor immunology have been shown to be associated with differential prognosis in various cancers. These attributes have fueled the development of prognostic assays that show promise in resolving questions relating to the probability of therapeutic response and the overall likelihood of disease recurrence. For example, it is known that there are patients who show an effect (responders) and patients who do not show an effect (non-responders) with respect to immune checkpoint inhibitors. Therefore, in clinical settings tests for predicting responsiveness to an immune checkpoint inhibitor (companion diagnostics) are conducted prior to administration. Despite carrying out companion diagnostics, however, the response rate of immune checkpoint inhibitors is reported to be 20% to 40%. Accordingly, stratifying responders and non-responders is an important task for reducing patient burden and reducing medical costs. An existing problem in the art is that immune cells (including granulocytes) in the TME are more vulnerable because they are activated, and therefore, it is very difficult to retain their viability after freeze-thaw. Commercially available sample preservation products are insufficient to maintain the viability and biomarkers in clinical specimens for flow cytometry, single cell RNAseq, and biobanking. Moreover is very difficult to cryopreserve with high viability cell mixtures of a variety of cell types existing in human tumor tissue, as many commercially available and conventional cell cryopreservation buffers can effectively store single types of cells (e.g. cultured cell strain). There is a need for compositions and methods capable of maintaining the cellular properties of tissue samples outside the body so that responsiveness to drugs (e.g., immune checkpoint inhibitors) can be more accurately predicted than in conventional companion diagnostics. There is a need for compositions and methods capable of maintaining one or more cellular properties and/or cell viability of a plurality of cells in a sample for a period of time under a storage condition (e.g. freezing storage conditions). There is a need for compositions and methods capable of storing various human specimens for long periods of time (e.g., years) for later analysis (e.g., gene and protein analysis).

### SUMMARY

Disclosed herein include cryopreservation reagents. In some embodiments, the cryopreservation reagent comprises: one or more cryoprotective agent(s) (e.g., dimethyl sulfoxide (DMSO) and/or ethylene glycol (EG)). In some embodiments, the cryopreservation reagent comprises: one or more culture media component(s) (e.g., FBS (Fetal Bovine Serum) and/or RPMI-1640). In some embodiments, the cryopreservation reagent comprises: one or more carbohydrate(s) (e.g., trehalose, sucrose, and/or fructose). In some embodiments, the cryopreservation reagent comprises: one or more amino acid(s) (e.g., taurine).

Disclosed herein include cryopreservation reagents. In some embodiments, the cryopreservation reagent comprises: albumin, optionally 0.01-2% (w/v); lactic acid or a salt thereof, optionally 0.1-100 mmol/L; and one or more cryoprotective agent(s), optionally dimethyl sulfoxide (DMSO) and/or ethylene glycol (EG). In some embodiments, the sodium ion concentration of the cryopreservation reagent is about 50-300 mmol/L. The albumin can be bovine serum albumin (BSA). The lactic acid or salt thereof can be sodium lactate. The cryopreservation reagent can comprise about 0.01-10 mmol/L of potassium dihydrogen phosphate. The cryopreservation reagent can comprise about 0.01-50 mmol/L of disodium hydrogen phosphate. The cryopreservation reagent can comprise a polysaccharide, optionally selected from the group comprising trehalose, a fructooligosaccharide, indigestible dextrin, or any combination thereof.

Disclosed herein include suspending buffers (e.g., TTS). In some embodiments, the suspending buffer comprises: one or more carbohydrate(s), optionally trehalose, sucrose, and/or fructose; one or more amino acid(s), optionally taurine; and/or one or more culture media component(s), optionally Fetal Bovine Serum (FBS) and/or RPMI-1640. In some embodiments, the suspending buffer comprises the one or more carbohydrate(s) at a concentration of about 10 mM to about 300 mM (or a number or a range between these values), optionally a first carbohydrate at a concentration of about 50 mM to about 200 mM and a second carbohydrate at a concentration of about 5 mM to about 100 mM, further optionally the first carbohydrate is trehalose and the second carbohydrate is sucrose. In some embodiments, the suspending buffer comprises about 0.1% (w/v) to about 10% (w/v) (or a number or a range between these values) of the one or more amino acid(s), optionally about 1.5% (w/v). In some embodiments, suspending buffer comprises about 0.1% (v/v) to about 80% (v/v) (or a number or a range between these values) of the one or more culture media component(s), optionally about 2% (v/v). In some embodiments, the suspending buffer comprises 100 mM Trehalose, 25 mM Sucrose, 1.5% (w/v) Taurine, 2% (v/v) FBS, and RPMI 1640.

In some embodiments, the cryopreservation reagent is capable of maintaining one or more cellular properties and/or cell viability of a plurality of cells in a sample for a period of time under a storage condition. In some embodiments, a contact of the cryopreservation reagent with a sample comprising a plurality of cells is capable of generating a shielded sample, and wherein one or more cellular properties and/or cell viability of said plurality of cells is maintained for a period of time under a storage condition.

Disclosed herein include cryopreservation reagents comprising one or more cryoprotective agent(s). In some embodiments, the cryopreservation reagent comprises about 6% (v/v) to about 14% (v/v) of the one or more cryoprotective agent(s), such as, for example, about 3% (v/v) to about 7% (v/v) of a first cryoprotective agent (e.g., DMSO) and about 3% (v/v) to about 7% (v/v) of a second cryoprotective agent (e.g., EG). In some embodiments, the one or more cryoprotective agent(s) are selected from the group comprising acetamide, albumin, ammonium acetate, anti-freeze proteins, butanediol, chloroform, choline, cyclohexanediols, cyclohexanediones, cyclohexanetriols, diethylene glycol, dimethyl acetamide, dimethyl formamide, dimethyl sulfoxide, ethanol, ethylene glycol, ethylene glycol monomethyl ether, formamide, glycerol, glyceryl monoacetate, glycoproteins, hydroxyethyl starch, magnesium chloride, magnesium sulfate, methanol, methoxy propanediol, methyl acetamide, methyl formamide, methyl ureas, methyl glycerol, phenol, pluronic polyols, polyethylene glycol, polyvinylpyrrolidone, propanediol, pyridine N-oxide, sodium bromide, sodium chloride, sodium iodide, sodium nitrate, sodium nitrite, sodium sulfate, triethylene glycol, trimethylamine acetate, urea, derivatives thereof, or any combination thereof. In some embodiments, cryopreservation reagents provided herein do not comprise carbohydrate(s), culture media component(s), and/or amino acid(s).

Disclosed herein include cryopreservation reagents comprising one or more carbohydrate(s). In some embodiments, the cryopreservation reagent comprises the one or more carbohydrate(s) at a concentration of about 10 mM to about 300 mM, such as, for example, a first carbohydrate (e.g., trehalose) at a concentration of about 50 mM to about 100 mM and a second carbohydrate (e.g., sucrose) at a concentration of about 20 mM to about 50 mM. In some embodiments, the one or more carbohydrate(s) are selected from the group comprising a sugar, a monosaccharide, a disaccharide, a polyol, an oligosaccharide, a malto-oligosaccharide, a non-malto-oligosaccharide, a polysaccharide, a starch, a non-starch polysaccharide, derivatives thereof, or any combination thereof. In some embodiments, the one or more carbohydrate(s) are selected from the group comprising trehalose, fructose, sucrose, allose, altrose, amylopectin, arabinose, adonitol, cellobiose, cyclodextrin, dextran, deoxyribose, dulcitol, dextrose, erythritol, erythrose, erythrulose, fucose, galactose, glucosamine, glucose, gulose, idose, inositol, invert sugar, isotrehalose, lactose, lyxose, maltodextrin, maltose, mannitol, mannose, mannosamine, melezitose, neotrehalose, palatinose (isomaltulose), psicose, raffinose, ribose, ribulose, rhamnose, sialic acid, sorbitol, sorbose, tagatose, talose, threitol, threose, turanose, xylitol, xylose, xylulose, derivatives thereof, or any combination thereof. In some embodiments, cryopreservation reagents provided herein do not comprise cryoprotective agent(s), culture media component(s), and/or amino acid(s).

In some embodiments, the cryopreservation reagent comprises about 10% (v/v) to about 80% (v/v) of the one or more culture media component(s), such as, for example, about 50% (v/v) of a first culture media component (e.g., FBS) and about 30% (v/v) to about 50% (v/v) of a second culture media component (e.g., RPMI-1640). In some embodiments, the one or more culture media component(s) are selected from the group comprising AIM-V, Alpha-Minimum Essential Medium (α-MEM), Basal Medium Eagle (BME), Brainphys, CTS KnockOut DMEM, CTS OpTimizer T Cell Expansion SFM, CellGro DC Medium, Dulbecco's Modified Eagle Medium (DMEM), Dulbecco's Modified Eagle Medium: Nutrient Mixture F-12 (DMEM/F-12), Dulbecco's Phosphate Buffered Saline (DPBS), Eagle's Basal Medium (EBM), Eagle's Essential Medium (EEM), Earle's Balanced Salt Solution (EBSS), F-10 Nutrient Mixture, F-12K Nutrient Mixture Medium (Kaighn's Modification, F-12K), Fetal Bovine Serum (FBS), Fischer's Medium, GMEM (Glasgow's Minimum Essential Medium), Hanks' Balanced Salt Solution (HBSS), Hanks' Salts Medium (HMEM), Ham's F-10, Ham's F-12, ImmunoCult-XF T Cell Expansion Medium, Iscove's Modified Dulbecco's Medium (IMDM), L-15 (Leibovitz's L-15), MCDB 131, MCDB 153, MDEM, MEM (Minimum Essential Medium), M199 (Medium 199), McCoy's 5A, Neurobasal, Neurobasal A, ObM (Osteoblast Medium), Opti-MEM I Reduced Serum Media, PBS (Phosphate-Buffered Saline), PRIME-XV T Cell Expansion Medium, Roswell Park Memorial Institute Medium (RPMI) 1640 Medium, Sodium Bicarbonate Buffers, StemLine II, StemPro-34, StemSpan-ACF, StemSpan-H3000, StemSpan-SFEM, StemXVivo, TexMACS Medium, X-VIVO 15, X-vivo 20, derivatives thereof, or any combination thereof.

In some embodiments, the one or more amino acid(s) is present at a concentration of about 5 mM to about 2 M (e.g., about 50 mM to about 200 mM). In some embodiments, the one or more amino acid(s) are selected from the group comprising taurine, alanine, arginine, asparagine, aspartic acid, cysteine, glutamic acid, glutamine, glycine, histidine, isoleucine, leucine, lysine, methionine, phenylalanine, proline, selenocysteine, serine, threonine, tyrosine, tryptophan, ornithine, citrulline, aminobenzoic acid, valine, derivatives thereof, or any combination thereof.

In some embodiments, the cryopreservation reagent comprises: about 3% (v/v) to about 7% (v/v) DMSO (e.g., about 5% (v/v)); about 3% (v/v) to about 7% (v/v) EG (e.g., about 5% (v/v)); about 10% (v/v) to about 80% (v/v) FBS (e.g., about 50% (v/v)); about 10% (v/v) to about 70% (v/v) RPMI-1640 (e.g., about 30% (v/v) to about 50% (v/v)); trehalose at a concentration of about 20 mM to about 200 mM (e.g., about 50 mM to about 100 mM); sucrose and/or fructose at a concentration of about 10 mM to about 100 mM (e.g., about 20 mM to about 50 mM); and/or taurine at a concentration of about 50 mM to about 200 mM.

In some embodiments, the plurality of cells comprises a plurality of cell types (e.g., at least about 2, 3, 4, 5, 6, 7, 8, 9, 10, 12, 14, 16, 18, 20, 25, or 50 distinct cell types). In some embodiments, the plurality of cell types are selected from the group comprising a tumor cell, an antigen-presenting cell, a dendritic cell, a fibroblast, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell (e.g., an embryonic stem cell, an induced pluripotent stem cell (iPSC), a hematopoietic stem/progenitor cell (HSPC), or any combination thereof), sustentacular cell, teloglial cell, a zymogenic cell, or any combination thereof. In some embodiments, the plurality of cell types are selected from the group comprising tumor-associated DCs (TADCs), tumor-associated neutrophils (TANs), myeloid-derived suppressor cells (MDSCs), tumor-associated macrophages (TAMs), CD45 low/neg cells, CD4+ memory T-cells, CD4+ naive T-cells, CD4+ T-cells, central memory T (Tcm) cells, effector memory T (Tem) cells, CD4+ Tcm, CD4+ Tem, CD8+ T-cells, CD8+ naive T-cells, CD8+ Tcm, CD8+ Tem, regulatory T cells (Tregs), T helper (Th) 1 cells, Th2 cells, gamma delta T (Tgd) cells, natural killer (NK) cells, natural killer T (NKT) cells, B cells, naive B cells, memory B cells, class-switched memory B-cells, pro B-cells, plasma cells, M1 macrophages, M2 macrophages, CD19+B cells, CD14+ monocytes, CD56+NK cells, CD8+ T cells, Treg cells, CD4+ T cells, granulocytes, dendritic cells, or any combination thereof. In some embodiments, the plurality of cells comprises one or more immune cells having a naive status, activated status, activation recovered status, terminally exhausted status, progenitor exhausted status, central memory status, effector memory status, stem cell memory status, or any combination thereof. In some embodiments, the sample comprises a tumor microenvironment.

In some embodiments, the sample is a biological sample or an environmental sample. In some embodiments, the environmental sample is, or is obtained from, a food sample, a beverage sample, a paper surface, a fabric surface, a metal surface, a wood surface, a plastic surface, a soil sample, a freshwater sample, a wastewater sample, a saline water sample, exposure to atmospheric air or other gas sample, cultures thereof, or any combination thereof. In some embodiments, the biological sample is, or is obtained from, a tissue sample, saliva, blood, plasma, sera, stool, urine, sputum, mucous, lymph, synovial fluid, cerebrospinal fluid, ascites, pleural effusion, seroma, pus, swab of skin or a mucosal membrane surface, cultures thereof, or any combination thereof. In some embodiments, the sample comprises or is derived from a tissue sample (e.g., a tumor tissue sample). In some embodiments, the tissue sample is a biopsy sample, and can be obtained by needle aspiration, fine needle aspiration, core needle biopsy, vacuum assisted biopsy, large core biopsy, incisional biopsy, excisional biopsy, punch biopsy, shave biopsy, or skin biopsy. In some embodiments, the sample is derived from a subject (e.g., a subject having or suspected of having a tumor, a subject having a stage I cancer, a stage II cancer, a stage III cancer, and/or a stage IV cancer). In some embodiments, the tumor is selected from the group comprising biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic (CLL), chronic myeloid (CML), chronic myelomonocytic (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, uterine sarcoma, or any combination thereof.

In some embodiments, the sample has been contacted with a sample preservation composition and/or a cell dispersion composition capable of generating a single cell suspension. In some embodiments, the sample has not been contacted with a sample preservation composition and/or a cell dispersion composition capable of generating a single cell suspension. In some embodiments, the sample is or comprises a single cell suspension. In some embodiments, the sample is or comprises an unprocessed tissue specimen. In some embodiments, the plurality of cells comprises at least about 10 cells, 50 cells, 100 cells, 200 cells, 300 cells, 400 cells, 500 cells, 600 cells, 700 cells, 800 cells, 900 cells, 1000 cells, 1250 cells, 1500 cells, 1750 cells, 2000 cells, 2250 cells, 2500 cells, 2750 cells, 3000 cells, 3250 cells, 3500 cells, 3750 cells, 4000 cells, 4250 cells, 4500 cells, 4750 cells, 5000 cells, 5250 cells, 5500 cells, 5750 cells, 6000 cells, 6250 cells, 6500 cells, 6750 cells, 7000 cells, 7250 cells, 7500 cells, 7750 cells, 8000 cells, 8250 cells, 8500 cells, 8750 cells, 9000 cells, 9250 cells, 9500 cells, 9750 cells, 10000 cells, 50000 cells, or 100000 cells. In some embodiments, the sample is at least about 0.01 mg, 0.05 mg, 0.10 mg, 0.20 mg, 0.50 mg, 1 mg, 2 mg, 5 mg, 10 mg, 15 mg, 20 mg, 25 mg, 30 mg, 40 mg, 50 mg, 75 mg, 100 mg, 150 mg, 200 mg, 300 mg, 400 mg, 500 mg, 600 mg, 700 mg, 800 mg, 900 mg, 1 g, or 10 g.

In some embodiments, the one or more cellular properties comprise at least about 2, 3, 4, 5, 6, 7, 8, 9, 10, 12, 14, 16, 18, 20, 25, 50, 75, 100, 250, or 1000 distinct cellular properties. In some embodiments, one or more cellular properties comprise physical properties, morphological properties, intracellular properties, biochemical properties, dynamic behavioral properties, or any combination thereof. In some embodiments, the physical properties comprise cell size, volume, conductivity, low and high angle scatter, density, or any combination thereof. In some embodiments, the morphological properties comprise one or more of: cell shape, area, size, and lobularity; nucleus shape area, size, and lobularity; mitochondria shape, area, size, and lobularity; and ratio of nuclear volume to cell volume. In some embodiments, the intracellular properties comprise nucleus centroid/cell centroid distance, nucleus lobe centroid distance, distribution of proteins with the cells, distribution of organelles within the cells, or any combination thereof. In some embodiments, the biochemical properties comprise expression level of cellular proteins, cell surface proteins, cytoplasmic proteins, nuclear proteins, cellular nucleic acids, cell surface nucleic acids, cytoplasmic nucleic acids, nuclear nucleic acids, cellular carbohydrates, cell surface carbohydrates, cytoplasmic carbohydrates, nuclear carbohydrates, or any combination thereof. In some embodiments, the dynamic behavioral properties comprises cellular activation, cellular inhibition, protein secretion, microvesicle secretion, exosome secretion, microparticle secretion, metabolite secretion, small molecule secretion, proton secretion, protein expression, or any combination thereof.

In some embodiments, one or more cellular properties comprise one or more cellular features indicating cell proliferation, stress pathway activation, organelle function, cell cycle state, apoptosis, DNA damage, metabolism, signal transduction, cell differentiation, or any combination thereof. In some embodiments, the cellular features indicating cell proliferation comprise nuclear count, cell count, total cell mass, total DNA, the phosphorylation state of cell cycle regulatory proteins, the post-translational modification state of any protein involved in cell growth or division, or any combination thereof. In some embodiments, the cellular features indicating stress pathway activation comprise transcription factor activation of NF-KB, API, ATF2, MSK1, CREB, or NFAT, and kinase activation of p38, JNK, ERK, RSK90, MEK, or any combination thereof. In some embodiments, the cellular features indicating organelle function comprise cytoskeletal organization, mitochondrial mass or membrane potential, peroxisome mass, golgi organization, plasma membrane permeability, or any combination thereof. In some embodiments, the cellular features indicating cell cycle state comprise DNA content, Histone H3 phosphorylation state, Rb phosphorylation state, cyclin B1 (CDKI) biosynthesis, cyclin DI (CDK4, 6) biosynthesis, cyclin E (CDK2) biosynthesis, or any combination thereof. In some embodiments, the cellular features indicating apoptosis comprise nuclear size and shape, DNA content and degradation, caspase activation, phosphatidyl-expression, Bax translocation, or any combination thereof. In some embodiments, the cellular features indicating DNA damage comprise repair protein (APE) expression, tumor suppressor (e.g., p53, Rb) expression, oxidative activity (e.g., 8-oxoguanine), transcription activity (e.g., Oct1), or any combination thereof. In some embodiments, the cellular features indicating metabolism comprise cAMP concentration, P-glycoprotein activity or CYP450 induction/inhibition, the concentration of an added substance, or any combination thereof. In some embodiments, the cellular features indicating signal transduction comprise Ca²⁺ ion concentration, pH, expression of a protein, activation of a protein, modification of a protein, translocation of a protein, interaction between proteins known to be associated with a specific pathway, or any combination thereof. In some embodiments, the cellular features indicating cell differentiation comprise expression of a tissue specific protein, exhibiting a tissue specific morphology, or any combination thereof. In some embodiments, the one or more cellular properties comprise one or more genomic properties, one or more expression properties, and/or one or more variant properties. In some embodiments, the one or more variant properties comprise a single nucleotide polymorphism (SNP), an insertion or deletion (indel), a copy number variant (CNV), a fusion, a splice variant, an isoform variant, a transversion, a translocation, a frame shift, a duplication, a repeat variant, or any combination thereof, at one or more loci of a plurality of loci; In some embodiments, the one or more genomic properties comprise chromatin accessibility, hypomethylation and/or hypermethylation at one or more loci of a plurality of loci. In some embodiments, the one or more expression properties comprise underexpression of one or more mRNAs of interest, underexpression of one or more proteins of interest, overexpression of one or more mRNAs of interest, and/or overexpression of one or more proteins of interest. In some embodiments, the one or more mRNAs of interest and/or one or more proteins of interest are derived from one or more loci of a plurality of loci.

In some embodiments, the storage condition comprises a temperature of about 4° C., 2° C., 0° C., −2° C., −4° C., −6° C., −8° C., −10° C., −12° C., −14° C., −16° C., −18° C., −20° C., −22° C., −24° C., −26° C., −28° C., −30° C., −32° C., −34° C., −36° C., −38° C., −40° C., −42° C., −44° C., −46° C., −48° C., −50° C., −52° C., −54° C., −56° C., −58° C., −60° C., −62° C., −64° C., −66° C., −68° C., −70° C., −72° C., −74° C., −76° C., −78° C., −80° C., −82° C., −84° C., −86° C., −88° C., −90° C., −92° C., −94° C., −96° C., −98° C., −100° C., −102° C., −104° C., −106° C., −108° C., −110° C., −112° C., −114° C., −116° C., −118° C., −120° C., −122° C., −124° C., −126° C., −128° C., −130° C., −132° C., −134° C., −136° C., −138° C., −140° C., −142° C., −144° C., −146° C., −148° C., −150° C., −152° C., −154° C., −156° C., −158° C., −160° C., −162° C., −164° C., −166° C., −168° C., −170° C., −172° C., −174° C., −176° C., −178° C., −180° C., −182° C., −184° C., −186° C., −188° C., −190° C., −192° C., −194° C., −196° C., or any combination thereof. In some embodiments, the storage condition comprises transport of a cryopreserved sample, thermal stress, one or more freeze-thaw cycles, agitation, pressure changes, light irradiation, or any combination thereof. In some embodiments, the period of time is at least about 1 day, 3 days, 5 days, 1 week, 2 weeks, 3 weeks, 1 month, 2 months, 3 months, 4 months, 5 months, 6 months, 7 months, 8 months, 9 months, 10 months, 11 months, 12 months, 14 months, 16 months, 18 months, 20 months, 22 months, 24 months, 26 months, 28 months, 30 months, 32 months, 34 months, 36 months, 38 months, 40 months, 42 months, 44 months, 46 months, or 48 months.

In some embodiments, at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the plurality of cells in the sample remain viable post-storage condition. In some embodiments, at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the plurality of cells in the sample maintain one or more cellular properties post-storage condition. In some embodiments, the value(s) of the one or more cellular properties post-storage condition are less than about 1.01-fold, 1.02-fold, 1.04-fold, 1.06-fold, 1.08-fold, 1.1-fold, 1.15-fold, 1.2-fold, 1.25-fold, 1.3-fold, 1.35-fold, 1.4-fold, 1.45-fold, or 1.5-fold, different than said value(s) prior to the storage condition. In some embodiments, the expression of one or more stress-associated genes is increased less than about 1.01-fold, 1.02-fold, 1.04-fold, 1.06-fold, 1.08-fold, 1.1-fold, 1.15-fold, 1.2-fold, 1.25-fold, 1.3-fold, 1.35-fold, 1.4-fold, 1.45-fold, or 1.5-fold post-storage condition. In some embodiments, the one or more stress-associated genes are selected from the group consisting of ADM, AKR1B1, AQP1, AQP2, AQP4, ARNT, ATF4, ATF6, ATF6B, ATG12, ATG5, ATG7, ATM, ATR, BBC3, BECN1, BID, BNIP3L, CA9, CALR, CASP1, CCL2, CD40LG, CDKN1A, CFTR, CHEK1, CHEK2, CRP, DDB2, DDIT3, DNAJC3, EDN1, EPO, FAS, FTH1, GADD45A, GADD45G, GCLC, GCLM, GRB2, GSR, GSTP1, HMOX1, HSP90AA1, HSP90B1, HSPA4, HSPA4L, HSPA5, HUS1, IFNG, IL1A, IL1B, IL6, CXCL8, LDHA, MCL1, MMP9, MRE11A, NBN, NFAT5, NQ01, PARP1, PRDX1, PVR, RAD17, RAD51, RAD9A, RIPK1, SERPINE1, SLC2A1, SLC5A3, SQSTM1, TLR4, TNF, TNFRSF10A, TNFRSF10B, TNFRSF1A, TP53, TXN, TXNL4B, TXNRD1, ULK1, VEGFA, XPC, or any combination thereof. In some embodiments, the cryopreservation reagent does not comprise: albumin (e.g., bovine serum albumin (BSA)); lactic acid or a salt thereof (e.g., sodium lactate); potassium dihydrogen phosphate; and/or disodium hydrogen phosphate.

Disclosed herein include shielded samples. In some embodiments, the shielded sample comprises: a sample contacted with a cryopreservation reagent disclosed herein. Disclosed herein include cryopreserved samples. In some embodiments, the cryopreserved sample comprises: a sample contacted with a cryopreservation reagent disclosed herein and exposed to a freezing storage condition. Disclosed herein include methods of sample cryopreservation. In some embodiments, the method comprises: providing a cryopreservation reagent disclosed herein. In some embodiments, the method comprises: contacting a sample with the cryopreservation reagent, thereby generating a shielded sample. In some embodiments, the method comprises: exposing the shielded sample to a freezing storage condition, thereby generating a cryopreserved sample. In some embodiments, the method comprises, prior to the contacting step: contacting the sample with a sample preservation composition; and/or contacting the sample with a cell dispersion composition capable of generating a single cell suspension. Disclosed herein include methods of sample analysis. In some embodiments, the method comprises: providing a sample; and providing a cryopreservation reagent disclosed herein. In some embodiments, the method comprises: contacting the sample with the cryopreservation reagent, thereby generating a shielded sample. In some embodiments, the method comprises: exposing the shielded sample to a freezing storage condition, thereby generating a cryopreserved sample. In some embodiments, the method comprises: thawing the cryopreserved sample. In some embodiments, the method comprises: conducting one or more measurements of one or more cellular properties. In some embodiments, the method comprises, prior to and/or after the contacting step: contacting the sample with a sample preservation composition; and/or contacting the sample with a cell dispersion composition capable of generating a single cell suspension. In some embodiments, the method comprises contacting the thawed sample with a suspending buffer provided herein (e.g., adding the thawed sample into the suspending buffer). The suspending buffer can be about 32° C.-42° C. (e.g., 37° C.) when contacted.

In some embodiments, the method comprises contacting the sample with the cell dispersion composition within about 24 hr to about 48 hr of contacting the sample with a sample preservation composition. In some embodiments, the method does not comprise fixation and/or cancelling crosslinking by enzyme and/or heat. In some embodiments, contacting comprises immersing. In some embodiments, the freezing storage condition comprises a temperature of about 0° C., −2° C., −4° C., −6° C., −8° C., −10° C., −12° C., −14° C., −16° C., −18° C., −20° C., −22° C., −24° C., −26° C., −28° C., −30° C., −32° C., −34° C., −36° C., −38° C., −40° C., −42° C., −44° C., −46° C., −48° C., −50° C., −52° C., −54° C., −56° C., −58° C., −60° C., −62° C., −64° C., −66° C., −68° C., −70° C., −72° C., −74° C., −76° C., −78° C., −80° C., −82° C., −84° C., −86° C., −88° C., −90° C., −92° C., −94° C., −96° C., −98° C., −100° C., −102° C., −104° C., −106° C., −108° C., −110° C., −112° C., −114° C., −116° C., −118° C., −120° C., −122° C., −124° C., −126° C., −128° C., −130° C., −132° C., −134° C., −136° C., −138° C., −140° C., −142° C., −144° C., −146° C., −148° C., −150° C., −152° C., −154° C., −156° C., −158° C., −160° C., −162° C., −164° C., −166° C., −168° C., −170° C., −172° C., −174° C., −176° C., −178° C., −180° C., −182° C., −184° C., −186° C., −188° C., −190° C., −192° C., −194° C., −196° C., or any combination thereof. In some embodiments, the freezing storage condition comprises liquid nitrogen. In some embodiments, the cell dispersion composition comprises one or more enzymes selected from the group comprising collagenase, elastase, hyaluronidase, dispase, glycosidase, deoxyribonuclease, trypsin, or any combination thereof. In some embodiments, the sample preservation composition comprises: albumin (optionally 0.01-2% (w/v)); and lactic acid or a salt thereof (optionally 0.1-100 mmol/L). In some embodiments, the sodium ion concentration of the sample preservation composition is about 50-300 mmol/L; the albumin is bovine serum albumin (BSA); the lactic acid or salt thereof is sodium lactate; the sample preservation composition comprises about 0.01-10 mmol/L of potassium dihydrogen phosphate; the sample preservation composition comprises about 0.01-50 mmol/L of disodium hydrogen phosphate the sample preservation composition comprises a polysaccharide (e.g., trehalose, a fructooligosaccharide, indigestible dextrin, or any combination thereof).

In some embodiments, after thawing the cryopreserved sample, at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the plurality of cells are viable. In some embodiments, the value(s) of the one or more cellular properties after thawing the cryopreserved sample are less than about 1.01-fold, 1.02-fold, 1.04-fold, 1.06-fold, 1.08-fold, 1.1-fold, 1.15-fold, 1.2-fold, 1.25-fold, 1.3-fold, 1.35-fold, 1.4-fold, 1.45-fold, or 1.5-fold, different than said value(s) prior to exposing the shielded sample to a freezing storage condition.

In some embodiments, conducting one or more measurements comprises conducting one or more measurements on a plurality of samples in parallel. In some embodiments, conducting one or more measurements comprises conducting a nucleic acid sequencing assay, a next generation nucleic acid sequencing (NGS) assay, a PCR assay, a quantitative PCR (qPCR) assay, a reverse transcription PCR (RT-PCR) assay, a miRNA assay, a microarray assay, a Northern blot assay, a Southern blot assay, a luciferase assay, a fluorescence immunoassay, a radio immunoassay, an enzyme-linked immunosorbent assay (ELISA), a flow cytometry assay, a mass spectrometry (MS) assay, a Selected Reaction Monitoring (SRM-MS) assay, a Sequential Windowed data independent Acquisition of the Total High resolution Mass Spectroscopy (SWATH-MS) assay, a Western blot assay, a genome wide methylation assay, a targeted methylation assay, a bisulfite methylation sequencing assay, a restriction enzyme methylation sequencing assay, a high performance liquid chromatography (HPLC) assay, an ultrahigh performance liquid chromatography (UHPLC) assay, a mass spectrometry (MS) assay, an ultrahigh performance liquid chromatography/tandem mass spectrometry (UHPLC/MS/MS2), a gas chromatography/mass spectrometry (GC/MS) assay, a lipidomics assay, a cell aging assay, an endocrine assay, a neuroendocrine assay, a cytokine assay, an immune cell assay, or any combination thereof. In some embodiments, conducting one or more measurements comprises measuring emissions of a detectable moiety with a flow cytometer (e.g., a conventional flow cytometer, a spectral flow cytometer, a hyperspectral flow cytometer, an imaging flow cytometer, or any combination thereof). In some embodiments, conducting one or more measurements comprises imaging of single cell(s). In some embodiments, imaging comprises microscopy, confocal microscopy, time-lapse imaging microscopy, fluorescence microscopy, multi-photon microscopy, quantitative phase microscopy, surface enhanced Raman spectroscopy, videography, manual visual analysis, automated visual analysis, or any combination thereof.

In some embodiments, the plurality of cells comprises a plurality of cellular component targets, wherein conducting one or more measurements comprises contacting a plurality of cellular component-binding reagents the plurality of cells. In some embodiments, each of the plurality of cellular component-binding reagents comprises a cellular component-binding reagent specific oligonucleotide comprising a unique identifier sequence for the cellular component-binding reagent, and wherein the cellular component-binding reagent is capable of specifically binding to at least one of the plurality of cellular component targets. In some embodiments, each of the plurality of cellular component-binding reagents comprises a detectable moiety, or a precursor thereof, wherein the cellular component-binding reagent is capable of specifically binding to at least one of the plurality of cellular component targets, wherein cellular component-binding reagents capable of binding the same cellular component target comprise the same detectable moiety, or a precursor thereof, and wherein cellular component-binding reagents capable of binding different cellular component targets comprise different detectable moieties, or precursors thereof. In some embodiments, the cellular component-binding reagent comprise an antibody or fragment thereof. In some embodiments, the plurality of cellular component targets are selected from the group comprising CD11b, CD11c, CD33, CD14, CD185 (CXCR5), CD19, CD25, CD27, CD278, CD279, CD3, CD163, CD357 (GITR), CD366 (Tim3), CD4, CD45RA, CD56, CD62L, CD197 (CCR7), CD186 (CXCR6), CD127, CD134, CD28, CD272, CD8, HLA-DR/DP/DQ, CD16, CD183, CD196 (CCR6), CD137, CD161, IgM, CD152, and IgD. In some embodiments, conducting one or more measurements comprises flow cytometrically sorting the plurality of cells, such as fluorescence-activated cell sorting (FACS), and said sorting can based on intracellular staining and/or surface marker staining (e.g., staining with cellular component-binding reagent(s)).

In some embodiments, the plurality of cells comprises a plurality of nucleic acid target molecules (e.g., ribonucleic acids (RNAs), messenger RNAs (mRNAs), microRNAs, small interfering RNAs (siRNAs), RNA degradation products, RNAs each comprising a poly(A) tail, and any combination thereof). In some embodiments, conducting one or more measurements comprises partitioning single cells of the plurality of cells to a plurality of partitions (e.g., a plurality of droplets or microwells of a microwell array). In some embodiments, conducting one or more measurements comprises conducting one or more sequencing assays to generate a plurality of sequence reads. In some embodiments, the sequencing assay(s) comprise single cell (sc) sequencing assay(s) and/or long read RNA-seq. In some embodiments, conducting one or more measurements comprises generating values for one or more cellular properties derived from the sequence reads, wherein the one or more cellular properties comprise one or more genomic properties, one or more expression properties, and/or one or more variant properties. In some embodiments, the one or more genomic properties are derived from one or more sequencing assays comprising a bisulfite sequencing assay, single cell bisulfite sequencing assay, assay for Transposase-Accessible Chromatin using sequencing (ATAC-seq), single cell (sc) ATAC-seq, or any combination thereof. In some embodiments, the one or more expression properties are derived from one or more sequencing assays comprising sequence-mediated protein profiling, single cell sequence-mediated protein profiling, RNA-sequencing (RNA-seq), single cell (sc) RNA-seq, or any combination thereof. In some embodiments, the one or more variant properties are derived from sequencing assays comprising barcoded sequencing, random sequencing, whole genome sequencing, targeted sequencing, next generation sequencing, or any combination thereof. In some embodiments, the one or more variant properties comprise a single nucleotide polymorphism (SNP), an insertion or deletion (indel), a copy number variant (CNV), a fusion, a splice variant, an isoform variant, a transversion, a translocation, a frame shift, a duplication, a repeat variant, or any combination thereof, at one or more loci of a plurality of loci. In some embodiments, the one or more genomic properties comprise chromatin accessibility, hypomethylation and/or hypermethylation at one or more loci of a plurality of loci. In some embodiments, the one or more expression properties comprise underexpression of one or more mRNAs of interest, underexpression of one or more proteins of interest, overexpression of one or more mRNAs of interest, and/or overexpression of one or more proteins of interest. In some embodiments, the one or more mRNAs of interest and/or one or more proteins of interest are derived from one or more loci of a plurality of loci.

In some embodiments, the plurality of loci is selected from a predetermined set of loci that includes less than all loci in the genome of the subject. In some embodiments, the predetermined set of loci comprises at least 10 loci. In some embodiments, the predetermined set of loci comprises from 100 to 100,000 loci, from 100 to 50,000 loci, from 100 to 25,000 loci, from 100 to 10,000 loci, from 100 to 5000 loci, from 100 to 2000 loci, from 100 to 1000 loci, from 500 to 100,000 loci, from 500 to 50,000 loci, from 500 to 25,000 loci, from 500 to 10,000 loci, from 500 to 5000 loci, from 500 to 2000 loci, from 500 to 1000 loci, from 1000 to 100,000 loci, from 1000 to 50,000 loci, from 1000 to 25,000 loci, from 1000 to 10,000 loci, from 1000 to 5000 loci, or from 1000 to 2000 loci. In some embodiments, the predetermined set of loci are known to be associated with cancer (e.g., the predetermined set of loci comprise tumor suppressor genes and/or oncogenes). In some embodiments, the predetermined set of loci comprise a cancer-related gene selected from the group consisting of: AKT1, ALK, APC, AR, ARAF, ARID 1 A, ARID2, ATM, B2M, BCL2, BCOR, BRAF, BRCA1, BRCA2, CARD11, CBFB, CCND1, CDH1, CDK4, CDKN2A, CIC, CREBBP, CTCF, CTNNB1, DICER 1, DIS3, DNMT3A, EGFR, EIF1AX, EP300, ERBB2, ERBB3, ERCC2, ESRI, EZH2, FBXW7, FGFR1, FGFR2, FGFR3, FGFR4, FLT3, FOXA1, FOXL2, FOXO1, FUBP1, GAT A3, GNA11, GNAQ, GNAS, H3F3A, HIST1H3B, HRAS, IDH1, IDH2, IKZF1, INPPL1, JAK1, KDM6A, KEAP1, KIT, KNSTRN, KRAS, MAP2K1, MAPK1, MAX, MED 12, MET, MLH1, MSH2, MSH3, MSH6, MTOR, MYC, MYCN, MYD88, MYODI, NF1, NFE2L2, NOTCH1, NRAS, NTRK1, NTRK2, NTRK3, NUP93, PAK7, PDGFRA, PIK3CA, PIK3CB, PIK3R1, PIK3R2, PMS2, POLE, PPP2R1A, PPP6C, PRKCI, PTCHI, PTEN, PTPN11, RAC1, RAFI, RBI, RET, RHOA, RIT1, ROS1, RRAS2, RXRA, SETD2, SF3B1, SMAD3, SMAD4, SMARCA4, SMARCB1, SOS1, SPOP, STAT3, STK11, STK19, TCF7L2, TERT, TGFBR1, TGFBR2, TP53, TP63, TSC1, TSC2, U2AF1, VHL, and XPO1.

In some embodiments, generating values for one or more variant properties derived from the sequence reads comprises aligning at least a portion of said sequence reads to the genome of a reference. In some embodiments, generating values for one or more expression properties derived from the sequence reads comprises a comparison to the mRNA expression levels of interest and/or protein expression levels of interest a reference. In some embodiments, generating values for one or more genomic properties derived from the sequence reads comprises a comparison to the methylation status and/or the chromatin accessibility at the one or more loci of a plurality of loci of a reference. In some embodiments, the reference comprises one or more patients having the same stage of cancer, the same type of cancer, or both, that the subject is suspect of having. In some embodiments, the reference comprises one or more unaffected individuals. In some embodiments, the reference comprises a biological sample obtained from the subject at an earlier time point. In some embodiments, the reference comprises a subject having cancer, a subject not having cancer, a subject having a stage I cancer, a subject having a stage II cancer, a subject having a stage III cancer, a subject having a stage IV cancer, or any combination thereof. In some embodiments, conducting the one or more sequencing assays comprise: stochastically barcoding the nucleic acid target molecules using a plurality of stochastic barcodes to generate a plurality of stochastically barcoded target nucleic acid molecules, wherein each of the plurality of stochastic barcodes comprises a cell label and a molecular label, wherein molecular labels of at least two stochastic barcodes of the plurality of stochastic barcodes comprise different molecular label sequences, wherein the stochastic barcodes associated with the same cell comprise the same cellular label, and wherein the cellular labels associated with different cells comprise different cellular labels.

In some embodiments, the method comprises: predicting a response or resistance to and/or a benefit from a therapy in a subject suffering from or being at risk of developing a neoplastic disease based on the measurement of the one or more cellular properties; and/or predicting the outcome of a therapy in a subject suffering from or being at risk of developing a neoplastic disease based on the measurement of the one or more cellular properties. In some embodiments, the response, resistance, benefit and/or outcome is the pathological complete response (pCR), loco-regional recurrence free interval (LRRFI), loco-regional invasive recurrence free interval (LRIRFI), distant-disease-free survival (DDFS), invasive disease-free survival (IDFS), event free survival (EFS) and/or overall survival (OS). In some embodiments, the therapy is an immunotherapy. In some embodiments, the immunotherapy comprises a checkpoint inhibitor, a chimeric antigen receptor T-cell therapy, an oncolytic vaccine, a cytokine agonist or a cytokine antagonist, or a combination thereof. In some embodiments, the immunotherapy comprises a PD-1 inhibitor, PD-L1 inhibitor, PD-L2 inhibitor, GITR agonist, 0X40 agonist, TIM3 agonist, LAG3 agonist, KIR agonist, CD28 agonist, CD137 agonist, CD27 agonist, CD40 agonist, CD70 agonist, CD276 agonist, ICOS agonist, HVEM agonist, NKG2D agonist, NKG2A agonist, MICA agonist, 2B4 agonist, 41BB agonist, CTLA4 antagonist, PD-1 axis antagonist, TIM3 antagonist, BTLA antagonist, VISTA antagonist, LAG3 antagonist, B7H4 antagonist, CD96 antagonist, TIGIT antagonist, CD226 antagonist or a combination thereof. In some embodiments, the cytokine agonist or cytokine antagonist is an agonist or antagonist of interferon, IL-2, GMCSF, IL-17E, IL-6, IL-la, IL-12, TFGB2, IL-15, IL-3, IL-13, IL-2R, IL-21, IL-4R, IL-7, M-CSF, MIF, myostatin, 11-10, 11-24, CEA, IL-11, IL-9, IL-15, IL-2Ra, TNF or a combination thereof.

### BRIEF DESCRIPTION OF THE DRAWINGS

FIG. 1 depicts a non-limiting exemplary workflow of sample cryopreservation and analysis.

FIG. 2 depicts a non-limiting exemplary workflow of sample cryopreservation and analysis.

FIGS. 3A-3G depict fluorescence-activated cell sorting (FACS) data related to Specimen No. 1 viability (using FVS780) at Day 0 (FIG. 3A) and at Cryopreservation Day 13 using conventional buffer (10% DMSO/FBS; FIG. 3B) and using commercially available products CELLBANKER® 1 (FIG. 3C), CELLBANKER® 1 plus (FIG. 3D), STEM-CellBanker® GMP grade (FIG. 3E), STEM-CellBanker® DMSO-Free GMP grade (FIG. 3F), as well as a quantification of said FACS data (FIG. 3G).

FIG. 4 depicts FACS data related to viability, CD45/SSC, FSC/SSC, CD3/CD45, and CD4/CD8 properties for Specimen No. 2 at Cryopreservation Day 13 using cryopreservation buffers CELLBANKER® 1, 10% DMSO/FBS, and 200T.

FIGS. 5A-5D depict FACS data related to viability, CD45/SSC, FSC/SSC properties for Specimen No. 3 at Day 0 and at Cryopreservation Day 14 using cryopreservation buffers CELLBANKER® 1, 100T, 10% EG100T, and CELLBANKER® 1 (FIG. 5A) and cell number quantifications derived therefrom (FIGS. 5B-5D) for CD45lo neg cells (FIG. 5B), granulocytes (FIG. 5C), and lymphocytes (lym) (FIG. 5D).

FIGS. 6A-6E depict FACS data related to viability, CD45/SSC, CD45/SSC, FSC/SSC, CD3/CD45, CD4/CD8, and CD25 properties for Specimen No. 4 at Day 0 and at Cryopreservation Day 11 using cryopreservation buffers CELLBANKER® 1, base (no sugars), 50T, and 100T25F (FIG. 6A) and cell number quantifications derived therefrom (FIGS. 6B-6E) for CD45neg (FIG. 6B), granulocytes (FIG. 6C), lymphocytes (Lym) (FIG. 6D), and CD25 (FIG. 6E) cells.

FIGS. 7A-7F depict FACS data related to viability, CD45/SSC, FSC/SSC, CD3/CD45, CD4/CD8, and CD25 properties for Specimen No. 5 at Day 0 and at Cryopreservation Day 13 using cryopreservation buffers CELLBANKER® 1, base (no sugars), 100T25F, and 40T10F (FIG. 7A) and cell number quantifications derived therefrom (FIGS. 7B-7F) for CD45neg (FIG. 7B), granulocytes (FIG. 7C), lymphocytes (Lym) (FIG. 7D), CD45CD3 (FIG. 7E), and CD25 (FIG. 7F) cells.

FIGS. 8A-8F depict FACS data related to viability, CD45/SSC, FSC/SSC, CD3/CD45, CD4/CD8, and CD25 properties for Specimen No. 6 at Day 0 and at Cryopreservation Day 12 using cryopreservation buffers CELLBANKER® 1, 100T, 75T25F, 75T25S, 50T50F, 50T50S, 50F50S (FIG. 8A) and cell number quantifications derived therefrom (FIGS. 8B-8F) for CD45lo neg (FIG. 8B), granulocytes (FIG. 8C), lymphocytes (Lym) (FIG. 8D), CD45CD3 (FIG. 8E), and CD25 (FIG. 8F) cells.

FIGS. 9A-9D depict cell number data for CD45lo neg (FIG. 9A), granulocytes (FIG. 9B), lymphocytes (Lym) (FIG. 9C), CD45CD3 (FIG. 9D) cells for Specimen No. 7 at Day 0 and at Cryopreservation Day 14 using cryopreservation buffers 100T-RPMI, 100T-DMEM, 100T25F-RPMI, 100T25F-DMEM, 100T25S-RPMI, 100T25S-DMEM, 50T50S-RPMI, and 50T50S-DMEM.

FIGS. 10A-10G depict FACS data related to viability, CD45/SSC, FSC/SSC, CD3/CD45, CD4/CD8, and CD25 properties for Specimen No. 8 at Day 0 and at Cryopreservation Day 10 using cryopreservation buffers CELLBANKER® 1, 100T25S, 100T, and 100T+ Tau (FIG. 10A) and cell number quantifications derived therefrom (FIGS. 10B-10G) for CD45lo neg (FIG. 10B), granulocytes (FIG. 10C), CD45CD3 (FIG. 10D), CD4 (FIG. 10E), CD25 (FIG. 10F), and CD8 (FIG. 10G) cells.

FIGS. 11A-11H depict FACS data related to viability, CD45/SSC, FSC/SSC, CD3/CD45, CD4/CD8, and CD25 properties for Specimen No. 9 at Day 0 and at Cryopreservation Day 10 using cryopreservation buffers 10% DMSO/FBS, 100T25F, and 100T25S (FIG. 11A) and cell number quantifications derived therefrom (FIGS. 11B-11H) for CD45lo neg (FIG. 11B), granulocytes (Gra) (FIG. 11C), lymphocytes (Lym) (FIG. 11D), CD45CD3 (FIG. 11E), CD4 (FIG. 11F), CD25 (FIG. 11G), and CD8 (FIG. 11H) cells.

FIGS. 12A-12H depict FACS data related to viability, CD45/SSC, FSC/SSC, CD3/CD45, CD4/CD8, and CD25 properties for Specimen No. 10 at Day 0 and at Cryopreservation Day 10 using cryopreservation buffers 10% DMSO/FBS, 100T25S, 100T25S+1% Tau, and 100T25S+2% Tau (FIG. 12A) and cell number quantifications derived therefrom (FIGS. 12B-12H) for CD45 low neg (FIG. 12B), granulocytes (Gra) (FIG. 12C), lymphocytes (Lym) (FIG. 12D), CD45CD3 (FIG. 12E), CD4 (FIG. 12F), CD25 (FIG. 12G), and CD8 (FIG. 12H) cells.

FIG. 13 depicts FACS data related to viability, lymphocyte (Lym), CD3/CD45, CD4/CD8, CD25+, CD45+, CD45+CD66b+, and CD45+CD14+ properties for Specimen Tissue-9 (tissue chunk) frozen at −80° C. for 7 days either using cryopreservation buffers 10% DMSO/TTPR, 10% AF/TTPR, and TTCR, or snap-frozen. 10% AF/TTPR appeared to perform better than 10% DMSO/TTPR.

FIGS. 14A-14I depict FACS data related to viability, lymphocyte (Lym), CD3/CD45, CD4/CD8, CD4+CD25+, CD45, CD45+CD66b+, and CD45+CD14+ properties for Specimen Tissue-10 (cell suspension, TTDR-dissociated) at Day 0 and after −80° C. for 16 days using cryopreservation buffers 5% DMSO+95% TTPR, 10% AF+90% TTPR, and TTCR (FIG. 14A) and cell number quantifications derived therefrom (FIGS. 14B-14I) for live (FIG. 14B), CD45+ (FIG. 14C), CD45− (FIG. 14D), lymphocyte (Lym) (FIG. 14E), CD45+CD3+ (FIG. 14F), CD4+ (FIG. 14G), CD25+ (FIG. 14H), and CD8+ (FIG. 14I) cells.

FIGS. 15A-15H depict FACS data related to viability, lymphocyte (Lym), CD3/CD45, CD4/CD8, CD4+CD25+, CD45, CD45+CD66b+, and CD45+CD14+ properties for Specimen Tissue-9 (cell suspension, TTDR-dissociated) at Day 0 and after −80° C. for 20 days using cryopreservation buffers 10% DMSO+90% FBS, 10% AF+90% TTPR, and TTCR (FIG. 15A) and cell number quantifications derived therefrom (FIGS. 15B-15H) for live (FIG. 15B), CD45− (FIG. 15C), lymphocyte (Lym) (FIG. 15D), CD45+CD3+ (FIG. 15E), CD4 (FIG. 15F), CD25 (FIG. 15G), and CD8 (FIG. 15H) cells.

FIGS. 16A-16I depict FACS data related to viability, lymphocyte (Lym), CD3/CD45, CD4/CD8, CD4+CD25+, CD45, and CD45+CD14+ properties for Specimen Tissue-11 (cell suspension, TTDR-dissociated) at Day 0 and after −80° C. for 20 days using cryopreservation buffers 5% DMSO+95% TTPR and TTCR (FIG. 16A) and cell number quantifications derived therefrom (FIGS. 16B-16I) for live (FIG. 16B), CD45− (FIG. 16C), CD14+ (FIG. 16D), lymphocyte (Lym) (FIG. 16E), CD45+CD3+ (FIG. 16F), CD4+ (FIG. 16G), CD25+ (FIG. 16H), and CD8+ (FIG. 16I) cells.

FIGS. 17A-17S depict FACS data related to viability, lymphocyte (Lym), CD45/CD3<Lym, CD4/CD8<CD45+CD3+, CD25<CD4+, CD45<Live, and CD14<CD45+ properties for Specimen Tissue-12 at Day 0 (fresh) and after −80° C. for 50 days using either cryopreservation buffer TTCR or snap-frozen (FIG. 17A), cell count quantifications derived therefrom (FIGS. 17B-17J) for live (FIG. 17B), CD45+ (FIG. 17C), CD14+ (FIG. 17D), lymphocyte (Lym) (FIG. 17E), CD45+CD3+ (FIG. 17F), CD4+ (FIG. 17G), CD25+ (FIG. 17H), CD8+ (FIG. 17I), and CD45+CD3− (FIG. 17J) cells, and frequency (%) quantifications derived therefrom (FIGS. 17K-17S) for live (FIG. 17K), CD45+ (FIG. 17L), CD14+ (FIG. 17M), lymphocyte (Lym) (FIG. 17N), CD45+CD3+ (FIG. 17O), CD4+ (FIG. 17P), CD25+ (FIG. 17Q), CD8+ (FIG. 17R), and CD45+CD3− (FIG. 17S) cells.

FIGS. 18A-18S depict FACS data related to viability, lymphocyte (Lym), CD45/CD3<Lym, CD4/CD8<CD45+CD3+, CD25<CD4+, CD45<Live, and CD14<CD45+ properties for Specimen Tissue-13 after −80° C. for 70 days using either cryopreservation buffer TTCR or snap-frozen (FIG. 18A), cell count quantifications derived therefrom (FIGS. 18B-18J) for live (FIG. 18B), CD45+ (FIG. 18C), CD14+ (FIG. 18D), lymphocyte (Lym) (FIG. 18E), CD45+CD3+ (FIG. 18F), CD4+ (FIG. 18G), CD25+ (FIG. 18H), CD8+ (FIG. 18I), and CD45+CD3− (FIG. 18J) cells, and frequency (%) quantifications derived therefrom (FIGS. 18K-18S) for live (FIG. 18K), CD45+ (FIG. 18L), CD14+ (FIG. 18M), lymphocyte (Lym) (FIG. 18N), CD45+CD3+ (FIG. 18O), CD4+ (FIG. 18P), CD25+ (FIG. 18Q), CD8+ (FIG. 18R), and CD45+CD3− (FIG. 18S) cells.

FIGS. 19A-19Q depict FACS data related to viability, lymphocyte (Lym), CD45/CD3<Lym, CD4/CD8<CD45+CD3+, CD25<CD4+, CD45<Live, and CD14<CD45+ properties for Specimen Tissue-14 at Day 0 (fresh) and after −80° C. for 30 days using cryopreservation buffers 10% DMSO/90% FBS and TTCR (FIG. 19A), cell count quantifications derived therefrom (FIGS. 19B-19I) for live (FIG. 19B), CD45+ (FIG. 19C), CD14+ (FIG. 19D), lymphocyte (Lym) (FIG. 19E), CD3+ (FIG. 19F), CD4+ (FIG. 19G), CD25+ (FIG. 19H), and CD8+ (FIG. 19I) cells, and frequency (%) quantifications derived therefrom (FIGS. 19J-19Q) for live (FIG. 19J), CD45+ (FIG. 19K), CD14+ (FIG. 19L), lymphocyte (Lym) (FIG. 19M), CD3+ (FIG. 19N), CD4+ (FIG. 19O), CD25+ (FIG. 19P), and CD8+ (FIG. 19Q) cells.

FIGS. 20A-20S depict FACS data related to viability, lymphocyte (Lym), CD45/CD3<Lym, CD4/CD8<CD45+CD3+, CD25<CD4+, CD45<Live, and CD14<CD45+ properties for Specimen Tissue-11 after −80° C. for 70 days using cryopreservation buffers 10% DMSO/90% FBS and TTCR (FIG. 20A), cell count quantifications derived therefrom (FIGS. 20B-20J) for live (FIG. 20B), CD45+ (FIG. 20C), CD14+ (FIG. 20D), lymphocyte (Lym) (FIG. 20E), CD45+CD3+ (FIG. 20F), CD4+ (FIG. 20G), CD25+ (FIG. 20H), CD8+ (FIG. 20I), and CD45+CD3− (FIG. 20J) cells, and frequency (%) quantifications derived therefrom (FIGS. 20K-20S) for live (FIG. 20K), CD45+ (FIG. 20L), CD14+ (FIG. 20M), lymphocyte (Lym) (FIG. 20N), CD45+CD3+ (FIG. 20O), CD4+ (FIG. 20P), CD25+ (FIG. 20Q), CD8+ (FIG. 20R), and CD45+CD3− (FIG. 20S) cells.

FIGS. 21A-21U depict FACS data related to viability, lymphocyte (Lym), CD3/CD45, CD4/CD8, CD25+, CD45+, CD14+CD11b+<CD45+, and CD56+<CD3-properties for Specimen Tissue-15 at Day 0 and after −80° C. for 60 days using cryopreservation buffer TTCR (Cell-S and Chunk) (FIG. 21A), cell count quantifications derived therefrom (FIGS. 21B-21K) for live (FIG. 21B), CD45+ (FIG. 21C), CD14+CD11b+ (FIG. 21D), lymphocyte (Lym) (FIG. 21E), CD45+CD3+ (FIG. 21F), CD4+ (FIG. 21G), CD25+ (FIG. 21H), CD8+ (FIG. 21I), CD45+CD3− (FIG. 21J), and CD56+ (FIG. 21K) cells, and frequency (%) quantifications derived therefrom (FIGS. 21L-21U) for live (FIG. 21L), CD45+ (FIG. 21M), CD14+CD11b+ (FIG. 21N), lymphocyte (Lym) (FIG. 21O), CD45+CD3+ (FIG. 21P), CD4+(FIG. 21Q), CD25+ (FIG. 21R), CD8+ (FIG. 21S), CD45+CD3− (FIG. 21T), and CD56+ (FIG. 21U) cells.

FIGS. 22A-22U depict FACS data related to viability, lymphocyte (Lym), CD3/CD45, CD4/CD8, CD25+, CD45+, CD14+CD11b+<CD45+, and CD56+<CD3-properties for Specimen Tissue-16 after −80° C. for 60 days using cryopreservation buffer TTCR (Cell-S and Chunk) (FIG. 22A), cell count quantifications derived therefrom (FIGS. 22B-22K) for live (FIG. 22B), CD45+ (FIG. 22C), CD14+CD11b+ (FIG. 22D), lymphocyte (Lym) (FIG. 22E), CD45+CD3+ (FIG. 22F), CD4+ (FIG. 22G), CD25+ (FIG. 22H), CD8+ (FIG. 22I), CD45+CD3− (FIG. 22J), and CD56+ (FIG. 22K) cells, and frequency (%) quantifications derived therefrom (FIGS. 22L-22U) for live (FIG. 22L), CD45+ (FIG. 22M), CD14+CD11b+ (FIG. 22N), lymphocyte (Lym) (FIG. 22O), CD45+CD3+ (FIG. 22P), CD4+ (FIG. 22Q), CD25+ (FIG. 22R), CD8+ (FIG. 22S), CD45+CD3− (FIG. 22T), and CD56+ (FIG. 22U) cells.

FIGS. 23A-23U depict FACS data related to viability, lymphocyte (Lym), CD3/CD45, CD4/CD8, CD25+, CD45+, CD14+CD11b+<CD45+, and CD56+<CD3-properties for Specimen Tissue-17 after −80° C. for 60 days using cryopreservation buffer TTCR (Cell-S and Chunk) (FIG. 23A), cell count quantifications derived therefrom (FIGS. 23B-23K) for live (FIG. 23B), CD45+ (FIG. 23C), CD14+CD11b+ (FIG. 23D), lymphocyte (Lym) (FIG. 23E), CD45+CD3+ (FIG. 23F), CD4+ (FIG. 23G), CD25+ (FIG. 23H), CD8+ (FIG. 23I), CD45+CD3− (FIG. 23J), and CD56+ (FIG. 23K) cells, and frequency (%) quantifications derived therefrom (FIGS. 23L-23U) for live (FIG. 23L), CD45+ (FIG. 23M), CD14+CD11b+ (FIG. 23N), lymphocyte (Lym) (FIG. 23O), CD45+CD3+ (FIG. 23P), CD4+ (FIG. 23Q), CD25+ (FIG. 23R), CD8+ (FIG. 23S), CD45+CD3− (FIG. 23T), and CD56+ (FIG. 23U) cells.

FIGS. 24A-24U depict FACS data related to viability, lymphocyte (Lym), CD3/CD45, CD4/CD8, CD25+, CD45+, CD14+CD11b+<CD45+, and CD56+<CD3-properties for Specimen Tissue-18 at Day 0 and after −80° C. for 60 days using cryopreservation buffer TTCR (Cell-S and Chunk) (FIG. 24A), cell count quantifications derived therefrom (FIGS. 24B-24K) for live (FIG. 24B), CD45+ (FIG. 24C), CD14+CD11b+ (FIG. 24D), lymphocyte (Lym) (FIG. 24E), CD45+CD3+ (FIG. 24F), CD4+ (FIG. 24G), CD25+ (FIG. 24H), CD8+ (FIG. 24I), CD45+CD3− (FIG. 24J), and CD56+ (FIG. 24K) cells, and frequency (%) quantifications derived therefrom (FIGS. 24L-24U) for live (FIG. 24L), CD45+ (FIG. 24M), CD14+CD11b+ (FIG. 24N), lymphocyte (Lym) (FIG. 24O), CD45+CD3+ (FIG. 24P), CD4+ (FIG. 24Q), CD25+ (FIG. 24R), CD8+ (FIG. 24S), CD45+CD3− (FIG. 24T), and CD56+ (FIG. 24U) cells.

FIGS. 25A-25U depict FACS data related to viability, lymphocyte (Lym), CD3/CD45, CD4/CD8, CD25+, CD45+, CD14+CD11b+<CD45+, and CD56+<CD3-properties for Specimen Tissue-19 at Day 0 and after −80° C. for 90 days using cryopreservation buffer TTCR (Chunk) (FIG. 25A), cell count quantifications derived therefrom (FIGS. 25B-25K) for live (FIG. 25B), CD45+ (FIG. 25C), CD14+CD11b+ (FIG. 25D), lymphocyte (Lym) (FIG. 25E), CD45+CD3+ (FIG. 25F), CD4+ (FIG. 25G), CD25+ (FIG. 25H), CD8+ (FIG. 25I), CD45+CD3− (FIG. 25J), and CD56+ (FIG. 25K) cells, and frequency (%) quantifications derived therefrom (FIGS. 25L-25U) for live (FIG. 25L), CD45+ (FIG. 25M), CD14+CD11b+ (FIG. 25N), lymphocyte (Lym) (FIG. 25O), CD45+CD3+ (FIG. 25P), CD4+ (FIG. 25Q), CD25+ (FIG. 25R), CD8+ (FIG. 25S), CD45+CD3− (FIG. 25T), and CD56+ (FIG. 25U) cells. Data shown for duplicates.

FIGS. 26A-26Q depict FACS data related to viability, lymphocyte (Lym), CD45/CD3<Lym, CD4/CD8<CD45+CD3+, CD25<CD4+, CD45<Live, and CD14<CD45+ properties for Specimen Tissue-20 at Day 0 (fresh) and after −80° C. for 25 days using cryopreservation buffer TTCR (FIG. 26A), cell count quantifications derived therefrom (FIGS. 26B-26I) for live (FIG. 26B), CD45+ (FIG. 26C), CD14+ (FIG. 26D), lymphocyte (Lym) (FIG. 26E), CD3+ (FIG. 26F), CD4+ (FIG. 26G), CD25+ (FIG. 26H), and CD8+ (FIG. 26I) cells, and frequency (%) quantifications derived therefrom (FIGS. 26J-26Q) for live (FIG. 26J), CD45+ (FIG. 26K), CD14+ (FIG. 26L), lymphocyte (Lym) (FIG. 26M), CD3+ (FIG. 26N), CD4+ (FIG. 26O), CD25+ (FIG. 26P), and CD8+ (FIG. 26Q) cells.

FIGS. 27A-27R depict FACS data related to viability, Lym<Live, CD3/CD45<Lym, CD56+<CD45+CD3−, CD4/CD8<CD45+CD3+, CD25+<CD4+, and CD14+CD11b<live properties for Specimen Tissue-19-LN at Day 0 (fresh) and after −80° C. for 54 days using cryopreservation buffer TTCR and suspending buffers 10% FBS/RPMI (cold and warm) and TTS (cold and warm) (FIG. 27A), cell count quantifications derived therefrom (FIGS. 27B-27J) for live (FIG. 27B), lymphocyte (Lym) (FIG. 27C), CD45+CD3+ (FIG. 27D), CD4+ (FIG. 27E), CD25+ (FIG. 27F), CD8+ (FIG. 27G), CD45+CD3− (FIG. 27H), CD56+ (FIG. 27I), and CD14+CD11b+ (FIG. 27J) cells, and frequency (%) quantifications derived therefrom (FIGS. 27K-27R) for live (FIG. 27K), lymphocyte (Lym) (FIG. 27L), CD45+CD3+ (FIG. 27M), CD4+ (FIG. 27N), CD25+ (FIG. 27O), CD8+ (FIG. 27P), CD45+CD3− (FIG. 27Q), and CD56+ (FIG. 27R) cells.

FIGS. 28A-28S depict FACS data related to viability, Lym<Live, CD3/CD45<Lym, CD56+<CD45+CD3−, CD4/CD8<CD45+CD3+, CD25+<CD4+, and CD14+CD11b<live properties for Specimen Tissue-21-LN at Day 0 (fresh) and after −80° C. for 16 days using cryopreservation buffer TTCR and suspending buffers 10% FBS/RPMI (cold and warm) and TTS (cold and warm) (FIG. 28A), cell count quantifications derived therefrom (FIGS. 28B-28J) for live (FIG. 28B), lymphocyte (Lym) (FIG. 28C), CD45+CD3+ (FIG. 28D), CD4+ (FIG. 28E), CD25+ (FIG. 28F), CD8+ (FIG. 28G), CD45+CD3− (FIG. 28H), CD56+ (FIG. 28I), and CD14+CD11b+ (FIG. 28J) cells, and frequency (%) quantifications derived therefrom (FIGS. 28K-28S) for live (FIG. 28K), lymphocyte (Lym) (FIG. 28L), CD45+CD3+ (FIG. 28M), CD4+ (FIG. 28N), CD25+ (FIG. 28O), CD8+ (FIG. 28P), CD45+CD3− (FIG. 28Q), CD56+ (FIG. 28R), and CD14+CD11b+ (FIG. 28S) cells.

FIGS. 29A-29S depict FACS data related to viability, Lym<Live, CD3/CD45<Lym, CD56+<CD45+CD3−, CD4/CD8<CD45+CD3+, CD25+<CD4+, and CD14+CD11b<live properties for Specimen Tissue-22-LN at Day 0 (fresh) and after −80° C. for 12 days using cryopreservation buffer TTCR and suspending buffers 10% FBS/RPMI (cold and warm) and TTS (cold and warm) (FIG. 29A), cell count quantifications derived therefrom (FIGS. 29B-29J) for live (FIG. 29B), lymphocyte (Lym) (FIG. 29C), CD45+CD3+ (FIG. 29D), CD4+ (FIG. 29E), CD25+ (FIG. 29F), CD8+ (FIG. 29G), CD45+CD3− (FIG. 29H), CD56+ (FIG. 29I), and CD14+CD11b+ (FIG. 29J) cells, and frequency (%) quantifications derived therefrom (FIGS. 29K-29S) for live (FIG. 29K), lymphocyte (Lym) (FIG. 29L), CD45+CD3+ (FIG. 29M), CD4+ (FIG. 29N), CD25+ (FIG. 29O), CD8+ (FIG. 29P), CD45+CD3− (FIG. 29Q), CD56+ (FIG. 29R), and CD14+CD11b+ (FIG. 29S) cells.

FIGS. 30A-30B depict non-limiting exemplary pre- and post-cryopreservation FACS data (FIG. 30A) and quantifications derived therefrom (FIG. 30B).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

There are provided, in some embodiments, compositions (e.g., cryopreservation reagents). The composition (e.g., cryopreservation reagent) can comprise: one or more cryoprotective agent(s) (e.g., dimethyl sulfoxide (DMSO) and/or ethylene glycol (EG)). The composition (e.g., cryopreservation reagent) can comprise: one or more culture media component(s) (e.g., FBS (Fetal Bovine Serum) and/or RPMI-1640). The composition (e.g., cryopreservation reagent) can comprise: one or more carbohydrate(s) (e.g., trehalose, sucrose, and/or fructose). The composition (e.g., cryopreservation reagent) can comprise: one or more amino acid(s) (e.g., taurine).

There are provided, in some embodiments, shielded samples. In some embodiments, the shielded sample comprises: a sample contacted with a composition (e.g., cryopreservation reagent) disclosed herein. There are provided, in some embodiments, cryopreserved samples. In some embodiments, the cryopreserved sample comprises: a sample contacted with a cryopreservation reagent disclosed herein and exposed to a freezing storage condition. There are provided, in some embodiments, preserved samples. In some embodiments, the preserved sample comprises: a sample contacted with a composition (e.g., cryopreservation reagent) disclosed herein and exposed to a storage condition.

There are provided, in some embodiments, methods of sample cryopreservation. The method can comprise: providing a cryopreservation reagent disclosed herein. The method can comprise: providing a sample (e.g., a sample obtained via biopsy). The method can comprise: contacting a sample with the cryopreservation reagent, thereby generating a shielded sample. The method can comprise: exposing the shielded sample to a freezing storage condition, thereby generating a cryopreserved sample. Disclosed herein include methods of sample analysis. The method can comprise: providing a sample comprising a plurality of cells; and providing a composition (e.g., cryopreservation reagent) disclosed herein. The method can comprise: contacting the sample with the composition (e.g., cryopreservation reagent), thereby generating a shielded sample. The method can comprise: exposing the shielded sample to a freezing storage condition, thereby generating a cryopreserved sample. The method can comprise: thawing the cryopreserved sample. The method can comprise: exposing the shielded sample to a storage condition, thereby generating a preserved sample. The method can comprise: removing the preserved sample from the storage condition (e.g., thawing, change in temperature). The method can comprise: conducting one or more measurements of one or more cellular properties. Disclosed herein include methods of sample preservation. The method can comprise: exposing the shielded sample to a storage condition, thereby generating a preserved sample. The method can comprise: removing the preserved sample from the storage condition (e.g., thawing, change in temperature).

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

There are provided, in some embodiments, cell cryopreservation reagents capable of preserving various types of cells existing in human tumor tissue for long periods of time with high viability and/or remaining amendable to gene and protein analysis after storage (e.g., upon thawing). Cell cryopreservation reagent (e.g., liquids) disclosed herein can preserve cell mixtures of various types of cells existing in human tumor tissue for a long-term period, retaining high viability as it was pre-freezing, which enables sorting viable target cells by a cell sorter even after freeze-thaw and/or long read RNA-seq analysis, which cannot have been done by using fixed cells (10× Genomics). In some embodiments, the methods and compositions provided herein can employed by biorepositories (e.g., Cancer Bio-Bank) providing medical doctors and researchers with cancer specimens for gene and protein analysis.

The tumor microenvironment (TME) in tumor tissue is quite complex, with various types of cells existing there, including fibroblasts and immune cells (e.g., granulocytes, lymphocytes, macrophages, etc.) in addition to tumor cells. Moreover, immune cells in TME can be activated and more vulnerable. Compositions provided herein (e.g., cryopreservation buffer/reagent) can preserve a mixture of various types of cells isolated from human tumor tissue (e.g., by TTDR/BD) for a long period of time, in some embodiments retaining as high viability as pre-freezing (e.g., Day 0). In contrast, such samples exhibit decreased cell viability when commercially available cryopreservation buffers (e.g., Cell Banker) and conventional cryopreservation buffers (e.g., 10% DMSO/FBS) are employed.

Cell viability after thawing can be very important for RNA-seq analysis. Cryopreservation compositions provided herein (e.g., cryopreservation reagents/buffers, also termed “Tumor Tissue Cryopreservation Reagent” (“TTCR”) herein) can be utilized by biorepositories (e.g., Cancer Bio-Bank) where there is a need to store and/or pool various human specimens for long periods of time (e.g., years) for later analysis (e.g., gene and protein analysis) as well as for clinical research and study. A great advantage of stocking samples in the research field is that it makes it possible to treat a lot of samples at once, which leads to reduced “batch-effects/data variation” in addition to savings of cost and time.

TTCR enables FACS-sorting viable target cells using Live/Dead staining and user-selected antibodies (e.g., target FL-antibodies) even after cryopreservation, and thus, advantageously, a user does not have to perform FACS-Sorting as soon as human specimen is collected. In addition, while current commercial providers (e.g., 10× Genomics) provide immediate PFA fixation methods that aim to keep cell conditions as they were in tumor tissue for RNA-seq analysis, they require the process of cancelling formaldehyde crosslinking by enzyme and heat, which precludes the use of long read RNA sequencing analysis. TTCR enables the preservation of not only tumor infiltrating lymphocytes (TILs) but also other types of cells in TME (tumor microenvironment) including granulocytes, tumor cells, and fibroblasts, with much higher viability after thawing, as compared to commercially available cell cryopreservation buffers and conventional buffers.

BD® Tumor & Tissue Preservation Reagent (TTPR, BD catalog no. 664648; See also BDR OMICS-Guard Sample Preservation Buffer; BD® OMICS-Guard Buffer) can preserve tumor tissue for 72 hours without freezing and can be utilized for clinical tests and diagnostic purposes in a user seeks to acquire results as soon as possible for judging the next treatment. On the other hand, TTCR provided herein can store cell suspension (or tumor tissue crumbles) treated by a cell dispersion composition (e.g., BD Horizon™ Dri Tumor & Tissue Dissociation Reagent (TTDR)) for a long period of time. In some embodiments provided herein TTCR is employed with TTDR, TTPR, or both, for tumor tissue analysis.

The compositions and methods provided herein address several needs in the art and exhibit advantageous properties over compositions and methods. First, it is very difficult to cryopreserve with high viability cell mixtures of a variety of cell types existing in human tumor tissue, as many commercially available and conventional cell cryopreservation buffers can effectively store single types of cells (e.g., cultured cell strain). Furthermore, an existing problem in the art is that immune cells (including granulocytes) in the TME are more vulnerable because they are activated, and therefore, it is very difficult to retain their viability after freeze-thaw. However, compositions provided herein (e.g., TTCR) can cryopreserve cell mixtures of a variety of cell types (immune cells, tumor cells, fibroblasts, etc.) with as high viability as they were before-freezing (e.g., Day 0, fresh) even after freeze-thaw. Second, due to the high viability in every cell type achieved after freeze-thaw in some embodiments, a user does not need to spend time performing RNA-seq processing steps (e.g. cell-sorting or library construction) immediately after a specimen is collected, but rather a user can process cryopreserved sample vials whenever needed, which enables savings of time and cost as well as a reduction in data batch-effects/data variation. Third, although methods and compositions employing PFA/formaldehyde fixation of a sample (e.g., 10× Genomics) are useful without a cryopreservation step, it requires the process of cancelling formaldehyde crosslinking by enzyme and heat, which precludes long read sequencing. In contrast, samples preserved with TTCR can undergo long read sequencing as it does not require formaldehyde fixation.

While commercially available and conventional cryopreservation buffers can be capable of cryopreserving samples comprising a single type of cells, including cell strain and lymphocytes from blood, with high viability, they are not suitable to cryopreserve cells from human tumor tissue because a variety of cell types (including lymphocytes, granulocytes, tumor cells, fibroblasts, etc.) exist in human tumor tissue and these cells are vulnerable in the TME context. The well-balanced buffer compositions provided herein are suitable to cryopreserve cell mixtures of a variety of cell types, and are capable of retaining the viability even if they are vulnerable.

Table 1 provides the components of the exemplary compositions (e.g., cryopreservation reagents/buffers) provided herein (e.g., “Tumor Tissue Cryopreservation Reagent” (“TTCR”)). In some embodiments, the balance of anti-freeze components (e.g., DMSO and EG) in the presence of sugar(s) enables the cryopreservation reagent to cryopreserve viable cells of interest (e.g., CD45 low/neg and granulocytes) while commercially available and conventional cryopreservation buffers (e.g., 10% DMSO buffer) cannot.

TABLE 1
Buffer Composition

| | | Exemplary |
|---|---|---|
| | Concentration | Concentrations |
| DMSO (anti-freeze) | 3~7% | 5% |
| Ethylene Glycol (EG) (anti-freeze) | 3~7% | 5% |
| FBS (Fetal Bovine Serum) | 10~80% | 50% |
| RPMI-1640 | 10~70% | 30~50% |
| Trehalose | 20~200 mM | 50~100 mM |
| Sucrose (or Fructose) | 10~100 mM | 20~50 mM |
| Taurine* | 50~200 mM | 50~200 mM |

*The composition may contain taurine
% can be (w/w), (v/v), or (w/v)

Disclosed herein include compositions (e.g., cryopreservation reagents). The composition (e.g., cryopreservation reagent) can comprise: one or more cryoprotective agent(s) (e.g., dimethyl sulfoxide (DMSO) and/or ethylene glycol (EG)). The composition (e.g., cryopreservation reagent) can comprise: one or more culture media component(s) (e.g., FBS (Fetal Bovine Serum) and/or RPMI-1640). The composition (e.g., cryopreservation reagent) can comprise: one or more carbohydrate(s) (e.g., trehalose, sucrose, and/or fructose). The composition (e.g., cryopreservation reagent) can comprise: one or more amino acid(s) (e.g., taurine).

There are provided, in some embodiments, cryopreservation reagents, comprising: albumin, optionally 0.01-2% (w/v); lactic acid or a salt thereof, optionally 0.1-100 mmol/L; and one or more cryoprotective agent(s), optionally dimethyl sulfoxide (DMSO) and/or ethylene glycol (EG). In some embodiments, the sodium ion concentration of the cryopreservation reagent is about 50-300 mmol/L. The albumin can be bovine serum albumin (BSA). The lactic acid or salt thereof can be sodium lactate. The cryopreservation reagent can comprise about 0.01-10 mmol/L of potassium dihydrogen phosphate. The cryopreservation reagent can comprise about 0.01-50 mmol/L of disodium hydrogen phosphate. The cryopreservation reagent can comprise a polysaccharide, optionally selected from the group comprising trehalose, a fructooligosaccharide, indigestible dextrin, or any combination thereof.

The composition (e.g., cryopreservation reagent) can be capable of maintaining one or more cellular properties and/or cell viability of a plurality of cells in a sample for a period of time under a storage condition. A contact of the composition (e.g., cryopreservation reagent) with a sample comprising a plurality of cells can be capable of generating a shieled sample, and one or more cellular properties and/or cell viability of said plurality of cells can be maintained for a period of time under a storage condition. The compositions provided herein can maintain the properties of target substances in a sample. The target substance can be any substance that can be included in a sample. Examples of the target substances include, but are not limited to, cells, genes, proteins including antibodies, amino acids, chemical substances including neurotransmitters, and so on.

The composition (e.g., cryopreservation reagent) can comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, or a number or a range between any two of these values, (v/v), (w/v), or (w/w), of the one or more cryoprotective agent(s), such as, for example, about 3% (v/v) to about 7% (v/v) of a first cryoprotective agent (e.g., DMSO) and about 3% (v/v) to about 7% (v/v) of a second cryoprotective agent (e.g., EG). The one or more cryoprotective agent(s) can be selected from the group comprising acetamide, albumin, ammonium acetate, anti-freeze proteins, butanediol, chloroform, choline, cyclohexanediols, cyclohexanediones, cyclohexanetriols, diethylene glycol, dimethyl acetamide, dimethyl formamide, dimethyl sulfoxide, ethanol, ethylene glycol, ethylene glycol monomethyl ether, formamide, glycerol, glyceryl monoacetate, glycoproteins, hydroxyethyl starch, magnesium chloride, magnesium sulfate, methanol, methoxy propanediol, methyl acetamide, methyl formamide, methyl ureas, methyl glycerol, phenol, pluronic polyols, polyethylene glycol, polyvinylpyrrolidone, propanediol, pyridine N-oxide, sodium bromide, sodium chloride, sodium iodide, sodium nitrate, sodium nitrite, sodium sulfate, triethylene glycol, trimethylamine acetate, urea, derivatives thereof, or any combination thereof.

The composition (e.g., cryopreservation reagent) can comprise the one or more carbohydrate(s) at a concentration of about 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM, 250 mM, 260 mM, 270 mM, 280 mM, 290 mM, 300 mM, or a number or a range between any two of these values, such as, for example, a first carbohydrate (e.g., trehalose) at a concentration of about 50 mM to about 100 mM and a second carbohydrate (e.g., sucrose) at a concentration of about 20 mM to about 50 mM. The one or more carbohydrate(s) can be selected from the group comprising a sugar, a monosaccharide, a disaccharide, a polyol, an oligosaccharide, a malto-oligosaccharide, a non-malto-oligosaccharide, a polysaccharide, a starch, a non-starch polysaccharide, derivatives thereof, or any combination thereof. The one or more carbohydrate(s) can be selected from the group comprising trehalose, fructose, sucrose, allose, altrose, amylopectin, arabinose, adonitol, cellobiose, cyclodextrin, dextran, deoxyribose, dulcitol, dextrose, erythritol, erythrose, erythrulose, fucose, galactose, glucosamine, glucose, gulose, idose, inositol, invert sugar, isotrehalose, lactose, lyxose, maltodextrin, maltose, mannitol, mannose, mannosamine, melezitose, neotrehalose, palatinose (isomaltulose), psicose, raffinose, ribose, ribulose, rhamnose, sialic acid, sorbitol, sorbose, tagatose, talose, threitol, threose, turanose, xylitol, xylose, xylulose, derivatives thereof, or any combination thereof.

The composition (e.g., cryopreservation reagent) can comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or a number or a range between any two of these values, (v/v), (w/v), or (w/w), of the one or more culture media component(s), such as, for example, about 50% (v/v) of a first culture media component (e.g., FBS) and about 30% (v/v) to about 50% (v/v) of a second culture media component (e.g., RPMI-1640). The one or more culture media component(s) can be selected from the group comprising AIM-V, Alpha-Minimum Essential Medium (α-MEM), Basal Medium Eagle (BME), Brainphys, CTS KnockOut DMEM, CTS OpTimizer T Cell Expansion SFM, CellGro DC Medium, Dulbecco's Modified Eagle Medium (DMEM), Dulbecco's Modified Eagle Medium: Nutrient Mixture F-12 (DMEM/F-12), Dulbecco's Phosphate Buffered Saline (DPBS), Eagle's Basal Medium (EBM), Eagle's Essential Medium (EEM), Earle's Balanced Salt Solution (EBSS), F-10 Nutrient Mixture, F-12K Nutrient Mixture Medium (Kaighn's Modification, F-12K), Fetal Bovine Serum (FBS), Fischer's Medium, GMEM (Glasgow's Minimum Essential Medium), Hanks' Balanced Salt Solution (HBSS), Hanks' Salts Medium (HMEM), Ham's F-10, Ham's F-12, ImmunoCult-XF T Cell Expansion Medium, Iscove's Modified Dulbecco's Medium (IMDM), L-15 (Leibovitz's L-15), MCDB 131, MCDB 153, MDEM, MEM (Minimum Essential Medium), M199 (Medium 199), McCoy's 5A, Neurobasal, Neurobasal A, ObM (Osteoblast Medium), Opti-MEM I Reduced Serum Media, PBS (Phosphate-Buffered Saline), PRIME-XV T Cell Expansion Medium, Roswell Park Memorial Institute Medium (RPMI) 1640 Medium, Sodium Bicarbonate Buffers, StemLine II, StemPro-34, StemSpan-ACF, StemSpan-H3000, StemSpan-SFEM, StemXVivo, TexMACS Medium, X-VIVO 15, X-vivo 20, derivatives thereof, or any combination thereof.

The one or more amino acid(s) can be present at a concentration of about 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 200 mM, 300 mM, 400 mM, 500 mM, 600 mM, 700 mM, 800 mM, 900 mM, 1 M, 1.2 M, 1.3 M, 1.4 M, 1.5 M, 1.6 M, 1.7 M, 1.8 M, 1.9 M, 2 M, or a number or a range between any two of these values (e.g., about 50 mM to about 200 mM). The one or more amino acid(s) can be selected from the group comprising taurine, alanine, arginine, asparagine, aspartic acid, cysteine, glutamic acid, glutamine, glycine, histidine, isoleucine, leucine, lysine, methionine, phenylalanine, proline, selenocysteine, serine, threonine, tyrosine, tryptophan, ornithine, citrulline, aminobenzoic acid, valine, derivatives thereof, or any combination thereof.

The cryopreservation reagent can comprise one or more cryoprotective agent(s) and one or more culture media component(s). The cryopreservation reagent can comprise one or more cryoprotective agent(s) and one or more carbohydrate(s). The cryopreservation reagent can comprise one or more cryoprotective agent(s) and one or more amino acid(s). The cryopreservation reagent can comprise one or more culture media component(s) and one or more carbohydrate(s). The cryopreservation reagent can comprise one or more culture media component(s) and one or more amino acid(s). The cryopreservation reagent can comprise one or more carbohydrate(s) and one or more amino acid(s). The cryopreservation reagent can comprise one or more cryoprotective agent(s), one or more culture media component(s), and one or more carbohydrate(s). The cryopreservation reagent can comprise one or more cryoprotective agent(s), one or more culture media component(s), and one or more amino acid(s). The cryopreservation reagent can comprise one or more cryoprotective agent(s), one or more carbohydrate(s), and one or more amino acid(s). The cryopreservation reagent can comprise one or more culture media component(s), one or more carbohydrate(s), and one or more amino acid(s).

The composition (e.g., cryopreservation reagent) can comprise about 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, or a number or a range between any two of these values, (v/v), (w/v), or (w/w), DMSO (e.g., about 5% (v/v)). The composition (e.g., cryopreservation reagent) can comprise about 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, or a number or a range between any two of these values, (v/v), (w/v), or (w/w), EG (e.g., about 5% (v/v)). The composition (e.g., cryopreservation reagent) can comprise about 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or a number or a range between any two of these values, (v/v), (w/v), or (w/w), FBS (e.g., about 50% (v/v)). The composition (e.g., cryopreservation reagent) can comprise about 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or a number or a range between any two of these values, (v/v), (w/v), or (w/w), RPMI-1640 (e.g., about 30% (v/v) to about 50% (v/v)). The composition (e.g., cryopreservation reagent) can comprise trehalose at a concentration of about 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM, 250 mM, 260 mM, 270 mM, 280 mM, 290 mM, 300 mM, or a number or a range between any two of these values (e.g., about 50 mM to about 100 mM). The composition (e.g., cryopreservation reagent) can comprise sucrose and/or fructose at a concentration of about 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM, 250 mM, 260 mM, 270 mM, 280 mM, 290 mM, 300 mM, or a number or a range between any two of these values (e.g., about 20 mM to about 50 mM). The composition (e.g., cryopreservation reagent) can comprise taurine at a concentration of about 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM, 250 mM, 260 mM, 270 mM, 280 mM, 290 mM, 300 mM, or a number or a range between any two of these values. In some embodiments, the composition (e.g., cryopreservation reagent) does not comprise: albumin (e.g., bovine serum albumin (BSA)); lactic acid or a salt thereof (e.g., sodium lactate); potassium dihydrogen phosphate; and/or disodium hydrogen phosphate.

In some embodiments, the compositions (e.g., cryopreservation reagent, sample preservation composition) comprise albumin. The albumin employed can be serum albumin found in vertebrate (e.g., humans, cows, horses, rabbits) serum, ovalbumin found in egg whites, milk albumin found in milk, and/or seed albumin found in plant seeds. Albumin can be included in the composition at a concentration of 0.01 to 2% (w/v), 0.05 to 1% (w/v), 0.1 to 0.5% (w/v), 0.1 to 0.3% (w/v), or a number or a range between any two of these values.

In some embodiments, the compositions provided herein (e.g., cryopreservation reagent, sample preservation composition) comprise lactic acid or a salt thereof. “Lactic acid or a salt thereof” can refer to lactic acid or any lactate salt that dissolves in water to yield lactate ions and cations. Examples of a lactate salt include lithium lactate, sodium lactate, potassium lactate, calcium lactate, ammonium lactate, aluminum lactate, and magnesium lactate. In some embodiments, the lactic acid or a salt thereof is included in the composition so as to have a lactic acid ion concentration in the composition of 0.1 to 100 mmol/L, 1.0 to 50 mmol/L, 1.0 to 30 mmol/L, 1.0 to 30 mmol/L, 3.0 to 20 mmol/L, or a number or a range between any two of these values. Lactic acid has two steric structures, D and L, which are in an enantiomeric relationship, and salts of lactate also have a steric structure corresponding to the structure of lactic acid. Enantiomers refer to stereoisomers whose stereostructures are non-superimposable and which are in an “image-mirror image” relationship. An equal mixture of D and L forms is called a racemic mixture, or a DL form. In some embodiments, L forms and/or DL forms of lactic acid or salts thereof are present in the composition.

In some embodiments the compositions provided herein (e.g., cryopreservation reagent, sample preservation composition) comprise sodium ions at a concentration of 50 to 300 mmol/L, 80 to 250 mmol/L, 100 to 200 mmol/L, or a number or a range between any two of these values. The sodium ion concentration can be adjusted by adding substances that dissolve in water to produce sodium ions. Examples of substances that produce sodium ions include, but are not limited to, sodium hydroxide, sodium chloride, sodium carbonate, sodium nitrate, sodium sulfate, sodium hydrogen sulfate, sodium acetate, sodium oxalate, sodium citrate, sodium dihydrogen phosphate, and dihydrogen phosphate.

In some embodiments the compositions provided herein (e.g., cryopreservation reagent, sample preservation composition) comprise potassium dihydrogen phosphate and/or disodium hydrogen phosphate. Potassium dihydrogen phosphate can be included in the composition at a concentration of 0.01 to 10 mmol/L, 0.05 to 5.0 mmol/L, 1.0 to 2.5 mmol/L, or a number or a range between any two of these values, and disodium hydrogen phosphate can included in the composition at a concentration of 0.01 to 50 mmol/L, 1.0 to 25 mmol/L, 5 to 15 mmol/L, or a number or a range between any two of these values.

In some embodiments the compositions provided herein (e.g., cryopreservation reagent, sample preservation composition) do not include calcium ions. In some embodiments the compositions provided herein (e.g., cryopreservation reagent, sample preservation composition) comprise a polysaccharide. A polysaccharide can be a compound formed by dehydration bonding of two or more monosaccharides, substituted derivatives thereof, or mixtures thereof. Polysaccharides include, but are not limited to, disaccharides such as sucrose, lactose, maltose and trehalose; trisaccharides such as raffinose and maltotriose; tetrasaccharides such as acarbose and stachyose; oligosaccharides such as malto-oligosaccharides and fructo-oligosaccharides; and glycogen, starch, dextrin, and others containing more molecules, for example. The polysaccharide can be selected from the group consisting of trehalose, a fructo-oligosaccharide, an indigestible dextrin, and mixtures thereof. In some embodiments, the composition includes a polysaccharide at a concentration of 0.01 to 3.0% (w/v), 0.03 to 1.0% (w/v), 0.05 to 0.5% (w/v), or a number or a range between any two of these values.

The plurality of cells can comprise a plurality of cell types (e.g., at least about 2, 3, 4, 5, 6, 7, 8, 9, 10, 12, 14, 16, 18, 20, 25, 50, or a number or a range between any two of the values, distinct cell types). The plurality of cell types can be selected from the group comprising a tumor cell, an antigen-presenting cell, a dendritic cell, a fibroblast, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell (e.g., an embryonic stem cell, an induced pluripotent stem cell (iPSC), a hematopoietic stem/progenitor cell (HSPC), or any combination thereof), sustentacular cell, teloglial cell, a zymogenic cell, or any combination thereof.

The plurality of cell types can be selected from the group comprising tumor-associated DCs (TADCs), tumor-associated neutrophils (TANs), myeloid-derived suppressor cells (MDSCs), tumor-associated macrophages (TAMs), CD45 low/neg cells, CD4+ memory T-cells, CD4+ naive T-cells, CD4+ T-cells, central memory T (Tcm) cells, effector memory T (Tem) cells, CD4+ Tcm, CD4+ Tem, CD8+ T-cells, CD8+ naive T-cells, CD8+ Tcm, CD8+ Tem, regulatory T cells (Tregs), T helper (Th) 1 cells, Th2 cells, gamma delta T (Tgd) cells, natural killer (NK) cells, natural killer T (NKT) cells, B cells, naive B cells, memory B cells, class-switched memory B-cells, pro B-cells, plasma cells, M1 macrophages, M2 macrophages, CD19+B cells, CD14+ monocytes, CD56+NK cells, CD8+ T cells, Treg cells, CD4+ T cells, granulocytes, dendritic cells, or any combination thereof. The plurality of cells can comprise one or more immune cells having a naive status, activated status, activation recovered status, terminally exhausted status, progenitor exhausted status, central memory status, effector memory status, stem cell memory status, or any combination thereof. The sample can comprise a tumor microenvironment.

The sample can be a biological sample or an environmental sample. In some embodiments, the environmental sample is, or is obtained from, a food sample, a beverage sample, a paper surface, a fabric surface, a metal surface, a wood surface, a plastic surface, a soil sample, a freshwater sample, a wastewater sample, a saline water sample, exposure to atmospheric air or other gas sample, cultures thereof, or any combination thereof. In some embodiments, the biological sample is, or is obtained from, a tissue sample, saliva, blood, plasma, sera, stool, urine, sputum, mucous, lymph, synovial fluid, cerebrospinal fluid, ascites, pleural effusion, seroma, pus, swab of skin or a mucosal membrane surface, cultures thereof, or any combination thereof. The sample can comprise or can be derived from a tissue sample (e.g., a tumor tissue sample). The term “tissue” shall be given its ordinary meaning and also refer to a population of many types of cells. Tissues that can be preserved by the compositions provided herein include connective tissue such as epithelial tissue, bone tissue, cartilage tissue, blood tissue; and nerve tissue, muscle tissue, and tumor tissue derived from these tissues. The tissue may be collected from an organism or artificially constituted by culturing cells. The cells, tissue, or mixtures thereof can be obtained from an animal (e.g., a mammal). The sample can comprise or can be derived from a tumor sample. A “tumor sample” is a biological sample containing tumor cells, whether intact or degraded. The sample can comprise a bodily fluid (e.g., the lymph of a lymph node sample).

The sample (e.g., tissue sample) can be a biopsy sample, and can be obtained by needle aspiration, fine needle aspiration, core needle biopsy, vacuum assisted biopsy, large core biopsy, incisional biopsy, excisional biopsy, punch biopsy, shave biopsy, or skin biopsy. The sample can be derived from a subject (e.g., a subject having or suspected of having a tumor, a subject having a stage I cancer, a stage II cancer, a stage III cancer, and/or a stage IV cancer). The tumor can be selected from the group comprising biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic (CLL), chronic myeloid (CML), chronic myelomonocytic (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, uterine sarcoma, or any combination thereof.

In some embodiments, the sample has been contacted with a sample preservation composition and/or a cell dispersion composition capable of generating a single cell suspension. The sample can be or can comprise a single cell suspension. The plurality of cells can comprise at least about 10 cells, 50 cells, 100 cells, 200 cells, 300 cells, 400 cells, 500 cells, 600 cells, 700 cells, 800 cells, 900 cells, 1000 cells, 1250 cells, 1500 cells, 1750 cells, 2000 cells, 2250 cells, 2500 cells, 2750 cells, 3000 cells, 3250 cells, 3500 cells, 3750 cells, 4000 cells, 4250 cells, 4500 cells, 4750 cells, 5000 cells, 5250 cells, 5500 cells, 5750 cells, 6000 cells, 6250 cells, 6500 cells, 6750 cells, 7000 cells, 7250 cells, 7500 cells, 7750 cells, 8000 cells, 8250 cells, 8500 cells, 8750 cells, 9000 cells, 9250 cells, 9500 cells, 9750 cells, 10000 cells, 50000 cells, 100000 cells, or a number or a range between any two of the values. The sample can be at least about 0.01 mg, 0.05 mg, 0.10 mg, 0.20 mg, 0.50 mg, 1 mg, 2 mg, 5 mg, 10 mg, 15 mg, 20 mg, 25 mg, 30 mg, 40 mg, 50 mg, 75 mg, 100 mg, 150 mg, 200 mg, 300 mg, 400 mg, 500 mg, 600 mg, 700 mg, 800 mg, 900 mg, 1 g, 10 g, or a number or a range between any two of the values.

The one or more cellular properties can comprise at least about 2, 3, 4, 5, 6, 7, 8, 9, 10, 12, 14, 16, 18, 20, 25, 50, 75, 100, 250, 1000, or a number or a range between any two of the values, distinct cellular properties. The one or more cellular properties can comprise physical properties, morphological properties, intracellular properties, biochemical properties, dynamic behavioral properties, or any combination thereof. The physical properties can comprise cell size, volume, conductivity, low and high angle scatter, density, or any combination thereof. The morphological properties can comprise one or more of: cell shape, area, size, and lobularity; nucleus shape area, size, and lobularity; mitochondria shape, area, size, and lobularity; and ratio of nuclear volume to cell volume. The intracellular properties can comprise nucleus centroid/cell centroid distance, nucleus lobe centroid distance, distribution of proteins with the cells, distribution of organelles within the cells, or any combination thereof. The biochemical properties can comprise expression level of cellular proteins, cell surface proteins, cytoplasmic proteins, nuclear proteins, cellular nucleic acids, cell surface nucleic acids, cytoplasmic nucleic acids, nuclear nucleic acids, cellular carbohydrates, cell surface carbohydrates, cytoplasmic carbohydrates, nuclear carbohydrates, or any combination thereof. The dynamic behavioral properties can comprise cellular activation, cellular inhibition, protein secretion, microvesicle secretion, exosome secretion, microparticle secretion, metabolite secretion, small molecule secretion, proton secretion, protein expression, or any combination thereof.

The one or more cellular properties can comprise one or more cellular features indicating cell proliferation, stress pathway activation, organelle function, cell cycle state, apoptosis, DNA damage, metabolism, signal transduction, cell differentiation, or any combination thereof. The cellular features indicating cell proliferation can comprise nuclear count, cell count, total cell mass, total DNA, the phosphorylation state of cell cycle regulatory proteins, the post-translational modification state of any protein involved in cell growth or division, or any combination thereof. The cellular features indicating stress pathway activation can comprise transcription factor activation of NF-KB, API, ATF2, MSK1, CREB, or NFAT, and kinase activation of p38, JNK, ERK, RSK90, MEK, or any combination thereof. The cellular features indicating organelle function can comprise cytoskeletal organization, mitochondrial mass or membrane potential, peroxisome mass, golgi organization, plasma membrane permeability, or any combination thereof. The cellular features indicating cell cycle state can comprise DNA content, Histone H3 phosphorylation state, Rb phosporylation state, cyclin B1 (CDKI) biosynthesis, cyclin DI (CDK4, 6) biosynthesis, cyclin E (CDK2) biosynthesis, or any combination thereof. The cellular features indicating apoptosis can comprise nuclear size and shape, DNA content and degradation, caspase activation, phosphatidyl-expression, Bax translocation, or any combination thereof. The cellular features indicating DNA damage can comprise repair protein (APE) expression, tumor suppressor (e.g., p53, Rb) expression, oxidative activity (e.g., 8-oxoguanine), transcription activity (e.g., Oct1), or any combination thereof. The cellular features indicating metabolism can comprise cAMP concentration, P-glycoprotein activity or CYP450 induction/inhibition, the concentration of an added substance, or any combination thereof. The cellular features indicating signal transduction can comprise Ca²⁺ ion concentration, pH, expression of a protein, activation of a protein, modification of a protein, translocation of a protein, interaction between proteins known to be associated with a specific pathway, or any combination thereof. The cellular features indicating cell differentiation can comprise expression of a tissue specific protein, exhibiting a tissue specific morphology, or any combination thereof.

The one or more cellular properties can comprise one or more genomic properties, one or more expression properties, and/or one or more variant properties. The one or more variant properties can comprise a single nucleotide polymorphism (SNP), an insertion or deletion (indel), a copy number variant (CNV), a fusion, a splice variant, an isoform variant, a transversion, a translocation, a frame shift, a duplication, a repeat variant, or any combination thereof, at one or more loci of a plurality of loci; The one or more genomic properties can comprise chromatin accessibility, hypomethylation and/or hypermethylation at one or more loci of a plurality of loci. The one or more expression properties can comprise underexpression of one or more mRNAs of interest, underexpression of one or more proteins of interest, overexpression of one or more mRNAs of interest, and/or overexpression of one or more proteins of interest. The one or more mRNAs of interest and/or one or more proteins of interest can be derived from one or more loci of a plurality of loci.

The storage condition can comprise a temperature of about 4° C., 2° C., 0° C., −2° C., −4° C., −6° C., −8° C., −10° C., −12° C., −14° C., −16° C., −18° C., −20° C., −22° C., −24° C., −26° C., −28° C., −30° C., −32° C., −34° C., −36° C., −38° C., −40° C., −42° C., −44° C., −46° C., −48° C., −50° C., −52° C., −54° C., −56° C., −58° C., −60° C., −62° C., −64° C., −66° C., −68° C., −70° C., −72° C., −74° C., −76° C., −78° C., −80° C., −82° C., −84° C., −86° C., −88° C., −90° C., −92° C., −94° C., −96° C., −98° C., −100° C., −102° C., −104° C., −106° C., −108° C., −110° C., −112° C., −114° C., −116° C., −118° C., −120° C., −122° C., −124° C., −126° C., −128° C., −130° C., −132° C., −134° C., −136° C., −138° C., −140° C., −142° C., −144° C., −146° C., −148° C., −150° C., −152° C., −154° C., −156° C., −158° C., −160° C., −162° C., −164° C., −166° C., −168° C., −170° C., −172° C., −174° C., −176° C., −178° C., −180° C., −182° C., −184° C., −186° C., −188° C., −190° C., −192° C., −194° C., −196° C., a number or a range between any two of the values, or any combination thereof. The storage condition can comprise transport of a cryopreserved sample, thermal stress, one or more freeze-thaw cycles, agitation, pressure changes, light irradiation, or any combination thereof. The period of time can be at least about 1 day, 3 days, 5 days, 1 week, 2 weeks, 3 weeks, 1 month, 2 months, 3 months, 4 months, 5 months, 6 months, 7 months, 8 months, 9 months, 10 months, 11 months, 12 months, 14 months, 16 months, 18 months, 20 months, 22 months, 24 months, 26 months, 28 months, 30 months, 32 months, 34 months, 36 months, 38 months, 40 months, 42 months, 44 months, 46 months, 48 months, or a number or a range between any two of the values.

In some embodiments, at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of the values, of the plurality of cells in the sample remain viable post-storage condition. In some embodiments, at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of the values, of the plurality of cells in the sample maintain one or more cellular properties post-storage condition. The value(s) of the one or more cellular properties post-storage condition can be less than about 1.01-fold, 1.02-fold, 1.04-fold, 1.06-fold, 1.08-fold, 1.1-fold, 1.15-fold, 1.2-fold, 1.25-fold, 1.3-fold, 1.35-fold, 1.4-fold, 1.45-fold, 1.5-fold, or a number or a range between any two of the values, different than said value(s) prior to the storage condition. The expression of one or more stress-associated genes can be increased less than about 1.01-fold, 1.02-fold, 1.04-fold, 1.06-fold, 1.08-fold, 1.1-fold, 1.15-fold, 1.2-fold, 1.25-fold, 1.3-fold, 1.35-fold, 1.4-fold, 1.45-fold, 1.5-fold, or a number or a range between any two of the values, post-storage condition. The one or more stress-associated genes can be selected from the group consisting of ADM, AKR1B1, AQP1, AQP2, AQP4, ARNT, ATF4, ATF6, ATF6B, ATG12, ATG5, ATG7, ATM, ATR, BBC3, BECN1, BID, BNIP3L, CA9, CALR, CASP1, CCL2, CD40LG, CDKN1A, CFTR, CHEK1, CHEK2, CRP, DDB2, DDIT3, DNAJC3, EDN1, EPO, FAS, FTH1, GADD45A, GADD45G, GCLC, GCLM, GRB2, GSR, GSTP1, HMOX1, HSP90AA1, HSP90B1, HSPA4, HSPA4L, HSPA5, HUS1, IFNG, IL1A, IL1B, IL6, CXCL8, LDHA, MCL1, MMP9, MRE11A, NBN, NFAT5, NQ01, PARP1, PRDX1, PVR, RAD17, RAD51, RAD9A, RIPK1, SERPINE1, SLC2A1, SLC5A3, SQSTM1, TLR4, TNF, TNFRSF10A, TNFRSF10B, TNFRSF1A, TP53, TXN, TXNL4B, TXNRD1, ULK1, VEGFA, XPC, or any combination thereof.

In some embodiments, the cryopreservation reagents provided herein are capable of cryopreserving cell suspensions (e.g., tumor tissue-dissociated single cells). In some embodiments, the cryopreservation reagents provided herein are capable of cryopreserving biopsy specimens and tissues (e.g., tissue chunks) without first dissociating (e.g., contacting with a cell dispersion composition) said specimens/tissues. Cryopreservation reagents provided herein can be capable of cryopreservation of native, intact, unprocessed, and/or untreated (e.g., not contacted with a cell dispersion composition) biopsy specimens and tissues (e.g., tissue chunks). In some embodiments provided herein, cryopreservation of native, intact, unprocessed, and/or untreated biopsy specimens and tissues (e.g., tissue chunks) yields greater maintenance of one or more cellular properties and/or cell viability of a plurality of cells in a sample for a period of time under a storage condition as compared to samples that have been snap-frozen or have been processed (e.g., contacted with a cell dispersion composition, dissociated into single cells) before cryopreservation. In some embodiments, more viable cells are yielded from cryopreserving biopsy specimens and tissues (e.g., tissue chunks) as compared to cryopreserving of samples first contacted with a cell dispersion composition. In some embodiments, and without being bound by any particular theory, the freeze-thaw process can make samples more amendable to treatment with a cell dispersion composition.

The systems, methods, compositions, and kits provided herein can, in some embodiments, be employed in concert with the systems, methods, compositions, and kits described in Kobayashi et al. (“Isolation of tumor-infiltrating lymphocytes from preserved human tumor tissue specimens for downstream characterization.” STAR protocols 3.3 (2022): 101557), Kumagai et al. (“The PD-1 expression balance between effector and regulatory T cells predicts the clinical efficacy of PD-1 blockade therapies.” Nature immunology 21.11 (2020): 1346-1358), and in U.S. Patent Application Publication No. 2023/0152303, the contents of which are hereby incorporated by reference in their entireties.

Disclosed herein include shielded samples. In some embodiments, the shielded sample comprises: a sample contacted with a composition (e.g., cryopreservation reagent) disclosed herein. Disclosed herein include cryopreserved samples. In some embodiments, the cryopreserved sample comprises: a sample contacted with a cryopreservation reagent disclosed herein and exposed to a freezing storage condition. Disclosed herein include preserved samples. In some embodiments, the preserved sample comprises: a sample contacted with a composition (e.g., cryopreservation reagent) disclosed herein and exposed to a storage condition.

There are provided, in some embodiments, suspending buffers (e.g., TTS). In some embodiments, the suspending buffer comprises: one or more carbohydrate(s), optionally trehalose, sucrose, and/or fructose; one or more amino acid(s), optionally taurine; and/or one or more culture media component(s), optionally Fetal Bovine Serum (FBS) and/or RPMI-1640. In some embodiments, the suspending buffer comprises the one or more carbohydrate(s) at a concentration of about 10 mM to about 300 mM (or a number or a range between these values), optionally a first carbohydrate at a concentration of about 50 mM to about 200 mM and a second carbohydrate at a concentration of about 5 mM to about 100 mM, further optionally the first carbohydrate is trehalose and the second carbohydrate is sucrose. In some embodiments, the suspending buffer comprises about 0.1% (w/v) to about 10% (w/v) (or a number or a range between these values) of the one or more amino acid(s), optionally about 1.5% (w/v). In some embodiments, suspending buffer comprises about 0.1% (v/v) to about 80% (v/v) (or a number or a range between these values) of the one or more culture media component(s), optionally about 2% (v/v). In some embodiments, the suspending buffer comprises 100 mM Trehalose, 25 mM Sucrose, 1.5% (w/v) Taurine, 2% (v/v) FBS, and RPMI 1640. In some embodiments, thawing the cryopreserved sample comprises thawing at a temperature between about 32° C. and 42° C. (e.g., thawing in a 37° C. water bath). In some embodiments, the method comprises contacting the thawed sample with a suspending buffer provided herein (e.g., adding the thawed sample into the suspending buffer). The suspending buffer can be about 32° C.-42° C. (e.g., 37° C.) when contacted. The method can further comprise one or more centrifugations steps (e.g., 400 g at 4° C.) and/or one or more washes (e.g., washes with cold 2% FBS/RPMI). In some embodiments provided herein, contacting the cryopreserved sample with a suspending buffer provided herein yields greater maintenance of one or more cellular properties and/or cell viability of the plurality of cells of a thawed cryopreserved sample.

Disclosed herein include methods of sample cryopreservation. The method can comprise: providing a cryopreservation reagent disclosed herein. The method can comprise: providing a sample (e.g., a sample obtained via biopsy). The method can comprise: contacting a sample with the cryopreservation reagent, thereby generating a shielded sample. The method can comprise: exposing the shielded sample to a freezing storage condition, thereby generating a cryopreserved sample. Disclosed herein include methods of sample preservation. The method can comprise: exposing a shielded sample to a storage condition, thereby generating a preserved sample. The method can comprise: removing the preserved sample from the storage condition (e.g., thawing, change in temperature). In some embodiments, the method comprises, prior to the contacting step: contacting the sample with a sample preservation composition; and/or contacting the sample with a cell dispersion composition capable of generating a single cell suspension. Disclosed herein include methods of sample analysis. The method can comprise: providing a sample; and providing a composition (e.g., cryopreservation reagent) disclosed herein. The method can comprise: contacting the sample with the composition (e.g., cryopreservation reagent), thereby generating a shielded sample. The method can comprise: exposing the shielded sample to a freezing storage condition, thereby generating a cryopreserved sample. The method can comprise: thawing the cryopreserved sample. The method can comprise: conducting one or more measurements of one or more cellular properties. In some embodiments, the method comprises, prior to and/or after the contacting step: contacting the sample with a sample preservation composition; and/or contacting the sample with a cell dispersion composition capable of generating a single cell suspension. In some embodiments, the method does not comprise fixation and/or cancelling crosslinking by enzyme and/or heat. In some embodiments, contacting comprises immersing. The method can comprise generating a single cell suspension (e.g., before or after contact with the composition (e.g., cryopreservation reagent)). Providing a sample can comprise obtaining a sample from a subject (e.g., a tissue sample obtained via biopsy) as described herein. The length of time of the thawing step and conditions of the thawing step (e.g., temperature, agitation) can vary depending on the needs of the user.

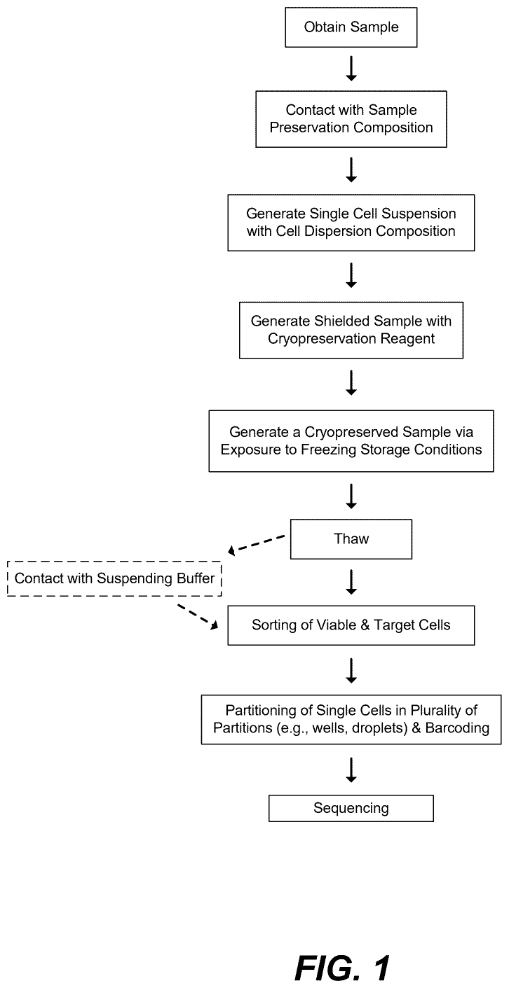

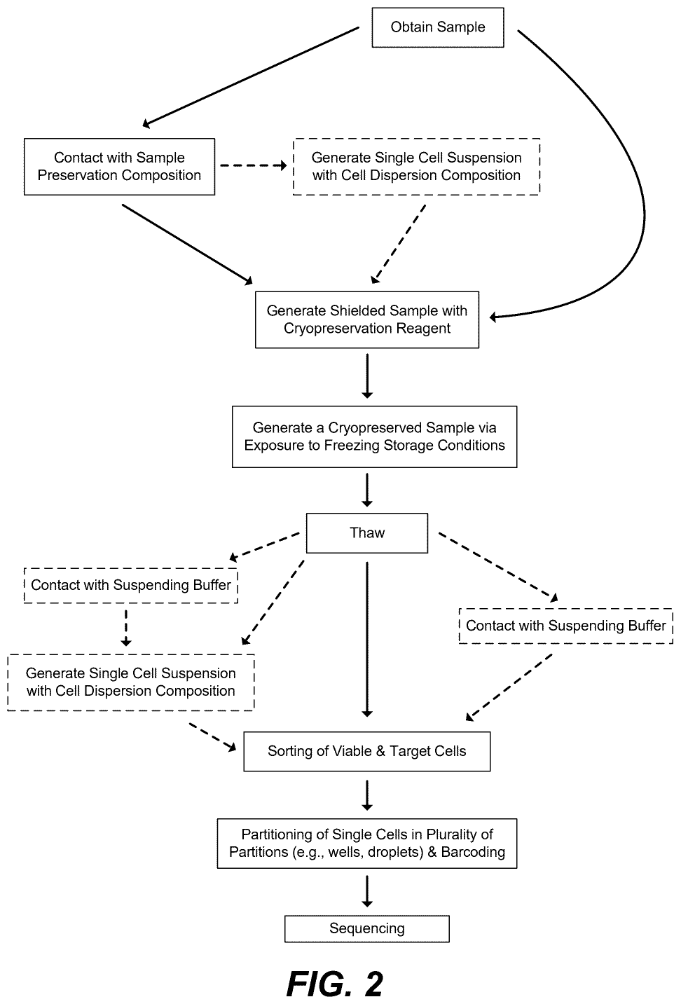

Commercially available sample preservation products are insufficient to maintain the viability and biomarkers in clinical specimens for flow cytometry, single cell RNAseq, and biobanking. Issues of currently available preservation methods and compositions include reduced cell viabilities, change in biomarkers, and a need fixation for long-term storage (which can cause RNA degradation). The samples capable of being preserved by the methods and compositions provided herein include Biobank and clinical specimens, and are capable of preservation of viable tissues and cells at cold temperatures (e.g., −80° C., −196° C.) for long periods of time (e.g., more than 1 year). FIGS. 1-2 depict a non-limiting exemplary workflows of sample cryopreservation and analysis. In some embodiments, the workflow comprises a sample (e.g., tumor tissue) being obtained by a clinician (e.g., at a hospital). In some embodiments, the workflow comprises the sample being contacted with (e.g., immersed in) in a sample preservation composition (e.g., BD® Tumor & Tissue Preservation Reagent (TTPR, BD catalog no. 664648); See also BD® OMICS-Guard Sample Preservation Buffer). In some embodiments, the workflow comprises the sample contacted with a cell dispersion solution (e.g., BD Horizon™ Dri Tumor & Tissue Dissociation Reagent (TTDR)) to generate a sample suspension comprising a plurality of single cells. In some embodiments, the sample is treated with TTDR within 24˜48 hours for cryopreservation and, in some embodiments, filtration is not necessary to exclude tissue crumbles. As depicted in FIG. 2, the workflow steps can vary, and in some embodiments, the sample is contacted with a cell dispersion composition before or after cryopreservation. In some embodiments, the sample or a derivation thereof (e.g., sample contacted with TTPR and/or TTDR) is cryopreserved using a composition provided herein (e.g., TTCR) to generate a cryopreserved sample (e.g., BioBank/Lab stock). The workflow can comprise thawing the cryopreserved sample. In some embodiments, thawing the cryopreserved sample comprises thawing at a temperature between about 32° C. and 42° C. (e.g., thawing in a 37° C. water bath). In some embodiments, the method comprises contacting the thawed sample with a suspending buffer provided herein (e.g., adding the thawed sample into the suspending buffer). The suspending buffer can be about 32° C.-42° C. (e.g., 37° C.) when contacted. The method can further comprise one or more centrifugations steps (e.g., 400 g at 4° C.) and/or one or more washes (e.g., washes with cold 2% FBS/RPMI). In some embodiments provided herein, contacting the cryopreserved sample with a suspending buffer provided herein yields greater maintenance of one or more cellular properties and/or cell viability of the plurality of cells of a thawed cryopreserved sample. The workflow can comprise TIL isolation and/or analysis (e.g., one or more steps described in Kobayashi et al., “Isolation of tumor-infiltrating lymphocytes from preserved human tumor tissue specimens for downstream characterization.” STAR protocols 3.3 (2022): 101557). The workflow can comprise sorting of live and target cells. The workflow can comprise partitioning of cells, barcoding of associated nucleic acids, and sequencing (e.g., long read of RNA-seq) for gene and/or protein analysis. The compositions and methods provided herein can be employed in solid tumor assays, flow cytometry-based assays, and tumor infiltrating lymphocytes (TILs) assays, and can be employed in immunotherapy clinical research, drug development, and diagnostics (e.g., companion diagnostics). TIL assays can be employed for biomarker discovery and for companion diagnostics. The efficacy of cancer immunotherapies, such as immune checkpoint inhibitor therapies (e.g., PD-(L)1 inhibitors), can be improved using the disclosed cryopreservation compositions and methods.

The methods provided herein can comprise the step of treating a sample with an enzyme to prepare the sample suspension (e.g., disperse the sample, in particular cells, tissues or mixtures thereof) and can be carried out by any method known to persons skilled in the art. Any enzyme can be used for the enzyme treatment. Examples of enzymes that can be used for the enzyme treatment include, but are not limited to, collagenase, elastase, hyaluronidase, neutral proteases including dispase, glycosidase, deoxyribonuclease (DNase), and trypsin. Enzyme treatment may also be performed using existing cell dispersion solutions (e.g., TTDR). The effect of dispersing cells or tissues by enzyme treatment varies depending on factors such as the type of enzyme used, enzyme concentration, treatment temperature, and treatment time. In particular, the type of enzyme used and the enzyme concentration significantly affect the dispersed state of the tissue. The enzyme treatment can be carried out at any temperature (e.g., 1 to 45° C., 10 to 40° C., 15 to 30° C.) in order to obtain sufficient dispersion without changing the state of the sample. Enzyme treatment time can vary (e.g., about 1 to 60 minutes, about 5 to 30 minutes about 10-20 minutes) in order to avoid changing the state of the sample and obtain adequate dispersion. The enzyme treatment may be performed in a still state or while stirring or shaking. The step of preparing the sample suspension may include any additional operations such as filtration, centrifugation, and staining. The method can comprise contacting the sample with the cell dispersion composition within about 24 hr to about 48 hr of contacting the sample with a sample preservation composition. The cell dispersion composition can comprise one or more enzymes selected from the group comprising collagenase, elastase, hyaluronidase, dispase, glycosidase, deoxyribonuclease, trypsin, or any combination thereof. In some embodiments, the sample preservation composition comprises: albumin (optionally 0.01-2% (w/v)); and lactic acid or a salt thereof (optionally 0.1-100 mmol/L). In some embodiments, the sodium ion concentration of the sample preservation composition is about 50-300 mmol/L; the albumin is bovine serum albumin (BSA); the lactic acid or salt thereof is sodium lactate; the sample preservation composition comprises about 0.01-10 mmol/L of potassium dihydrogen phosphate; the sample preservation composition comprises about 0.01-50 mmol/L of disodium hydrogen phosphate the sample preservation composition comprises a polysaccharide (e.g., trehalose, a fructooligosaccharide, indigestible dextrin, or any combination thereof).

The freezing storage condition can comprise a temperature of about 0° C., −2° C., −4° C., −6° C., −8° C., −10° C., −12° C., −14° C., −16° C., −18° C., −20° C., −22° C., −24° C., −26° C., −28° C., −30° C., −32° C., −34° C., −36° C., −38° C., −40° C., −42° C., −44° C., −46° C., −48° C., −50° C., −52° C., −54° C., −56° C., −58° C., −60° C., −62° C., −64° C., −66° C., −68° C., −70° C., −72° C., −74° C., −76° C., −78° C., −80° C., −82° C., −84° C., −86° C., −88° C., −90° C., −92° C., −94° C., −96° C., −98° C., −100° C., −102° C., −104° C., −106° C., −108° C., −110° C., −112° C., −114° C., −116° C., −118° C., −120° C., −122° C., −124° C., −126° C., −128° C., −130° C., −132° C., −134° C., −136° C., −138° C., −140° C., −142° C., −144° C., −146° C., −148° C., −150° C., −152° C., −154° C., −156° C., −158° C., −160° C., −162° C., −164° C., −166° C., −168° C., −170° C., −172° C., −174° C., −176° C., −178° C., −180° C., −182° C., −184° C., −186° C., −188° C., −190° C., −192° C., −194° C., −196° C., a number or a range between any two of the values, or any combination thereof. After thawing the cryopreserved sample, at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of the values, of the plurality of cells can be viable. The value(s) of the one or more cellular properties after thawing the cryopreserved sample can be less than about 1.01-fold, 1.02-fold, 1.04-fold, 1.06-fold, 1.08-fold, 1.1-fold, 1.15-fold, 1.2-fold, 1.25-fold, 1.3-fold, 1.35-fold, 1.4-fold, 1.45-fold, 1.5-fold, or a number or a range between any two of the values, different than said value(s) prior to exposing the shielded sample to a freezing storage condition. The plurality of cells can be all the cells present in the sample or a subset of the cells in the same.

Conducting one or more measurements can comprise conducting one or more measurements on a plurality of samples in parallel. Conducting one or more measurements can comprise conducting a nucleic acid sequencing assay, a next generation nucleic acid sequencing (NGS) assay, a PCR assay, a quantitative PCR (qPCR) assay, a reverse transcription PCR (RT-PCR) assay, a miRNA assay, a microarray assay, a Northern blot assay, a Southern blot assay, a luciferase assay, a fluorescence immunoassay, a radio immunoassay, an enzyme-linked immunosorbent assay (ELISA), a flow cytometry assay, a mass spectrometry (MS) assay, a Selected Reaction Monitoring (SRM-MS) assay, a Sequential Windowed data independent Acquisition of the Total High resolution Mass Spectroscopy (SWATH-MS) assay, a Western blot assay, a genome wide methylation assay, a targeted methylation assay, a bisulfite methylation sequencing assay, a restriction enzyme methylation sequencing assay, a high performance liquid chromatography (HPLC) assay, an ultrahigh performance liquid chromatography (UHPLC) assay, a mass spectrometry (MS) assay, an ultrahigh performance liquid chromatography/tandem mass spectrometry (UHPLC/MS/MS2), a gas chromatography/mass spectrometry (GC/MS) assay, a lipidomics assay, a cell aging assay, an endocrine assay, a neuroendocrine assay, a cytokine assay, an immune cell assay, or any combination thereof. Conducting one or more measurements can comprise measuring emissions of a detectable moiety with a flow cytometer (e.g., a conventional flow cytometer, a spectral flow cytometer, a hyperspectral flow cytometer, an imaging flow cytometer, or any combination thereof). Suitable flow cytometry systems may include, but are not limited to those described in Ormerod (ed.), Flow Cytometry: A Practical Approach, Oxford Univ. Press (1997); Jaroszeski et al. (eds.), Flow Cytometry Protocols, Methods in Molecular Biology No. 91, Humana Press (1997); Practical Flow Cytometry, 3rd ed., Wiley-Liss (1995); Virgo, et al. (2012) Ann Clin Biochem. January; 49 (pt 1): 17-28; Linden, et. al., Semin Throm Hemost. 2004 October; 30 (5): 502-11; Alison, et al. J Pathol, 2010 December; 222 (4): 335-344; and Herbig, et al. (2007) Crit Rev Ther Drug Carrier Syst. 24 (3): 203-255; the disclosures of which are incorporated herein by reference. In certain instances, flow cytometry systems of interest include BD Biosciences FACSCanto™ flow cytometer, BD Biosciences FACSCanto™ II flow cytometer, BD Accuri™ flow cytometer, BD Accuri™ C6 Plus flow cytometer, BD Biosciences FACSCelesta™ flow cytometer, BD Biosciences FACSLyric™ flow cytometer, BD Biosciences FACSVerse™ flow cytometer, BD Biosciences FACSymphony™ flow cytometer, BD Biosciences LSRFortessa™ flow cytometer, BD Biosciences LSRFortessa™ X-20 flow cytometer, BD Biosciences FACSPresto™ flow cytometer, BD Biosciences FACSVia™ flow cytometer and BD Biosciences FACSCalibur™ cell sorter, a BD Biosciences FACSCount™ cell sorter, BD Biosciences FACSLyric™ cell sorter, BD Biosciences Via™ cell sorter, BD Biosciences Influx™ cell sorter, BD Biosciences Jazz™ cell sorter, BD Biosciences Aria™ cell sorter, BD Biosciences FACSAria™ II cell sorter, BD Biosciences FACSAria™ III cell sorter, BD Biosciences FACSAria™ Fusion cell sorter and BD Biosciences FACSMelody™ cell sorter, BD Biosciences FACSymphony™ S6 cell sorter or the like. In some embodiments, the subject systems are flow cytometric systems, such those described in U.S. Pat. Nos. 10,663,476; 10,620,111; 10,613,017; 10,605,713; 10,585,031; 10,578,542; 10,578,469; 10,481,074; 10,302,545; 10,145,793; 10,113,967; 10,006,852; 9,952,076; 9,933,341; 9,726,527; 9,453,789; 9,200,334; 9,097,640; 9,095,494; 9,092,034; 8,975,595; 8,753,573; 8,233,146; 8,140,300; 7,544,326; 7,201,875; 7,129,505; 6,821,740; 6,813,017; 6,809,804; 6,372,506; 5,700,692; 5,643,796; 5,627,040; 5,620,842; 5,602,039; 4,987,086; 4,498,766; the disclosures of which are herein incorporated by reference in their entirety. Conducting one or more measurements can comprise imaging of single cell(s). Imaging can comprise microscopy, confocal microscopy, time-lapse imaging microscopy, microscopy, multi-photon microscopy, quantitative phase microscopy, surface enhanced Raman spectroscopy, videography, manual visual analysis, automated visual analysis, or any combination thereof.

The plurality of cells can comprise a plurality of cellular component targets, and conducting one or more measurements can comprise contacting a plurality of cellular component-binding reagents the plurality of cells. Each of the plurality of cellular component-binding reagents can comprise a cellular component-binding reagent specific oligonucleotide comprising a unique identifier sequence for the cellular component-binding reagent, and wherein the cellular component-binding reagent is capable of specifically binding to at least one of the plurality of cellular component targets. Each of the plurality of cellular component-binding reagents can comprise a detectable moiety, or a precursor thereof. In some embodiments, the cellular component-binding reagent is capable of specifically binding to at least one of the plurality of cellular component targets, wherein cellular component-binding reagents capable of binding the same cellular component target comprise the same detectable moiety, or a precursor thereof, and wherein cellular component-binding reagents capable of binding different cellular component targets comprise different detectable moieties, or precursors thereof. The cellular component-binding reagent can comprise an antibody or fragment thereof. The plurality of cellular component targets can be selected from the group comprising CD11b, CD11c, CD33, CD14, CD185 (CXCR5), CD19, CD25, CD27, CD278, CD279, CD3, CD163, CD357 (GITR), CD366 (Tim3), CD4, CD45RA, CD56, CD62L, CD197 (CCR7), CD186 (CXCR6), CD127, CD134, CD28, CD272, CD8, HLA-DR/DP/DQ, CD16, CD183, CD196 (CCR6), CD137, CD161, IgM, CD152, and IgD.

Conducting one or more measurements can comprise flow cytometrically sorting the plurality of cells, such as fluorescence-activated cell sorting (FACS), and said sorting can based on intracellular staining and/or surface marker staining (e.g., staining with cellular component-binding reagent(s)). The nucleic acid target can comprise a sample indexing oligonucleotide. The sample indexing oligonucleotide can comprise a sample indexing sequence. The sample indexing sequences of at least two sample indexing compositions of a plurality of sample indexing compositions provided herein can comprise different sequences. The nucleic acid target can comprise a cellular component-binding reagent specific oligonucleotide. A cellular component-binding reagent specific oligonucleotide can comprise a unique identifier sequence for a cellular component-binding reagent. In some embodiments of the methods and compositions provided herein the nucleic acid target is a binding reagent oligonucleotide (e.g., antibody oligonucleotide (“AbOligo” or “AbO”), binding reagent oligonucleotide, cellular component-binding reagent specific oligonucleotide, sample indexing oligonucleotide). Some embodiments disclosed herein provide a plurality of compositions each comprising a cellular component binding reagent (such as a protein binding reagent) that is conjugated with an oligonucleotide (e.g., a binding reagent oligonucleotide), wherein the oligonucleotide comprises a unique identifier for the cellular component binding reagent that it is conjugated with. Conducting one or more measurements can comprise protein profiling and/or sample indexing. Cellular component binding reagents (such as barcoded antibodies) and their uses (such as sample indexing of cells) have been described in U.S. Patent Application Publication No. US2018/0088112 and U.S. Patent Application Publication No. US2018/0346970; the content of each of these is incorporated herein by reference in its entirety.

Conducting one or more measurements can comprise analysis by any means described herein of the following target molecules: (i) an agonist of a co-stimulatory receptor and/or (ii) an antagonist of an inhibitory signal on T cells, both of which can result in amplifying antigen-specific T cell responses (immune checkpoint regulators). Exemplary targets of analysis include, for example, members of the immunoglobulin super family (IgSF); a member of a family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6; a member of the TNF family of molecules that bind to cognate TNF receptor family members, which include CD40 and CD40L, OX-40, OX-40L, CD70, CD27L, CD30, CD30L, 4-1BBL, CD137, GITR, TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LT.beta.R, LIGHT, DcR3, HVEM, VEGI/TL1A, TRAMP/DR3, EDAR, EDA1, XEDAR, EDA2, TNFR1, Lymphotoxin a/TNFβ, TNFR2, TNFα, LTβR, Lymphotoxin a 1 (32, FAS, FASL, RELT, DR6, TROY, NGFR (see, e.g., Tansey (2009) Drug Discovery Today 00:1); CTLA-4, PD-1, PD-L1, PD-L2, and LAG-3, TIM-3, Galectin 9, CEACAM-1, BTLA, CD69, Galectin-1, TIGIT, CD113, GPR56, VISTA, 2B4, CD48, GARP, CD73, PD1H, LAIR1, TIM-1, TIM-4, CD39, B7-1, B7-2, CD28, 4-1BB (CD137), 4-1BBL, GITR, GITRL, ICOS, ICOS-L, OX40, OX40L, CD70, CD27, CD40, DR3 and CD28H; inhibitory receptors on NK cells or agonists of activating receptors on NK cells, e.g., KIR; CSF-1; CSF-R; TGF-beta; molecules on immune cells, e.g., T reg cells, macrophages, monocytes, CD73, BTLA, VISTA, LAG-3, B7-H3, B7-H4, IDO, TIGIT, MICA/B, or arginase; CD40, CD226, CD28, OX40, GITR, CD137, CD27, HVEM, or CD127; OX40, CD2, CD27, CDS, ICAM-1, LFA-1 (CD11a/CD18), ICOS (CD278), 4-1BB (CD137), 20 GITR, CD30, CD40, BAFFR, HVEM, CD7, LIGHT, NKG2C, SLAMF7, NKp80, CD160, B7-H3 or CD83 ligand. PD-1, PD-L1, PD-L2, CTLA4, TIM3, LAG3, VISTA, BTLA, TIGIT, LAIR1, CD160, 2B4 and/or TGFR beta, and HVEM.

The plurality of cells can comprise a plurality of nucleic acid target molecules (e.g., ribonucleic acids (RNAs), messenger RNAs (mRNAs), microRNAs, small interfering RNAs (siRNAs), RNA degradation products, RNAs each comprising a poly(A) tail, and any combination thereof). Conducting one or more measurements can comprise partitioning single cells of the plurality of cells to a plurality of partitions (e.g., a plurality of droplets or microwells of a microwell array). Conducting one or more measurements can comprise conducting one or more sequencing assays to generate a plurality of sequence reads. The sequencing assay(s) can comprise single cell (sc) sequencing assay(s) and/or long read RNA-seq. In some embodiments, conducting the one or more sequencing assays comprises: stochastically barcoding the nucleic acid target molecules using a plurality of stochastic barcodes to generate a plurality of stochastically barcoded target nucleic acid molecules, wherein each of the plurality of stochastic barcodes comprises a cell label and a molecular label, wherein molecular labels of at least two stochastic barcodes of the plurality of stochastic barcodes comprise different molecular label sequences, wherein the stochastic barcodes associated with the same cell comprise the same cellular label, and wherein the cellular labels associated with different cells comprise different cellular labels. Barcoding, such as stochastic barcoding, has been described in, for example, Fu et al., Proc Natl Acad Sci U.S.A., 2011 May 31,108 (22): 9026-31; U.S. Patent Application Publication No. US2011/0160078; Fan et al., Science, 2015 Feb. 6, 347 (6222): 1258367; US Patent Application Publication No. US2015/0299784; and PCT Application Publication No. WO2015/031691; the content of each of these, including any supporting or supplemental information or material, is incorporated herein by reference in its entirety. As used herein, the term “stochastic barcoding” can refer to the random labeling (e.g., barcoding) of nucleic acids. Stochastic barcoding can utilize a recursive Poisson strategy to associate and quantify labels associated with targets. As used herein, the term “stochastic barcoding” can be used interchangeably with “stochastic labeling.” As used herein, the term “stochastic barcode” can refer to a polynucleotide sequence comprising labels. A stochastic barcode can be a polynucleotide sequence that can be used for stochastic barcoding. Stochastic barcodes can be used to quantify targets within a sample. Stochastic barcodes can be used to control for errors which may occur after a label is associated with a target. For example, a stochastic barcode can be used to assess amplification or sequencing errors. A stochastic barcode associated with a target can be called a stochastic barcode-target or stochastic barcode-tag-target. As used here, the term “target” can refer to a composition which can be associated with a barcode (e.g., a stochastic barcode). Exemplary suitable targets for analysis include oligonucleotides, DNA, RNA, mRNA, microRNA, tRNA, and the like. Targets can be single or double stranded. In some embodiments, targets can be proteins, peptides, or polypeptides. As used herein, “target” can be used interchangeably with “species.” Methods for determining the sequences of a nucleic acid target (e.g., the V (D) J region of an immune receptor) using 5′ barcoding and/or 3′ barcoding are described in US2020/0109437; the content of which is incorporated herein by reference in its entirety. Systems, methods, compositions, and kits for molecular barcoding on the 5′-end of a nucleic acid target have been described in US2019/0338278, the content of which is incorporated herein by reference in its entirety. The compositions and methods provided herein can, in some embodiments, be employed in concert with random priming and extension (RPE)-based whole transcriptome analysis methods and compositions have been described in U.S. patent application Ser. No. 16/677,012; the content of which is incorporated herein by reference in its entirety.

Conducting one or more measurements can comprise generating values for one or more cellular properties derived from the sequence reads. The one or more cellular properties can comprise one or more genomic properties, one or more expression properties, and/or one or more variant properties. The one or more genomic properties can be derived from one or more sequencing assays comprising a bisulfite sequencing assay, single cell bisulfite sequencing assay, assay for Transposase-Accessible Chromatin using sequencing (ATAC-seq), single cell (sc) ATAC-seq, or any combination thereof. Systems, methods, compositions, and kits for high throughput multiomics sample analysis (e.g. including analysis of genome, genomic accessibility (e.g., chromatin accessibility), and methylome) have been described in US2023/0295723, the content of which is incorporated herein by reference in its entirety. The one or more expression properties can be derived from one or more sequencing assays comprising sequence-mediated protein profiling, single cell sequence-mediated protein profiling, RNA-sequencing (RNA-seq), single cell (sc) RNA-seq, or any combination thereof. The one or more variant properties can be derived from sequencing assays comprising barcoded sequencing, random sequencing, whole genome sequencing, targeted sequencing, next generation sequencing, or any combination thereof. The one or more variant properties can comprise a single nucleotide polymorphism (SNP), an insertion or deletion (indel), a copy number variant (CNV), a fusion, a splice variant, an isoform variant, a transversion, a translocation, a frame shift, a duplication, a repeat variant, or any combination thereof, at one or more loci of a plurality of loci. The one or more genomic properties can comprise chromatin accessibility, hypomethylation and/or hypermethylation at one or more loci of a plurality of loci. The one or more expression properties can comprise underexpression of one or more mRNAs of interest, underexpression of one or more proteins of interest, overexpression of one or more mRNAs of interest, and/or overexpression of one or more proteins of interest. The one or more mRNAs of interest and/or one or more proteins of interest can be derived from one or more loci of a plurality of loci.

The plurality of loci can be selected from a predetermined set of loci that includes less than all loci in the genome of the subject. The predetermined set of loci can comprise at least 10 loci. The predetermined set of loci can comprise from 100 to 100,000 loci, from 100 to 50,000 loci, from 100 to 25,000 loci, from 100 to 10,000 loci, from 100 to 5000 loci, from 100 to 2000 loci, from 100 to 1000 loci, from 500 to 100,000 loci, from 500 to 50,000 loci, from 500 to 25,000 loci, from 500 to 10,000 loci, from 500 to 5000 loci, from 500 to 2000 loci, from 500 to 1000 loci, from 1000 to 100,000 loci, from 1000 to 50,000 loci, from 1000 to 25,000 loci, from 1000 to 10,000 loci, from 1000 to 5000 loci, from 1000 to 2000 loci, or a number or a range between any two of the values. The predetermined set of loci can be known to be associated with cancer (e.g., the predetermined set of loci comprise tumor suppressor genes and/or oncogenes). The predetermined set of loci can comprise a cancer-related gene selected from the group consisting of: AKT1, ALK, APC, AR, ARAF, ARID 1 A, ARID2, ATM, B2M, BCL2, BCOR, BRAF, BRCA1, BRCA2, CARD11, CBFB, CCND1, CDH1, CDK4, CDKN2A, CIC, CREBBP, CTCF, CTNNB1, DICER 1, DIS3, DNMT3A, EGFR, EIF1AX, EP300, ERBB2, ERBB3, ERCC2, ESRI, EZH2, FBXW7, FGFR1, FGFR2, FGFR3, FGFR4, FLT3, FOXA1, FOXL2, FOXO1, FUBP1, GAT A3, GNA11, GNAQ, GNAS, H3F3A, HIST1H3B, HRAS, IDH1, IDH2, IKZF1, INPPL1, JAK1, KDM6A, KEAP1, KIT, KNSTRN, KRAS, MAP2K1, MAPK1, MAX, MED 12, MET, MLH1, MSH2, MSH3, MSH6, MTOR, MYC, MYCN, MYD88, MYODI, NF1, NFE2L2, NOTCH1, NRAS, NTRK1, NTRK2, NTRK3, NUP93, PAK7, PDGFRA, PIK3CA, PIK3CB, PIK3R1, PIK3R2, PMS2, POLE, PPP2R1A, PPP6C, PRKCI, PTCHI, PTEN, PTPN11, RAC1, RAFI, RBI, RET, RHOA, RIT1, ROS1, RRAS2, RXRA, SETD2, SF3B1, SMAD3, SMAD4, SMARCA4, SMARCB1, SOS1, SPOP, STAT3, STK11, STK19, TCF7L2, TERT, TGFBR1, TGFBR2, TP53, TP63, TSC1, TSC2, U2AF1, VHL, and XPO1.

In some embodiments, generating values for one or more variant properties derived from the sequence reads can comprise aligning at least a portion of said sequence reads to the genome of a reference. In some embodiments, generating values for one or more expression properties derived from the sequence reads can comprise a comparison to the mRNA expression levels of interest and/or protein expression levels of interest a reference. In some embodiments, generating values for one or more genomic properties derived from the sequence reads can comprise a comparison to the methylation status and/or the chromatin accessibility at the one or more loci of a plurality of loci of a reference. The reference can comprise one or more patients having the same stage of cancer, the same type of cancer, or both, that the subject is suspect of having. The reference can comprise one or more unaffected individuals. The reference can comprise a biological sample obtained from the subject at an earlier time point. The reference can comprise a subject having cancer, a subject not having cancer, a subject having a stage I cancer, a subject having a stage II cancer, a subject having a stage III cancer, a subject having a stage IV cancer, or any combination thereof.

The method can comprise predicting a response or resistance to and/or a benefit from a therapy in a subject suffering from or being at risk of developing a neoplastic disease based on the measurement of the one or more cellular properties; and/or predicting the outcome of a therapy in a subject suffering from or being at risk of developing a neoplastic disease based on the measurement of the one or more cellular properties. The response, resistance, benefit and/or outcome can be the pathological complete response (pCR), loco-regional recurrence free interval (LRRFI), loco-regional invasive recurrence free interval (LRIRFI), distant-disease-free survival (DDFS), invasive disease-free survival (IDFS), event free survival (EFS) and/or overall survival (OS). The therapy can be an immunotherapy. The immunotherapy can comprise a checkpoint inhibitor, a chimeric antigen receptor T-cell therapy, an oncolytic vaccine, a cytokine agonist or a cytokine antagonist, or a combination thereof. The immunotherapy can comprise a PD-1 inhibitor, PD-L1 inhibitor, PD-L2 inhibitor, GITR agonist, 0X40 agonist, TIM3 agonist, LAG3 agonist, KIR agonist, CD28 agonist, CD137 agonist, CD27 agonist, CD40 agonist, CD70 agonist, CD276 agonist, ICOS agonist, HVEM agonist, NKG2D agonist, NKG2A agonist, MICA agonist, 2B4 agonist, 41BB agonist, CTLA4 antagonist, PD-1 axis antagonist, TIM3 antagonist, BTLA antagonist, VISTA antagonist, LAG3 antagonist, B7H4 antagonist, CD96 antagonist, TIGIT antagonist, CD226 antagonist or a combination thereof. The cytokine agonist or cytokine antagonist can be an agonist or antagonist of interferon, IL-2, GMCSF, IL-17E, IL-6, IL-la, IL-12, TFGB2, IL-15, IL-3, IL-13, IL-2R, IL-21, IL-4R, IL-7, M-CSF, MIF, myostatin, 11-10, 11-24, CEA, IL-11, IL-9, IL-15, IL-2Ra, TNF or a combination thereof. In some embodiments the therapy comprises an immune checkpoint modulatory agent. An immune checkpoint modulatory agent in some cases is at least one of a small molecule, an antibody, a nucleic acid encoding an antibody, an antigen binding fragment, a RNA interfering agent, a peptide, a peptidomimetic, a synthetic ligand, and an aptamer. The immune checkpoint inhibitor can be an antibody or antigen binding fragment that binds an immune checkpoint target, such as an anti-PD-1 antibody or and anti-PD-L1 antibody. Examples of immune checkpoint inhibitors are Enoblituzumab (e.g., MGA271), Ipilimumab (e.g, BMS-734016, MDX-010), Tremelimumab (e.g., CP-675, CP-675,206), Lirilumab (e.g, BMS-986015, IPH2102), BMS986016, Pembrolizumab (e.g, MK-3475, SCH 900475), Nivolumab (e.g, BMS-936558, MDX-1106, ONO-4538), Pidilizumab (e.g, CT-011, MDV9300), Atezolizumab (e.g, MPDL3280A, RG7446, R05541267), BMS-936559 (e.g, MDX-1 105), Durvalumab, Avelumab, and Bavituximab.

### EXAMPLES

Some aspects of the embodiments discussed above are disclosed in further detail in the following examples, which are not in any way intended to limit the scope of the present disclosure.

### Example 1

### Cell Cryopreservation Buffers

In this Example (1) tumor tissue specimens were treated with TTDR to generate a single cell suspension, and the cell suspension is equally divided in test vials and (2) for Day 0, samples were stained immediately after Step 1. Cells were stained with FVS780, CD3, CD4, CD8, CD45 and/or CD25, and fixation & permeabilization were performed. The composition of cryopreservation buffers tested and representative FACS data before/after cryopreservation is described below. Specimens employed are provided in Table 1.

TABLE 2
Specimens

| | | Immersing hours | |
|---|---|---|---|
| | | of tumor tissue in | |
| | | TTPR until tumor | |
| Specimen | | tissue is | Cryopreserve |
| No. | Sample ID | processed | Days |
| 1 | Jurkat (cell strain) | — | Day 13 (−80° C.) |
| 2 | Lung cancer (Tissue-1) | within 24 hours | Day 14 (−80° C.) |
| 3 | Lung cancer (Tissue-2) | 2 days | Day 14 (−80° C.) |
| 4 | Lung cancer (Tissue-3) | within 24 hours | Day 11 (−80° C.) |
| 5 | Lung cancer (Tissue-4) | within 24 hours | Day 13 (−80° C.) |
| 6 | Lung cancer (Tissue-5) | within 24 hours | Day 12 (−80° C.) |
| 7 | Lung cancer (Tissue-5) | within 24 hours | Day 14 (−80° C.) |
| 8 | Lung cancer (Tissue-6) | 3 days | Day 10 (−80° C.) |
| 9 | Lung cancer (Tissue-7) | within 24 hours | Day 10 (−80° C.) |
| 10 | Lung cancer (Tissue-8) | within 24 hours | Day 10 (−80° C.) |

### Specimen No. 1

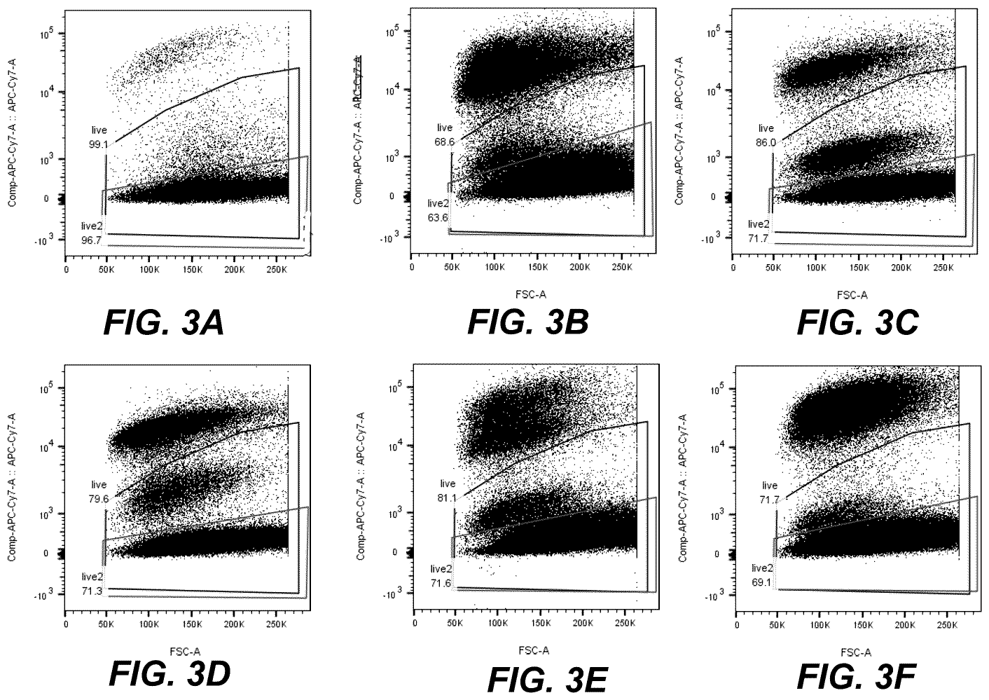

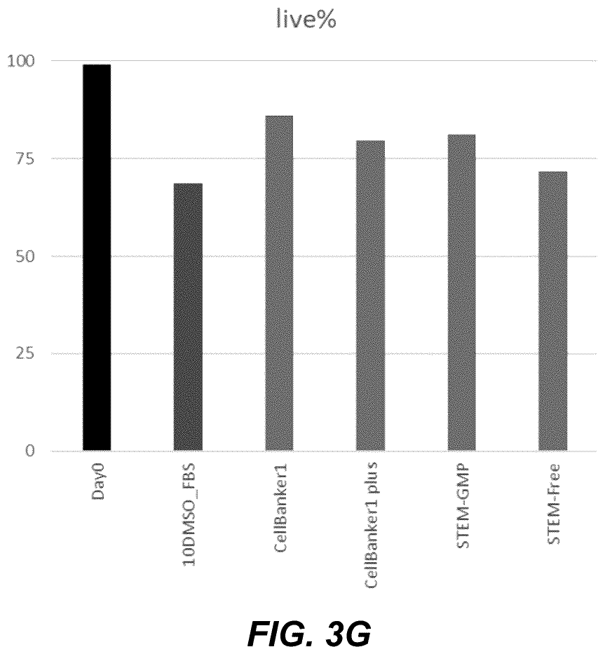

Commercially available products (CELLBANKER® series: 4 products) and the conventional buffer (10% DMSO/FBS) were compared to analyze the viability using FVS780 before and after freezing. FIGS. 3A-3G depict fluorescence-activated cell sorting (FACS) data related to Specimen No. 1 viability (using FVS780) at Day 0 (FIG. 3A) and at Cryopreservation Day 13 using conventional buffer (10% DMSO/FBS; FIG. 3B) and using commercially available products CELLBANKER® 1 (FIG. 3C), CELLBANKER® 1 plus (FIG. 3D), STEM-CellBanker® GMP grade (FIG. 3E), STEM-CellBanker® DMSO-Free GMP grade (FIG. 3F), as well as a quantification of said FACS data (FIG. 3G). It was found that CELLBANKER® 1 is better than 10% DMSO/FBS.

### Specimen No. 2

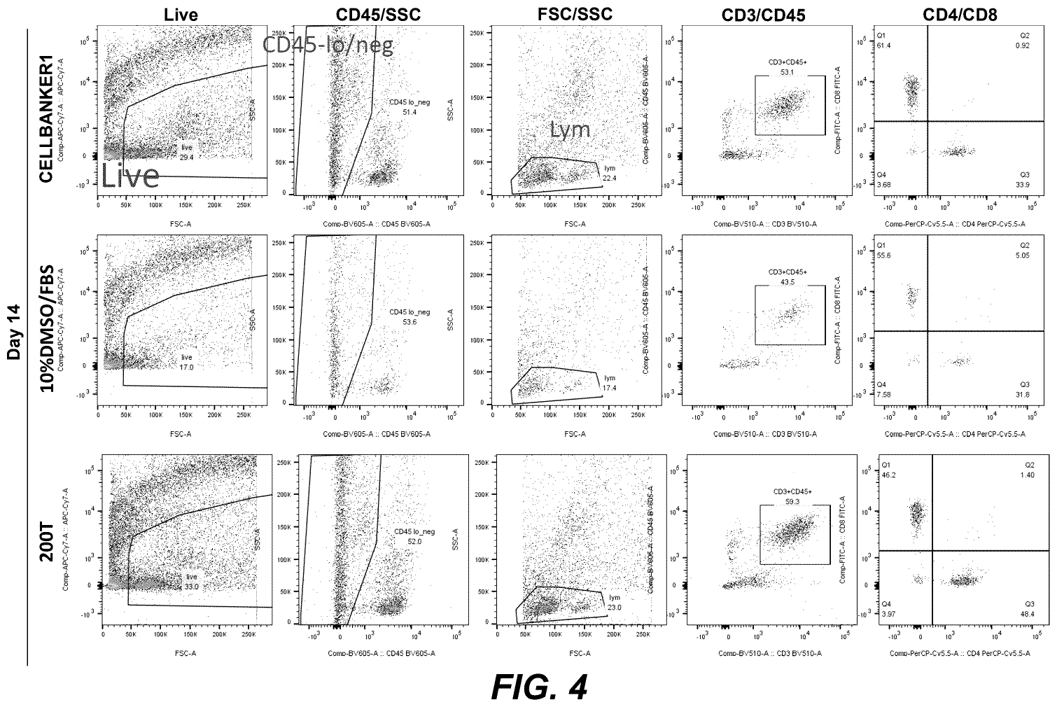

Cryopreservation buffers tested using Specimen No. 2 are described in Table 3. FIG. 4 depicts FACS data related to viability, CD45/SSC, FSC/SSC, CD3/CD45, and CD4/CD8 properties for Specimen No. 2 at Cryopreservation Day 13 using cryopreservation buffers CELLBANKER® 1, 10% DMSO/FBS, and 200T. It was found that 10% DMSO/FBS <CELLBANKER® 1<200T. CELLBANKER® 1 or 10% DMSO/FBS were used as a control in the following experiments.

TABLE 3
Cryopreservation Buffers

| Cryopreservation | |
|---|---|
| Buffer | Composition |
| CELLBANKER ® 1 | Commercial product |
| 10% DMSO/FBS | 10% DMSO, 90% FBS |
| 200T | 5% DMSO, 5% EG, 50% FBS, 200 mM |
| | Trehalose/RPMI-based |

### Specimen No. 3

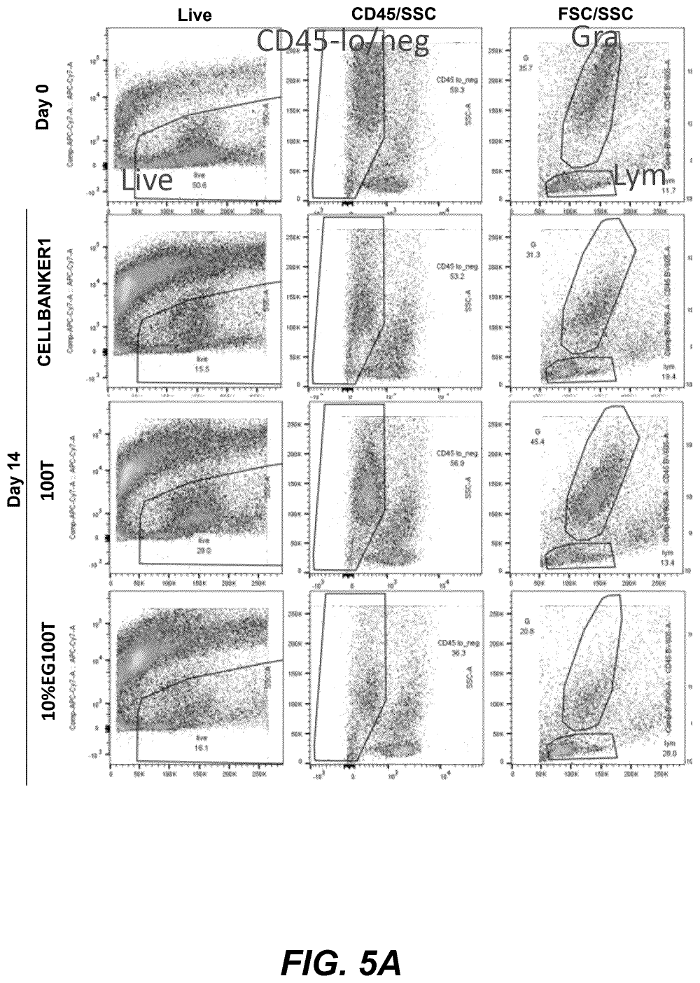

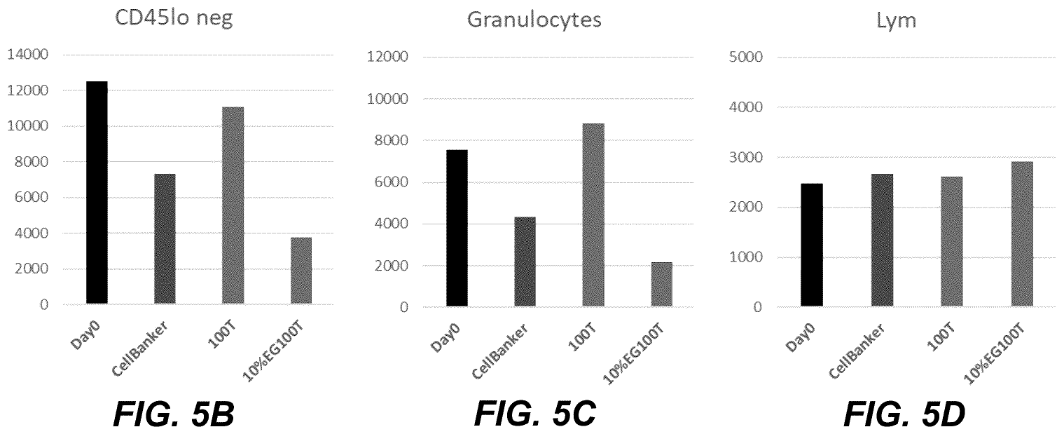

Cryopreservation buffers tested using Specimen No. 3 are described in Table 4. FIGS. 5A-5D depict FACS data related to viability, CD45/SSC, FSC/SSC properties for Specimen No. 3 at Day 0 and at Cryopreservation Day 14 using cryopreservation buffers CELLBANKER® 1, 100T, 10% EG100T, and CELLBANKER® 1 (FIG. 5A) and cell number quantifications derived therefrom (FIGS. 5B-5D) for CD45lo neg cells (FIG. 5B), granulocytes (FIG. 5C), and lymphocytes (lym) (FIG. 5D). It was found that CELLBANKER® 1 and 10% EG100T can preserve lymphocytes, but are not good to preserve CD45-lo/neg cells and granulocytes. 100T was able to store cells at the Day 0/Fresh level. These results indicate that the balance of the anti-freeze components (DMSO&EG) is important.

TABLE 4
Cryopreservation Buffers

| Cryopreservation | |
|---|---|
| Buffer | Composition |
| CELLBANKER ® 1 | Commercial product |
| 100T | 5% DMSO, 5% EG, 50% FBS, 100 mM |
| | Trehalose/RPMI-based |
| 10% EG100T | 10% EG, 50% FBS, 100 mM |
| | Trehalose/RPMI-based |

### Specimen No. 4

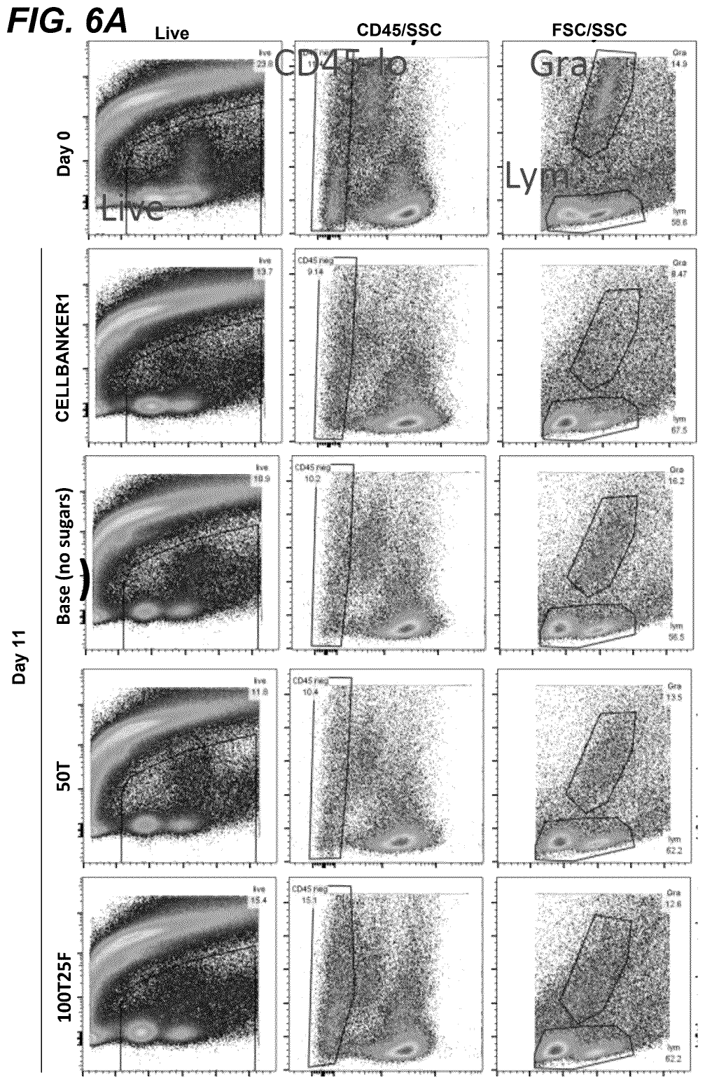

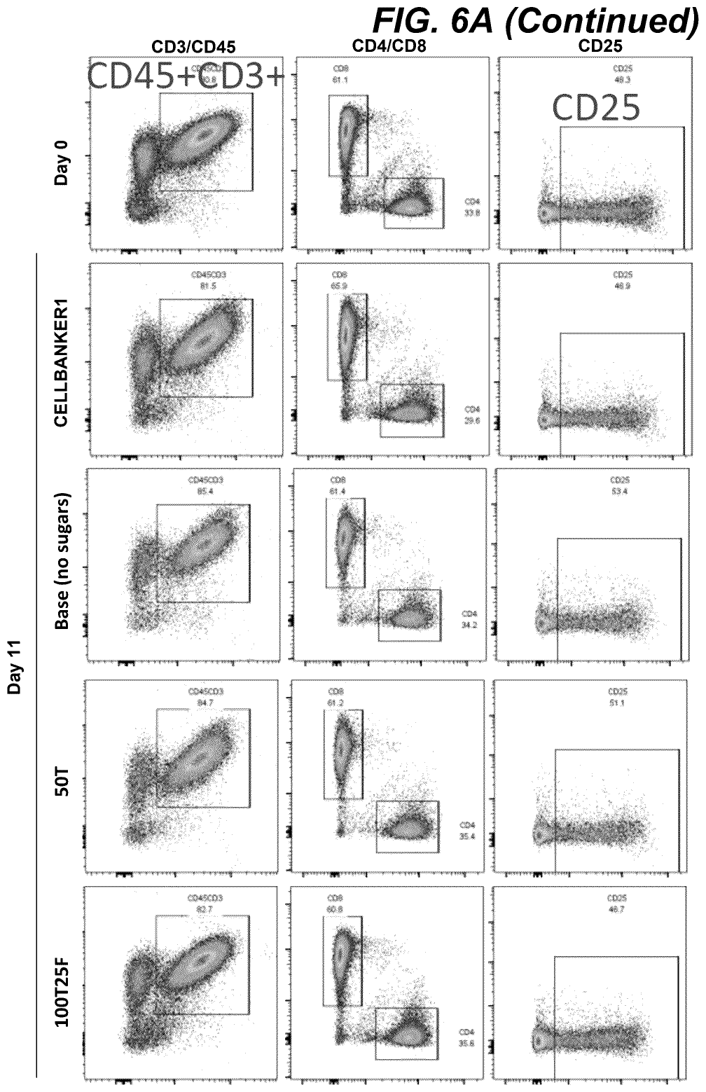

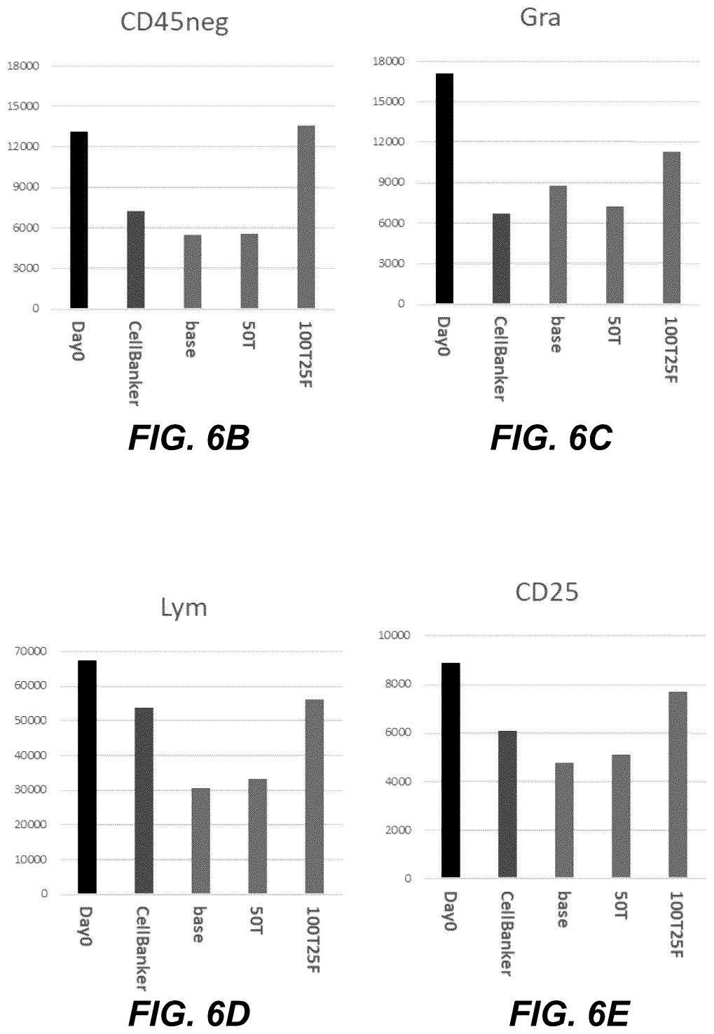

Cryopreservation buffers tested using Specimen No. 4 are described in Table 5. FIGS. 6A-6E depict FACS data related to viability, CD45/SSC, CD45/SSC, FSC/SSC, CD3/CD45, CD4/CD8, and CD25 properties for Specimen No. 4 at Day 0 and at Cryopreservation Day 11 using cryopreservation buffers CELLBANKER® 1, base (no sugars), 50T, and 100T25F (FIG. 6A) and cell number quantifications derived therefrom (FIGS. 6B-6E) for CD45neg (FIG. 6B), granulocytes (FIG. 6C), lymphocytes (Lym) (FIG. 6D), and CD25 (FIG. 6E) cells.

TABLE 5
Cryopreservation Buffers

| Cryopreservation | |
|---|---|
| Buffer | Composition |
| CELLBANKER ® 1 | Commercial product |
| Base (no sugars) | 5% DMSO, 5% EG, 50% FBS/RPMI-based |
| 50T | 5% DMSO, 5% EG, 50% FBS, 50 mM |
| | Trehalose/RPMI-based |
| 100T25F | 5% DMSO, 5% EG, 50% FBS, 100 mM |
| | Trehalose + 25 mM Fructose/RPMI-based |

### Specimen No. 5

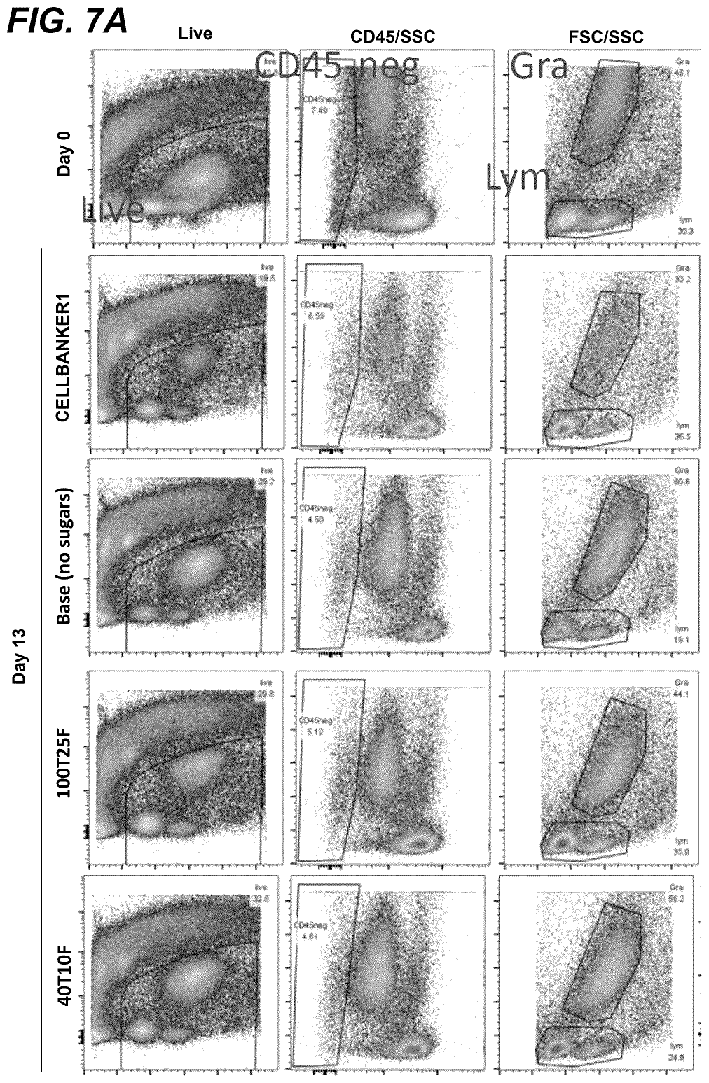

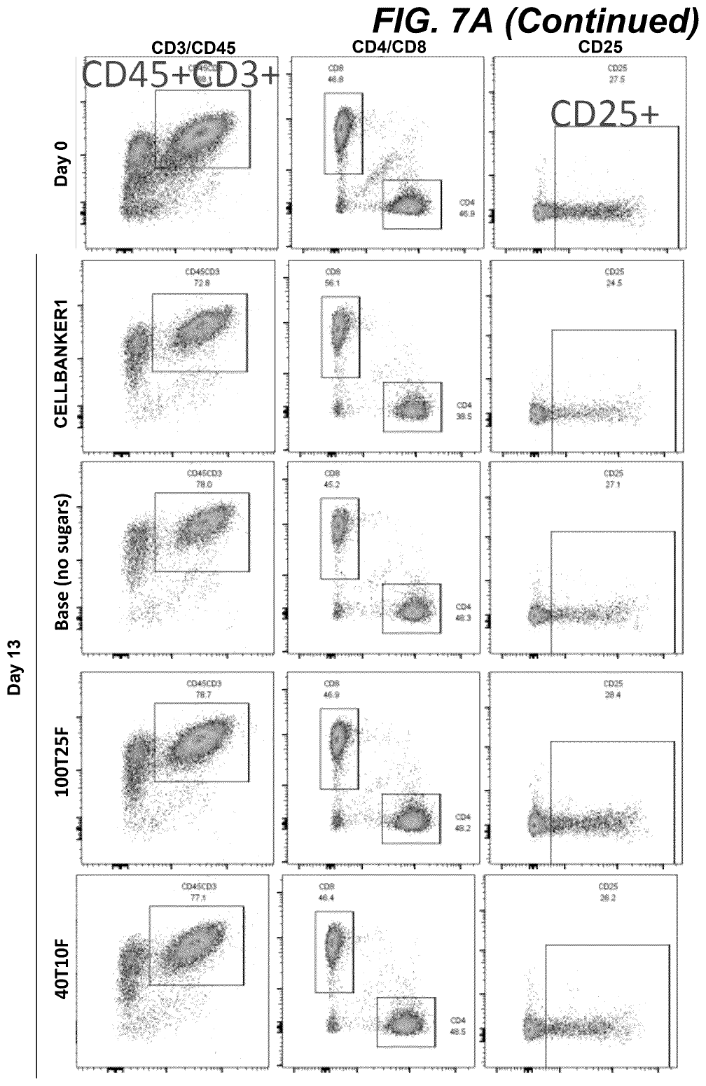

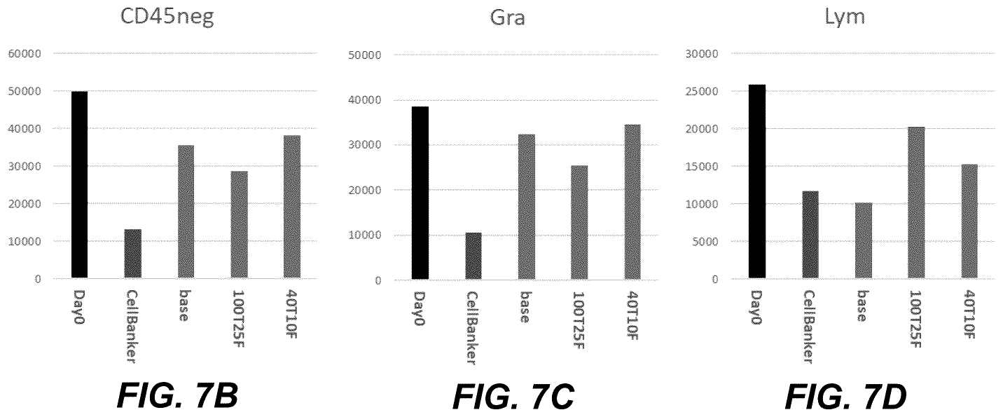

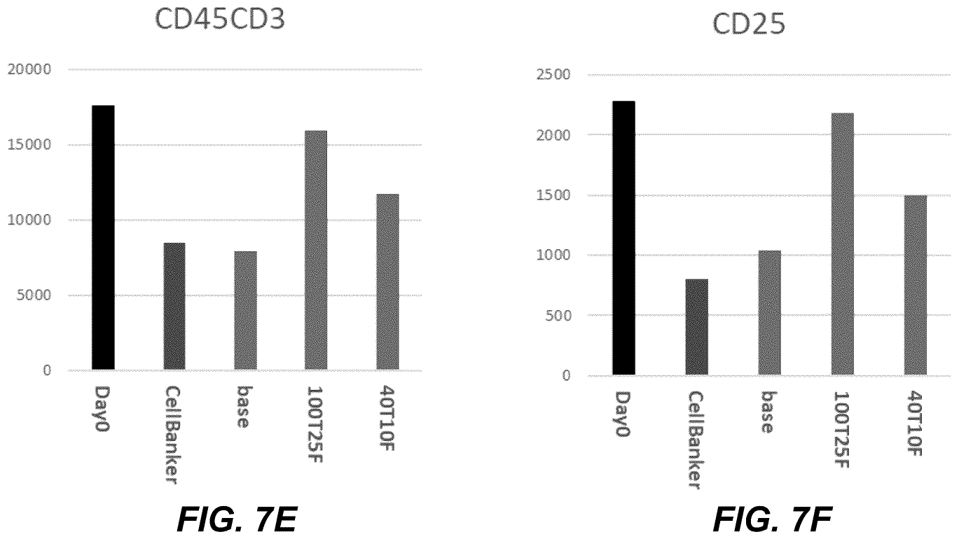

Cryopreservation buffers tested using Specimen No. 5 are described in Table 6. FIGS. 7A-7F depict FACS data related to viability, CD45/SSC, FSC/SSC, CD3/CD45, CD4/CD8, and CD25 properties for Specimen No. 5 at Day 0 and at Cryopreservation Day 13 using cryopreservation buffers CELLBANKER® 1, base (no sugars), 100T25F, and 40T10F (FIG. 7A) and cell number quantifications derived therefrom (FIGS. 7B-7F) for CD45neg (FIG. 7B), granulocytes (FIG. 7C), lymphocytes (Lym) (FIG. 7D), CD45CD3 (FIG. 7E), and CD25 (FIG. 7F) cells. It was found that base (no sugars: 5% DMSO5% EG) is good to preserve CD45lo/neg and Granulocytes, but not good enough for CD45+ cells. Buffer compositions wherein sugars are added to the base can preserve the both types of cells.

TABLE 6
Cryopreservation Buffers

| Cryopreservation | |
|---|---|
| Buffer | Composition |
| CELLBANKER ® 1 | Commercial product |
| Base (no sugars) | 5% DMSO, 5% EG, 50% FBS/RPMI-based |
| 100T25F | 5% DMSO, 5% EG, 50% FBS, 100 mM |
| | Trehalose + 25 mM Fructose/RPMI-based |
| 40T10F | 5% DMSO, 5% EG, 50% FBS, 40 mM |
| | Trehalose + 10 mM Fructose/RPMI-based |

### Specimen No. 6

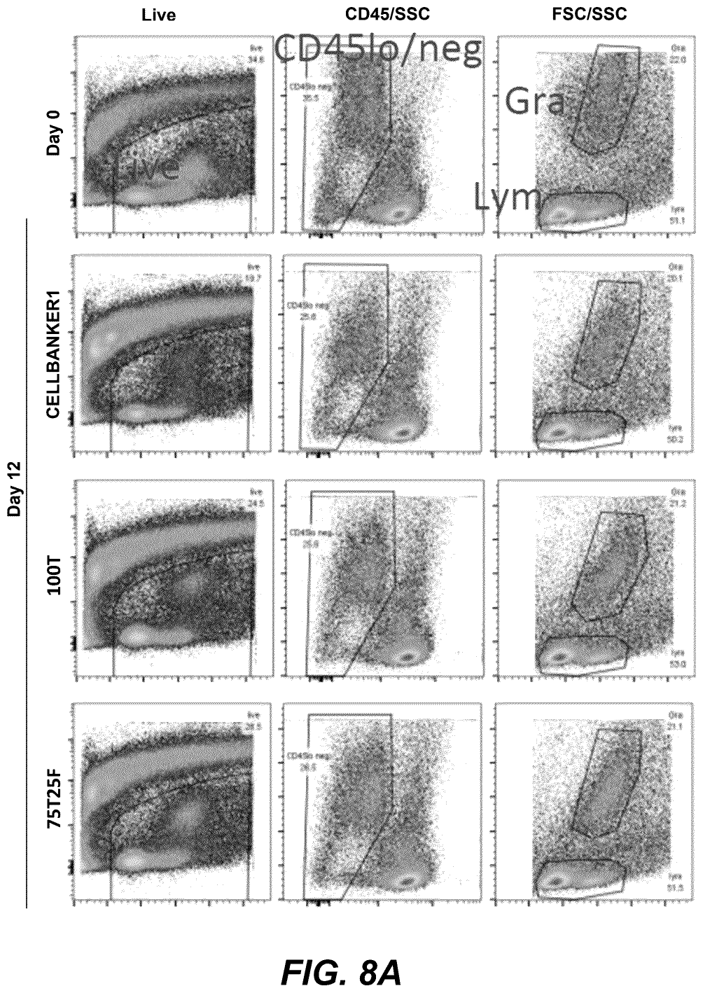

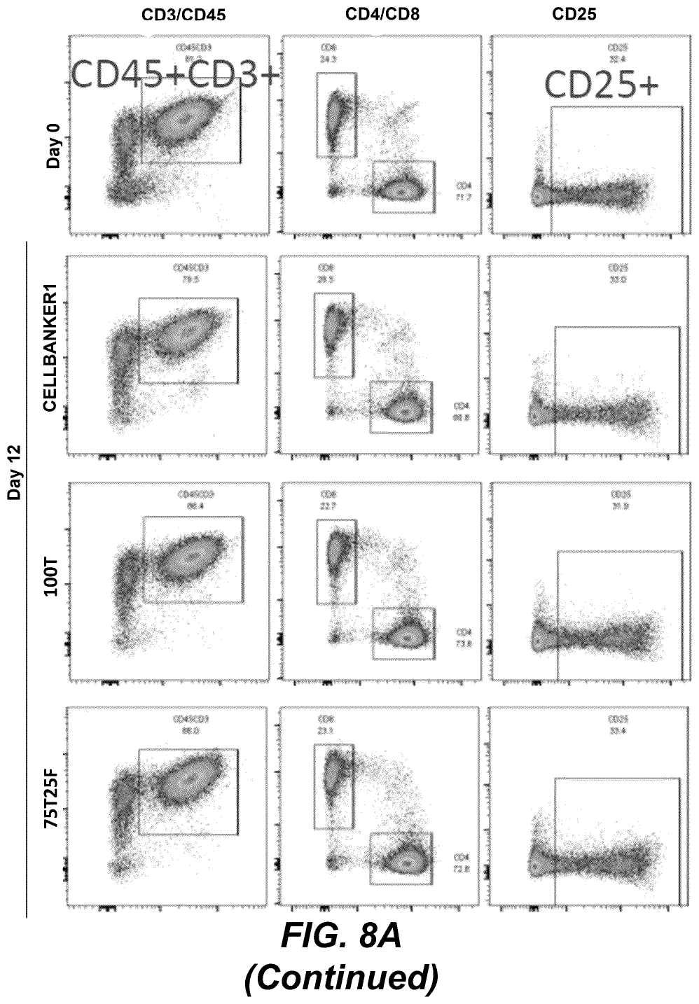

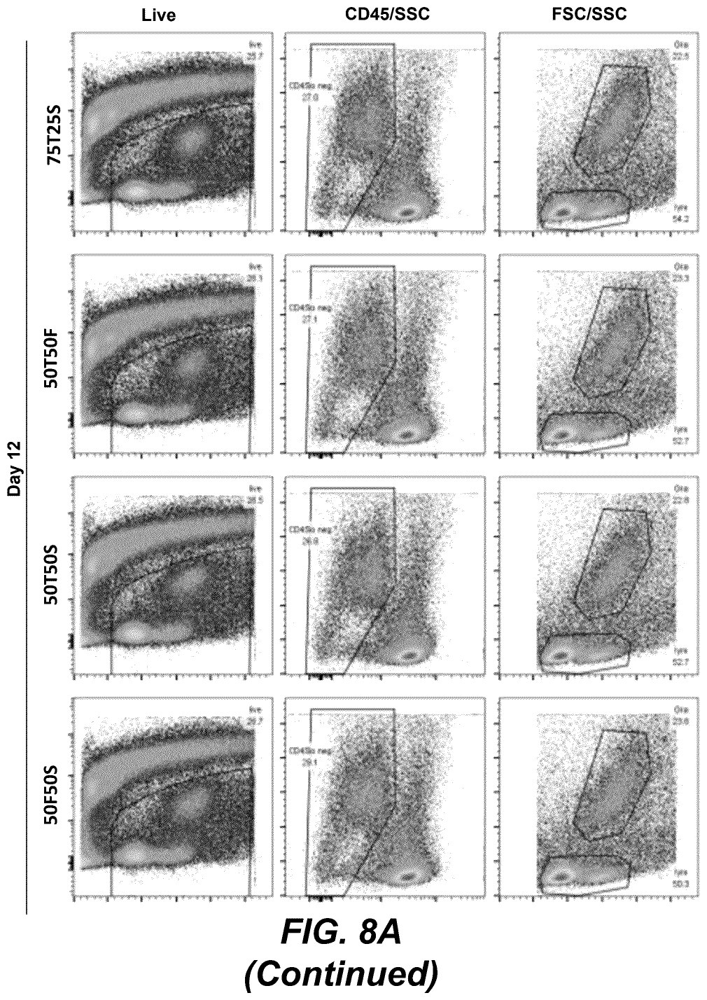

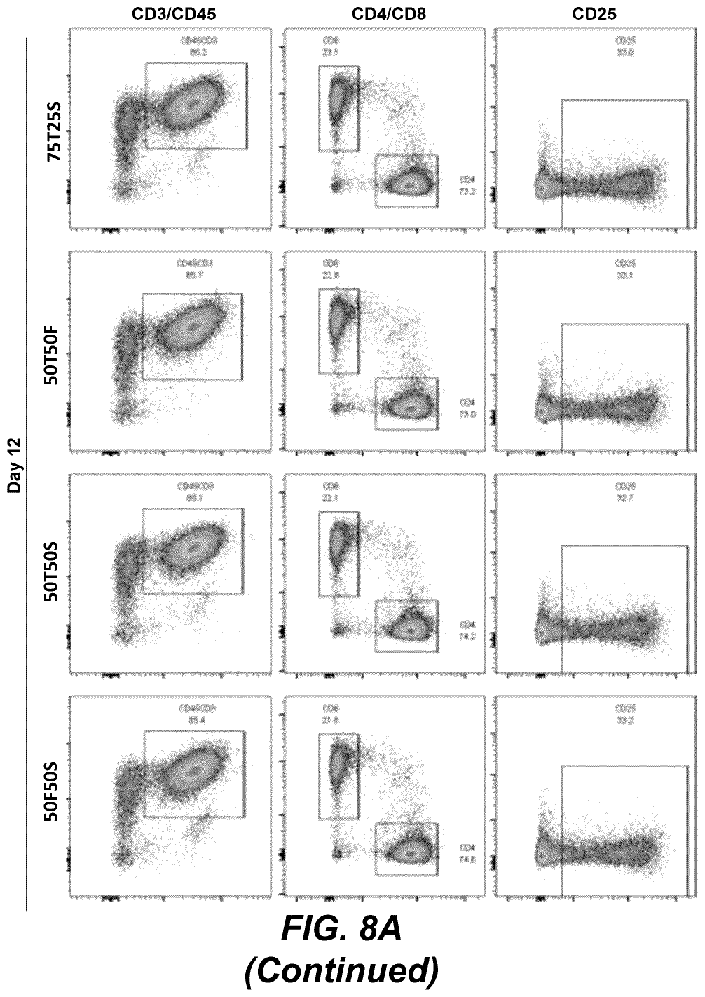

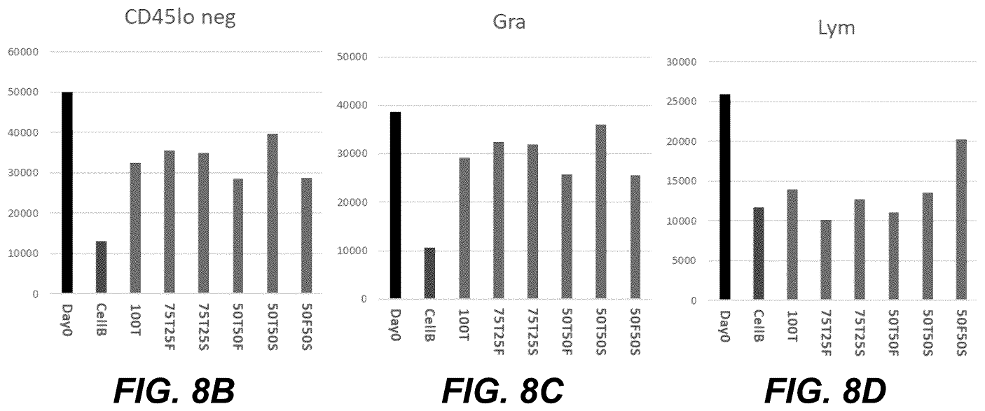

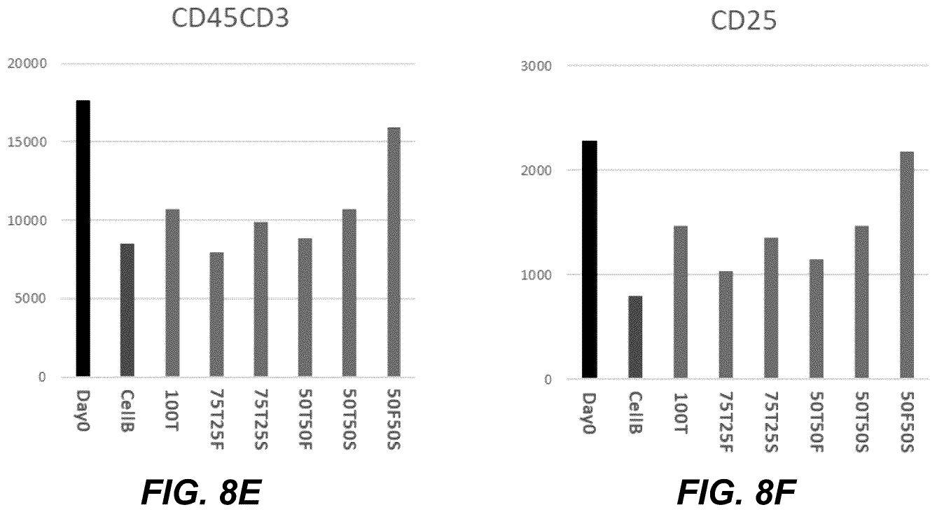

Cryopreservation buffers tested using Specimen No. 6 are described in Table 7. FIGS. 8A-8F depict FACS data related to viability, CD45/SSC, FSC/SSC, CD3/CD45, CD4/CD8, and CD25 properties for Specimen No. 6 at Day 0 and at Cryopreservation Day 12 using cryopreservation buffers CELLBANKER® 1, 100T, 75T25F, 75T25S, 50T50F, 50T50S, 50F50S (FIG. 8A) and cell number quantifications derived therefrom (FIGS. 8B-8F) for CD45lo neg (FIG. 8B), granulocytes (FIG. 8C), lymphocytes (Lym) (FIG. 8D), CD45CD3 (FIG. 8E), and CD25 (FIG. 8F) cells.

TABLE 7
Cryopreservation Buffers

| Cryopreservation | |
|---|---|
| Buffer | Composition |
| CELLBANKER ® 1 | Commercial product |
| 100T | 5% DMSO, 5% EG, 50% FBS, 100 mM |
| | Trehalose/RPMI-based |
| 75T25F | 5% DMSO, 5% EG, 50% FBS, 75 mM |
| | Trehalose + 25 mM Fructose/RPMI-based |
| 75T25S | 5% DMSO, 5% EG, 50% FBS, 75 mM |
| | Trehalose + 25 mM Sucrose/RPMI-based |
| 50T50F | 5% DMSO, 5% EG, 50% FBS, 50 mM |
| | Trehalose + 50 mM Fructose/RPMI-based |
| 50T50S | 5% DMSO, 5% EG, 50% FBS, 50 mM |
| | Trehalose + 50 mM Sucrose/RPMI-based |
| 50F50S | 5% DMSO, 5% EG, 50% FBS, 50 mM |
| | Fructose + 50 mM Sucrose/RPMI-based |

### Specimen No. 7

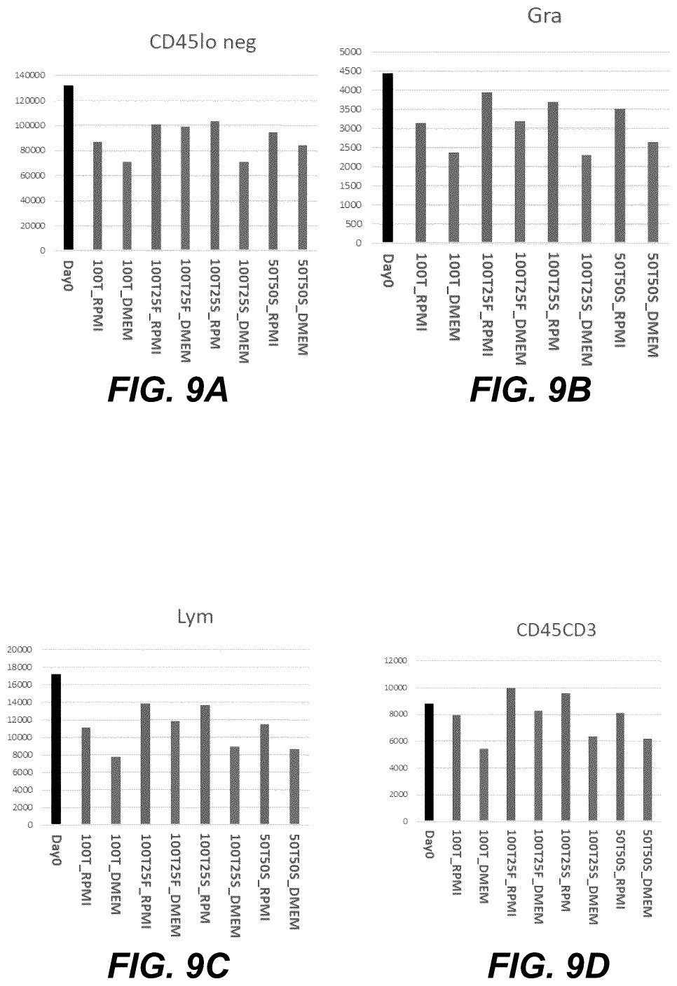

Cryopreservation buffers tested using Specimen No. 7 are described in Table 8. It was examined if DMEM-based or RPMI based buffers perform better. FIGS. 9A-9D depict cell number data for CD45lo neg (FIG. 9A), granulocytes (FIG. 9B), lymphocytes (Lym) (FIG. 9C), CD45CD3 (FIG. 9D) cells for Specimen No. 7 at Day 0 and at Cryopreservation Day 14 using cryopreservation buffers 100T-RPMI, 100T-DMEM, 100T25F-RPMI, 100T25F-DMEM, 100T25S-RPMI, 100T25S-DMEM, 50T50S-RPMI, and 50T50S-DMEM. RPIM-1640 inclusion achieved better results than the use of DMEM as a buffer component.

TABLE 8
Cryopreservation Buffers

| Cryopreservation | |
|---|---|
| Buffer | Composition |
| CELLBANKER ® 1 | Commercial product |
| 100T-RPMI | 5% DMSO, 5% EG, 50% FBS, 100 mM |
| | Trehalose/RPMI-based |
| 100T-DMEM | 5% DMSO, 5% EG, 50% FBS, 100 mM |
| | Trehalose/DMEM-based |
| 100T25F-RPMI | 5% DMSO, 5% EG, 50% FBS, 100 mM |
| | Trehalose + 25 mM Fructose/RPMI-based |
| 100T25F-DMEM | 5% DMSO, 5% EG, 50% FBS, 100 mM |
| | Trehalose + 25 mM Fructose/DMEM-based |
| 100T25S-RPMI | 5% DMSO, 5% EG, 50% FBS, 100 mM |
| | Trehalose + 25 mM Sucrose//RPMI-based |
| 100T25S-DMEM | 5% DMSO, 5% EG, 50% FBS, 100 mM |
| | Trehalose + 25 mM Sucrose/DMEM-based |
| 50T50S-RPMI | 5% DMSO, 5% EG, 50% FBS, 50 mM |
| | Trehalose + 50 mM Sucrose//RPMI-based |
| 50T50S-DMEM | 5% DMSO, 5% EG, 50% FBS, 50 mM |
| | Trehalose + 50 mM Sucrose/DMEM-based |

### Specimen No. 8

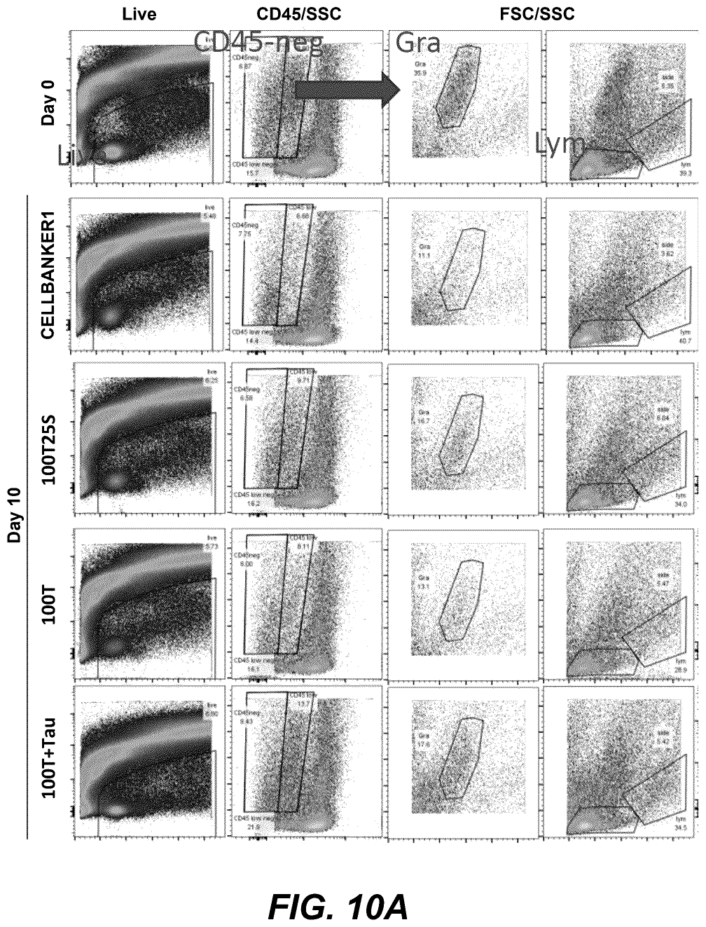

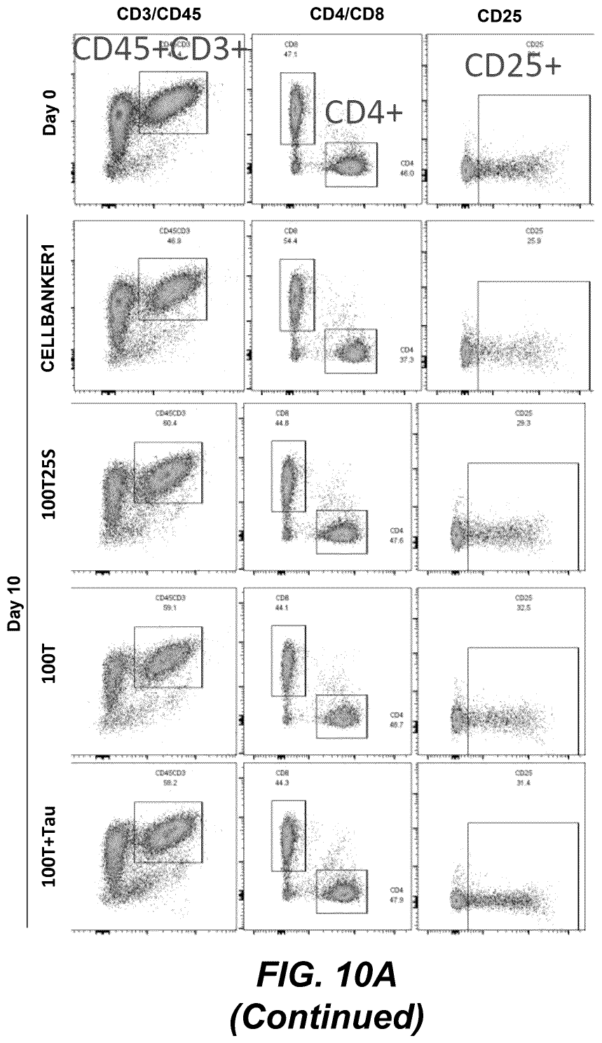

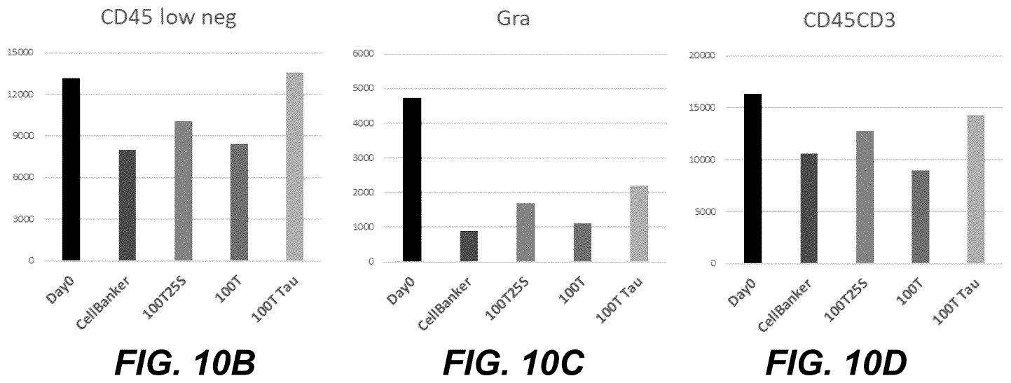

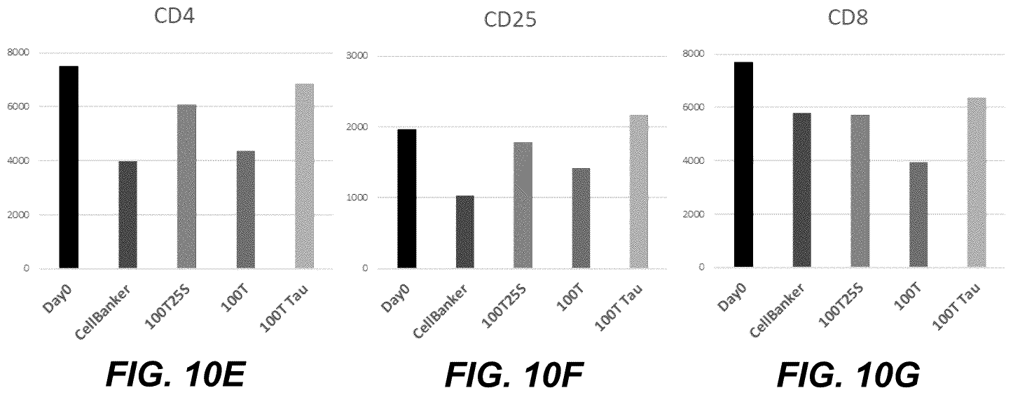

Cryopreservation buffers tested using Specimen No. 8 are described in Table 9. FIGS. 10A-10G depict FACS data related to viability, CD45/SSC, FSC/SSC, CD3/CD45, CD4/CD8, and CD25 properties for Specimen No. 8 at Day 0 and at Cryopreservation Day 10 using cryopreservation buffers CELLBANKER® 1, 100T25S, 100T, and 100T+ Tau (FIG. 10A) and cell number quantifications derived therefrom (FIGS. 10B-10G) for CD45lo neg (FIG. 10B), granulocytes (FIG. 10C), CD45CD3 (FIG. 10D), CD4 (FIG. 10E), CD25 (FIG. 10F), and CD8 (FIG. 10G) cells. The addition of Taurine to 100T achieved better results than that of buffer 100T25S. In some embodiments, tumor tissue should be treated within 24 hours for the cryopreservation to get better cell recovery.

TABLE 9
Cryopreservation Buffers

| Cryopreservation | |
|---|---|
| Buffer | Composition |
| CELLBANKER ® 1 | Commercial product |
| 100T25S | 5% DMSO, 5% EG, 50% FBS, 100 mM |
| | Trehalose + 25 mM Sucrose/RPMI-based |
| 100T | 5% DMSO, 5% EG, 50% FBS, 100 mM |
| | Trehalose/RPMI-based |
| 100T + Tau | 5% DMSO, 5% EG, 50% FBS, 100 mM |
| | Trehalose + 100 mM Taurine/RPMI-based |

### Specimen No. 9

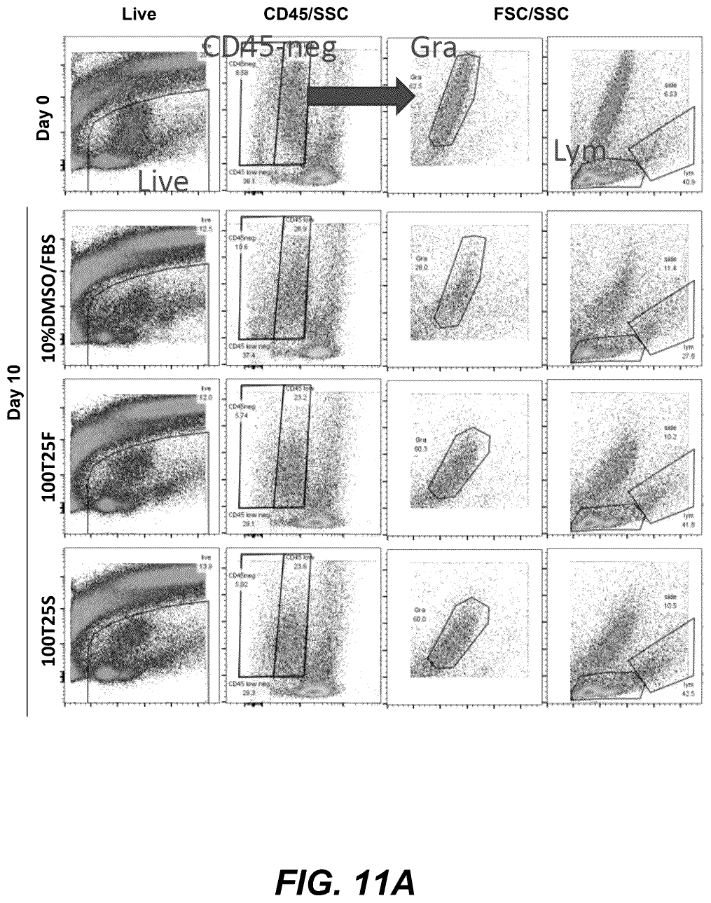

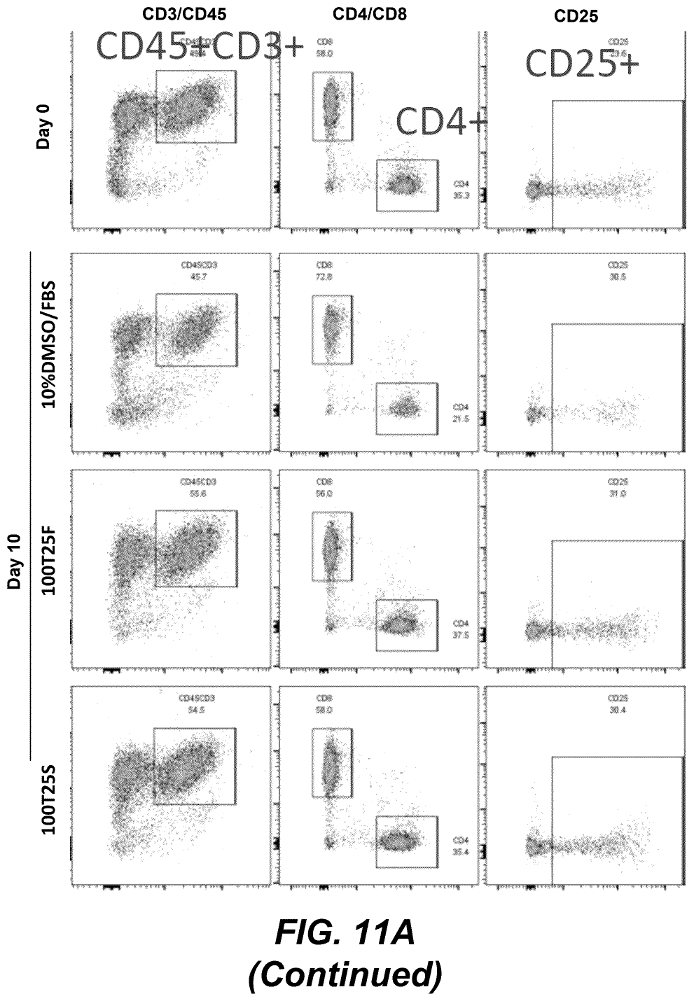

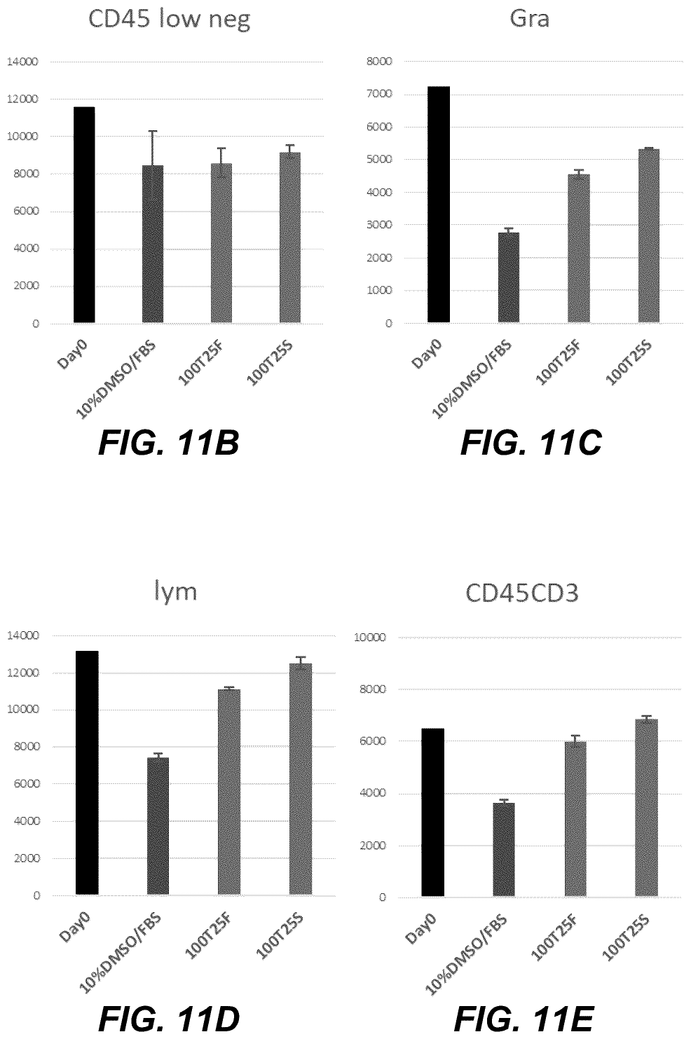

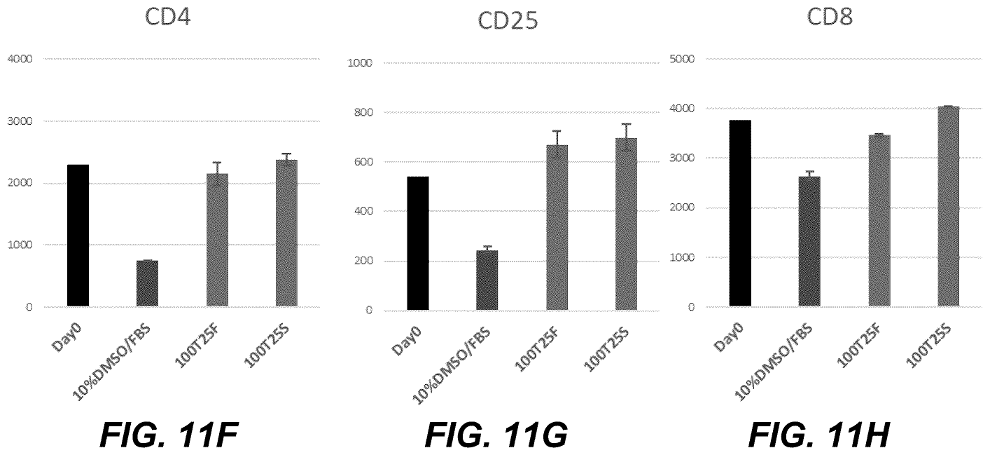

Cryopreservation buffers tested using Specimen No. 9 are described in Table 10 (n=2 for each condition (Day 0: n=1)). FIGS. 11A-11H depict FACS data related to viability, CD45/SSC, FSC/SSC, CD3/CD45, CD4/CD8, and CD25 properties for Specimen No. 9 at Day 0 and at Cryopreservation Day 10 using cryopreservation buffers 10% DMSO/FBS, 100T25F, and 100T25S (FIG. 11A) and cell number quantifications derived therefrom (FIGS. 11B-11H) for CD45lo neg (FIG. 11B), granulocytes (Gra) (FIG. 11C), lymphocytes (Lym) (FIG. 11D), CD45CD3 (FIG. 11E), CD4 (FIG. 11F), CD25 (FIG. 11G), and CD8 (FIG. 11H) cells.

TABLE 10
Cryopreservation Buffers

| Cryopreservation | |
|---|---|
| Buffer | Composition |
| 10% DMSO/FBS | 10% DMSO, 90% FBS |
| 100T25F | 5% DMSO, 5% EG, 50% FBS, 100 mM |
| | Trehalose + 25 mM Fructose/RPMI-based |
| 100T25S | 5% DMSO, 5% EG, 50% FBS, 100 mM |
| | Trehalose + 25 mM Sucrose/RPMI-based |

### Specimen No. 10

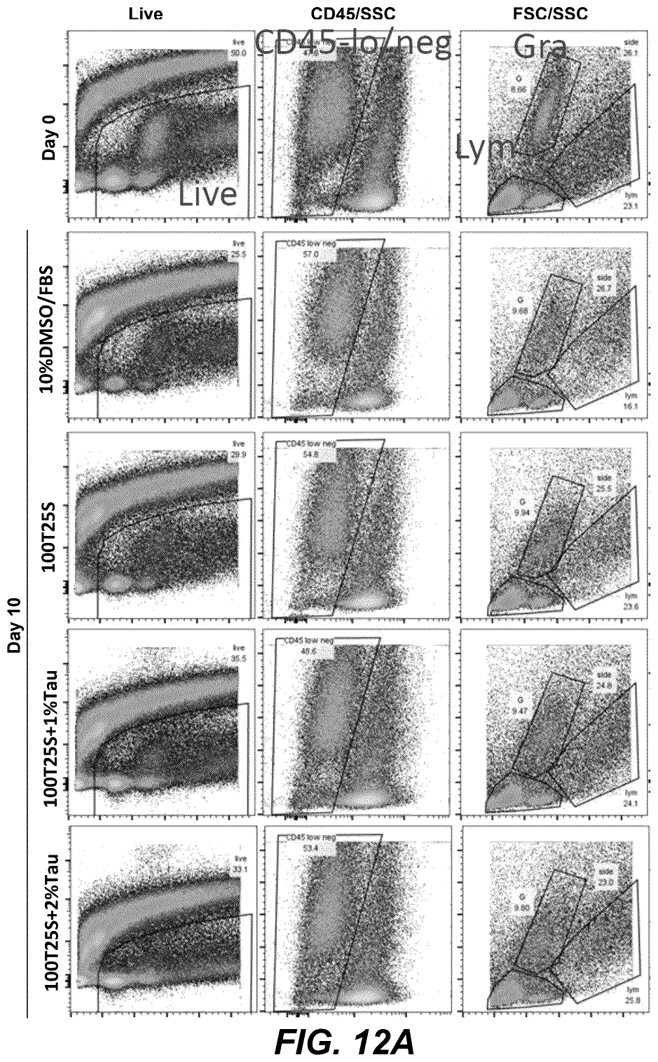

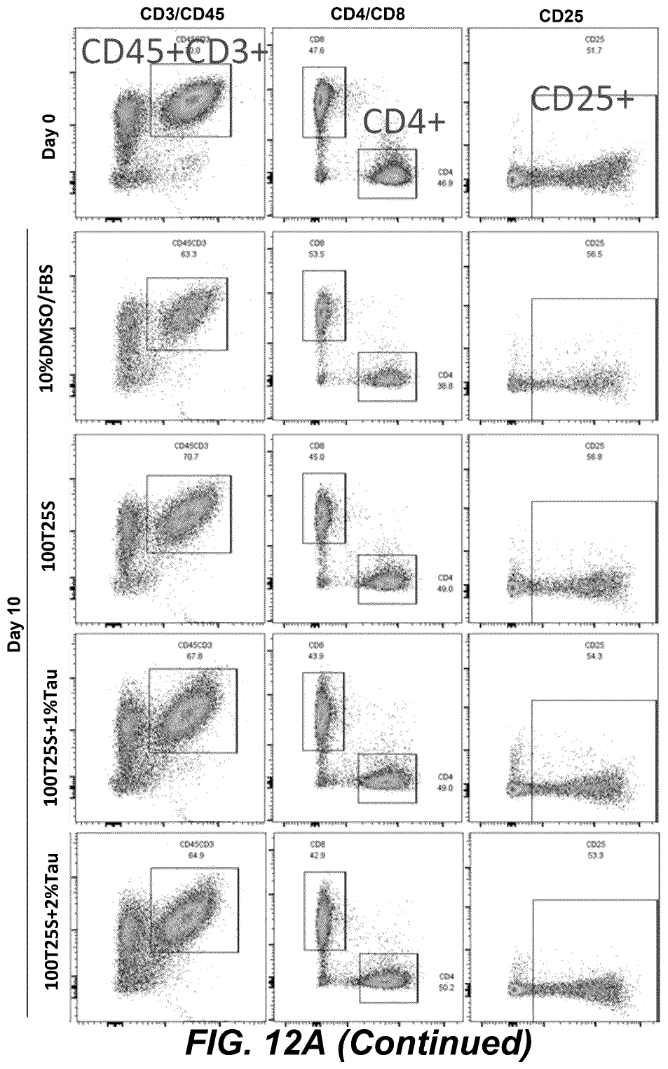

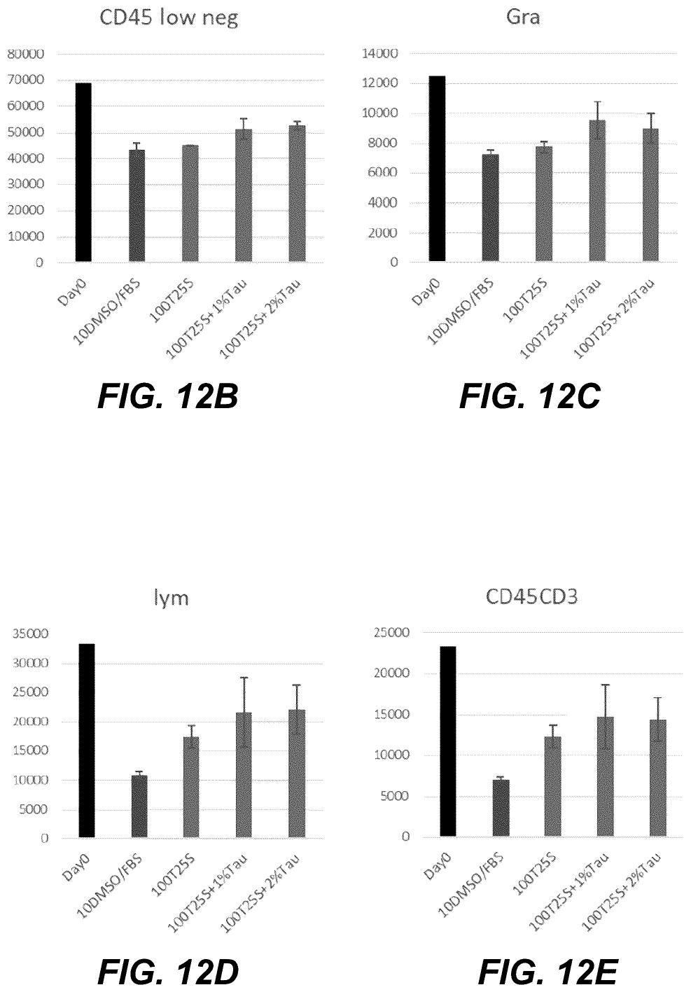

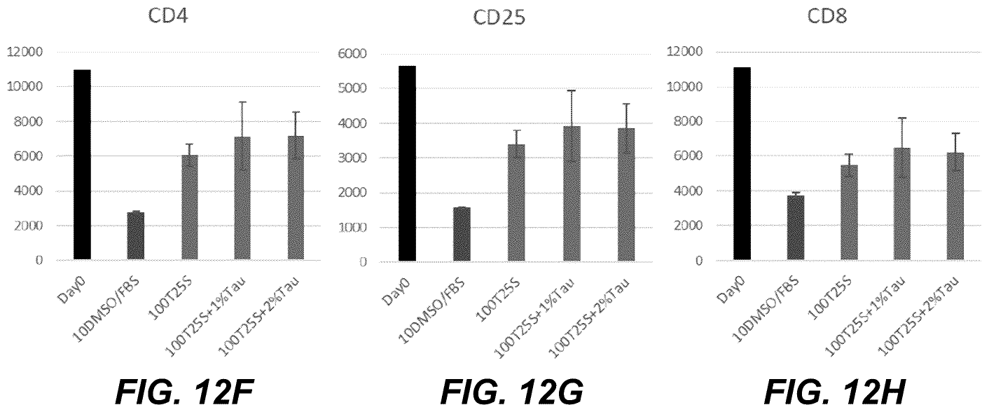

Cryopreservation buffers tested using Specimen No. 10 are described in Table 11 (n=2 for each condition (Day 0: n=1)). FIGS. 12A-12H depict FACS data related to viability, CD45/SSC, FSC/SSC, CD3/CD45, CD4/CD8, and CD25 properties for Specimen No. 10 at Day 0 and at Cryopreservation Day 10 using cryopreservation buffers 10% DMSO/FBS, 100T25S, 100T25S+1% Tau, and 100T25S+2% Tau (FIG. 12A) and cell number quantifications derived therefrom (FIGS. 12B-12H) for CD45 low neg (FIG. 12B), granulocytes (Gra) (FIG. 12C), lymphocytes (Lym) (FIG. 12D), CD45CD3 (FIG. 12E), CD4 (FIG. 12F), CD25 (FIG. 12G), and CD8 (FIG. 12H) cells.

TABLE 11
Cryopreservation Buffers

| Cryopreservation | |
|---|---|
| Buffer | Composition |
| 10% DMSO/FBS | 10% DMSO, 90% FBS |
| 100T25S | 5% DMSO, 5% EG, 50% FBS, 100 mM |
| | Trehalose + 25 mM Sucrose/RPMI-based |
| 100T25S + 1% Tau | 5% DMSO, 5% EG, 50% FBS, 100 mM |
| | Trehalose + 25 mM Sucrose/RPMI-based |
| | 1% (80 mM) Taurine |
| 100T25S + 2% Tau | 5% DMSO, 5% EG, 50% FBS, 100 mM |
| | Trehalose + 25 mM Sucrose/RPMI-based |
| | 2% (160 mM) Taurine |

### Conclusions

Cryopreservation buffers provided herein can cryopreserve cells from tumor tissue with much higher viability as compared to commercially available and conventional cryopreservation buffers. In some embodiments it is preferable to treat tumor tissue with a cell dispersion solution (e.g., BD Horizon™ Dri Tumor & Tissue Dissociation Reagent (TTDR)) to generate a cell suspension within 24 hours (˜48 hr) of immersion in a sample preservation solution (e.g., BD® Tumor & Tissue Preservation Reagent (TTPR)) for cryopreservation, as this can lead to better cell viability after thawing.

### Example 2

### TTPR-Based Cryopreservation Reagent Studies

In this Example cryopreservation studies including TTPR-based cryopreservation reagents were performed using the Specimens Tissue-9, Tissue-10, and Tissue-11 (Table 12).

TABLE 12
Specimens

| Specimen | Cell Suspension or Tissue Chunk | Condition |
|---|---|---|
| Tissue-9 | Tissue chunk | at −80° C. for 7 days |
| Tissue-10 | Cell suspension (TTDR-dissociated) | at −80° C. for 16 days |
| Tissue-9 | Cell suspension (TTDR-dissociated) | at −80° C. for 20 days |
| Tissue-11 | Cell suspension (TTDR-dissociated) | at −80° C. for 20 days |

### Tissue Chunk (Tissue-9)

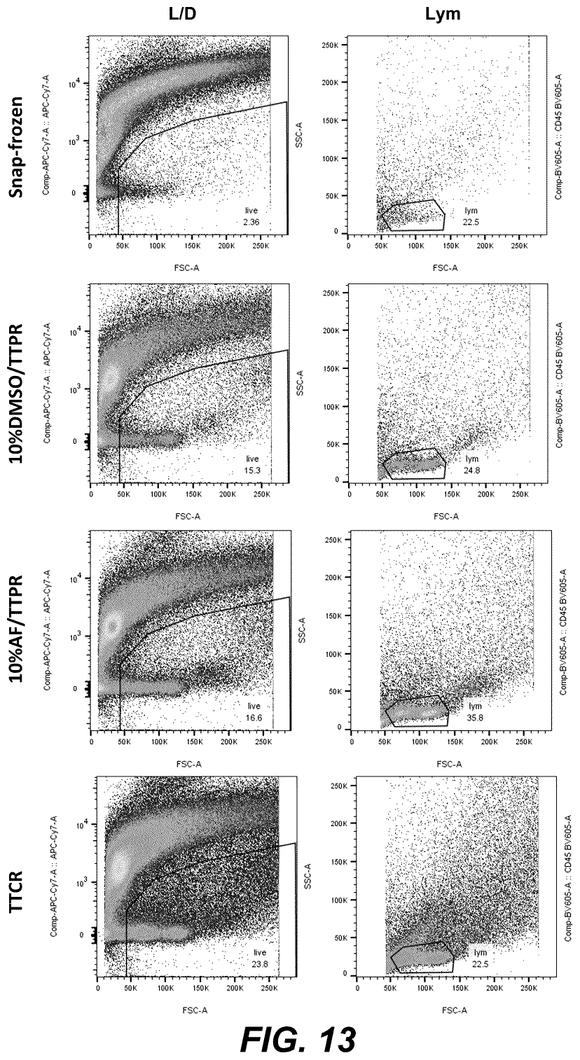

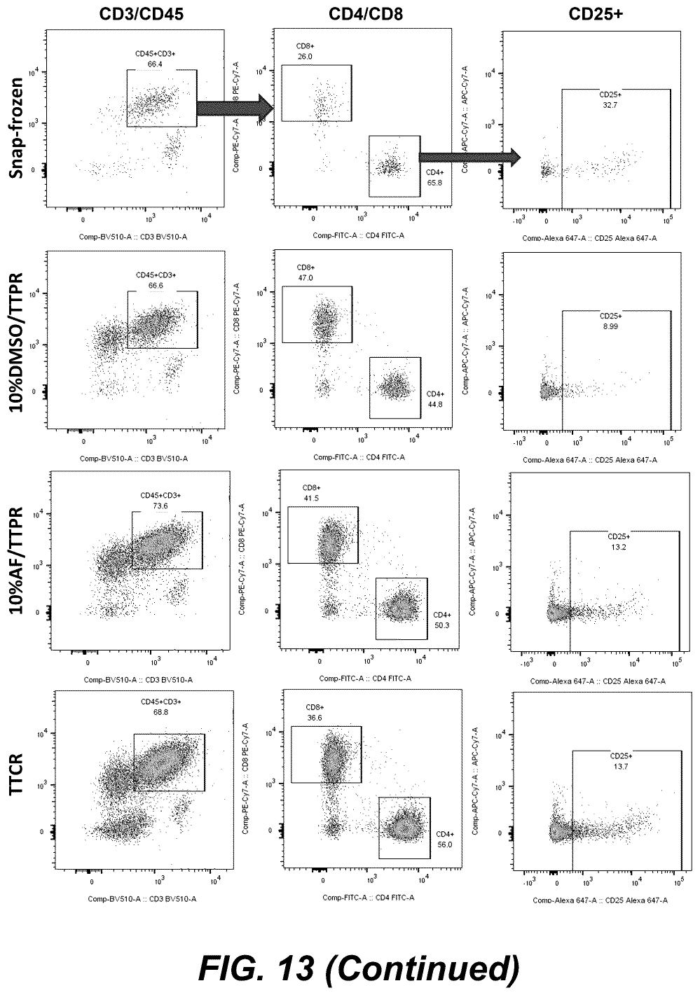

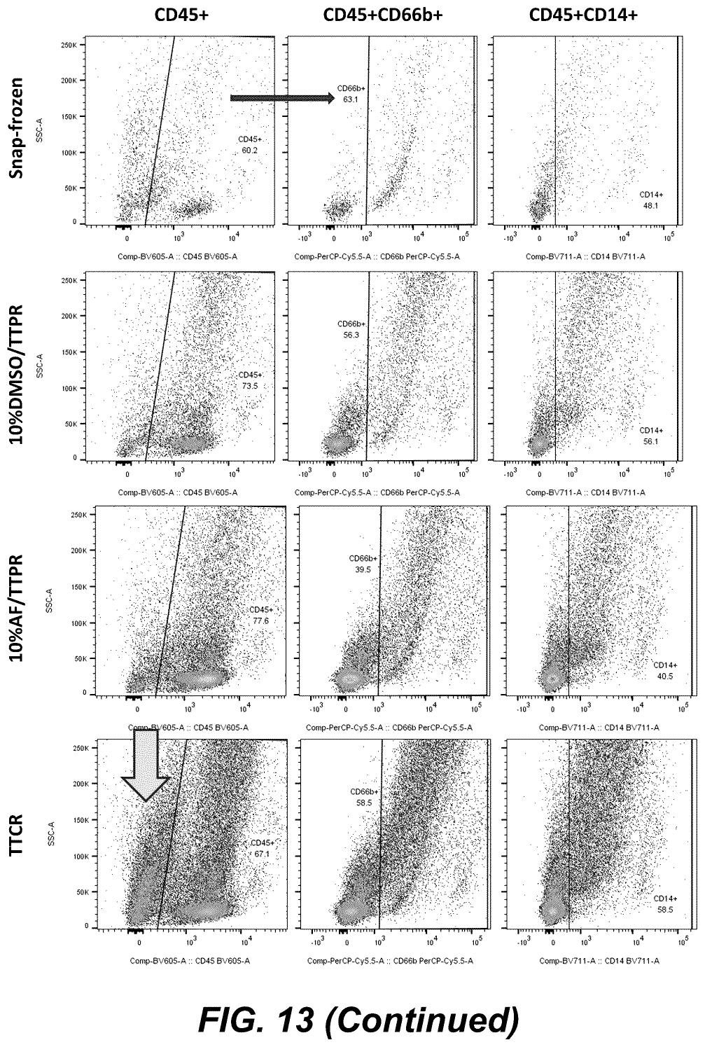

The specimen was collected in TTPR. Approximately 30 mg (3 pieces, approximately 10 mg/piece) were added to separate tubes and four conditions were evaluated as shown in Table 13-snap-frozen or immersed in a cryopreservation buffer. The four containers were frozen at −80° C. for 7 days, after which a single cell suspension was generated using TTDR and the cells underwent FACS analysis. FACS analysis was subsequently performed. FIG. 13 depicts FACS data related to viability, lymphocyte (Lym), CD3/CD45, CD4/CD8, CD25+, CD45+, CD45+CD66b+, and CD45+CD14+ properties for Specimen Tissue-9 (tissue chunk) frozen at −80° C. for 7 days either using cryopreservation buffers 10% DMSO/TTPR, 10% AF/TTPR, and TTCR, or snap-frozen. 10% AF/TTPR appeared to perform better than 10% DMSO/TTPR. The data indicated that TTCR can also preserve more CD45-cells (tumor cells and some others).

TABLE 13
Tissue chunk (Tissue-9) Testing

| Condition No. | Biopsy Weight (3 Pieces) | Cryopreservation Buffer |
|---|---|---|
| 1 | 35 mg | N/A - snap-frozen (dry ice) |
| 2 | 36 mg | 10% DMSO/TTPR |
| 3 | 37 mg | 10% AF/TTPR |
| 4 | 37 mg | TTCR |

AF: mixture of anti-freezes
TTCR contains 10% AF

### Cell Suspension (Tissue-10)

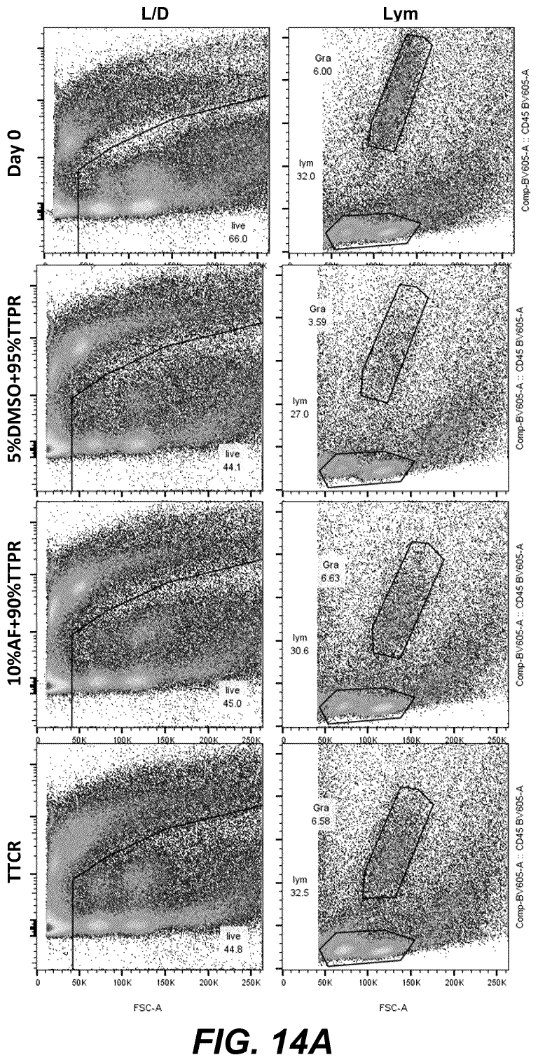

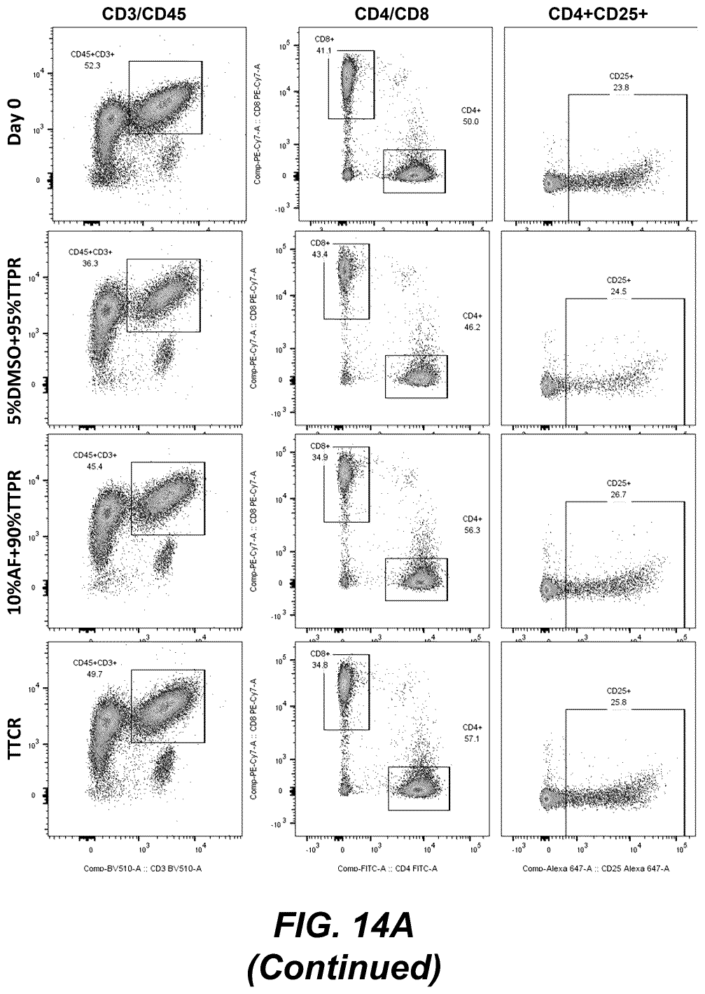

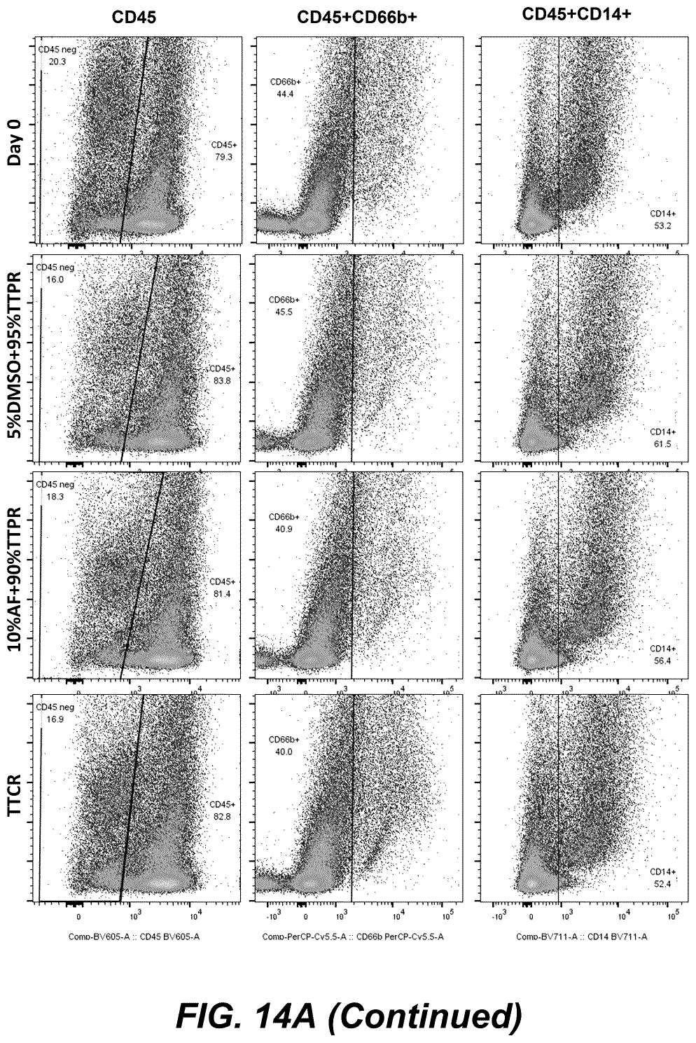

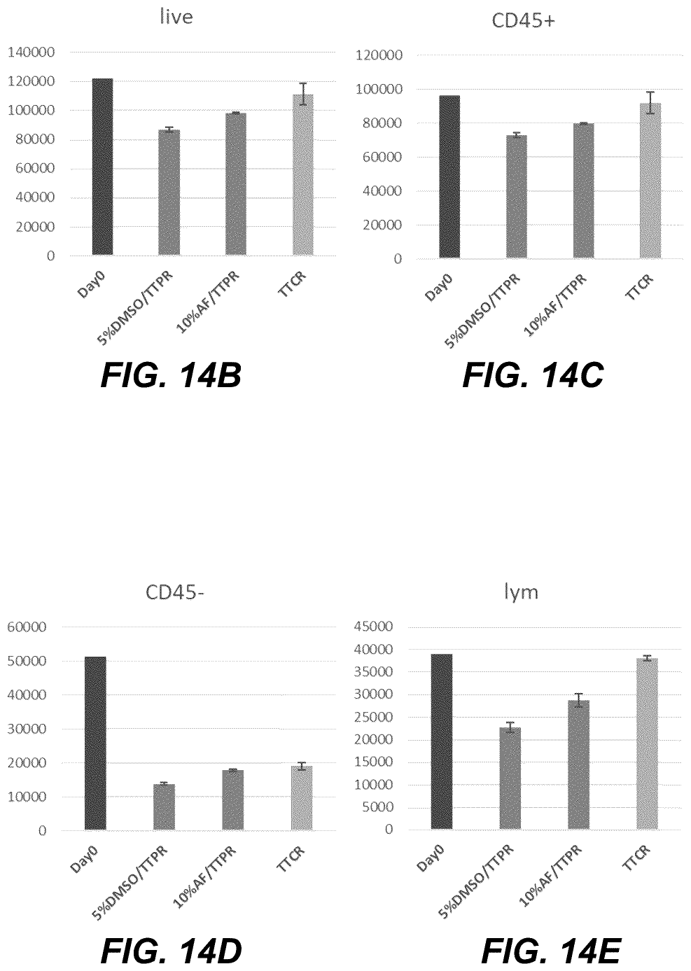

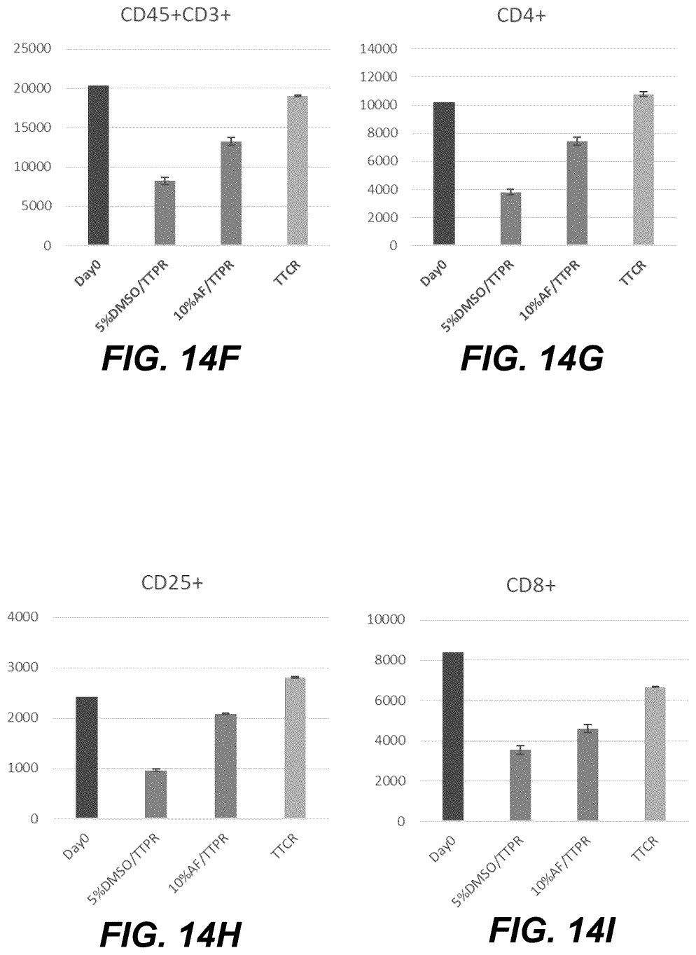

The specimen was collected in TTPR and a single cell suspension was generated using TTDR. 100 μL of cell suspension was aliquoted into tubes. Four conditions were evaluated as shown in Table 14-fresh (Day 0, not frozen) or immersed in a cryopreservation buffer and frozen at −80° C. for 16 days. FACS analysis was subsequently performed. FIGS. 14A-14I depict FACS data related to viability, lymphocyte (Lym), CD3/CD45, CD4/CD8, CD4+CD25+, CD45, CD45+CD66b+, and CD45+CD14+ properties for Specimen Tissue-10 (cell suspension, TTDR-dissociated) at Day 0 and after −80° C. for 16 days using cryopreservation buffers 5% DMSO+95% TTPR, 10% AF+90% TTPR, and TTCR (FIG. 14A) and cell number quantifications derived therefrom (FIGS. 14B-14I) for live (FIG. 14B), CD45+ (FIG. 14C), CD45− (FIG. 14D), lymphocyte (Lym) (FIG. 14E), CD45+CD3+ (FIG. 14F), CD4+ (FIG. 14G), CD25+ (FIG. 14H), and CD8+ (FIG. 14I) cells.

TABLE 14
Cell suspension (Tissue-10) Testing

| Condition No. | Cryopreservation Buffer |
|---|---|
| 1 | N/A - Fresh (Day 0, not frozen) |
| 2 | 5% DMSO + 95% TTPR |
| 3 | 10% AF + 90% TTPR |
| 4 | TTCR |

Two tubes for each of Conditions Nos. 2-4

### Cell Suspension (Tissue-9)

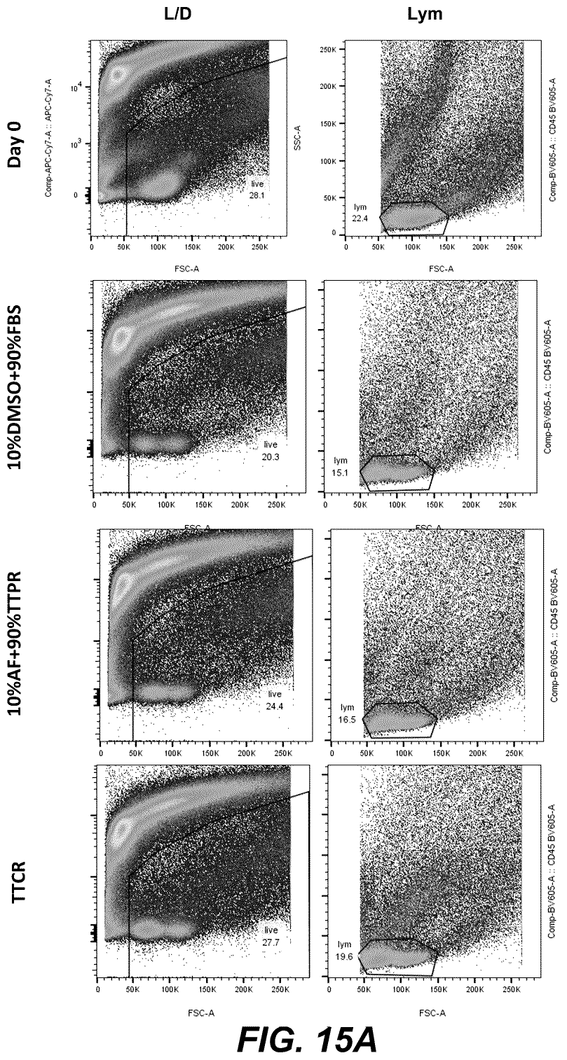

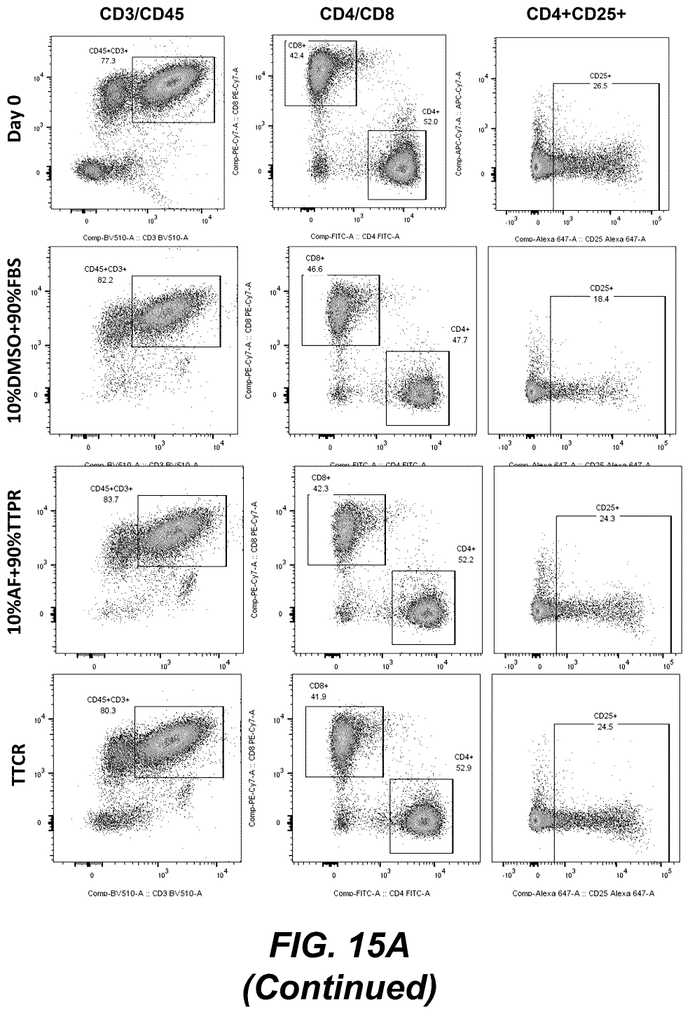

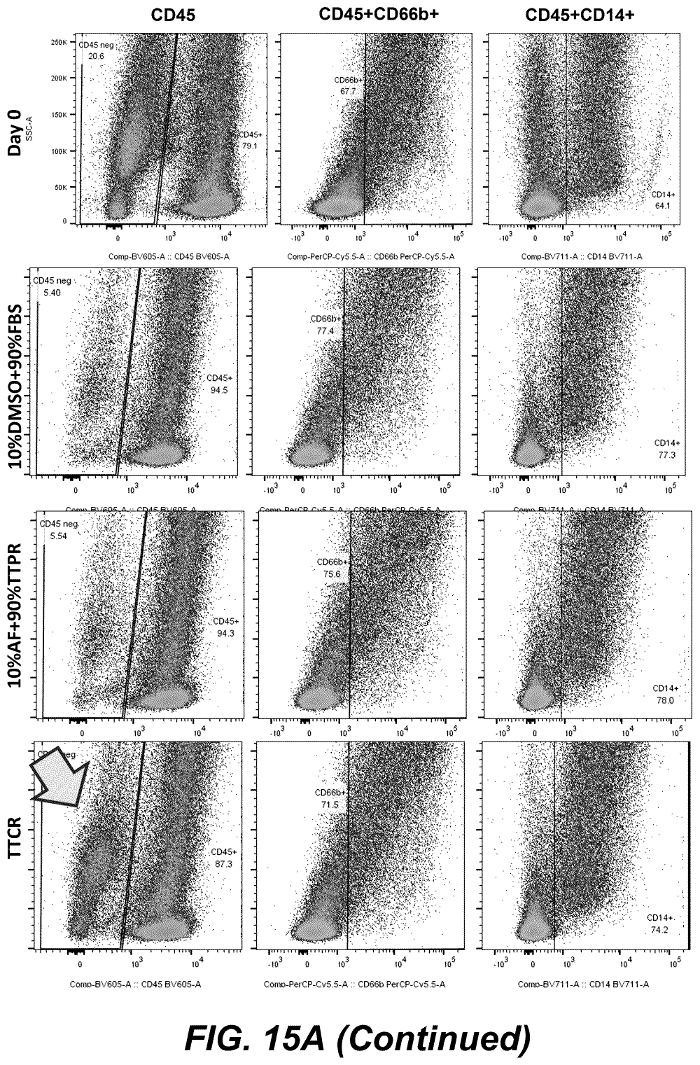

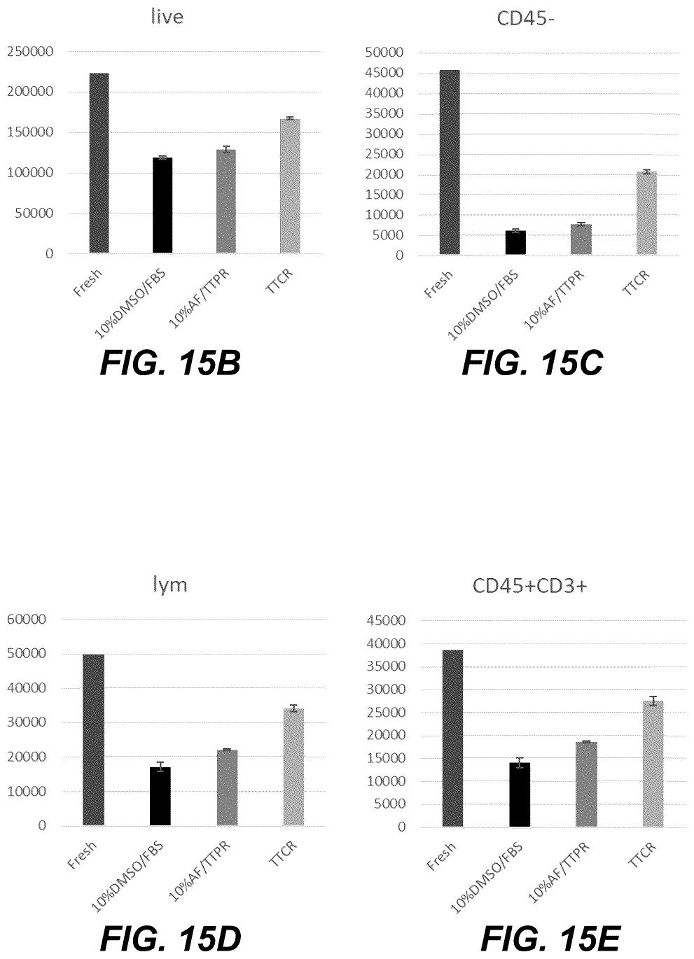

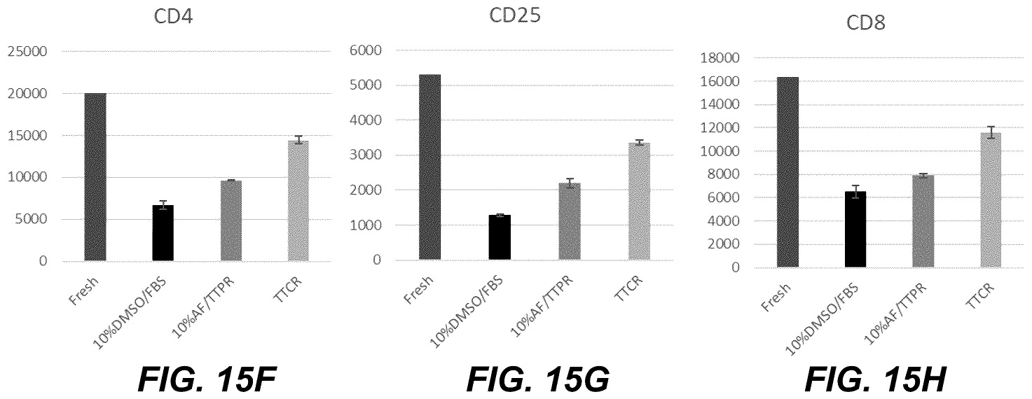

The specimen was collected in TTPR and a single cell suspension was generated using TTDR. 100 μL of cell suspension was aliquoted into tubes. Four conditions were evaluated as shown in Table 15-fresh (Day 0, not frozen) or immersed in a cryopreservation buffer and frozen at −80° C. for 20 days. FACS analysis was subsequently performed. FIGS. 15A-15H depict FACS data related to viability, lymphocyte (Lym), CD3/CD45, CD4/CD8, CD4+CD25+, CD45, CD45+CD66b+, and CD45+CD14+ properties for Specimen Tissue-9 (cell suspension, TTDR-dissociated) at Day 0 and after −80° C. for 20 days using cryopreservation buffers 10% DMSO+90% FBS, 10% AF+90% TTPR, and TTCR (FIG. 15A) and cell number quantifications derived therefrom (FIGS. 15B-15H) for live (FIG. 15B), CD45− (FIG. 15C), lymphocyte (Lym) (FIG. 15D), CD45+CD3+ (FIG. 15E), CD4 (FIG. 15F), CD25 (FIG. 15G), and CD8 (FIG. 15H) cells. The data indicated that TTCR can also preserve more CD45-cells (tumor cells and some others).

TABLE 15
Cell suspension (Tissue-9) Testing

| Condition No. | Cryopreservation Buffer |
|---|---|
| 1 | N/A - Fresh (Day 0, not frozen) |
| 2 | 10% DMSO + 90% FBS |
| 3 | 10% AF + 90% TTPR |
| 4 | TTCR |

Two tubes for each of Conditions Nos. 2-4

### Cell Suspension (Tissue-11)

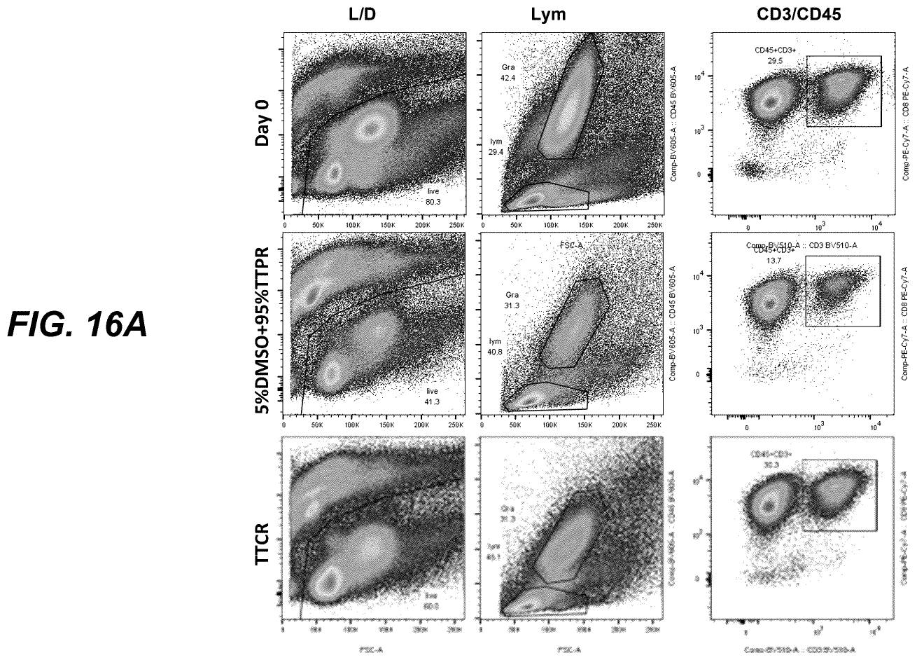

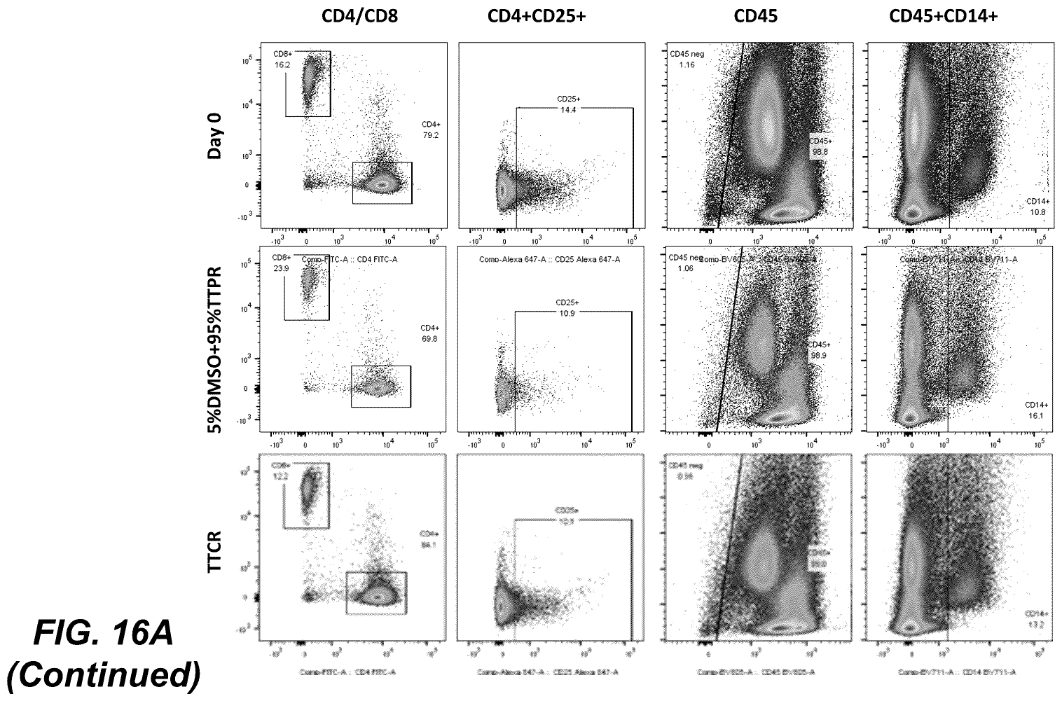

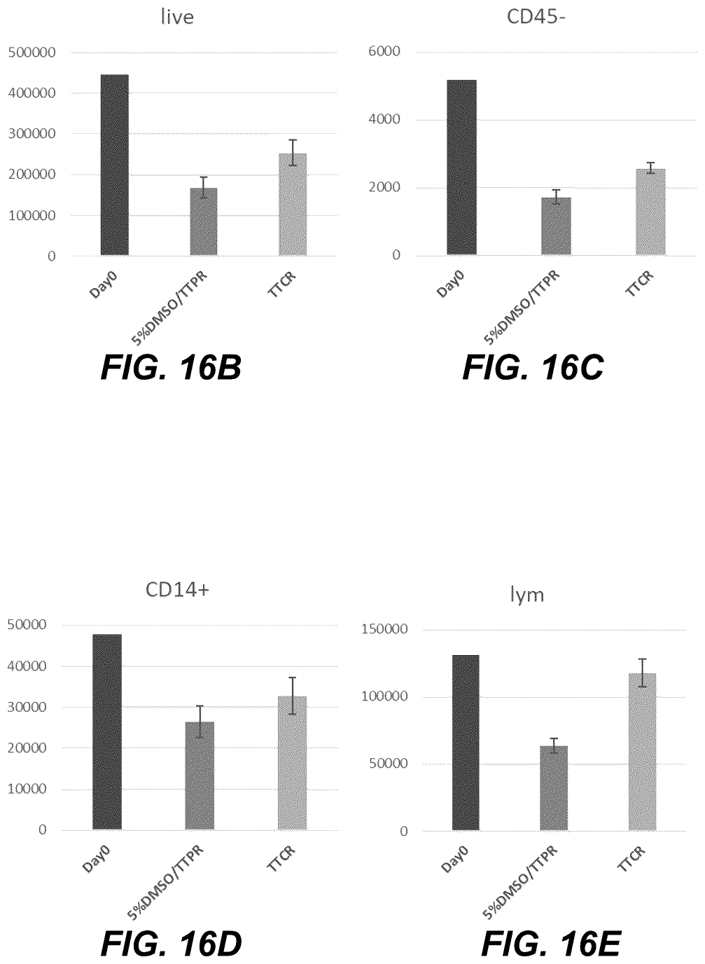

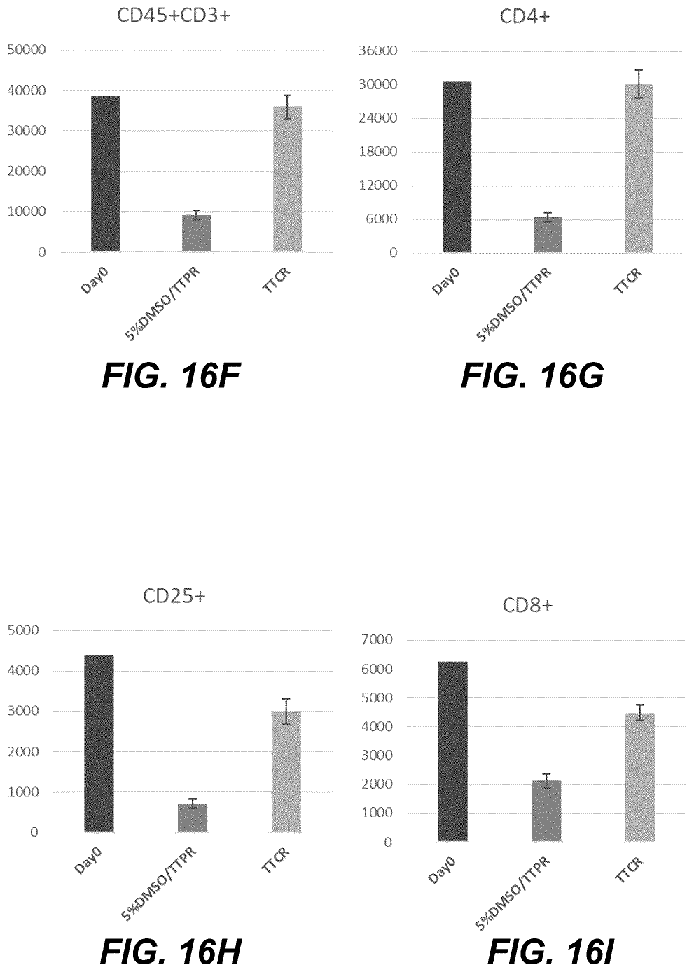

The specimen was collected in TTPR and a single cell suspension was generated using TTDR. 100 μL of cell suspension was aliquoted into tubes. Three conditions were evaluated as shown in Table 16-fresh (Day 0, not frozen) or immersed in a cryopreservation buffer and frozen at −80° C. for 20 days. FIGS. 16A-16I depict FACS data related to viability, lymphocyte (Lym), CD3/CD45, CD4/CD8, CD4+CD25+, CD45, and CD45+CD14+ properties for Specimen Tissue-11 (cell suspension, TTDR-dissociated) at Day 0 and after −80° C. for 20 days using cryopreservation buffers 5% DMSO+95% TTPR and TTCR (FIG. 16A) and cell number quantifications derived therefrom (FIGS. 16B-16I) for live (FIG. 16B), CD45− (FIG. 16C), CD14+ (FIG. 16D), lymphocyte (Lym) (FIG. 16E), CD45+CD3+ (FIG. 16F), CD4+ (FIG. 16G), CD25+ (FIG. 16H), and CD8+ (FIG. 16I) cells.

TABLE 16
Cell suspension (Tissue-11) Testing

| Condition No. | Cryopreservation Buffer |
|---|---|
| 1 | N/A - Fresh (Day 0, not frozen) |
| 2 | 5% DMSO + 95% TTPR |
| 3 | TTCR |

Two tubes for each of Conditions Nos. 2-3

### Example 3

### Tumor Tissue Chunk/Biopsy Sample Cryopreservation Studies

In this Example tissue chunks frozen in TTCR were compared to fresh (Day 0), snap-frozen, and other cell preparations. TTCR (cryopreservation buffer) as shown herein is capable of cryopreserving not only cell suspension/tumor tissue-dissociated single cells as indicated in the Examples above, but also a tissue chunk itself, such as a biopsy specimen, without dissociating tissue. As described in “Wu et al. Genome Medicine (2021) 13:81”, cryopreserving tumor tissue chunks themselves is of paramount importance in very busy clinical sites, as this allows biopsy samples to be maintained in their original fresh condition in a very simple, hassle-free procedure. This Example provides representative data of tissue chunks cryopreserved in TTCR compared to fresh cells (Day 0, before freezing), cells processed using 10% DMSO/90% FBS buffer, and the snap-frozen method many clinical institutes/hospitals currently use. Specimens evaluated are provided in Table 17.

TABLE 17
Specimens for Cryopreservation Tests with Tissue Chunk

| | | TTPR-immersing | | |
|---|---|---|---|---|
| | | hours after specimen | | |
| | Tumor | collection before | | Frozen |
| Specimen | Tissue | freezing | Test tubes | condition |
| Tissue-12 | Lung cancer | 48 hours | Day 0 (Fresh) | at −80° C. for |
| | tissue | | Snap frozen | 50 days |
| | | | TTCR | |
| Tissue-13 | Lung cancer | within 24 hours | Snap frozen | at −80° C. for |
| | tissue | | TTCR | 70 days |
| Tissue-14 | Lung cancer | within 24 hours | Day 0 (Fresh) | at −80° C. for |
| | tissue | | 10% DMSO/90% FBS | 30 days |
| | | | TTCR | |
| Tissue-11 | Lung cancer | within 24 hours | 10% DMSO/90% FBS | at −80° C. for |
| | tissue | | TTCR | 70 days |

### Specimen Tissue-12

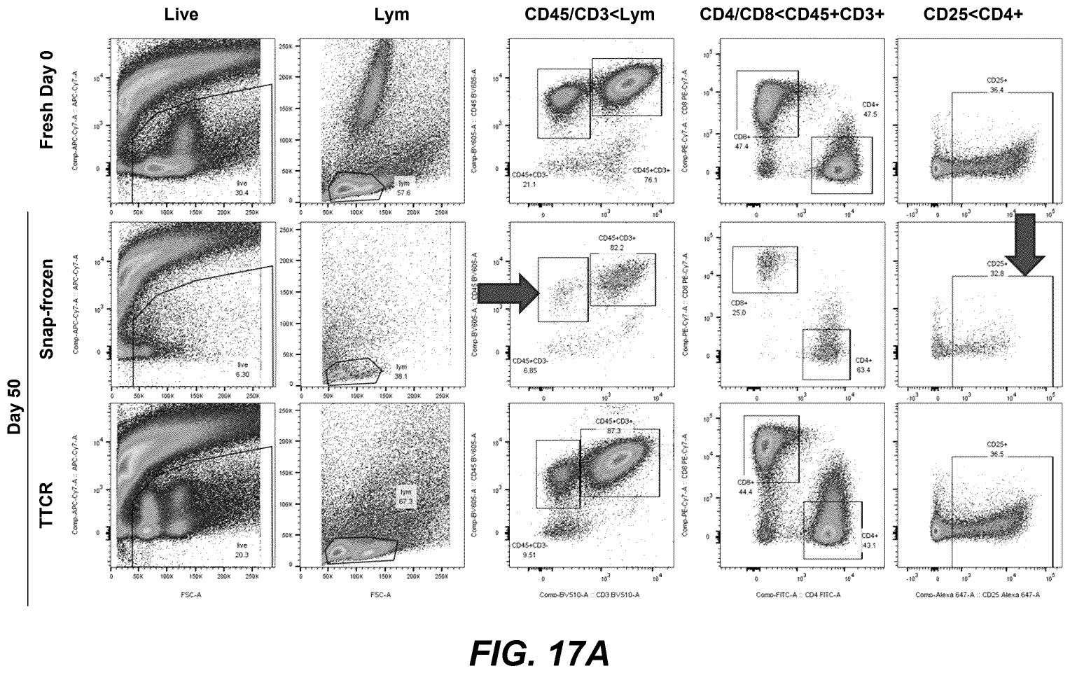

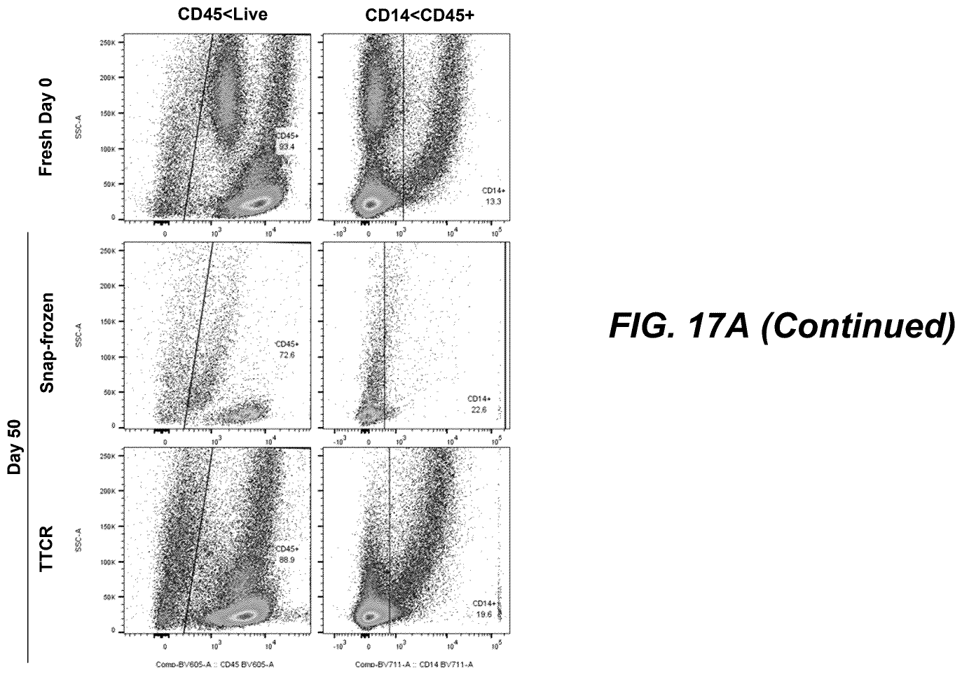

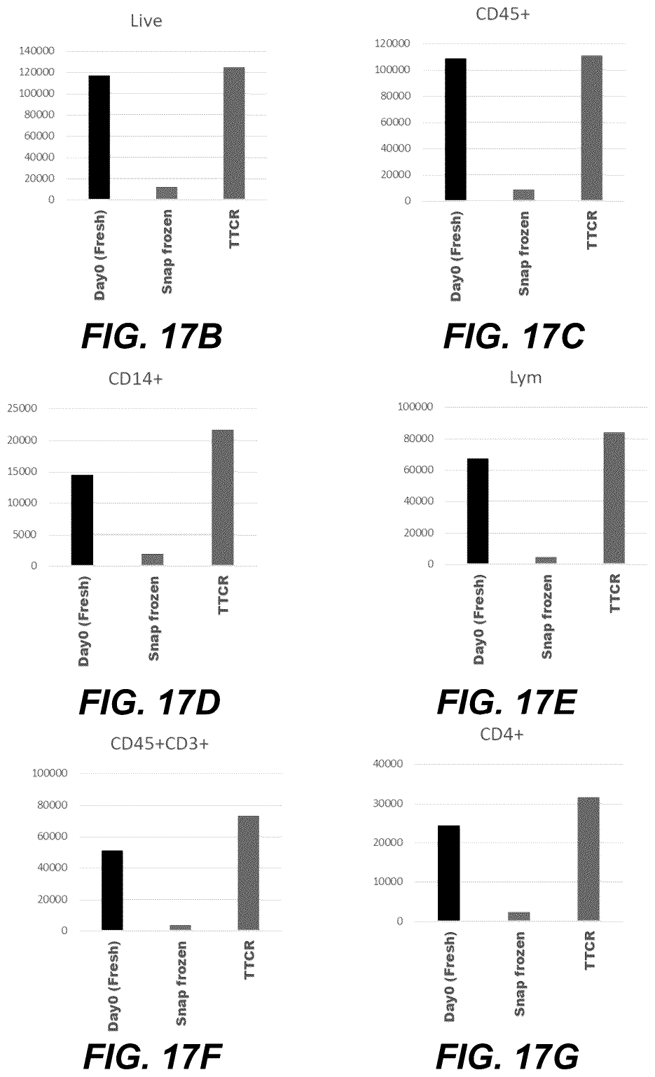

The surgical specimen was collected in TTPR (48 hours immersion in TTPR after specimen collection before freezing) and was cut to biopsy size (approximately 10 mg/piece). 3 pieces of cut tissue was put in each of the following test tubes: Tube 1 (21 mg, snap-frozen (on dry ice)); Tube 2 (20 mg, TTCR cryopreservation buffer); and Tube 3 (20 mg, fresh, Day 0, not frozen). Tubes 1 and 2 were frozen at −80° C. for 50 days. A single cell suspension was generated for each of Tubes 1-3 using TTDR and the cells underwent FACS analysis. FIGS. 17A-17S depict FACS data related to viability, lymphocyte (Lym), CD45/CD3<Lym, CD4/CD8<CD45+CD3+, CD25<CD4+, CD45<Live, and CD14<CD45+ properties for Specimen Tissue-12 at Day 0 (fresh) and after −80° C. for 50 days using either cryopreservation buffer TTCR or snap-frozen (FIG. 17A), cell count quantifications derived therefrom (FIGS. 17B-17J) for live (FIG. 17B), CD45+ (FIG. 17C), CD14+ (FIG. 17D), lymphocyte (Lym) (FIG. 17E), CD45+CD3+ (FIG. 17F), CD4+ (FIG. 17G), CD25+ (FIG. 17H), CD8+ (FIG. 17I), and CD45+CD3− (FIG. 17J) cells, and frequency (%) quantifications derived therefrom (FIGS. 17K-17S) for live (FIG. 17K), CD45+ (FIG. 17L), CD14+ (FIG. 17M), lymphocyte (Lym) (FIG. 17N), CD45+CD3+ (FIG. 17O), CD4+ (FIG. 17P), CD25+ (FIG. 17Q), CD8+ (FIG. 17R), and CD45+CD3− (FIG. 17S) cells. It was found that tissue chunk cryopreservation using TTCR can yield much more live cells as compared to snap-frozen tissue chunks.

### Specimen Tissue-13

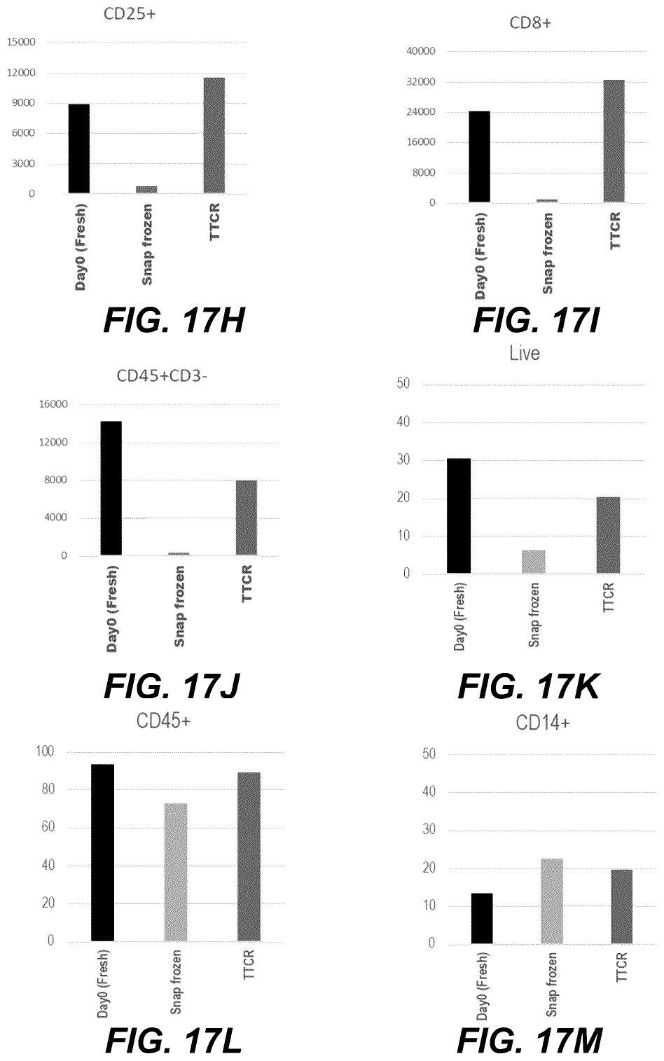

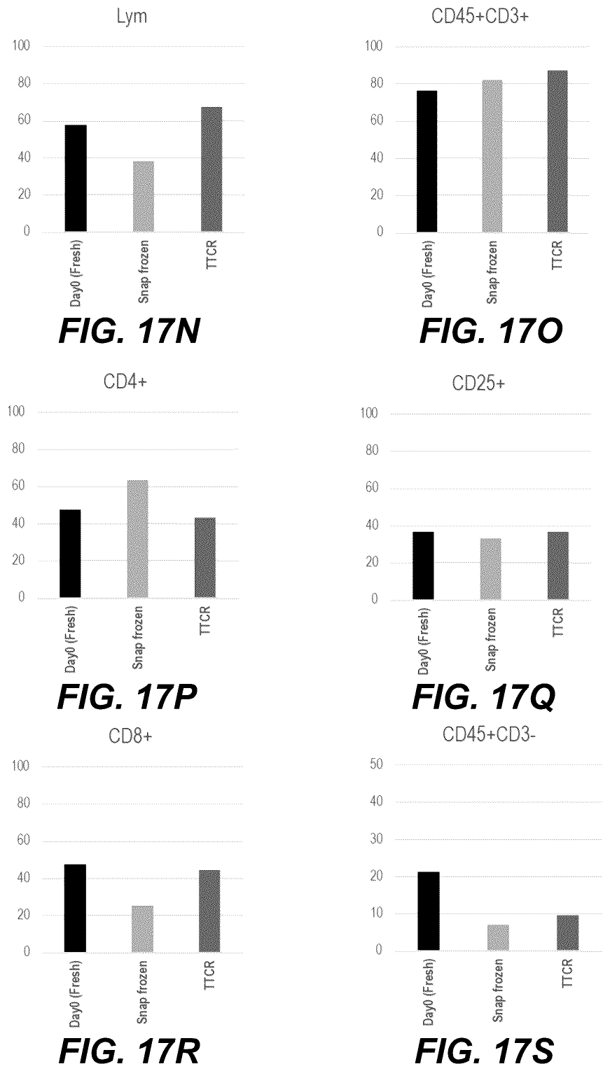

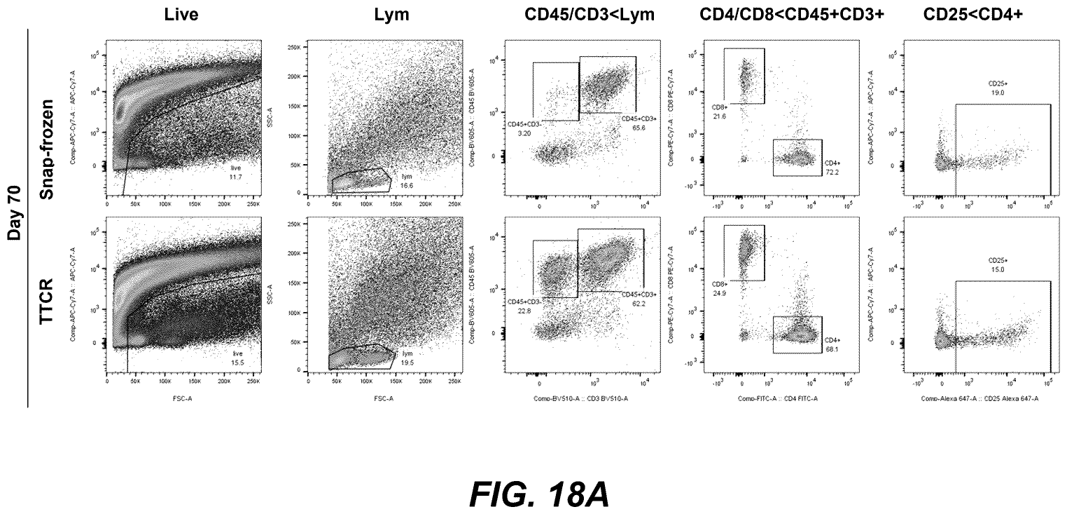

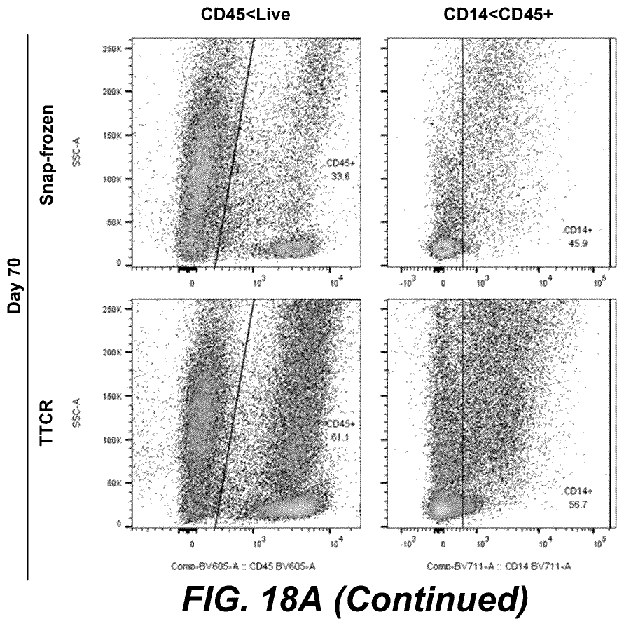

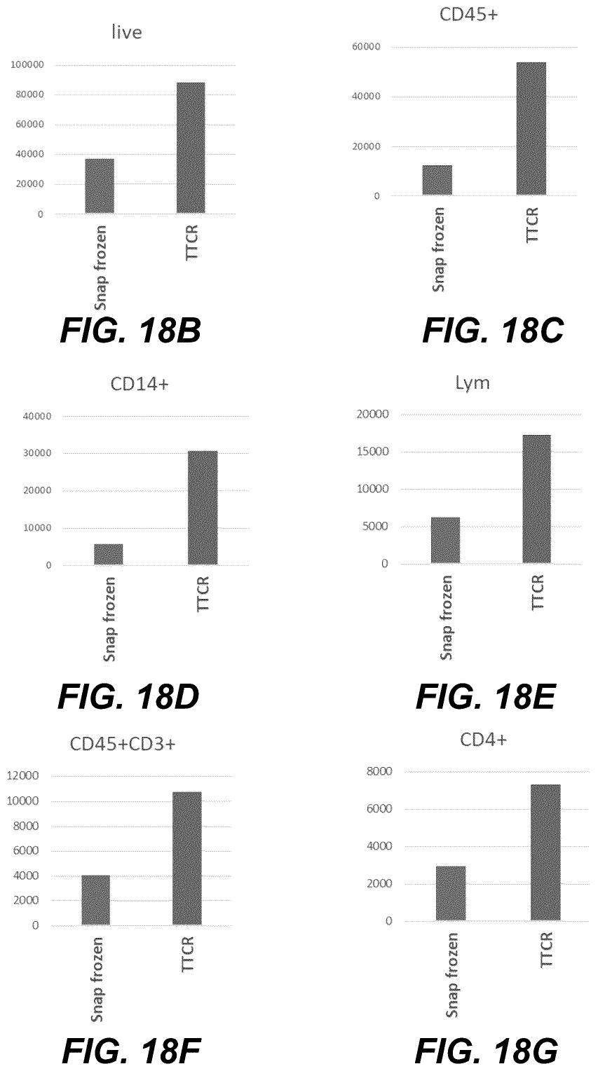

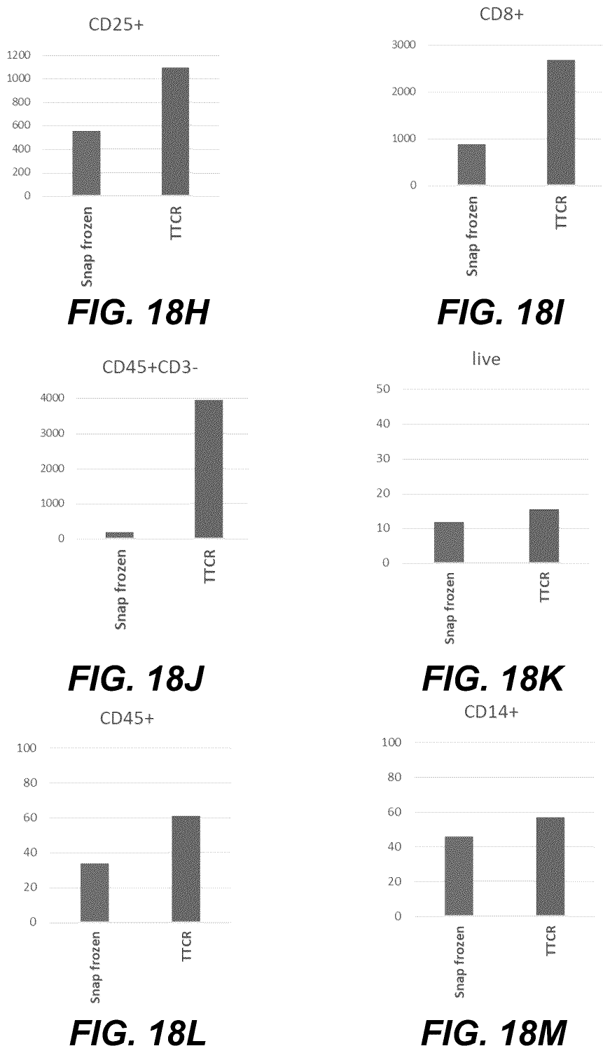

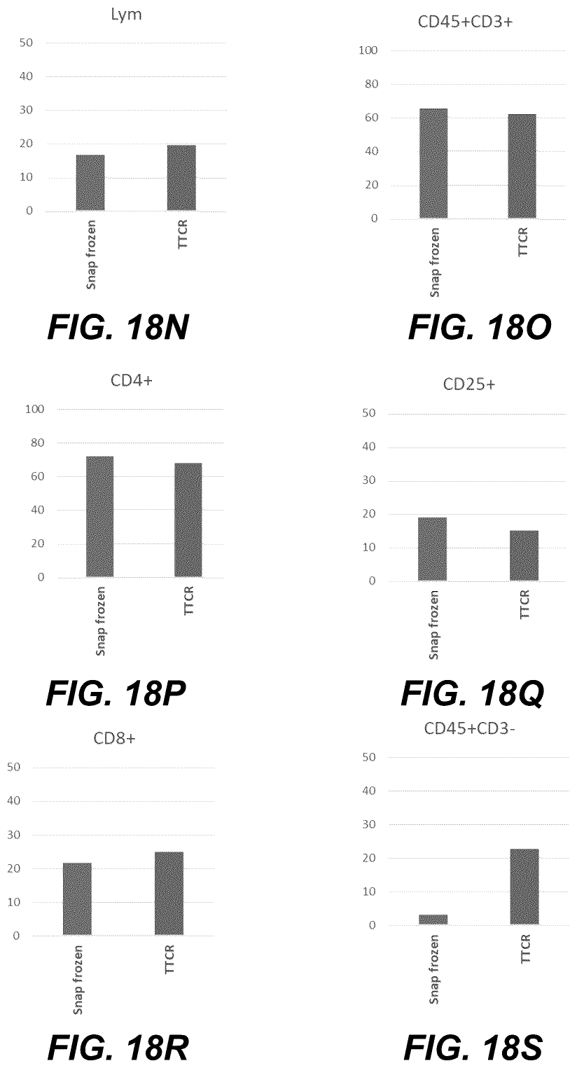

The surgical specimen was collected in TTPR (freezing within 24 hours of immersion in TTPR after specimen collection) and was cut to biopsy size (approximately 10 mg/piece). 2 pieces of cut tissue was put in each of the following test tubes: Tube 1 (18 mg, snap-frozen); and Tube 2 (17 mg, TTCR cryopreservation buffer). Tubes 1 and 2 were frozen at −80° C. for 70 days. A single cell suspension was generated for each of Tubes 1-2 using TTDR and the cells underwent FACS analysis. FIGS. 18A-18S depict FACS data related to viability, lymphocyte (Lym), CD45/CD3<Lym, CD4/CD8<CD45+CD3+, CD25<CD4+, CD45<Live, and CD14<CD45+ properties for Specimen Tissue-13 after −80° C. for 70 days using either cryopreservation buffer TTCR or snap-frozen (FIG. 18A), cell count quantifications derived therefrom (FIGS. 18B-18J) for live (FIG. 18B), CD45+ (FIG. 18C), CD14+ (FIG. 18D), lymphocyte (Lym) (FIG. 18E), CD45+CD3+ (FIG. 18F), CD4+ (FIG. 18G), CD25+ (FIG. 18H), CD8+ (FIG. 18I), and CD45+CD3− (FIG. 18J) cells, and frequency (%) quantifications derived therefrom (FIGS. 18K-18S) for live (FIG. 18K), CD45+ (FIG. 18L), CD14+ (FIG. 18M), lymphocyte (Lym) (FIG. 18N), CD45+CD3+ (FIG. 18O), CD4+ (FIG. 18P), CD25+ (FIG. 18Q), CD8+ (FIG. 18R), and CD45+CD3− (FIG. 18S) cells.

### Specimen Tissue-14

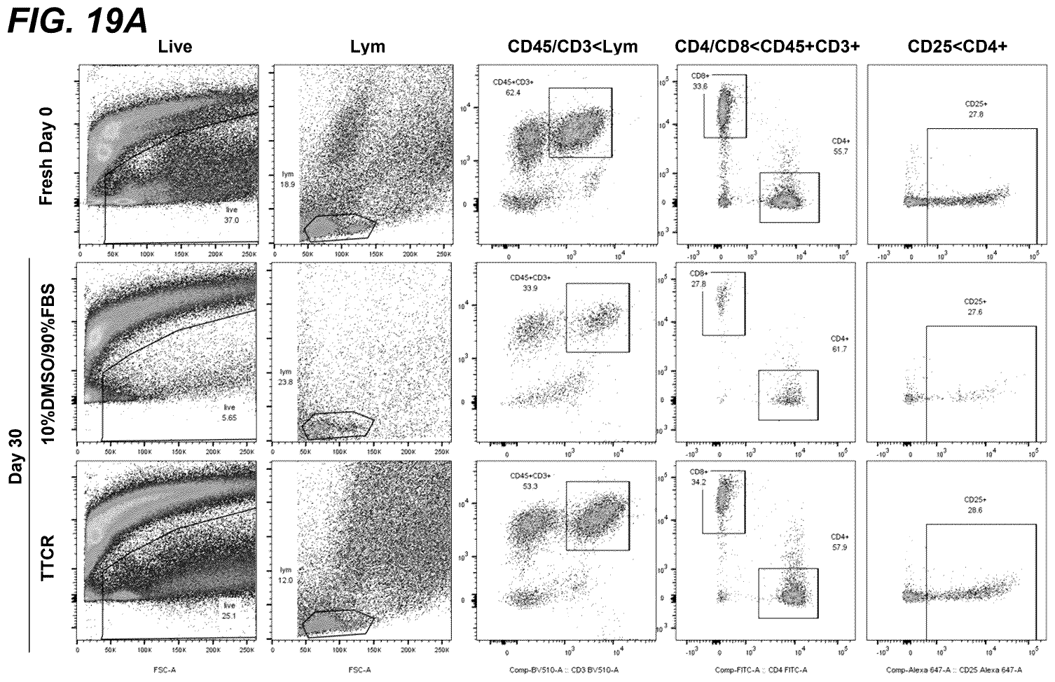

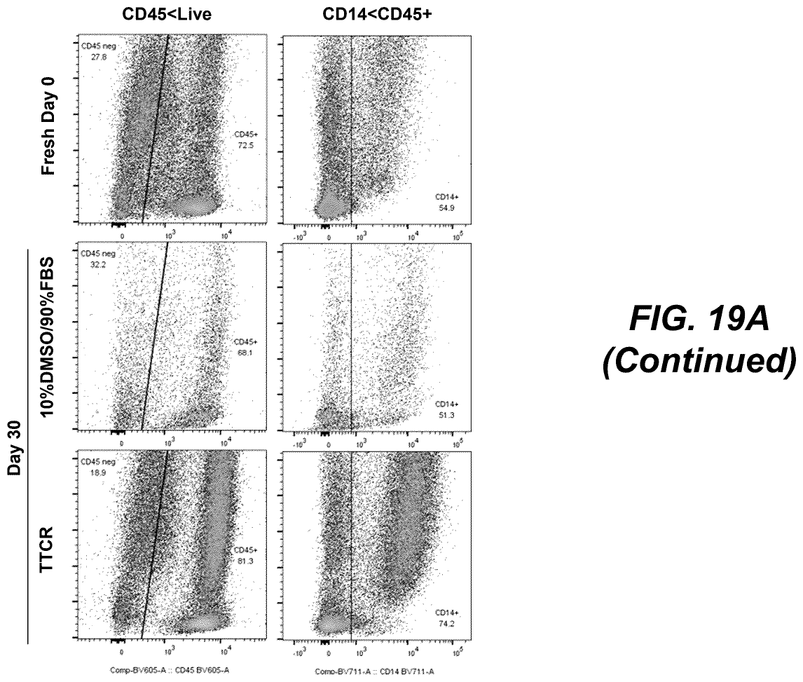

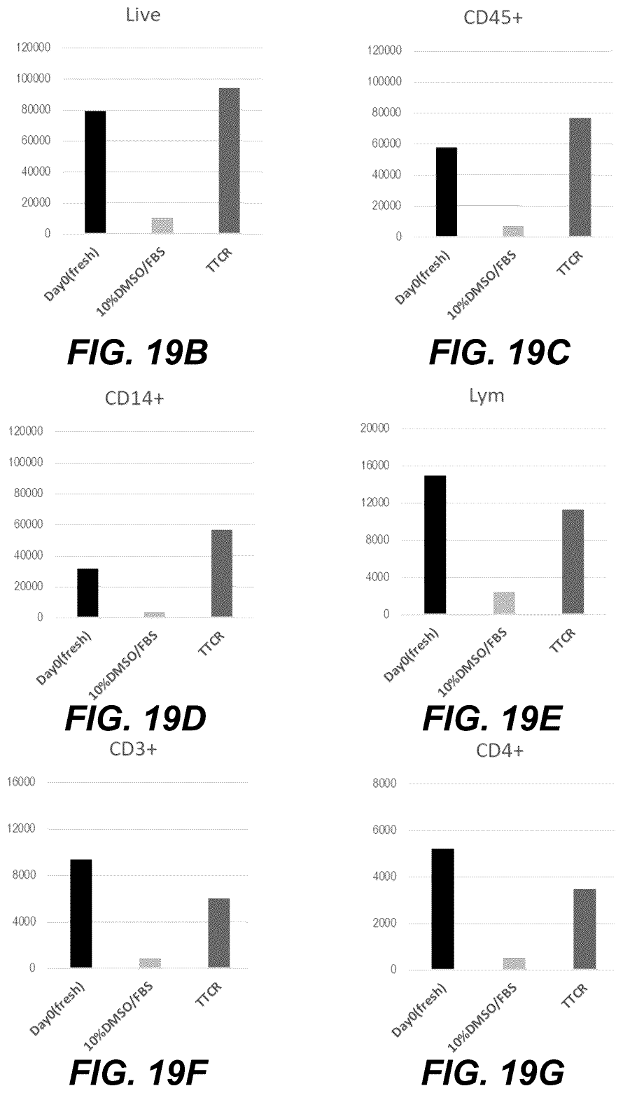

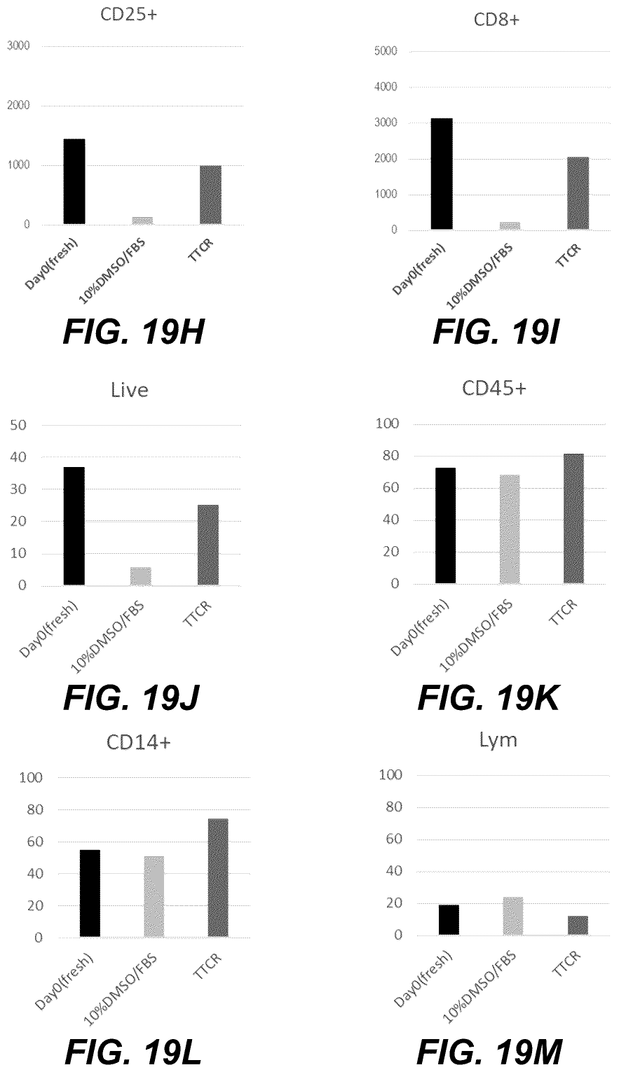

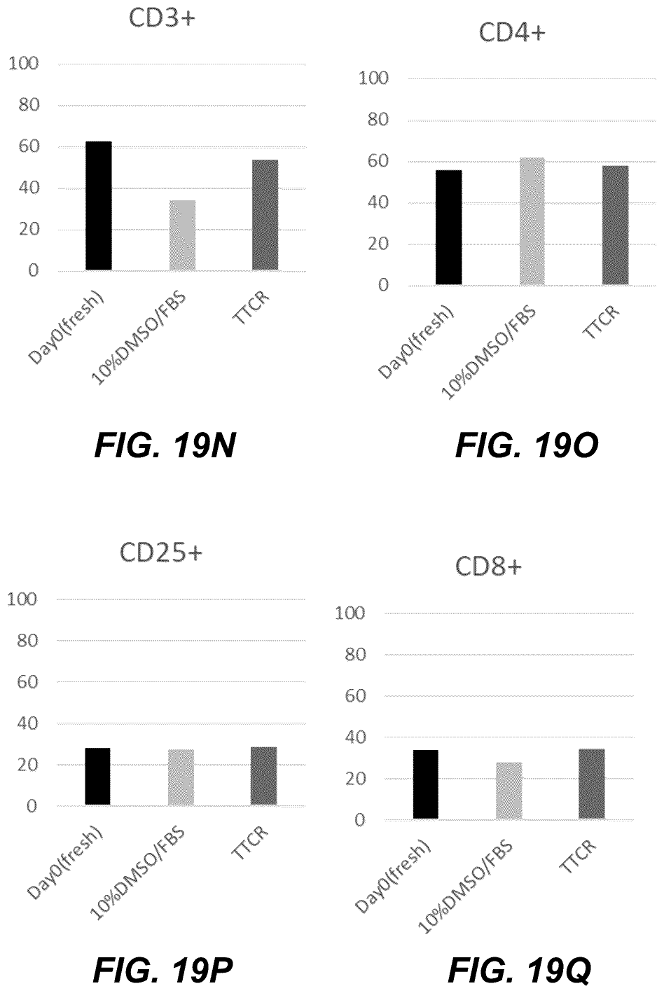

The surgical specimen was collected in TTPR (freezing within 24 hours of immersion in TTPR after specimen collection) and was cut to biopsy size (approximately 10 mg/piece). 2 pieces of cut tissue was put in each of the following test tubes: Tube 1 (14 mg, 10% DMSO/FBS (conventional buffer)); Tube 2 (14 mg, TTCR cryopreservation buffer); and Tube 3 (14 mg, fresh, Day 0, not frozen). Tubes 1 and 2 were frozen at −80° C. for 30 days. A single cell suspension was generated for each of Tubes 1-3 using TTDR and the cells underwent FACS analysis. FIGS. 19A-19Q depict FACS data related to viability, lymphocyte (Lym), CD45/CD3<Lym, CD4/CD8<CD45+CD3+, CD25<CD4+, CD45<Live, and CD14<CD45+ properties for Specimen Tissue-14 at Day 0 (fresh) and after −80° C. for 30 days using cryopreservation buffers 10% DMSO/90% FBS and TTCR (FIG. 19A), cell count quantifications derived therefrom (FIGS. 19B-19I) for live (FIG. 19B), CD45+ (FIG. 19C), CD14+ (FIG. 19D), lymphocyte (Lym) (FIG. 19E), CD3+ (FIG. 19F), CD4+ (FIG. 19G), CD25+ (FIG. 19H), and CD8+ (FIG. 19I) cells, and frequency (%) quantifications derived therefrom (FIGS. 19J-19Q) for live (FIG. 19J), CD45+ (FIG. 19K), CD14+ (FIG. 19L), lymphocyte (Lym) (FIG. 19M), CD3+ (FIG. 19N), CD4+ (FIG. 19O), CD25+ (FIG. 19P), and CD8+ (FIG. 19Q) cells. It was found that tissue chunk cryopreservation using TTCR can yield much more live cells as compared to tissue chunk cryopreservation using 10% DMSO/FBS.

### Specimen Tissue-11

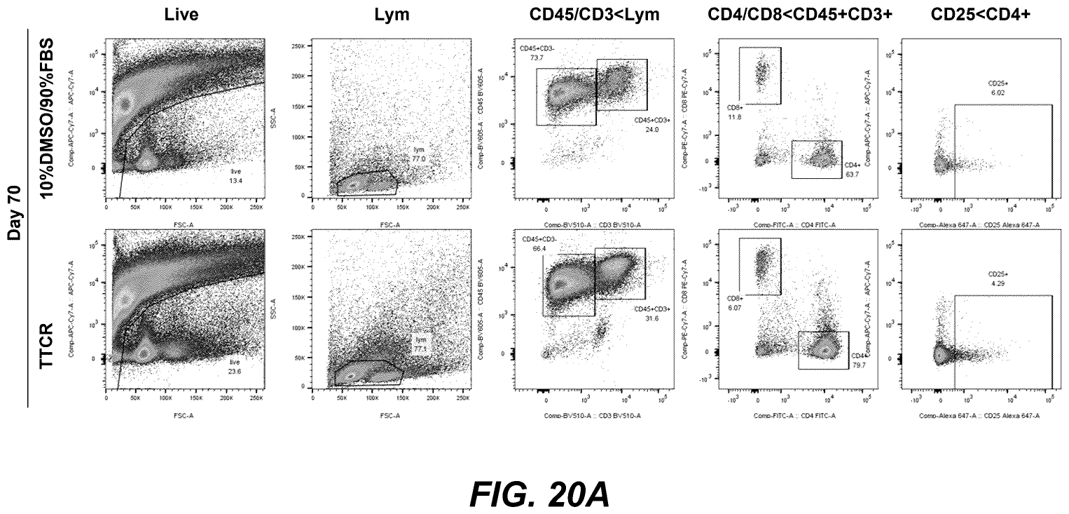

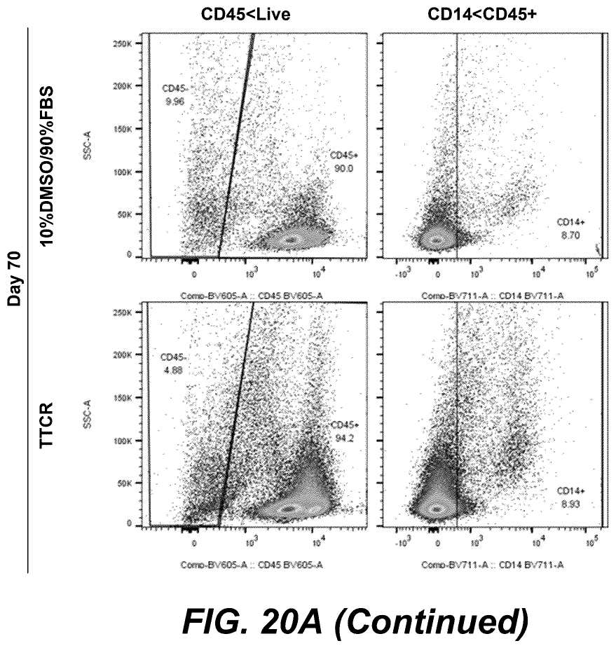

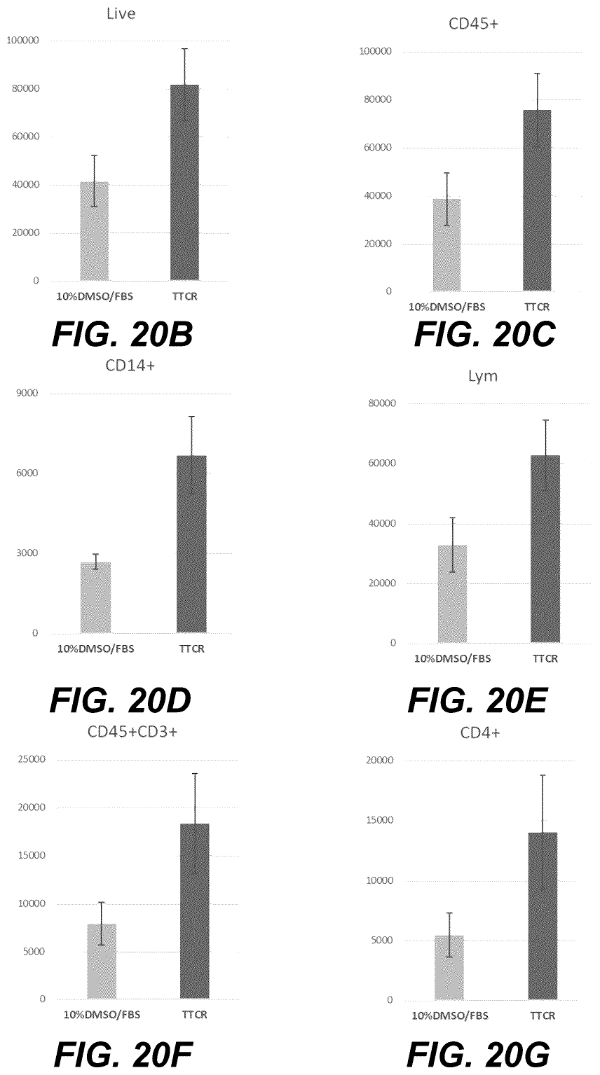

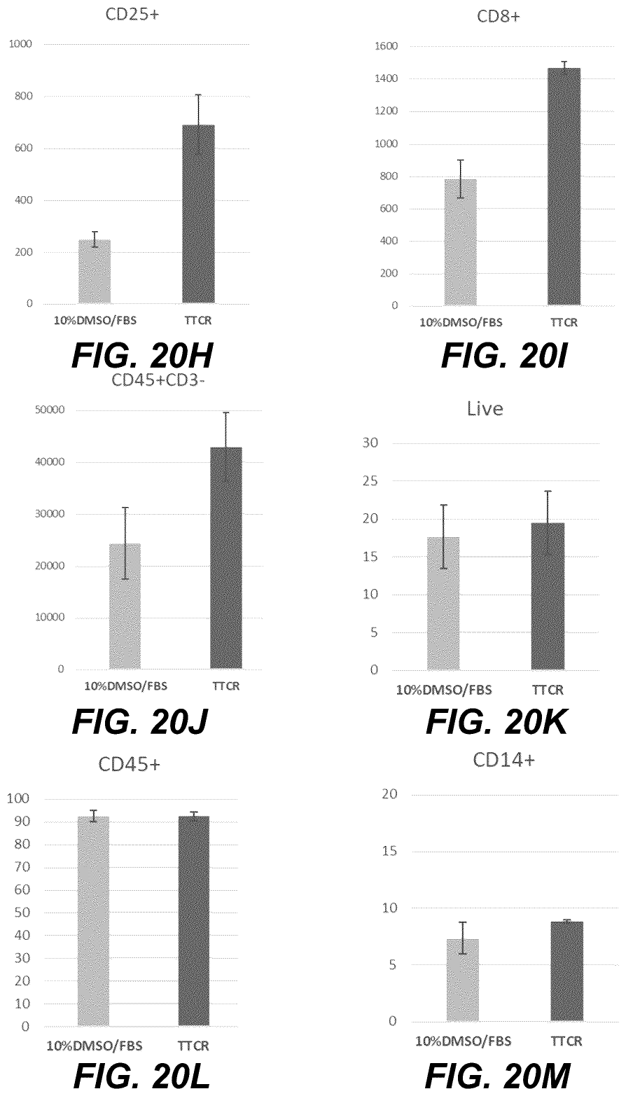

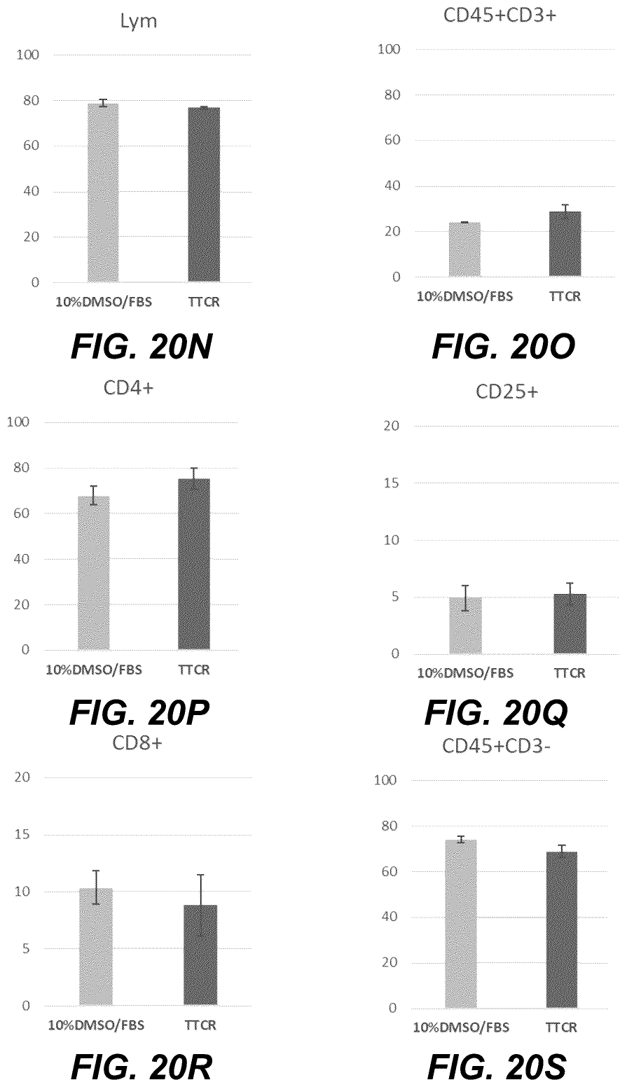

The surgical specimen was collected in TTPR (freezing within 24 hours of immersion in TTPR after specimen collection) and was cut to biopsy size (approximately 10 mg/piece). 3 pieces of cut tissue was put in each of the following test tubes: Tube 1a (23 mg, 10% DMSO/FBS (commonly used)); Tube 1b (24 mg, 10% DMSO/FBS); Tube 2a (22 mg, TTCR cryopreservation buffer); and Tube 2b (23 mg, TTCR). The a and b tubes served as duplicates. Tubes 1a, 1b, 2a, and 2b were frozen at −80° C. for 70 days. A single cell suspension was generated for each of Tubes 1a, 1b, 2a, and 2b using TTDR and the cells underwent FACS analysis. FIGS. 20A-20S depict FACS data related to viability, lymphocyte (Lym), CD45/CD3<Lym, CD4/CD8<CD45+CD3+, CD25<CD4+, CD45<Live, and CD14<CD45+ properties for Specimen Tissue-11 after −80° C. for 70 days using cryopreservation buffers 10% DMSO/90% FBS and TTCR (FIG. 20A), cell count quantifications derived therefrom (FIGS. 20B-20J) for live (FIG. 20B), CD45+ (FIG. 20C), CD14+ (FIG. 20D), lymphocyte (Lym) (FIG. 20E), CD45+CD3+ (FIG. 20F), CD4+ (FIG. 20G), CD25+ (FIG. 20H), CD8+ (FIG. 20I), and CD45+CD3− (FIG. 20J) cells, and frequency (%) quantifications derived therefrom (FIGS. 20K-20S) for live (FIG. 20K), CD45+ (FIG. 20L), CD14+ (FIG. 20M), lymphocyte (Lym) (FIG. 20N), CD45+CD3+ (FIG. 20O), CD4+ (FIG. 20P), CD25+ (FIG. 20Q), CD8+ (FIG. 20R), and CD45+CD3− (FIG. 20S) cells.

### Example 4

### Cryopreservation Studies Comparing Tissue Chunk and Tissue-Dissociated Cell Suspension

In this example TTCR-cryopreserved tissue chunks and TTCR-cryopreserved tissue-dissociated cell suspensions were compared. As described in Example 3, the ability to cryopreserve tissue chunks is quite advantageous, especially in a clinical setting. It was found that many more viable cells can be collected in TTCR-cryopreserved tissue chunks (tissue chunk frozen in TTCR) compared to TTCR-cryopreserved tissue-dissociated cell suspension-frozen, and surprisingly, TTCR-cryopreserved tissue chunks can yield more viable cells than Fresh cells (Day 0) depending on the features of tumor tissues. In some embodiments, without being bound by any particular theory, the freeze-thaw process may make tumor tissues softer (or some strong tissue structures might be broken by it) such that TTDR can work especially well in tissue dissociation. Accordingly, TTCR-based cryopreservation of tissue chunks can be more effective in embodiments wherein harder tissues are being studied to yield more viable cells from tissue.

### Experimental Workflow

Surgical specimens (described in Table 18) were collected in TTPR, cut into biopsy size, and then divided with equal tissue weight (2˜3 chunks/sample) into following samples: a first sample (Day 0 fresh); a second sample (“Cell-S”); and a third sample (“Chunk”). The first sample was not frozen, but instead underwent dissociation with TTDR to generate a single cell suspension which then underwent FACS analysis. The second sample underwent dissociation with TTDR to generate a single cell suspension, was frozen using TTCR, thawed, and then underwent FACS analysis. The third sample was frozen using TTCR, thawed, underwent dissociation with TTDR to generate a single cell suspension, and then underwent FACS analysis.

TABLE 18
Specimens

| Specimen | | TTPR | | |
|---|---|---|---|---|
| ID | Tumor Tissue | immersing | Preservation | Periods |
| Tissue-15 | Lung cancer tissue | Within 24 hours | −80° C.⇒Liquid Nitrogen | 60 days |
| Tissue-16 | Lung cancer tissue | Within 24 hours | −80° C.⇒Liquid Nitrogen | 60 days |
| Tissue-17 | Lung cancer tissue | Within 24 hours | −80° C.⇒Liquid Nitrogen | 60 days |
| Tissue-18 | Lung cancer tissue | Within 24 hours | −80° C.⇒Liquid Nitrogen | 60 days |
| Tissue-19 | Lung cancer tissue | Within 24 hours | −80° C.⇒Liquid Nitrogen | 90 days |
| Tissue-20 | Lymph Node from lung | Within 24 hours | at −80° C. | 25 days |
| | cancer patient | | | |

### Specimen Tissue-15

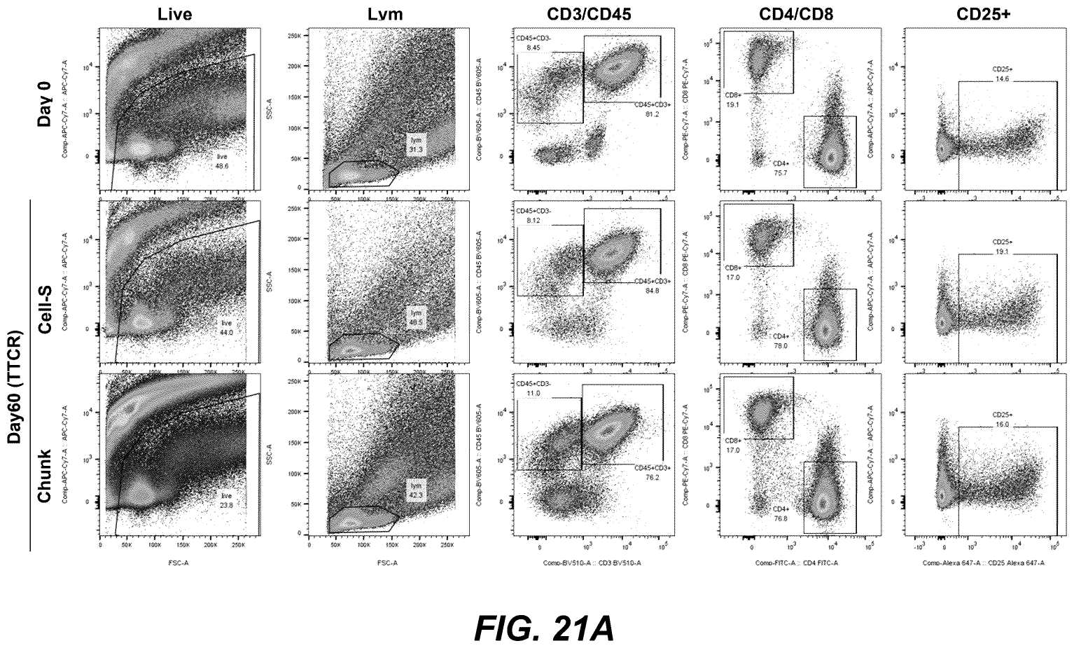

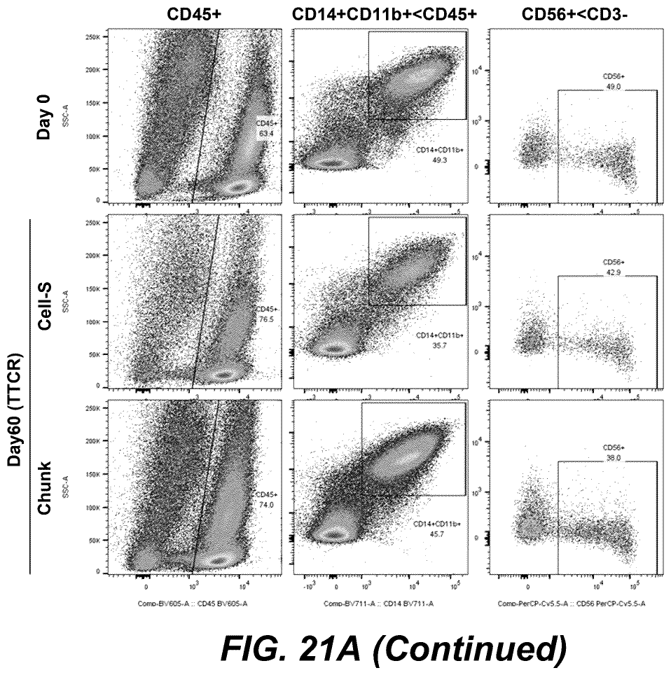

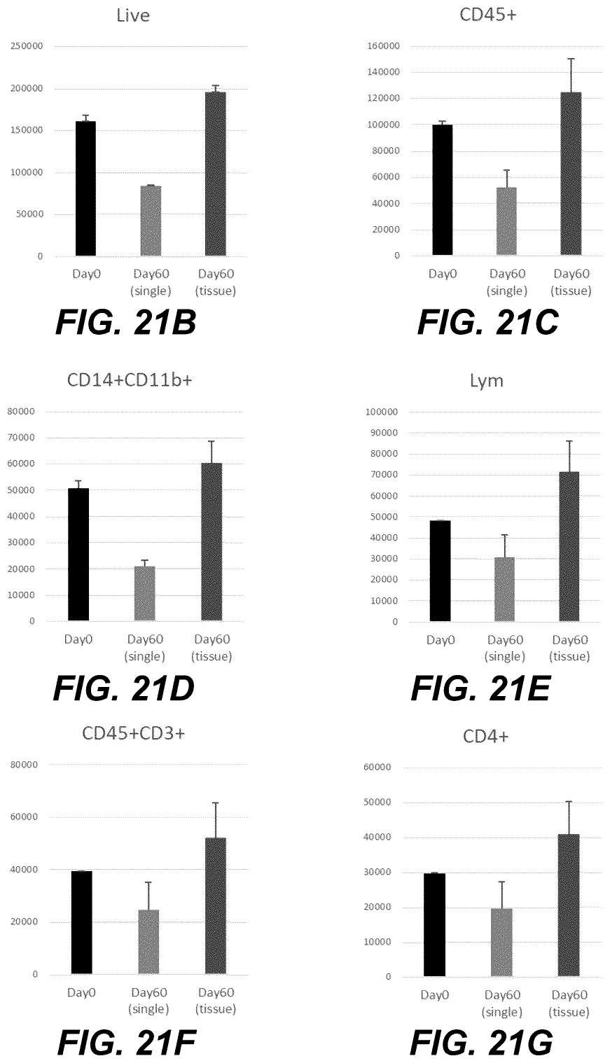

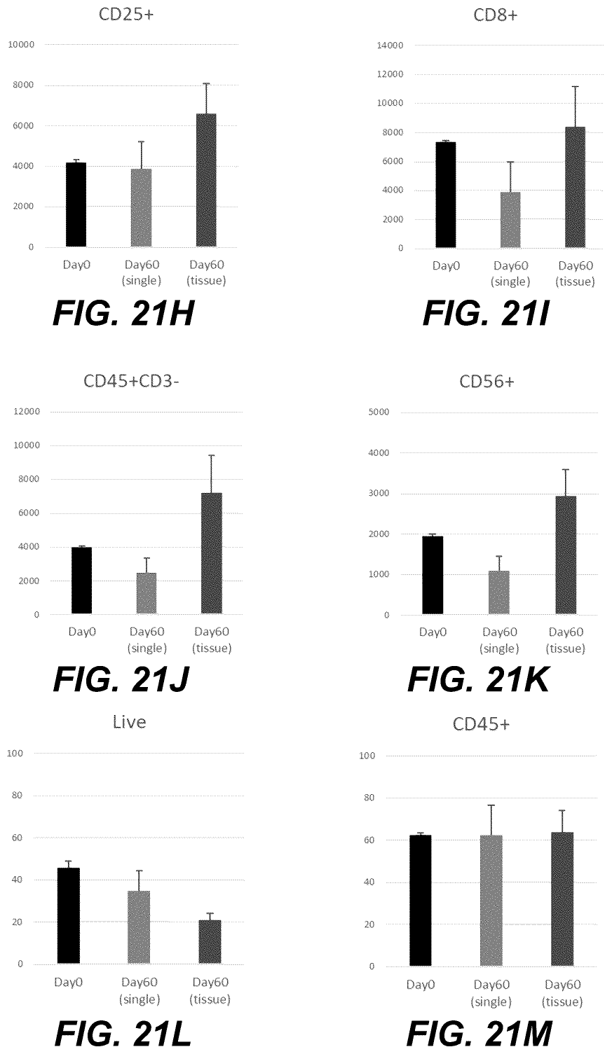

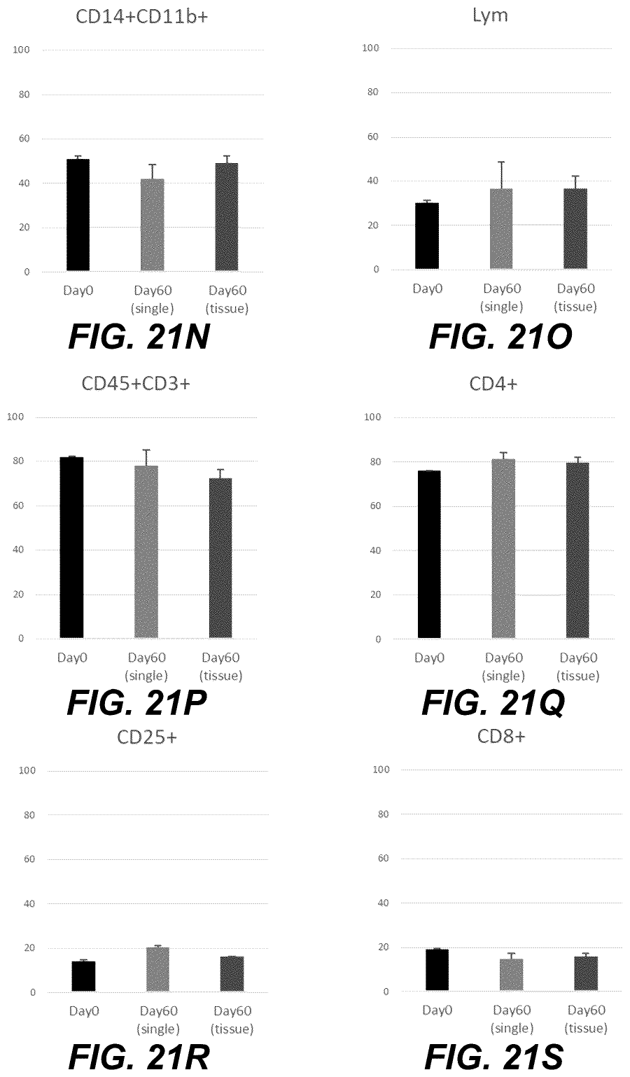

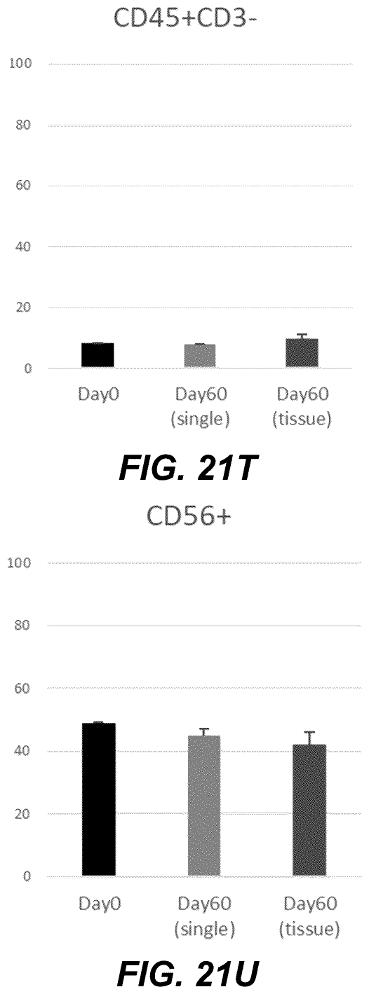

FIGS. 21A-21U depict FACS data related to viability, lymphocyte (Lym), CD3/CD45, CD4/CD8, CD25+, CD45+, CD14+CD11b+<CD45+, and CD56+<CD3-properties for Specimen Tissue-15 at Day 0 and after −80° C. for 60 days using cryopreservation buffer TTCR (Cell-S and Chunk) (FIG. 21A), cell count quantifications derived therefrom (FIGS. 21B-21K) for live (FIG. 21B), CD45+ (FIG. 21C), CD14+CD11b+ (FIG. 21D), lymphocyte (Lym) (FIG. 21E), CD45+CD3+ (FIG. 21F), CD4+ (FIG. 21G), CD25+ (FIG. 21H), CD8+ (FIG. 21I), CD45+CD3− (FIG. 21J), and CD56+ (FIG. 21K) cells, and frequency (%) quantifications derived therefrom (FIGS. 21L-21U) for live (FIG. 21L), CD45+ (FIG. 21M), CD14+CD11b+ (FIG. 21N), lymphocyte (Lym) (FIG. 21O), CD45+CD3+ (FIG. 21P), CD4+ (FIG. 21Q), CD25+ (FIG. 21R), CD8+ (FIG. 21S), CD45+CD3− (FIG. 21T), and CD56+ (FIG. 21U) cells. Data shown for duplicates.

### Specimen Tissue-16

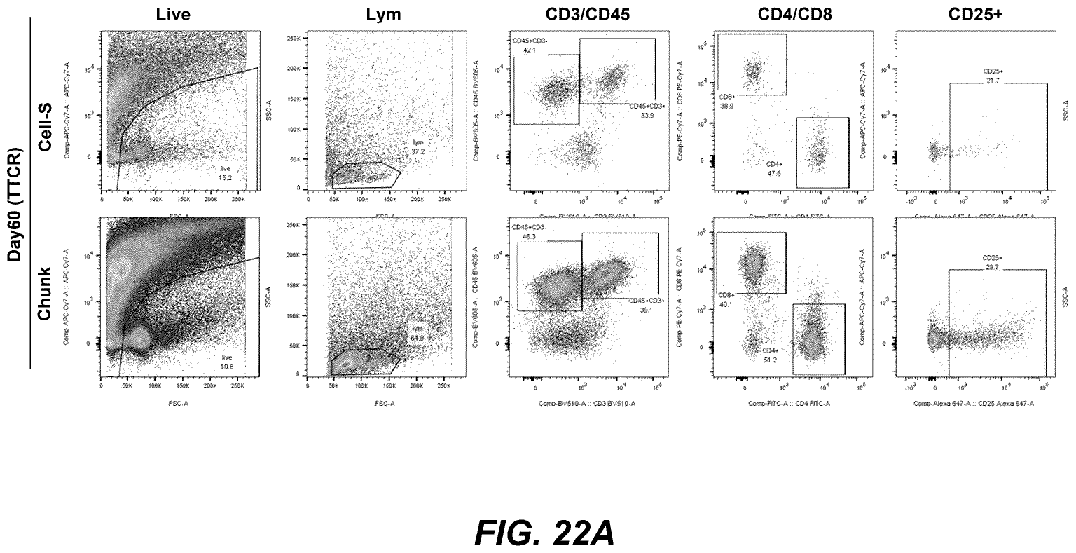

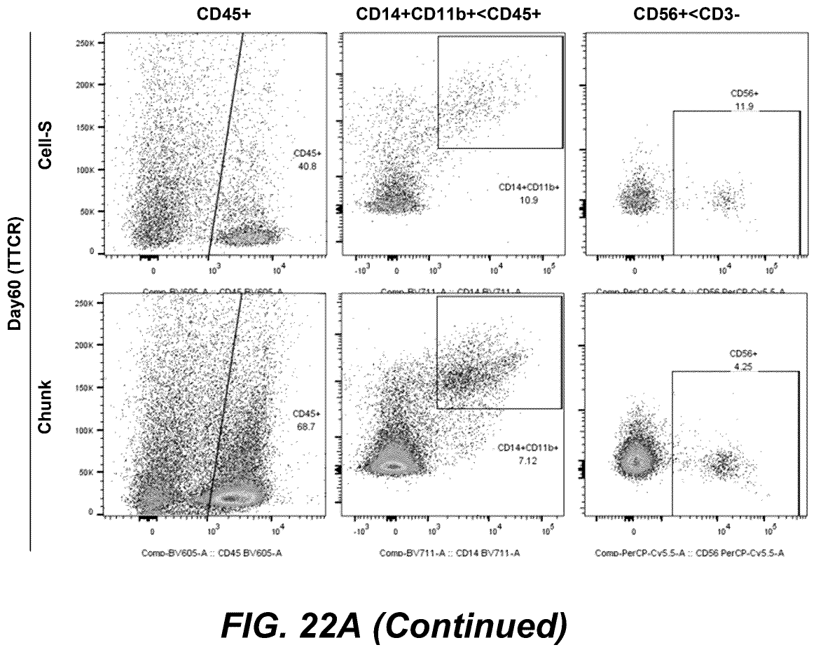

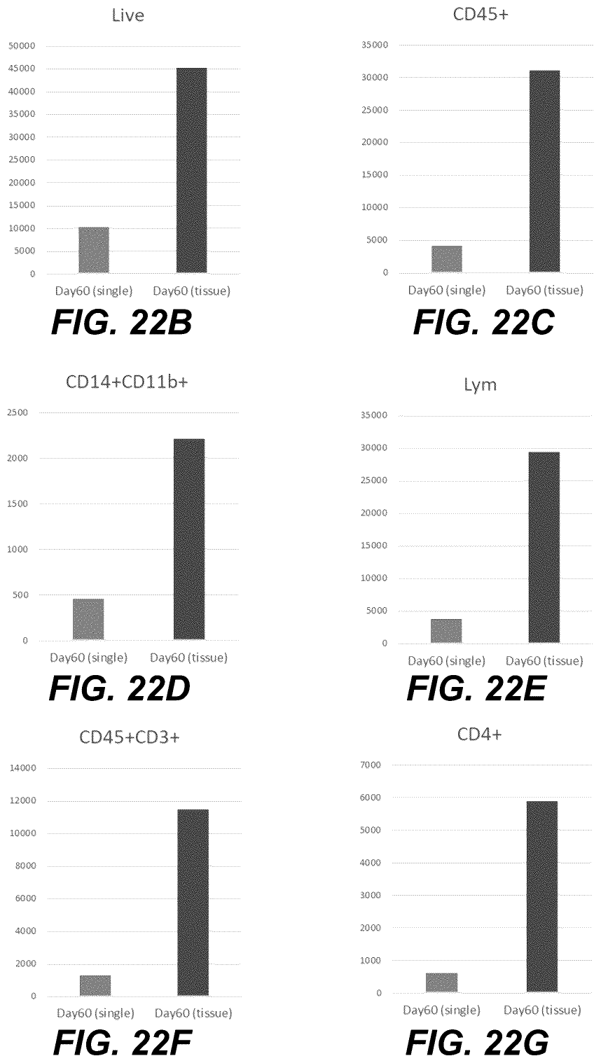

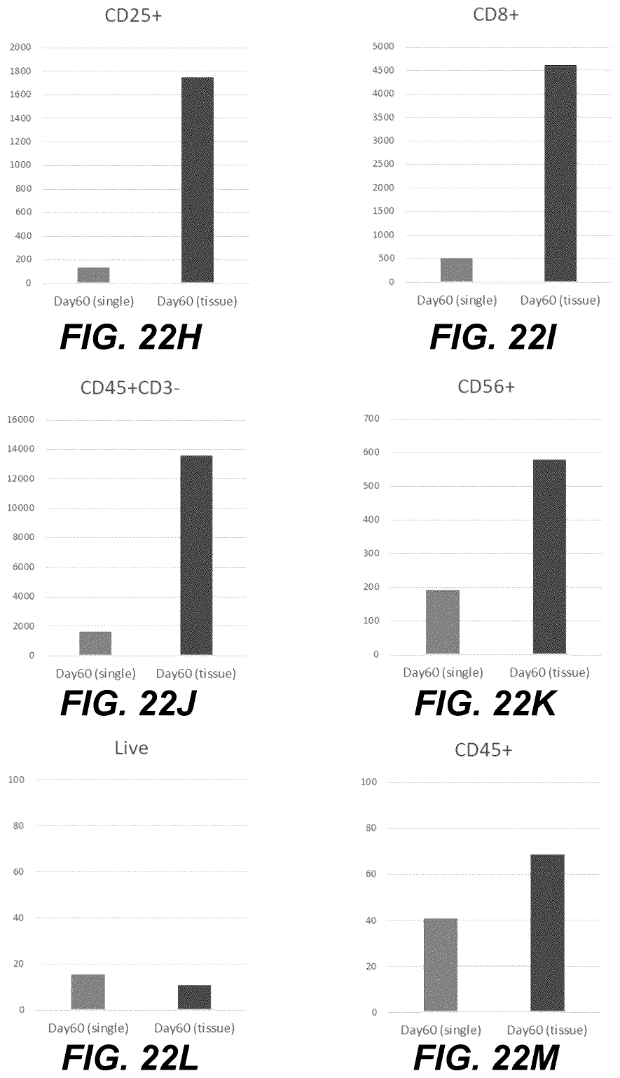

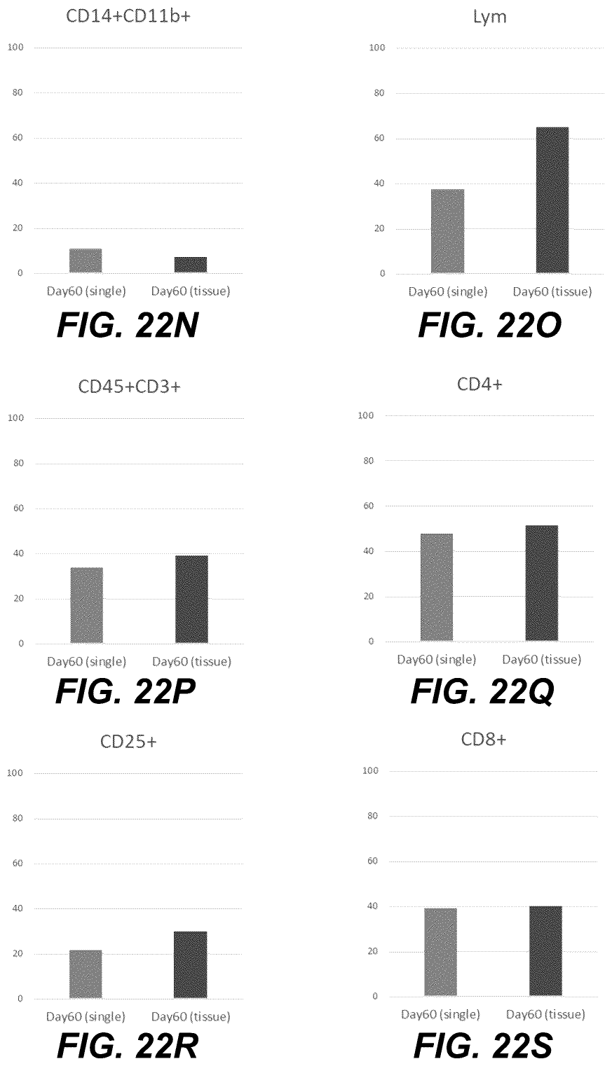

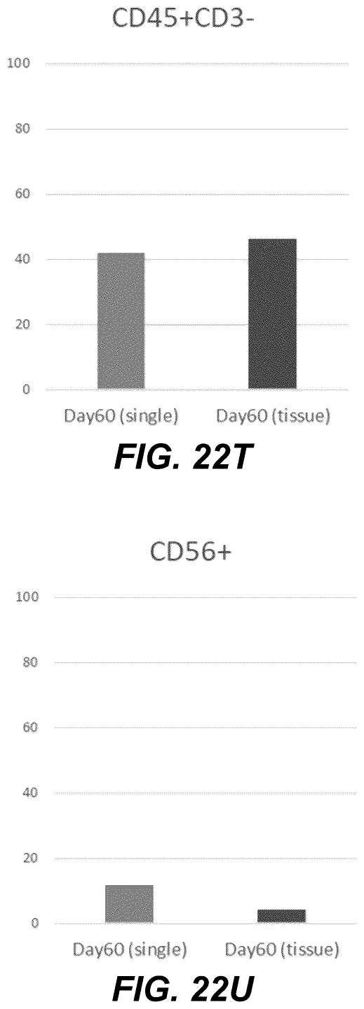

FIGS. 22A-22U depict FACS data related to viability, lymphocyte (Lym), CD3/CD45, CD4/CD8, CD25+, CD45+, CD14+CD11b+<CD45+, and CD56+<CD3-properties for Specimen Tissue-16 after −80° C. for 60 days using cryopreservation buffer TTCR (Cell-S and Chunk) (FIG. 22A), cell count quantifications derived therefrom (FIGS. 22B-22K) for live (FIG. 22B), CD45+ (FIG. 22C), CD14+CD11b+ (FIG. 22D), lymphocyte (Lym) (FIG. 22E), CD45+CD3+ (FIG. 22F), CD4+ (FIG. 22G), CD25+ (FIG. 22H), CD8+ (FIG. 22I), CD45+CD3− (FIG. 22J), and CD56+ (FIG. 22K) cells, and frequency (%) quantifications derived therefrom (FIGS. 22L-22U) for live (FIG. 22L), CD45+ (FIG. 22M), CD14+CD11b+ (FIG. 22N), lymphocyte (Lym) (FIG. 22O), CD45+CD3+ (FIG. 22P), CD4+ (FIG. 22Q), CD25+ (FIG. 22R), CD8+ (FIG. 22S), CD45+CD3− (FIG. 22T), and CD56+ (FIG. 22U) cells.

### Specimen Tissue-17

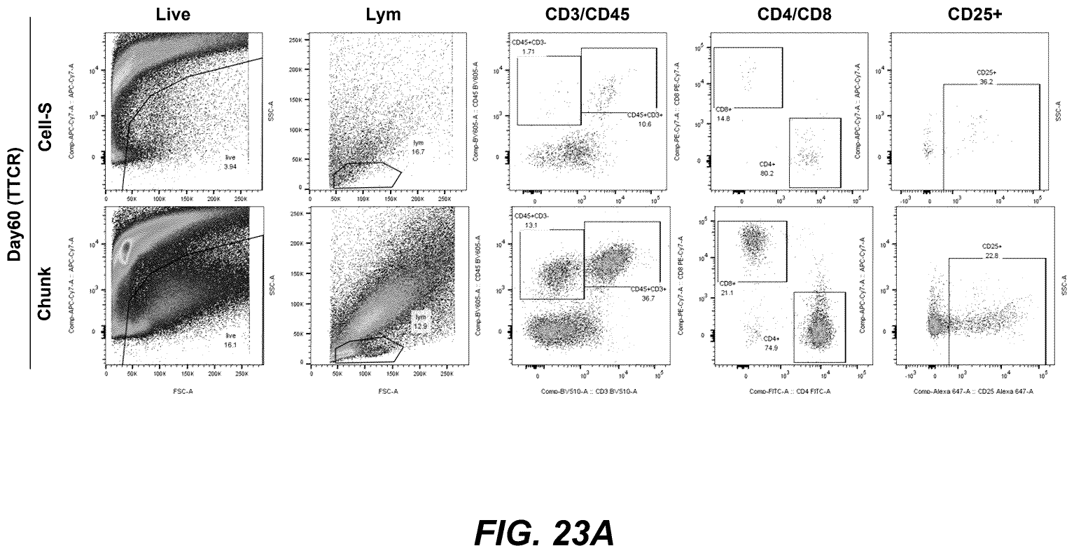

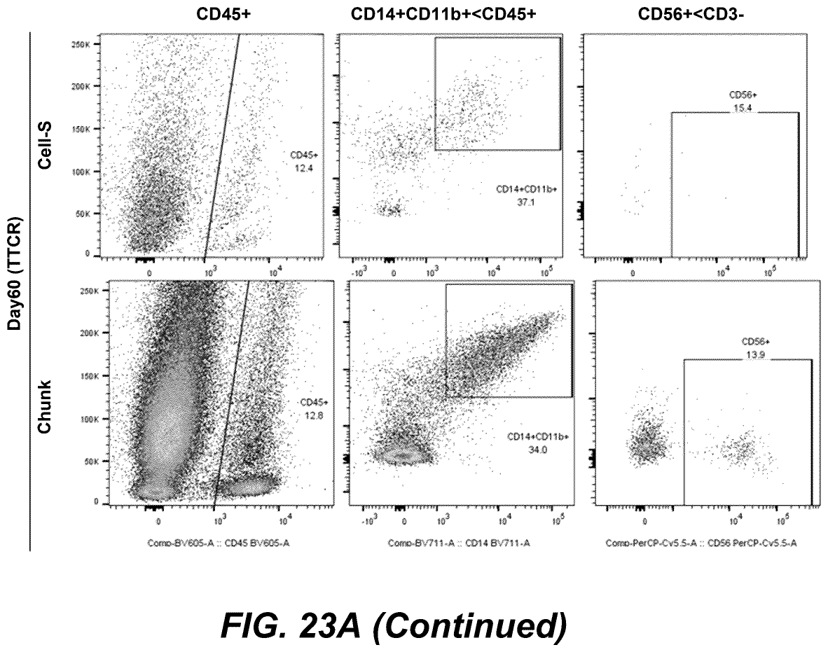

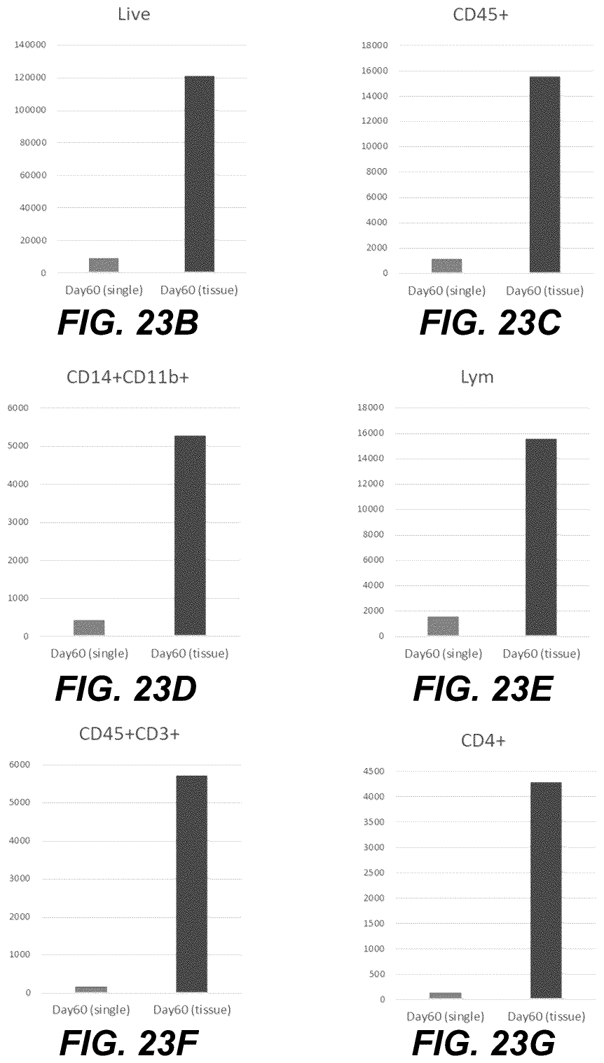

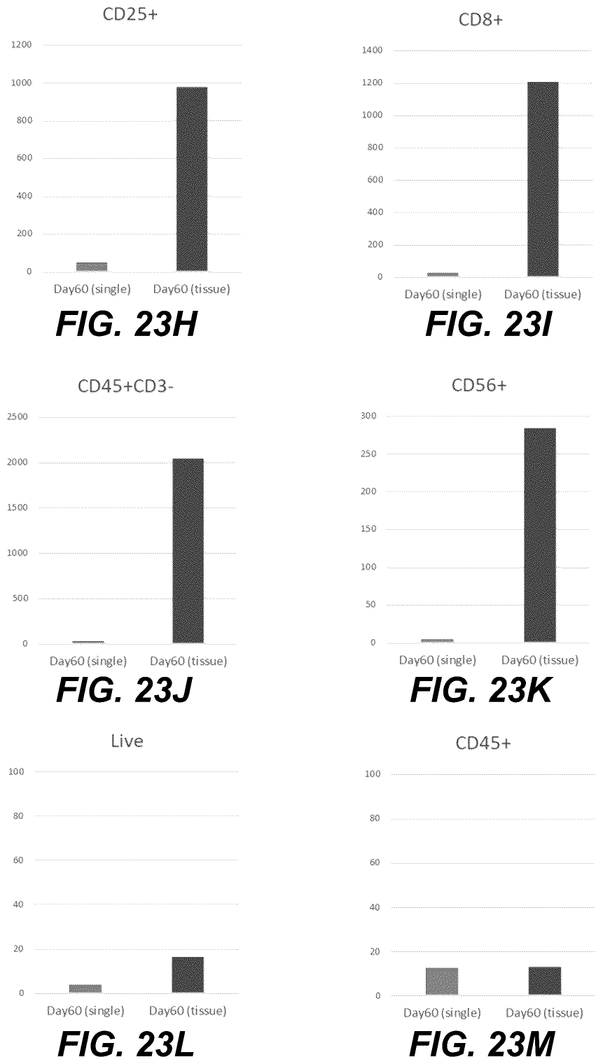

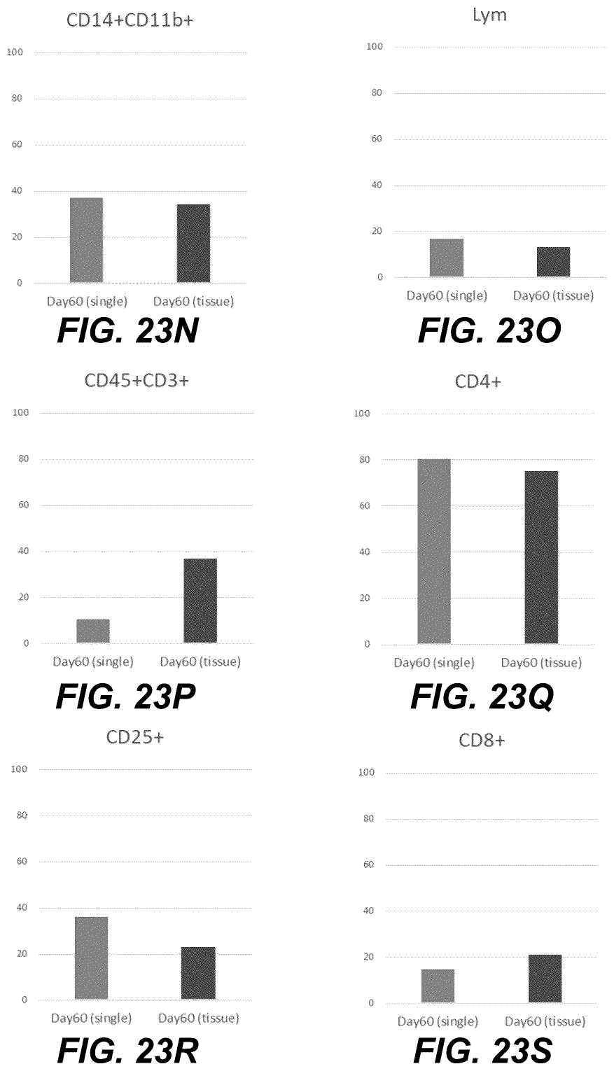

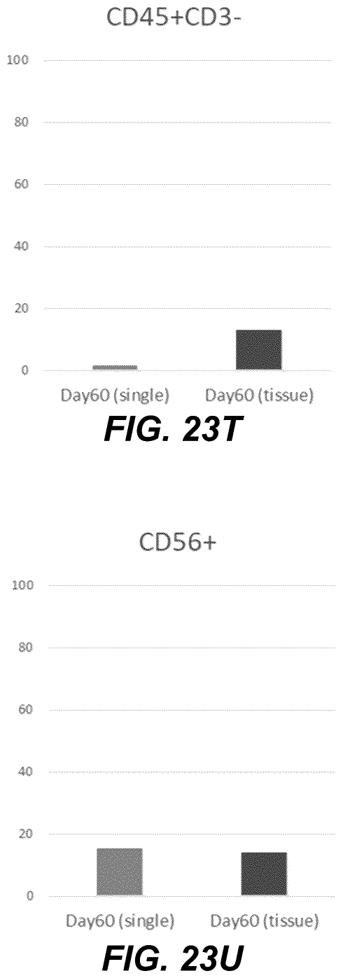

FIGS. 23A-23U depict FACS data related to viability, lymphocyte (Lym), CD3/CD45, CD4/CD8, CD25+, CD45+, CD14+CD11b+<CD45+, and CD56+<CD3-properties for Specimen Tissue-17 after −80° C. for 60 days using cryopreservation buffer TTCR (Cell-S and Chunk) (FIG. 23A), cell count quantifications derived therefrom (FIGS. 23B-23K) for live (FIG. 23B), CD45+ (FIG. 23C), CD14+CD11b+ (FIG. 23D), lymphocyte (Lym) (FIG. 23E), CD45+CD3+ (FIG. 23F), CD4+ (FIG. 23G), CD25+ (FIG. 23H), CD8+ (FIG. 23I), CD45+CD3− (FIG. 23J), and CD56+ (FIG. 23K) cells, and frequency (%) quantifications derived therefrom (FIGS. 23L-23U) for live (FIG. 23L), CD45+ (FIG. 23M), CD14+CD11b+ (FIG. 23N), lymphocyte (Lym) (FIG. 23O), CD45+CD3+ (FIG. 23P), CD4+ (FIG. 23Q), CD25+ (FIG. 23R), CD8+ (FIG. 23S), CD45+CD3− (FIG. 23T), and CD56+ (FIG. 23U) cells.

### Specimen Tissue-18

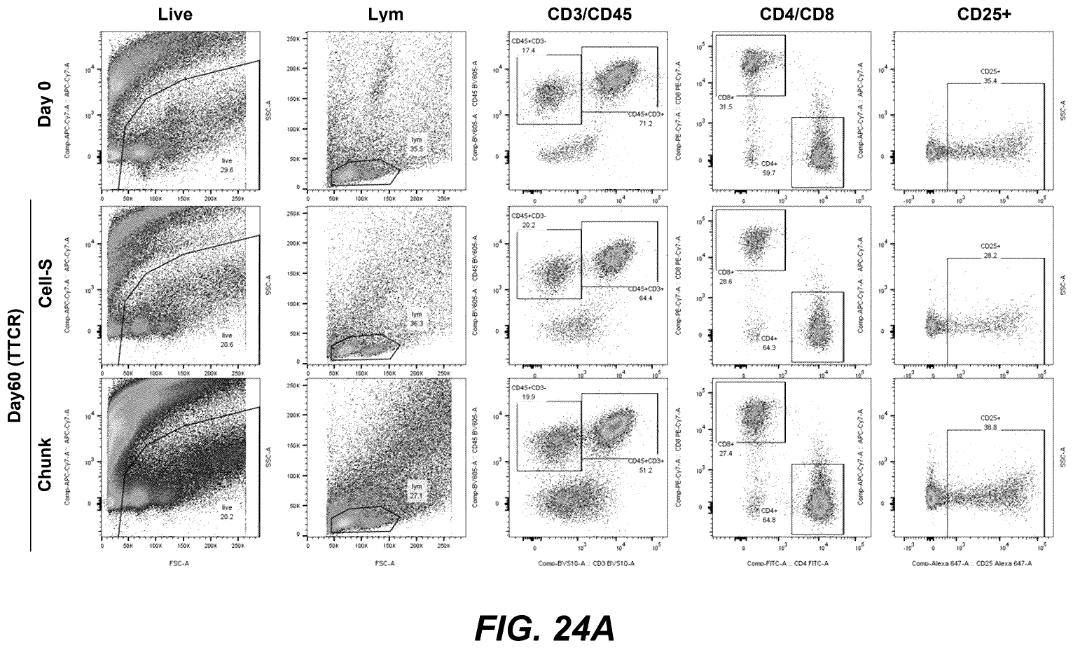

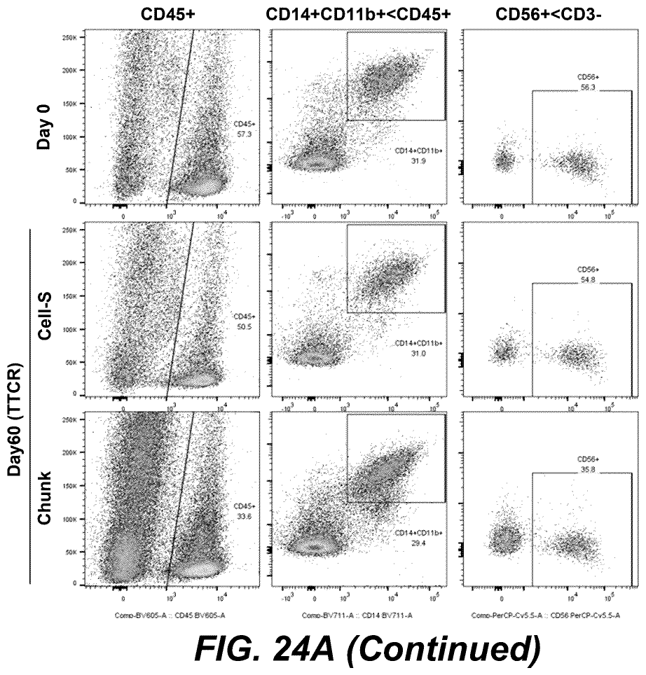

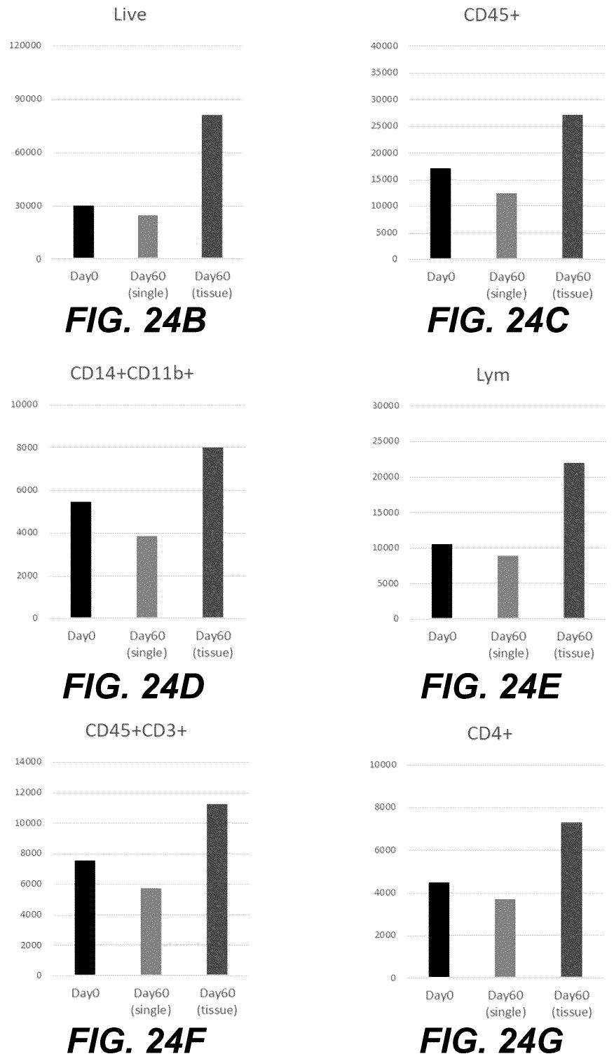

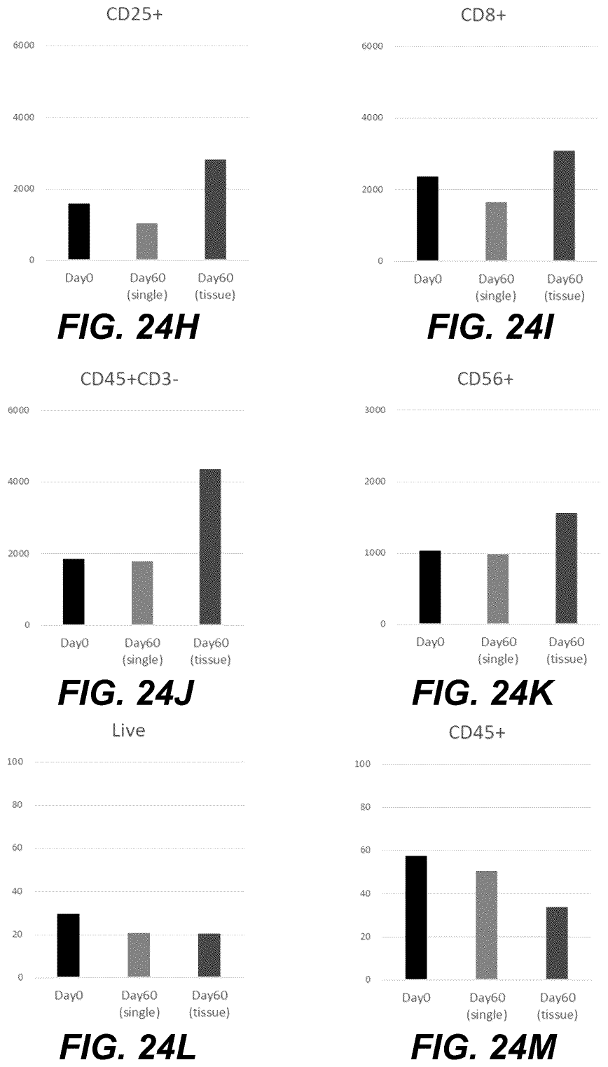

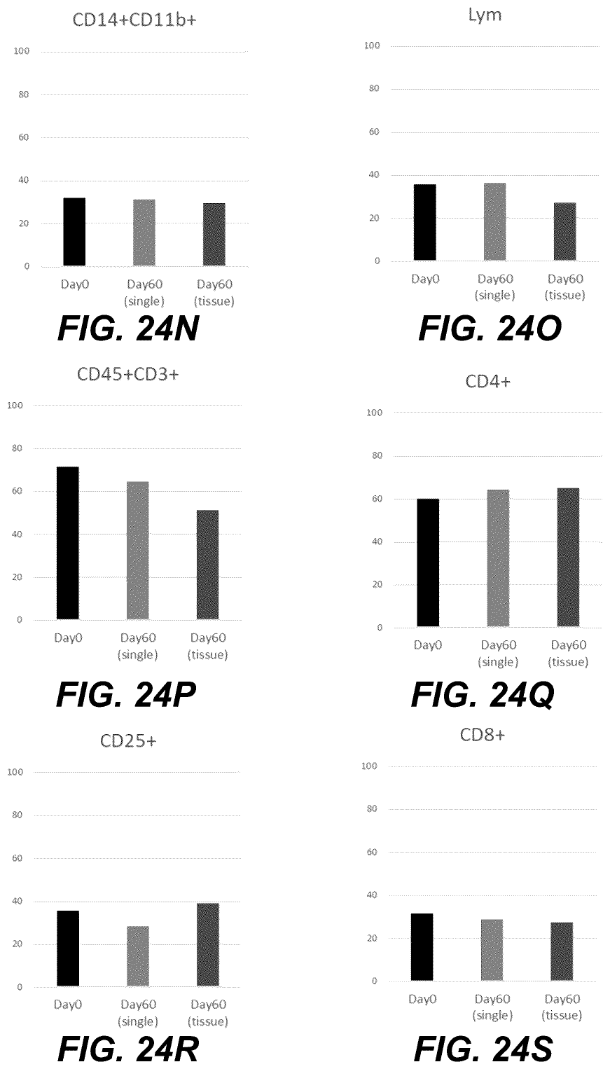

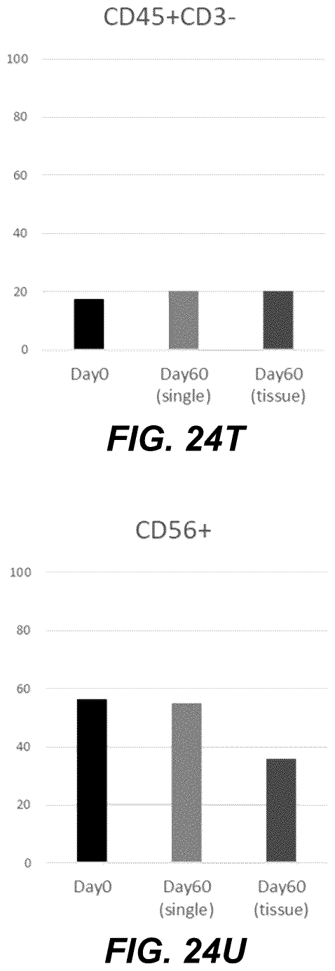

FIGS. 24A-24U depict FACS data related to viability, lymphocyte (Lym), CD3/CD45, CD4/CD8, CD25+, CD45+, CD14+CD11b+<CD45+, and CD56+<CD3-properties for Specimen Tissue-18 at Day 0 and after −80° C. for 60 days using cryopreservation buffer TTCR (Cell-S and Chunk) (FIG. 24A), cell count quantifications derived therefrom (FIGS. 24B-24K) for live (FIG. 24B), CD45+ (FIG. 24C), CD14+CD11b+ (FIG. 24D), lymphocyte (Lym) (FIG. 24E), CD45+CD3+ (FIG. 24F), CD4+ (FIG. 24G), CD25+ (FIG. 24H), CD8+ (FIG. 24I), CD45+CD3− (FIG. 24J), and CD56+ (FIG. 24K) cells, and frequency (%) quantifications derived therefrom (FIGS. 24L-24U) for live (FIG. 24L), CD45+ (FIG. 24M), CD14+CD11b+ (FIG. 24N), lymphocyte (Lym) (FIG. 24O), CD45+CD3+ (FIG. 24P), CD4+ (FIG. 24Q), CD25+ (FIG. 24R), CD8+ (FIG. 24S), CD45+CD3− (FIG. 24T), and CD56+ (FIG. 24U) cells.

### Specimen Tissue-19

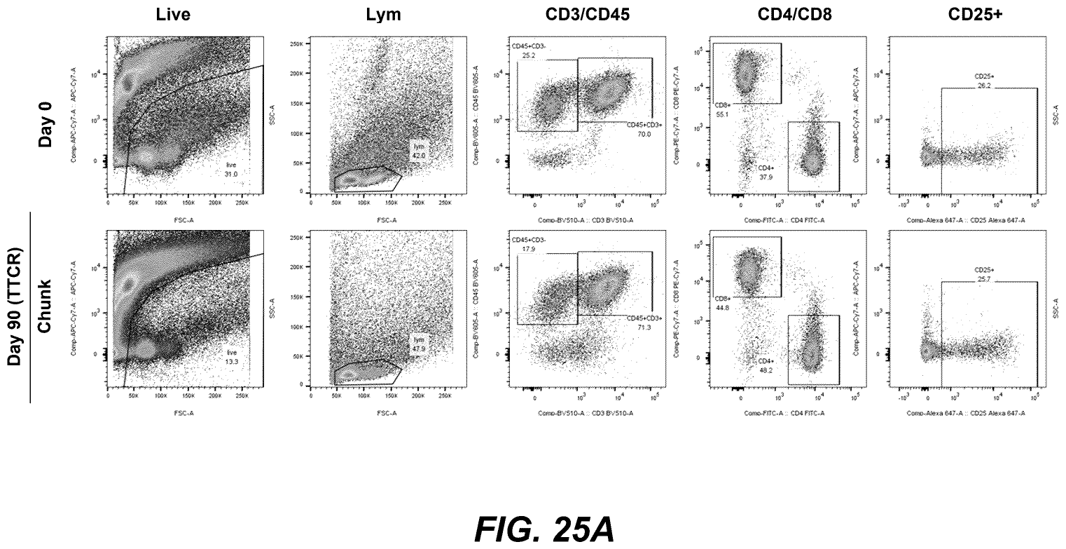

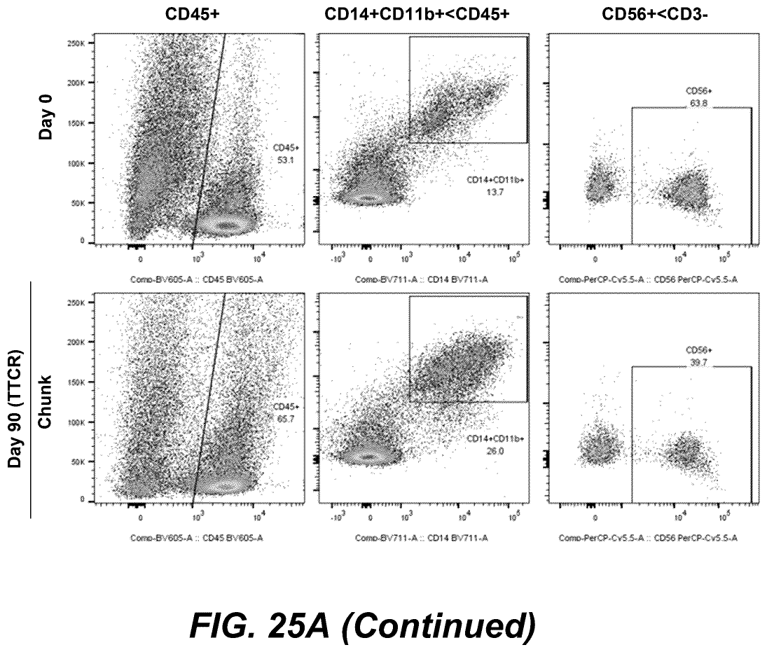

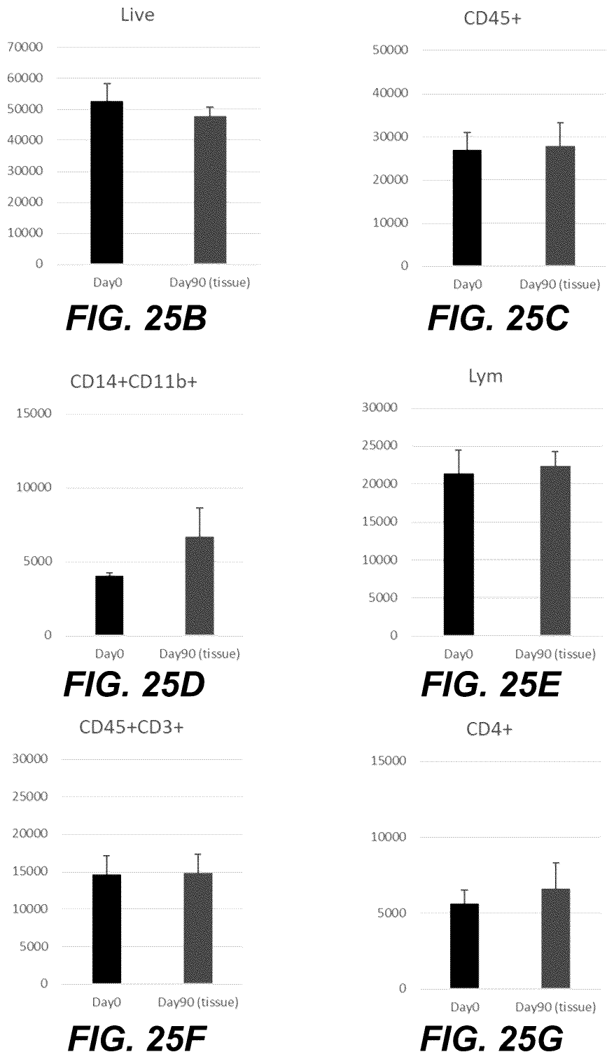

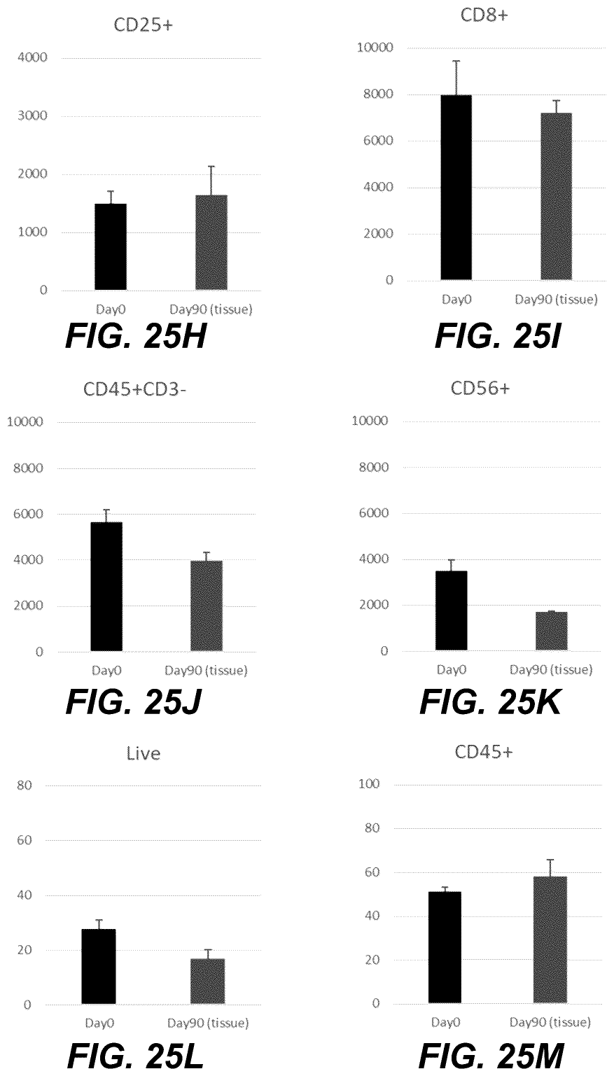

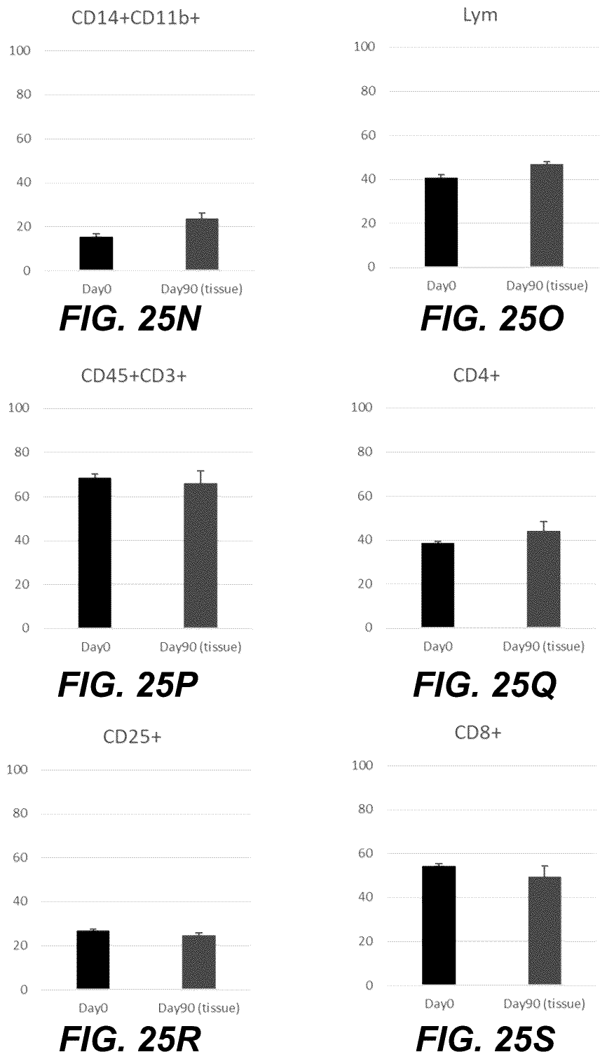

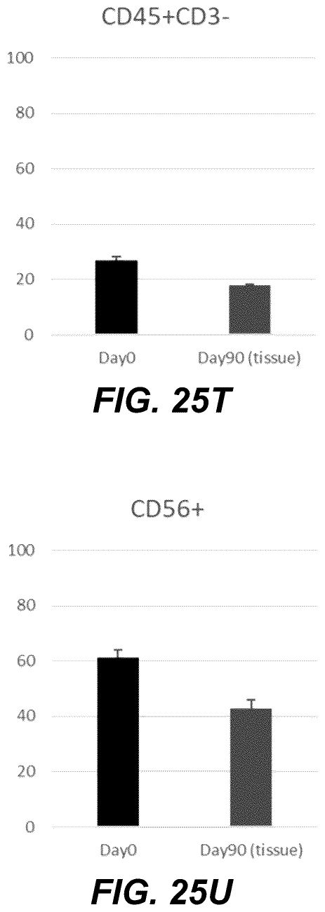

FIGS. 25A-25U depict FACS data related to viability, lymphocyte (Lym), CD3/CD45, CD4/CD8, CD25+, CD45+, CD14+CD11b+<CD45+, and CD56+<CD3-properties for Specimen Tissue-19 at Day 0 and after −80° C. for 90 days using cryopreservation buffer TTCR (Chunk) (FIG. 25A), cell count quantifications derived therefrom (FIGS. 25B-25K) for live (FIG. 25B), CD45+ (FIG. 25C), CD14+CD11b+ (FIG. 25D), lymphocyte (Lym) (FIG. 25E), CD45+CD3+ (FIG. 25F), CD4+ (FIG. 25G), CD25+ (FIG. 25H), CD8+ (FIG. 25I), CD45+CD3− (FIG. 25J), and CD56+ (FIG. 25K) cells, and frequency (%) quantifications derived therefrom (FIGS. 25L-25U) for live (FIG. 25L), CD45+ (FIG. 25M), CD14+CD11b+ (FIG. 25N), lymphocyte (Lym) (FIG. 25O), CD45+CD3+ (FIG. 25P), CD4+ (FIG. 25Q), CD25+ (FIG. 25R), CD8+ (FIG. 25S), CD45+CD3− (FIG. 25T), and CD56+ (FIG. 25U) cells. Data shown for duplicates.

### Specimen Tissue-20

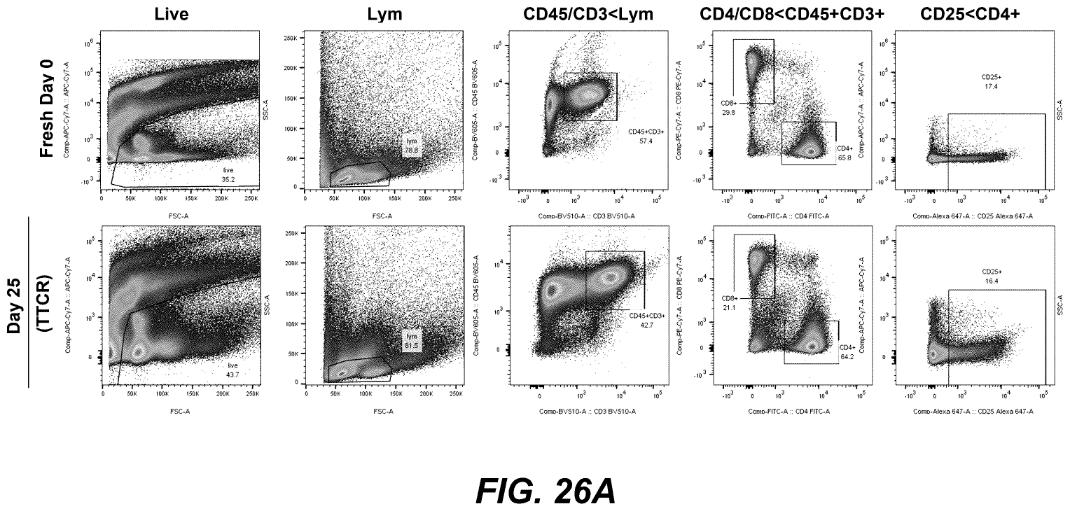

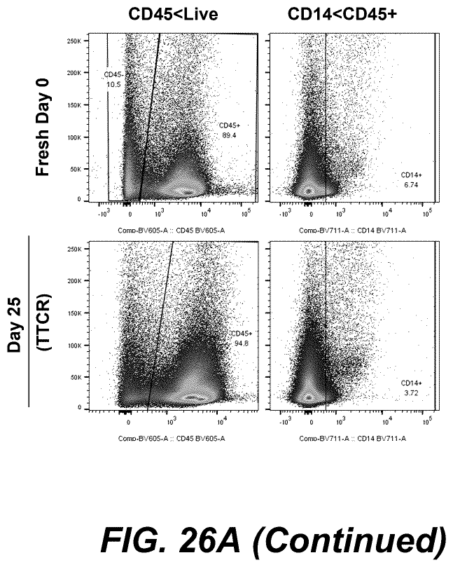

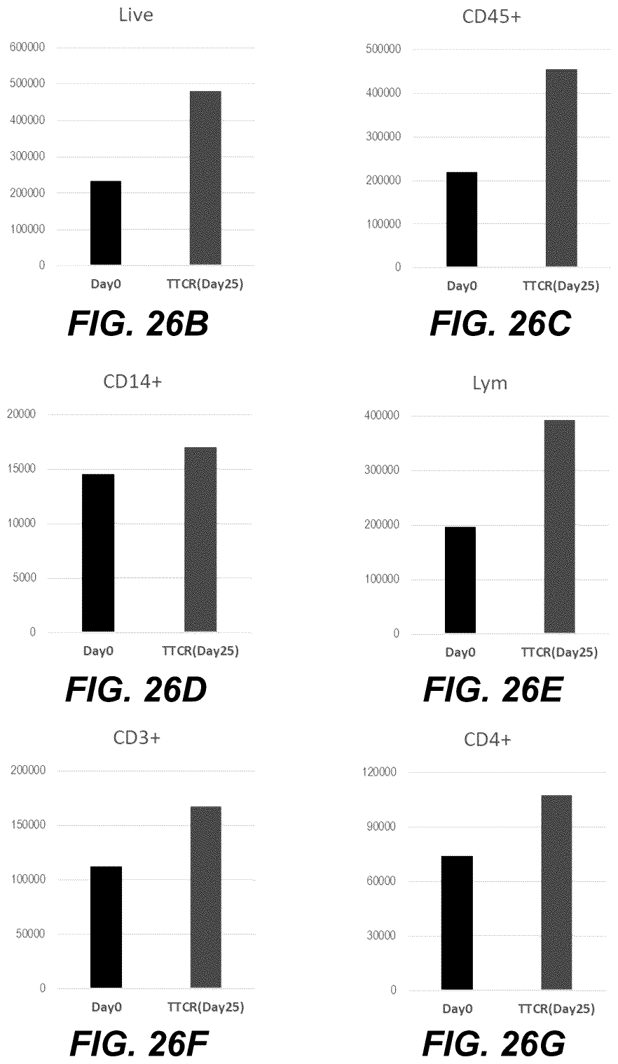

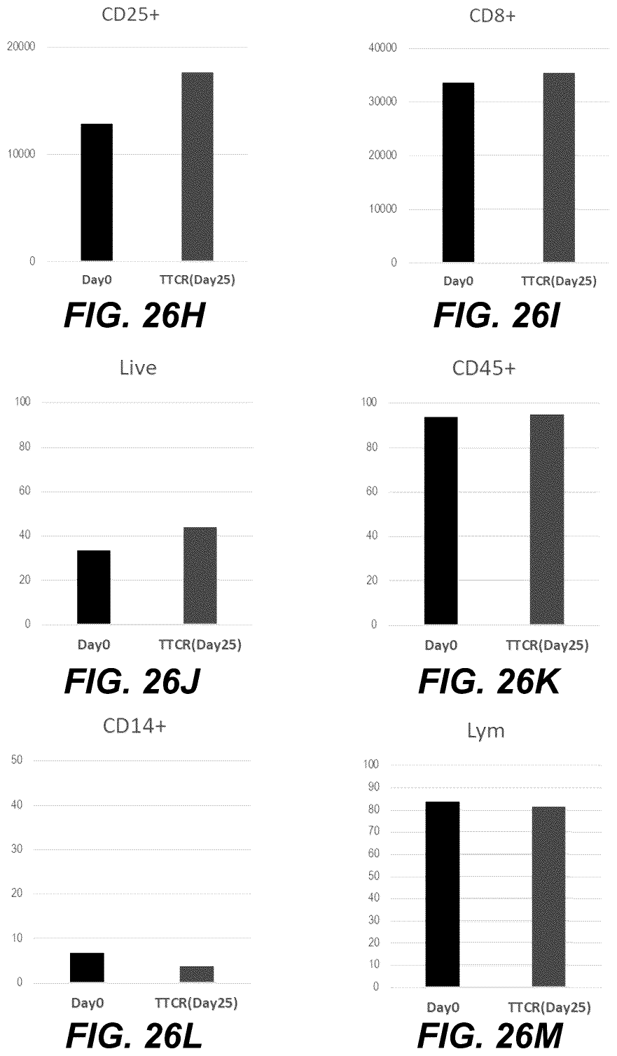

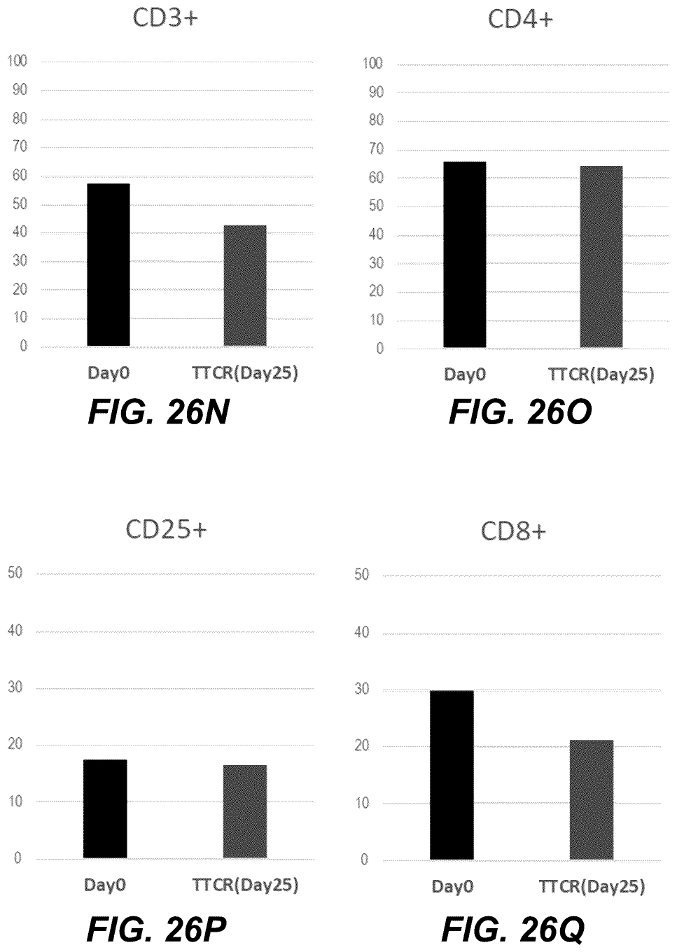

FIGS. 26A-26Q depict FACS data related to viability, lymphocyte (Lym), CD45/CD3<Lym, CD4/CD8<CD45+CD3+, CD25<CD4+, CD45<Live, and CD14<CD45+ properties for Specimen Tissue-20 at Day 0 (fresh) and after −80° C. for 25 days using cryopreservation buffer TTCR (FIG. 26A), cell count quantifications derived therefrom (FIGS. 26B-26I) for live (FIG. 26B), CD45+ (FIG. 26C), CD14+ (FIG. 26D), lymphocyte (Lym) (FIG. 26E), CD3+ (FIG. 26F), CD4+ (FIG. 26G), CD25+ (FIG. 26H), and CD8+ (FIG. 26I) cells, and frequency (%) quantifications derived therefrom (FIGS. 26J-26Q) for live (FIG. 26J), CD45+ (FIG. 26K), CD14+ (FIG. 26L), lymphocyte (Lym) (FIG. 26M), CD3+ (FIG. 26N), CD4+ (FIG. 26O), CD25+ (FIG. 26P), and CD8+ (FIG. 26Q) cells.

### Example 5

### TTS Suspending Buffer Studies

The use of TTS (comprising 100 mM Trehalose, 25 mM Sucrose, 1.5% (w/v) Taurine, 2% (v/v) FBS, and RPMI 1640 medium) as a suspending buffer/medium immediately after the thawing of frozen vials was studied in this Example. The effect of the temperature of the “suspending buffer/medium” (Cold or Warm) immediately after thawing the TTCR-frozen vial was also investigated. It was found that when used in “Cold” conditions, TTS yielded high cell viability. These data indicate TTS is very effective in protecting against cell damage.

### Experimental Workflow

TTCR cryo-tubes (TTCR-cryopreserved samples) were quickly thawed in a 37° C. water bath and then added into “suspending buffer” (See Table 19). Following centrifugation at 400 g at 4° C., cells were washed again with cold 2% FBS/RPMI. Another centrifugation at 400 g at 4° C. was performed, followed by staining.

TABLE 19
Suspending Buffers Tested

| Suspending buffer | Test temperature |
|---|---|
| 10% FBS/RPMI (generally used) | Cold (Ave. 4° C.) |
| | Warm (pre-incubation at 37° C.) |
| TTS | Cold (Ave. 4° C.) |
| | Warm (pre-incubation at 37° C.) |

### Specimen Tissue-19-LN

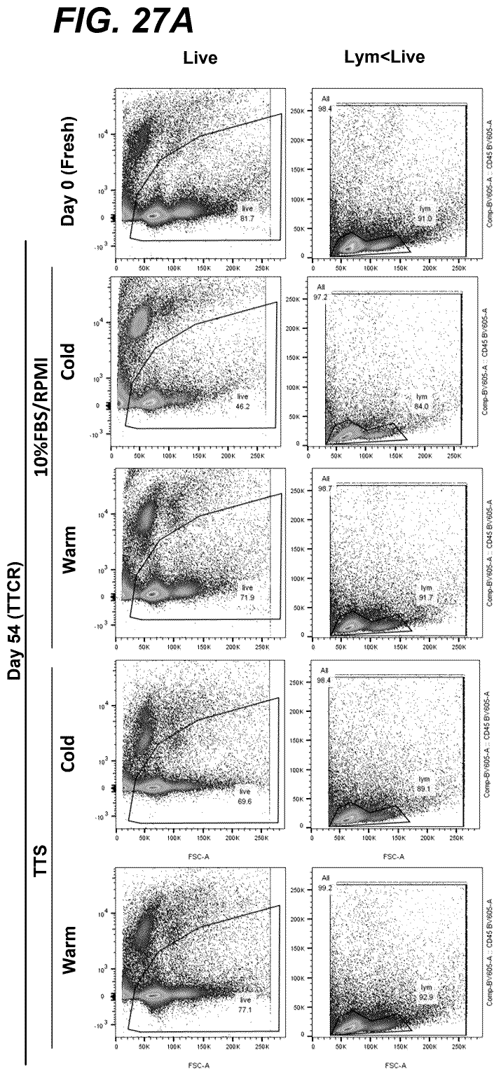

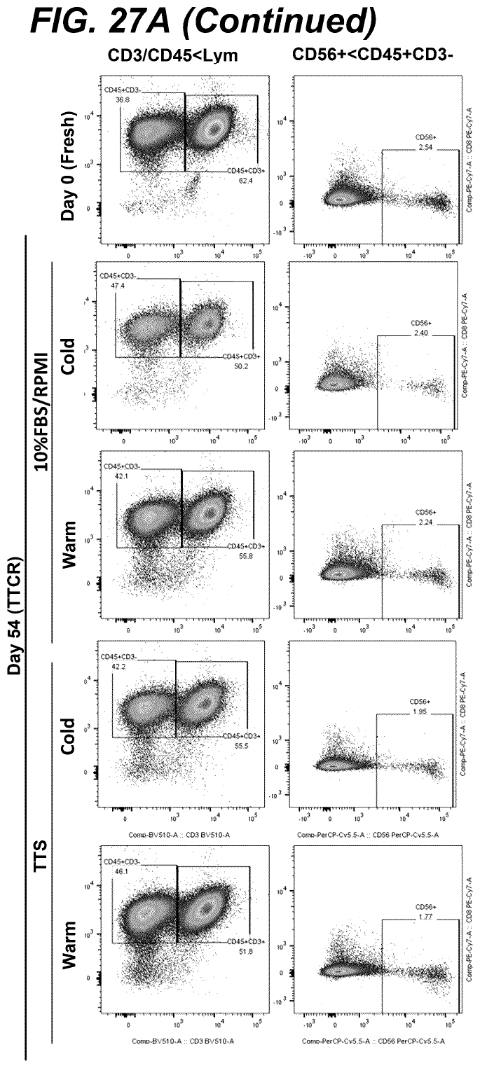

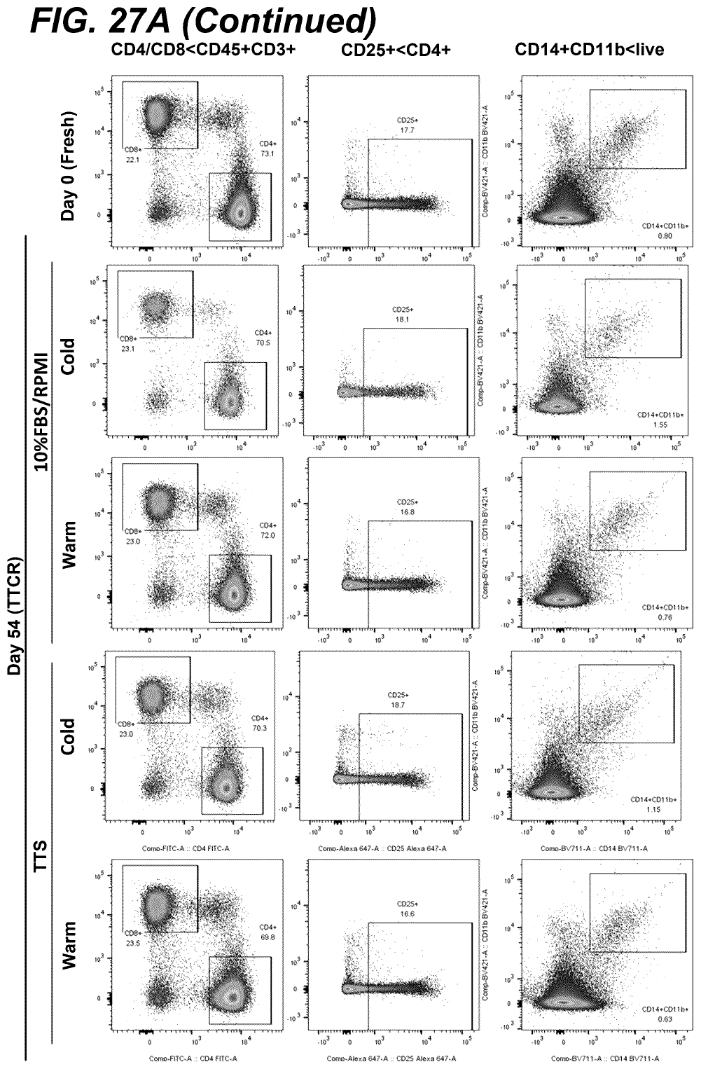

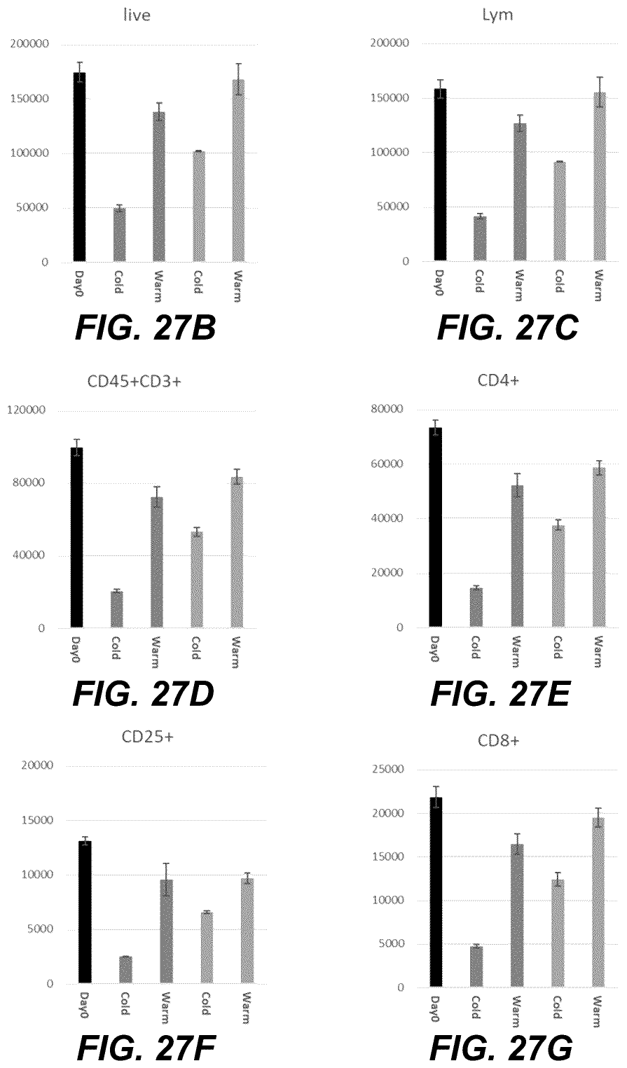

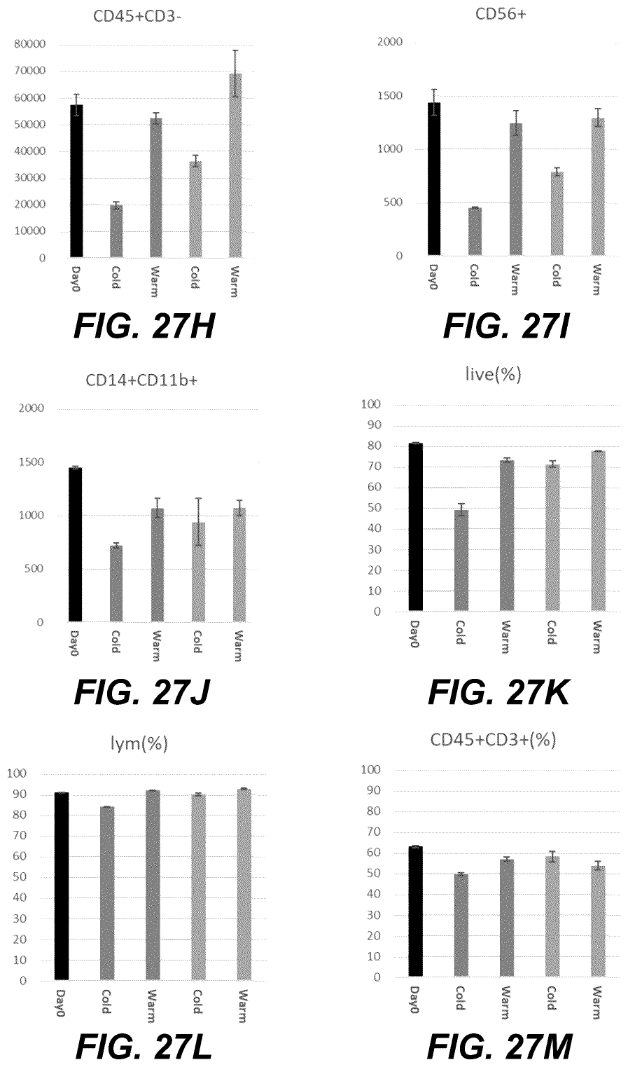

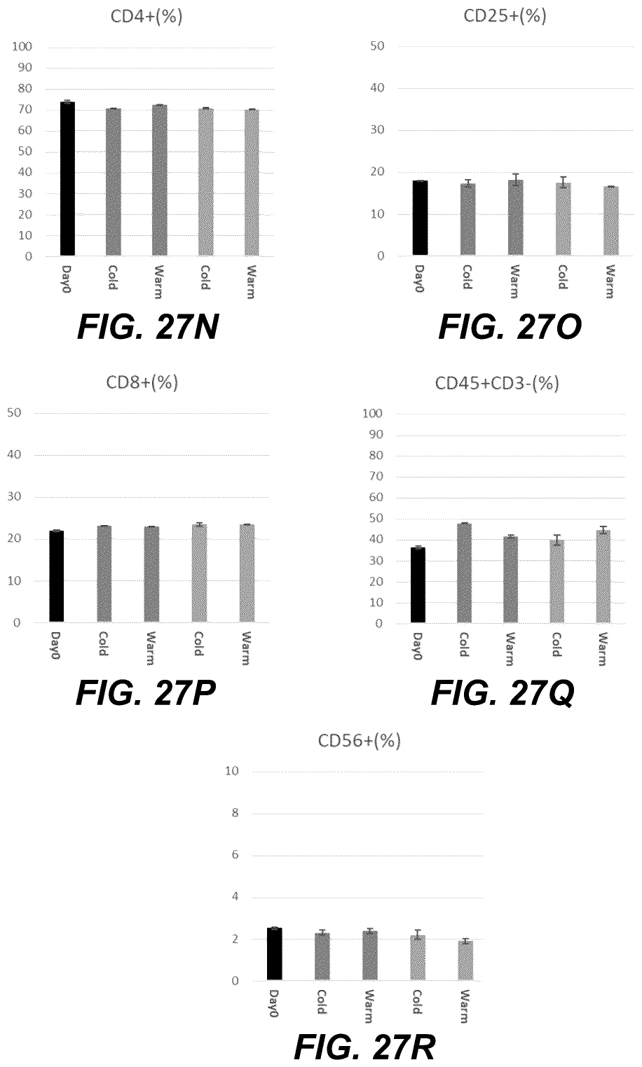

In a first experiment, Specimen Tissue-19-LN (Lymph-node from lung cancer patient) was dissociated with TTDR and equal volumes of cell suspension were cryopreserved in each TTCR tube (Table 20). FIGS. 27A-27R depict FACS data related to viability, Lym<Live, CD3/CD45<Lym, CD56+<CD45+CD3−, CD4/CD8<CD45+CD3+, CD25+<CD4+, and CD14+CD11b<live properties for Specimen Tissue-19-LN at Day 0 (fresh) and after −80° C. for 54 days using cryopreservation buffer TTCR and suspending buffers 10% FBS/RPMI (cold and warm) and TTS (cold and warm) (FIG. 27A), cell count quantifications derived therefrom (FIGS. 27B-27J) for live (FIG. 27B), lymphocyte (Lym) (FIG. 27C), CD45+CD3+ (FIG. 27D), CD4+ (FIG. 27E), CD25+ (FIG. 27F), CD8+ (FIG. 27G), CD45+CD3− (FIG. 27H), CD56+ (FIG. 27I), and CD14+CD11b+ (FIG. 27J) cells, and frequency (%) quantifications derived therefrom (FIGS. 27K-27R) for live (FIG. 27K), lymphocyte (Lym) (FIG. 27L), CD45+CD3+ (FIG. 27M), CD4+ (FIG. 27N), CD25+ (FIG. 27O), CD8+ (FIG. 27P), CD45+CD3− (FIG. 27Q), and CD56+ (FIG. 27R) cells. It was found that when used in the “Cold” condition, TTS yielded higher cell viability, while the “Warm” condition did not show a significant difference between the suspending buffers.

TABLE 20
First Experiment

| | Suspending Solution | |
|---|---|---|
| Cryopreservation | Composition | Temperature |
| TTCR (in liquid nitrogen for | 10% FBS/RPMI | Cold (duplicate tube) |
| 54 days) | | Warm (duplicate tube) |
| | TTS | Cold (duplicate tube) |
| | | Warm (duplicate tube) |

### Specimen Tissue-21-LN

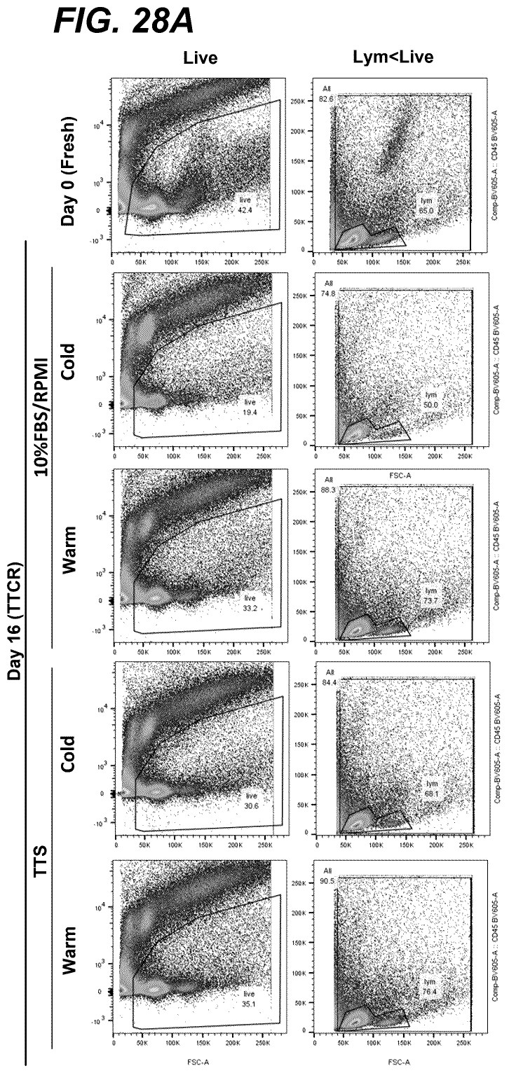

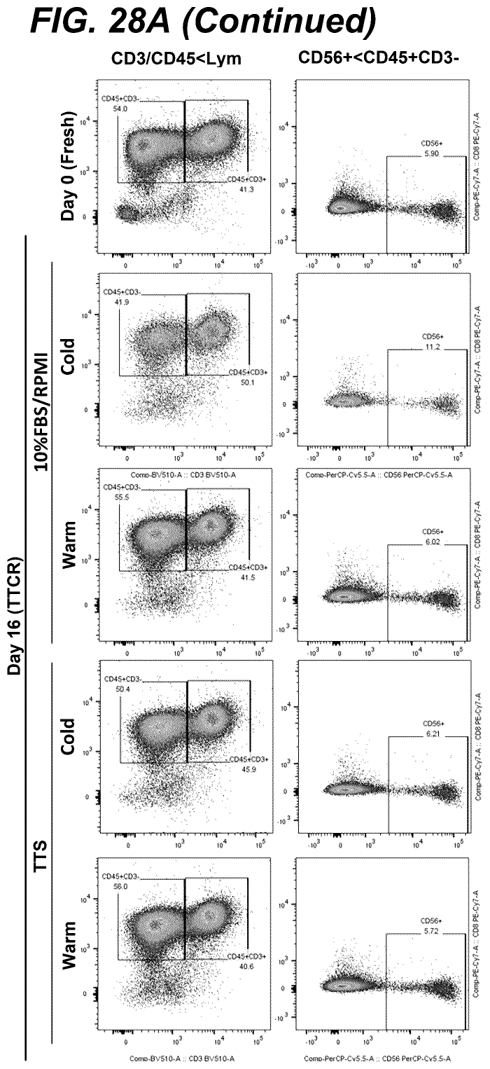

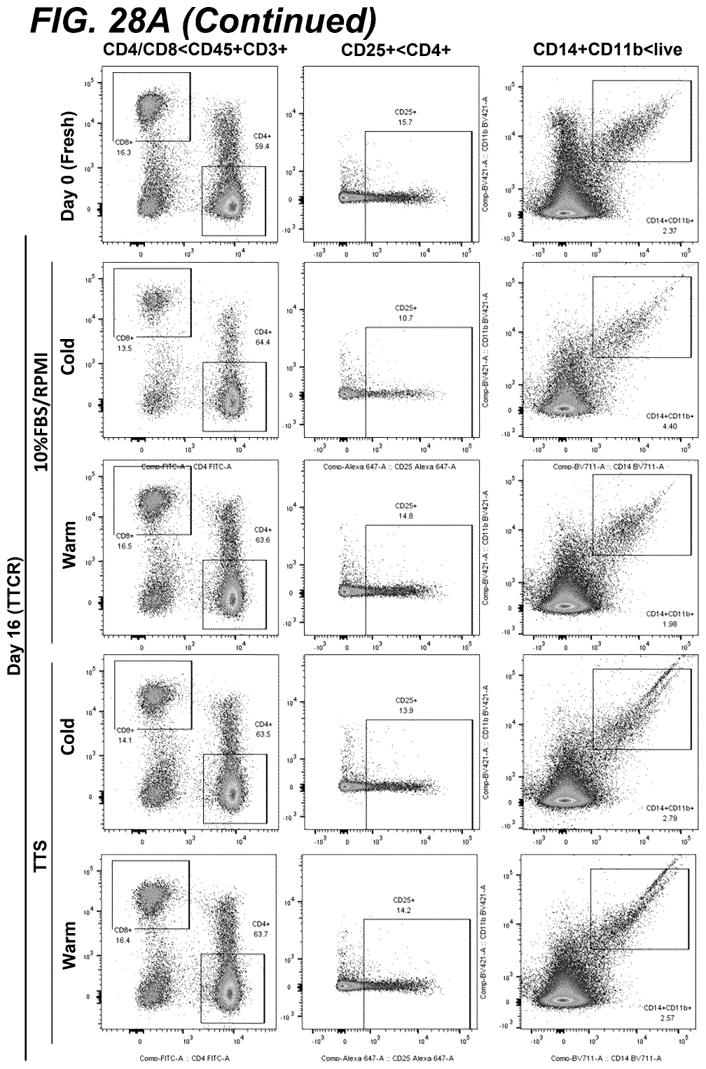

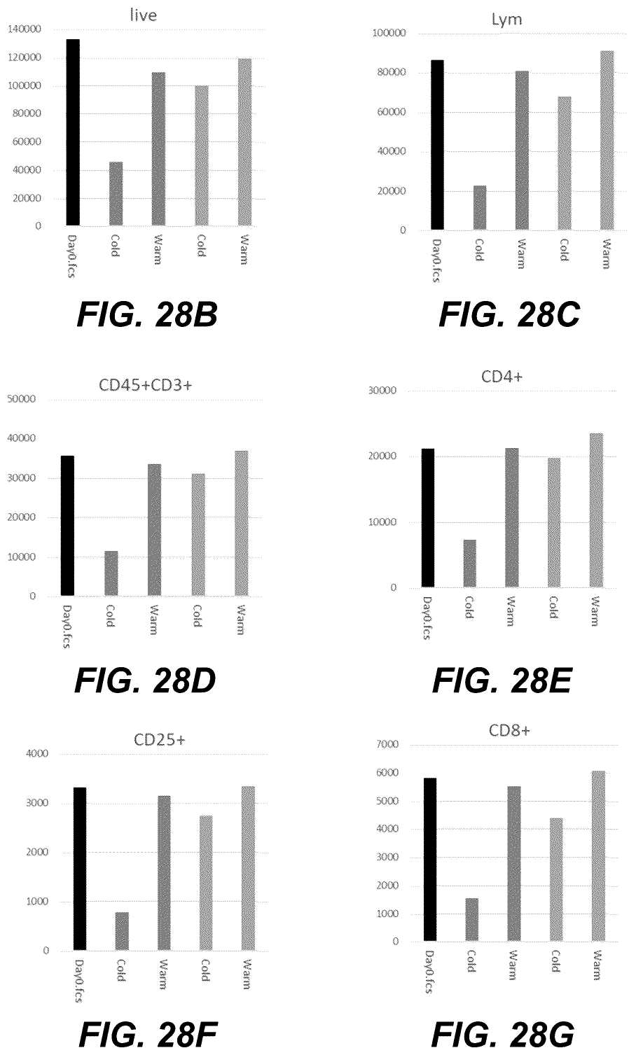

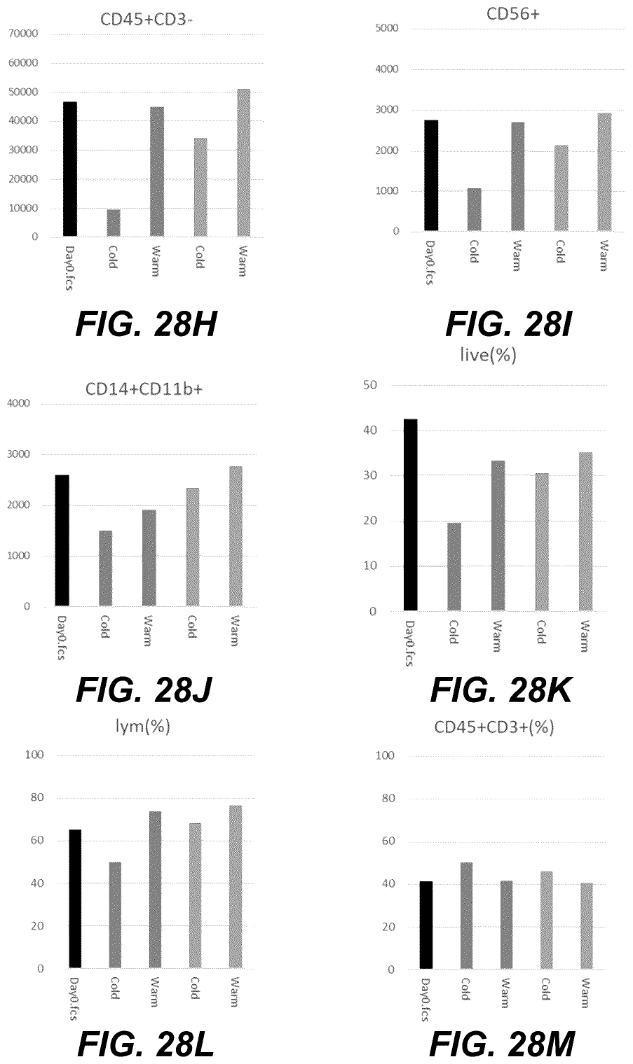

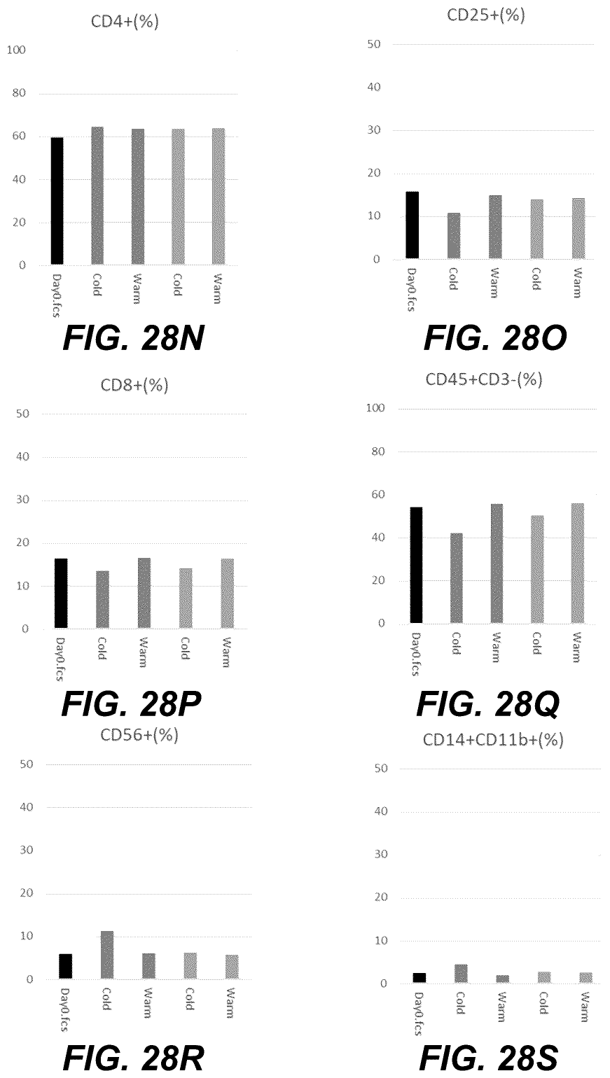

In a second experiment, Specimen Tissue-21-LN (Lymph-node from lung cancer patient) was dissociated with TTDR and equal volumes of cell suspension were cryopreserved in each TTCR tube (Table 21). FIGS. 28A-28S depict FACS data related to viability, Lym<Live, CD3/CD45<Lym, CD56+<CD45+CD3−, CD4/CD8<CD45+CD3+, CD25+<CD4+, and CD14+CD11b<live properties for Specimen Tissue-21-LN at Day 0 (fresh) and after −80° C. for 16 days using cryopreservation buffer TTCR and suspending buffers 10% FBS/RPMI (cold and warm) and TTS (cold and warm) (FIG. 28A), cell count quantifications derived therefrom (FIGS. 28B-28J) for live (FIG. 28B), lymphocyte (Lym) (FIG. 28C), CD45+CD3+ (FIG. 28D), CD4+ (FIG. 28E), CD25+ (FIG. 28F), CD8+ (FIG. 28G), CD45+CD3− (FIG. 28H), CD56+ (FIG. 28I), and CD14+CD11b+ (FIG. 28J) cells, and frequency (%) quantifications derived therefrom (FIGS. 28K-28S) for live (FIG. 28K), lymphocyte (Lym) (FIG. 28L), CD45+CD3+ (FIG. 28M), CD4+ (FIG. 28N), CD25+ (FIG. 28O), CD8+ (FIG. 28P), CD45+CD3− (FIG. 28Q), CD56+ (FIG. 28R), and CD14+CD11b+ (FIG. 28S) cells. It was found that when used in the “Cold” condition, TTS yielded higher cell viability, while the “Warm” condition did not show a significant difference between the suspending buffers.

TABLE 21
Second Experiment

| | Suspending Solution | |
|---|---|---|
| Cryopreservation | Composition | Temperature |
| TTCR (at −80° C. for 16 days) | 10% FBS/RPMI | Cold (single tube) |
| | | Warm (single tube) |
| | TTS | Cold (single tube) |
| | | Warm (single tube) |

### Specimen Tissue-22-LN

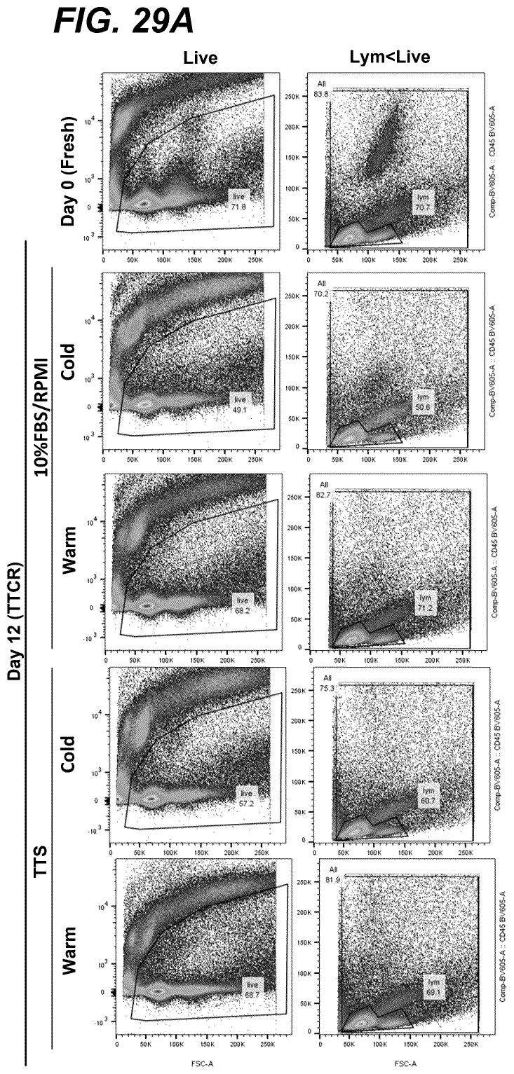

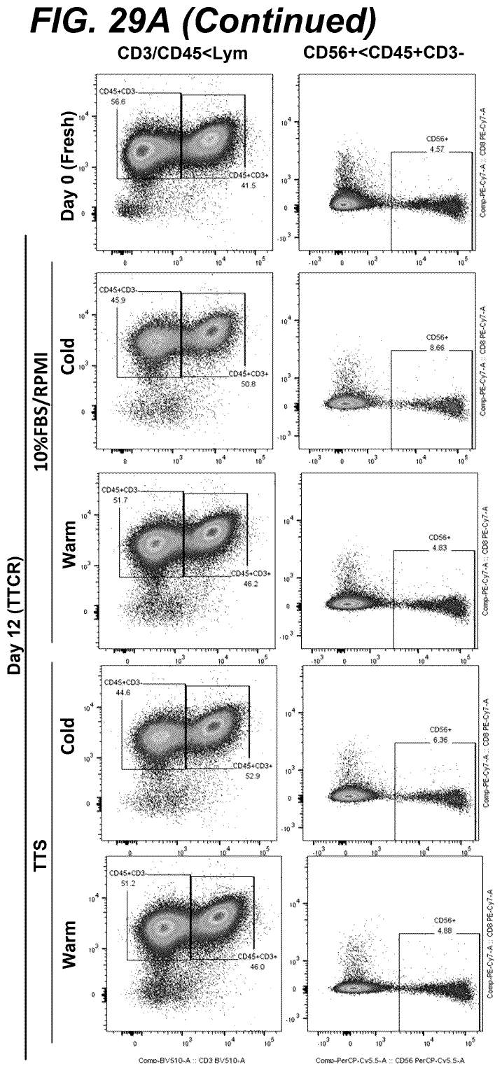

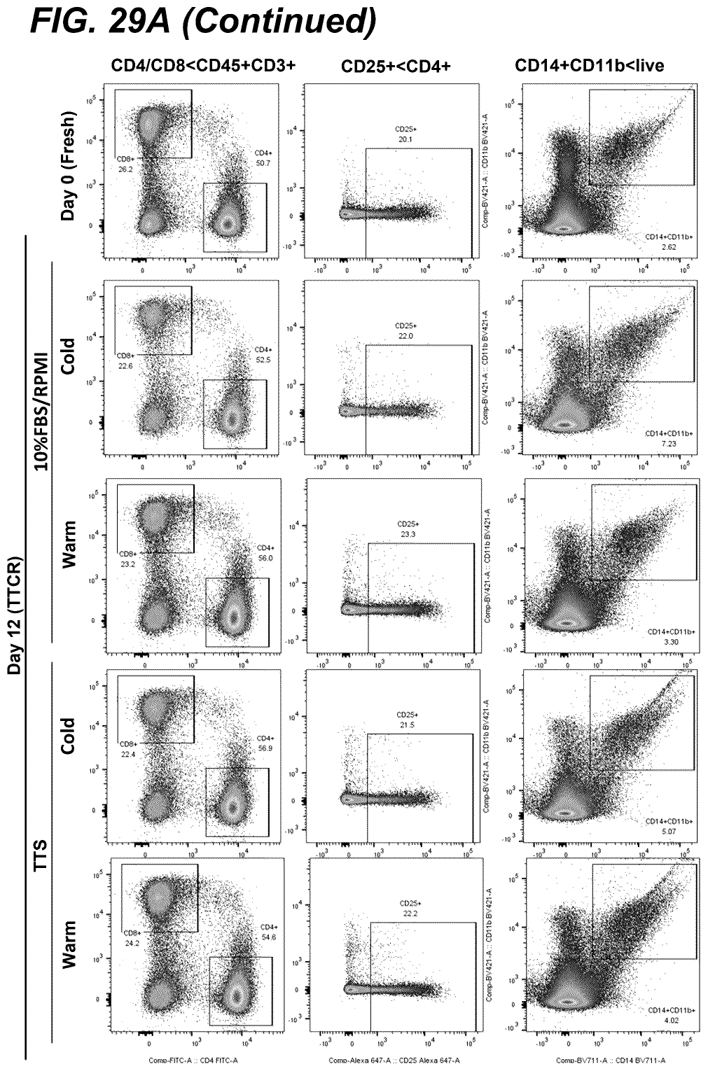

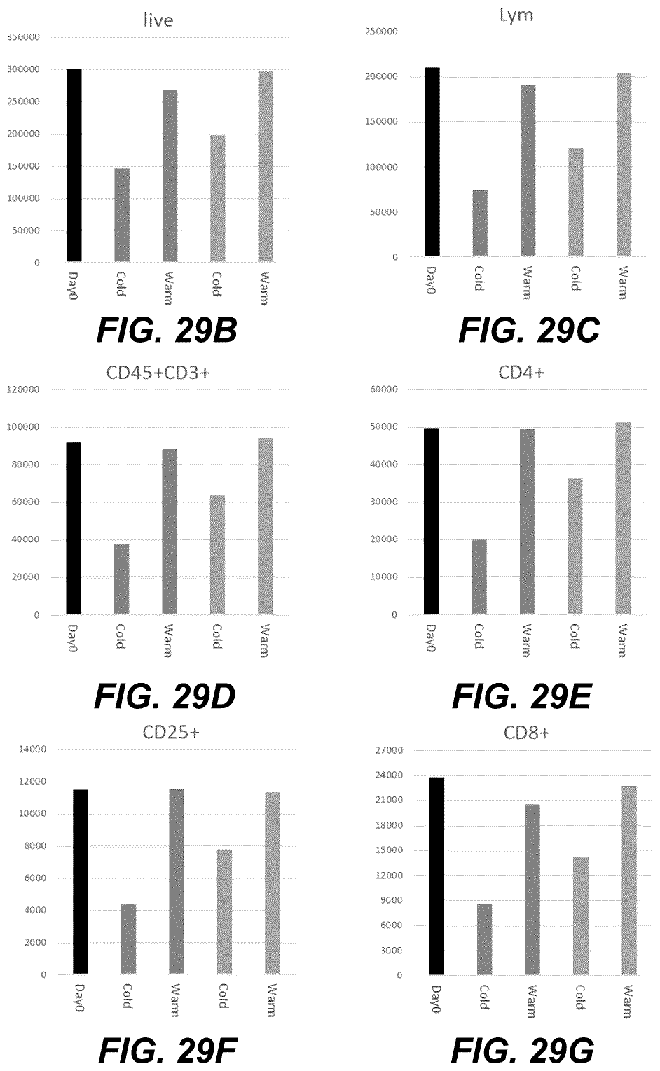

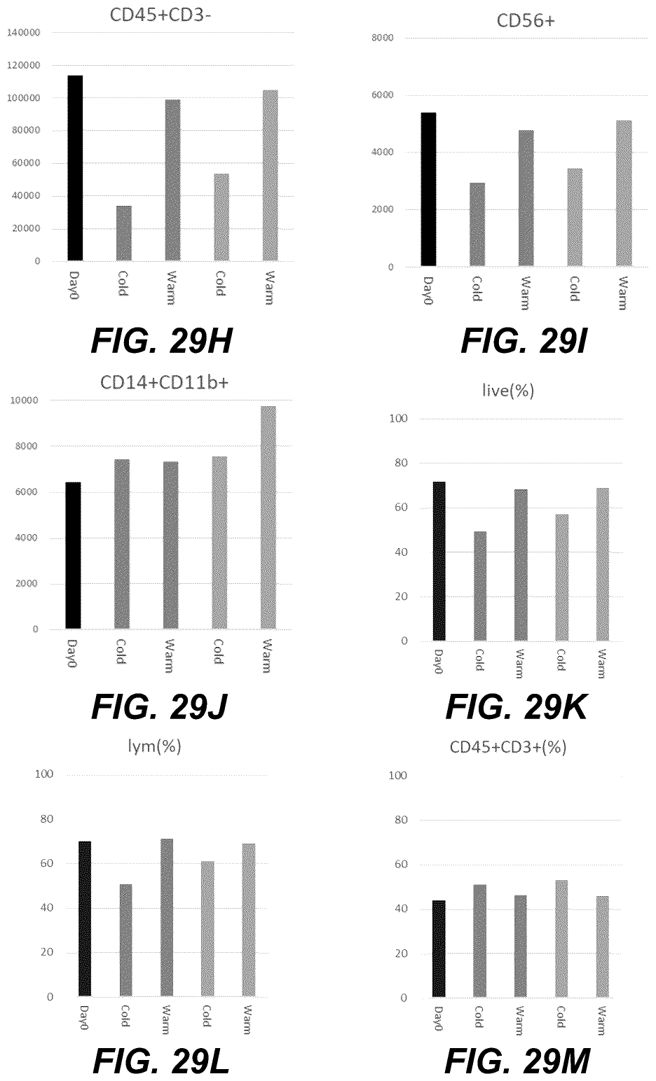

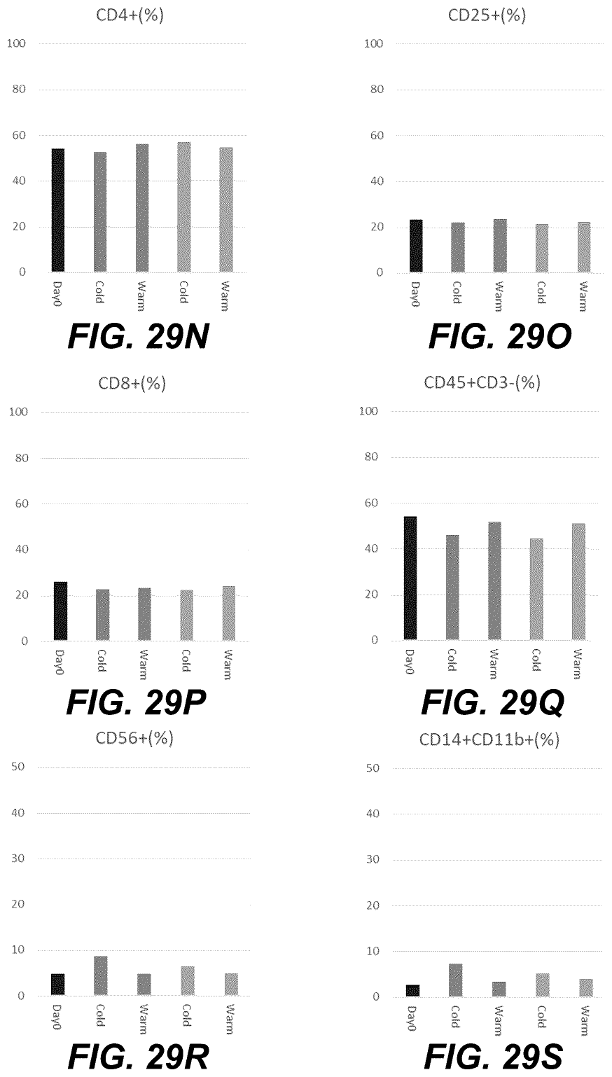

In a third experiment, Specimen Tissue-22-LN (Lymph-node from lung cancer patient) was dissociated with TTDR and equal volumes of cell suspension were cryopreserved in each TTCR tube (Table 22). FIGS. 29A-29S depict FACS data related to viability, Lym<Live, CD3/CD45<Lym, CD56+<CD45+CD3−, CD4/CD8<CD45+CD3+, CD25+<CD4+, and CD14+CD11b<live properties for Specimen Tissue-22-LN at Day 0 (fresh) and after −80° C. for 12 days using cryopreservation buffer TTCR and suspending buffers 10% FBS/RPMI (cold and warm) and TTS (cold and warm) (FIG. 29A), cell count quantifications derived therefrom (FIGS. 29B-29J) for live (FIG. 29B), lymphocyte (Lym) (FIG. 29C), CD45+CD3+ (FIG. 29D), CD4+ (FIG. 29E), CD25+ (FIG. 29F), CD8+ (FIG. 29G), CD45+CD3− (FIG. 29H), CD56+ (FIG. 29I), and CD14+CD11b+ (FIG. 29J) cells, and frequency (%) quantifications derived therefrom (FIGS. 29K-29S) for live (FIG. 29K), lymphocyte (Lym) (FIG. 29L), CD45+CD3+ (FIG. 29M), CD4+ (FIG. 29N), CD25+ (FIG. 29O), CD8+ (FIG. 29P), CD45+CD3− (FIG. 29Q), CD56+ (FIG. 29R), and CD14+CD11b+ (FIG. 29S) cells. It was found that when used in the “Cold” condition, TTS yielded higher cell viability, while the “Warm” condition did not show a significant difference between the suspending buffers.

TABLE 22
Third Experiment

| | Suspending Solution | |
|---|---|---|
| Cryopreservation | Composition | Temperature |
| TTCR (at −80° C. for 12 days) | 10% FBS/RPMI | Cold (single tube) |
| | | Warm (single tube) |
| | TTS | Cold (single tube) |
| | | Warm (single tube) |

### Example 6

### Assaying Effect of TTCR-Based Cryopreservation on Cell Function

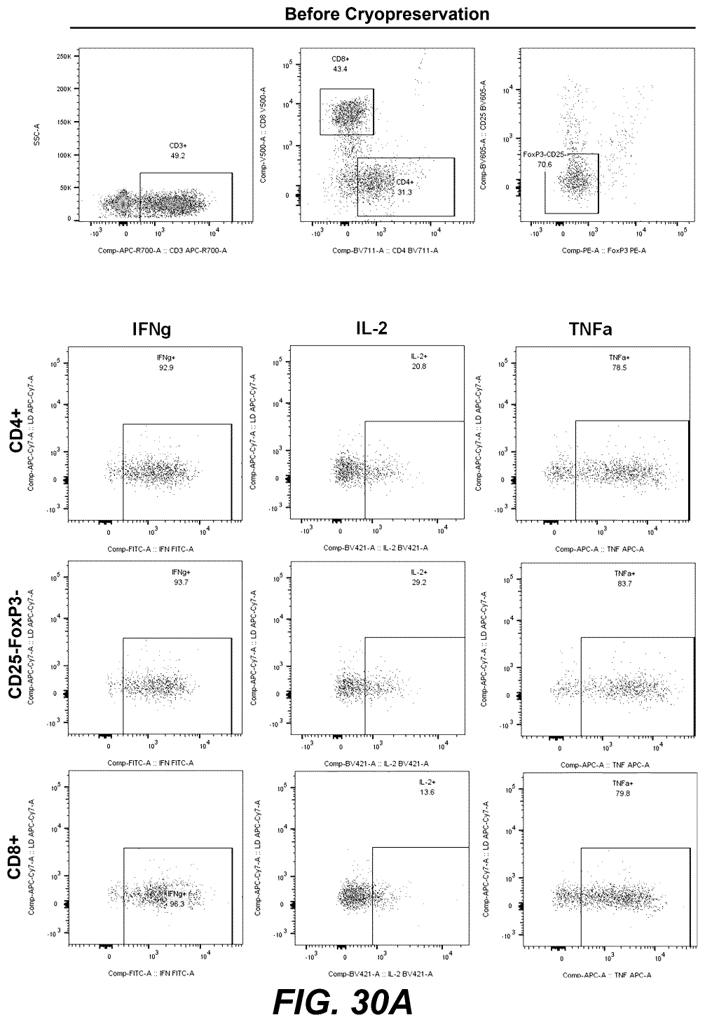

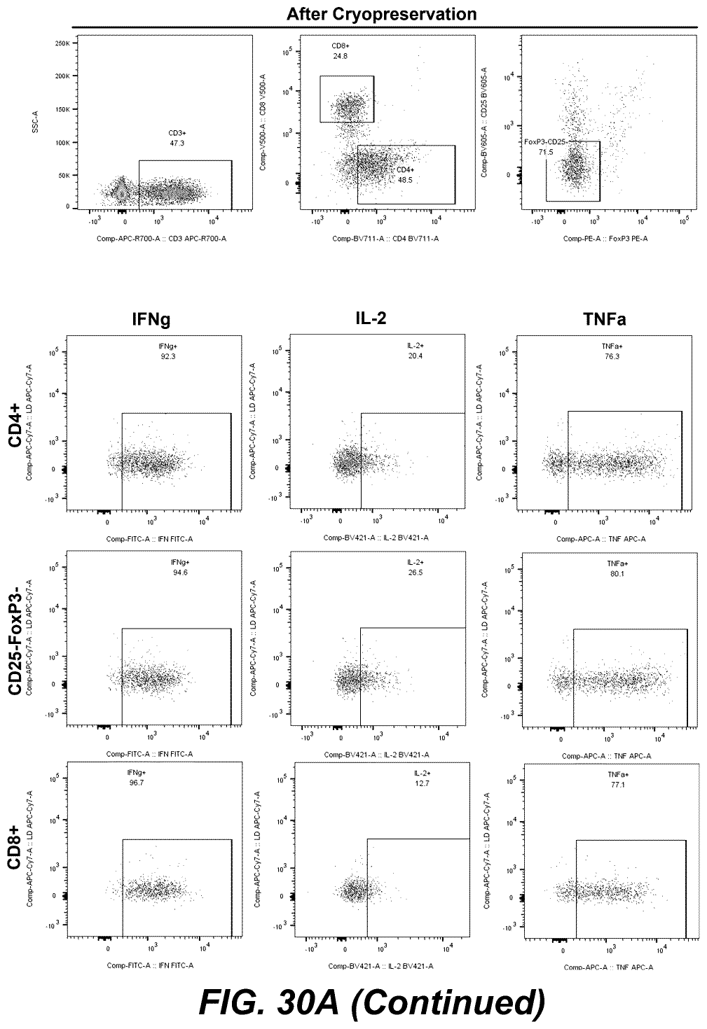

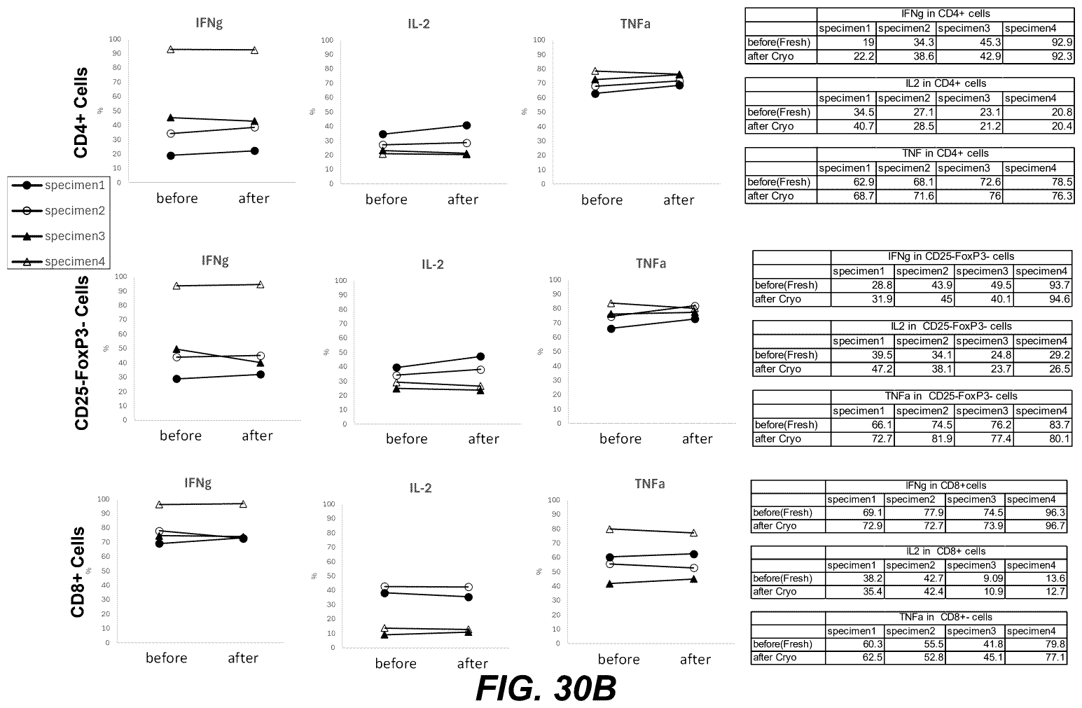

The ability of cells to produce cytokines before (fresh) versus after (cryo) cryopreservation was evaluated. Four lung cancer specimens were employed as test samples, with fresh cells versus cryopreserved (for 5-14 days) cells being compared. The specimens were collected in TTPR and treated with TTDR to extract single cell TILs. The single cells either underwent the cytokine assay (fresh) or were frozen at −80° C. (for 5˜14 days) with TTCR (cryo). The cryo cells were subsequently thawed, washed, and underwent the cytokine assay. The cytokine assay comprised culturing for 16 hours in 10% FBS/RPMI, stimulation with PMA (20 ng/mL), Ionomycin (1 ug/mL), and Monensin (2.6 uM), an additional incubation for 3 hours, staining (Table 23), and FACS analysis. FIGS. 30A-30B depict non-limiting exemplary pre- and post-cryopreservation FACS data (FIG. 30A) and quantifications derived therefrom (FIG. 30B). Each cytokine gate was decided by the isotype control. The cell function data indicates that the cells' ability to produce cytokines do not change even after cryopreservation (TTCR) compared with fresh (before cryo).

TABLE 23
Staining panel for FACS analysis

| | | Isotype | |
|---|---|---|---|
| | FL dye | control | Staining location |
| LD (Live | FVS780 | | Live (membrane)/ |
| dead) | | | dead(membrane&intracellular) |
| CD3 | AlexaFluor700 | | membrane |
| CD4 | PerCP-CY5.5 | | membrane |
| CD8 | BV605 | | membrane |
| CD25 | BV786 | | membrane |
| FoxP3 | PE | | intracellular |
| IFN- | APC or FITC | Mouse IgG1 | intracellular |
| gamma | | | |
| IL-2 | BV421 | Rat IgG2a | intracellular |
| TNF-alpha | FITC or APC | Mouse IgG1 | intracellular |

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1-81. (canceled)

82. A cryopreservation reagent, comprising:
one or more cryoprotective agent(s);
one or more culture media component(s);
one or more carbohydrate(s); and
one or more amino acid(s).

83. The cryopreservation reagent of claim 82, wherein:
the one or more cryoprotective agent(s) comprise dimethyl sulfoxide (DMSO) and/or ethylene glycol (EG);
the one or more culture media component(s) comprise Fetal Bovine Serum (FBS) and/or RPMI-1640;
the one or more carbohydrate(s) comprise trehalose, sucrose, and/or fructose;
and/or the one or more amino acid(s) comprise taurine.

84. The cryopreservation reagent of claim 82, wherein the cryopreservation reagent comprises:
about 6% (v/v) to about 14% (v/v) of the one or more cryoprotective agent(s); and/or
about 3% (v/v) to about 7% (v/v) of a first cryoprotective agent and about 3% (v/v) to about 7% (v/v) of a second cryoprotective agent.

85. The cryopreservation reagent of claim 82, wherein the one or more cryoprotective agent(s) are selected from the group comprising acetamide, albumin, ammonium acetate, anti-freeze proteins, butanediol, chloroform, choline, cyclohexanediols, cyclohexanediones, cyclohexanetriols, diethylene glycol, dimethyl acetamide, dimethyl formamide, dimethyl sulfoxide, ethanol, ethylene glycol, ethylene glycol monomethyl ether, formamide, glycerol, glyceryl monoacetate, glycoproteins, hydroxyethyl starch, magnesium chloride, magnesium sulfate, methanol, methoxy propanediol, methyl acetamide, methyl formamide, methyl ureas, methyl glycerol, phenol, pluronic polyols, polyethylene glycol, polyvinylpyrrolidone, propanediol, pyridine N-oxide, sodium bromide, sodium chloride, sodium iodide, sodium nitrate, sodium nitrite, sodium sulfate, triethylene glycol, trimethylamine acetate, urea, derivatives thereof, or any combination thereof.

86. The cryopreservation reagent of claim 82, wherein the cryopreservation reagent comprises:
the one or more carbohydrate(s) at a concentration of about 10 mM to about 300 mM; and/or
a first carbohydrate at a concentration of about 50 mM to about 100 mM and a second carbohydrate at a concentration of about 20 mM to about 50 mM.

87. The cryopreservation reagent of claim 82, wherein the one or more carbohydrate(s) are selected from the group comprising a sugar, a monosaccharide, a disaccharide, a polyol, an oligosaccharide, a malto-oligosaccharide, a non-malto-oligosaccharide, a polysaccharide, a starch, a non-starch polysaccharide, derivatives thereof, or any combination thereof.

88. The cryopreservation reagent of claim 82, wherein the one or more carbohydrate(s) are selected from the group comprising trehalose, fructose, sucrose, allose, altrose, amylopectin, arabinose, adonitol, cellobiose, cyclodextrin, dextran, deoxyribose, dulcitol, dextrose, erythritol, erythrose, erythrulose, fucose, galactose, glucosamine, glucose, gulose, idose, inositol, invert sugar, isotrehalose, lactose, lyxose, maltodextrin, maltose, mannitol, mannose, mannosamine, melezitose, neotrehalose, palatinose (isomaltulose), psicose, raffinose, ribose, ribulose, rhamnose, sialic acid, sorbitol, sorbose, tagatose, talose, threitol, threose, turanose, xylitol, xylose, xylulose, derivatives thereof, or any combination thereof.

89. The cryopreservation reagent of claim 82, wherein the cryopreservation reagent comprises:
about 10% (v/v) to about 80% (v/v) of the one or more culture media component(s); and/or
about 50% (v/v) of a first culture media component and about 30% (v/v) to about 50% (v/v) of a second culture media component.

90. The cryopreservation reagent of claim 82, wherein the one or more culture media component(s) are selected from the group comprising AIM-V, Alpha-Minimum Essential Medium (α-MEM), Basal Medium Eagle (BME), Brainphys, CTS KnockOut DMEM, CTS OpTimizer T Cell Expansion SFM, CellGro DC Medium, Dulbecco's Modified Eagle Medium (DMEM), Dulbecco's Modified Eagle Medium: Nutrient Mixture F-12 (DMEM/F-12), Dulbecco's Phosphate Buffered Saline (DPBS), Eagle's Basal Medium (EBM), Eagle's Essential Medium (EEM), Earle's Balanced Salt Solution (EBSS), F-10 Nutrient Mixture, F-12K Nutrient Mixture Medium (Kaighn's Modification, F-12K), Fetal Bovine Serum (FBS), Fischer's Medium, GMEM (Glasgow's Minimum Essential Medium), Hanks' Balanced Salt Solution (HBSS), Hanks' Salts Medium (HMEM), Ham's F-10, Ham's F-12, ImmunoCult-XF T Cell Expansion Medium, Iscove's Modified Dulbecco's Medium (IMDM), L-15 (Leibovitz's L-15), MCDB 131, MCDB 153, MDEM, MEM (Minimum Essential Medium), M199 (Medium 199), McCoy's 5A, Neurobasal, Neurobasal A, ObM (Osteoblast Medium), Opti-MEM I Reduced Serum Media, PBS (Phosphate-Buffered Saline), PRIME-XV T Cell Expansion Medium, Roswell Park Memorial Institute Medium (RPMI) 1640 Medium, Sodium Bicarbonate Buffers, StemLine II, StemPro-34, StemSpan-ACF, StemSpan-H3000, StemSpan-SFEM, StemXVivo, TexMACS Medium, X-VIVO 15, X-vivo 20, derivatives thereof, or any combination thereof.

91. The cryopreservation reagent of claim 82, wherein the one or more amino acid(s) is present at a concentration of about 50 mM to about 200 mM.

92. The cryopreservation reagent of claim 82, wherein the one or more amino acid(s) are selected from the group comprising taurine, alanine, arginine, asparagine, aspartic acid, cysteine, glutamic acid, glutamine, glycine, histidine, isoleucine, leucine, lysine, methionine, phenylalanine, proline, selenocysteine, serine, threonine, tyrosine, tryptophan, ornithine, citrulline, aminobenzoic acid, valine, derivatives thereof, or any combination thereof.

93. The cryopreservation reagent of claim 82, wherein the cryopreservation reagent comprises:
about 3% (v/v) to about 7% (v/v) DMSO;
about 3% (v/v) to about 7% (v/v) EG;
about 10% (v/v) to about 80% (v/v) FBS;
about 10% (v/v) to about 70% (v/v) RPMI-1640;
trehalose at a concentration of about 20 mM to about 200 mM;
sucrose and/or fructose at a concentration of about 10 mM to about 100 mM; and/or
taurine at a concentration of about 50 mM to about 200 mM.

94. The cryopreservation reagent of claim 82, wherein the cryopreservation reagent comprises:
about 5% (v/v) DMSO;
about 5% (v/v) EG;
about 50% (v/v) FBS;
about 30% (v/v) to about 50% (v/v) RPMI-1640;
trehalose at a concentration of about 50 mM to about 100 mM;
sucrose and/or fructose at a concentration of about 20 mM to about 50 mM; and/or
taurine at a concentration of about 50 mM to about 200 mM.

95. The cryopreservation reagent of claim 82, wherein the cryopreservation reagent does not comprise:
albumin;
lactic acid or a salt thereof;
potassium dihydrogen phosphate; and/or
disodium hydrogen phosphate.

96. The cryopreservation reagent of claim 82, wherein:
the sodium ion concentration of the cryopreservation reagent is about 50-300 mmol/L;
the cryopreservation reagent comprises albumin;
the cryopreservation reagent comprises lactic acid or salt thereof;
the cryopreservation reagent comprises about 0.01-10 mmol/L of potassium dihydrogen phosphate;
the cryopreservation reagent comprises about 0.01-50 mmol/L of disodium hydrogen phosphate; and/or
the cryopreservation reagent comprises a polysaccharide selected from the group comprising trehalose, a fructooligosaccharide, indigestible dextrin, or any combination thereof.

97. The cryopreservation reagent of claim 82, wherein the cryopreservation reagent is capable of maintaining one or more cellular properties and/or cell viability of a plurality of cells in a sample for a period of time under a storage condition.

98. The cryopreservation reagent of claim 97, wherein the storage condition comprises:
a temperature of about 4° C., 2° C., 0° C., −2° C., −4° C., −6° C., −8° C., −10° C., −12° C., −14° C., −16° C., −18° C., −20° C., −22° C., −24° C., −26° C., −28° C., −30° C., −32° C., −34° C., −36° C., −38° C., −40° C., −42° C., −44° C., −46° C., −48° C., −50° C., −52° C., −54° C., −56° C., −58° C., −60° C., −62° C., −64° C., −66° C., −68° C., −70° C., −72° C., −74° C., −76° C., −78° C., −80° C., −82° C., −84° C., −86° C., −88° C., −90° C., −92° C., −94° C., −96° C., −98° C., −100° C., −102° C., −104° C., −106° C., −108° C., −110° C., −112° C., −114° C., −116° C., −118° C., −120° C., −122° C., −124° C., −126° C., −128° C., −130° C., −132° C., −134° C., −136° C., −138° C., −140° C., −142° C., −144° C., −146° C., −148° C., −150° C., −152° C., −154° C., −156° C., −158° C., −160° C., −162° C., −164° C., −166° C., −168° C., −170° C., −172° C., −174° C., −176° C., −178° C., −180° C., −182° C., −184° C., −186° C., −188° C., −190° C., −192° C., −194° C., −196° C., or any combination thereof; and/or
transport of a cryopreserved sample, thermal stress, one or more freeze-thaw cycles, agitation, pressure changes, light irradiation, or any combination thereof.

99. The cryopreservation reagent of claim 97, wherein the period of time is at least about 1 day, 3 days, 5 days, 1 week, 2 weeks, 3 weeks, 1 month, 2 months, 3 months, 4 months, 5 months, 6 months, 7 months, 8 months, 9 months, 10 months, 11 months, 12 months, 14 months, 16 months, 18 months, 20 months, 22 months, 24 months, 26 months, 28 months, 30 months, 32 months, 34 months, 36 months, 38 months, 40 months, 42 months, 44 months, 46 months, or 48 months.

100. The cryopreservation reagent of claim 97, wherein the expression of one or more stress-associated genes is increased less than about 1.01-fold, 1.02-fold, 1.04-fold, 1.06-fold, 1.08-fold, 1.1-fold, 1.15-fold, 1.2-fold, 1.25-fold, 1.3-fold, 1.35-fold, 1.4-fold, 1.45-fold, or 1.5-fold post-storage condition, wherein the one or more stress-associated genes selected from the group consisting of ADM, AKR1B1, AQP1, AQP2, AQP4, ARNT, ATF4, ATF6, ATF6B, ATG12, ATG5, ATG7, ATM, ATR, BBC3, BECN1, BID, BNIP3L, CA9, CALR, CASP1, CCL2, CD40LG, CDKN1A, CFTR, CHEK1, CHEK2, CRP, DDB2, DDIT3, DNAJC3, EDN1, EPO, FAS, FTH1, GADD45A, GADD45G, GCLC, GCLM, GRB2, GSR, GSTP1, HMOX1, HSP90AA1, HSP90B1, HSPA4, HSPA4L, HSPA5, HUS1, IFNG, IL1A, IL1B, IL6, CXCL8, LDHA, MCL1, MMP9, MRE11A, NBN, NFAT5, NQ01, PARP1, PRDX1, PVR, RAD17, RAD51, RAD9A, RIPK1, SERPINE1, SLC2A1, SLC5A3, SQSTM1, TLR4, TNF, TNFRSF10A, TNFRSF10B, TNFRSF1A, TP53, TXN, TXNL4B, TXNRD1, ULK1, VEGFA, XPC, or any combination thereof.

101. A method of sample cryopreservation, comprising:
providing a sample;
providing the cryopreservation reagent of claim 82;
contacting the sample with the cryopreservation reagent, thereby generating a shielded sample; and
exposing the shielded sample to a freezing storage condition, thereby generating a cryopreserved sample.
